(19) United States
(12) Patent Application Publication
(10) Pub. No.: US 2024/0246913 A1
(43) Pub. Date: Jul. 25, 2024

(54) 2-CARBOXYL-INDOLE INHIBITORS OF METALLO-BETA-LACTAMASES

(71) Applicant: Oxford University Innovation Limited, Oxford (GB)

(72) Inventors: Christopher SCHOFIELD, Oxford (GB); Jurgen BREM, Oxford (GB); Tharindi PANDUWAWALA, Oxford (GB); Alistair FARLEY, Oxford (GB); Edgars SUNA, Oxford (GB)

(21) Appl. No.: 18/563,606

(22) PCT Filed: May 27, 2022
(86) PCT No.: PCT/GB2022/051370

§ 371 Date: Nov. 22, 2023

(30) Foreign Application Priority Data
May 27, 2021 (GB) 2107612.0

(51) Int. Cl. C07D 209/42; A61K 31/405; A61K 31/421; A61K 31/4439; A61P 31/04; C07D 401/04; C07D 403/06; C07D 409/06; C07D 413/06; C07D 413/14; C07D 417/06

(52) U.S. Cl. CPC C07D 209/42; A61K 31/405; A61K 31/421; A61K 31/4439; A61P 31/04; C07D 401/04; C07D 403/06; C07D 409/06; C07D 413/06; C07D 413/14; C07D 417/06

(57) ABSTRACT

The present invention relates to certain compounds that function as inhibitors of bacterial metallo-beta-lactamases. The present invention also relates to processes for the preparation of these compounds, to pharmaceutical compositions comprising them, and to their use in the treatment of a bacterial infection.

### INTRODUCTION

The present invention relates to compounds that function as inhibitors of metallo-beta-lactamases. The present invention also relates to processes for the preparation of these compounds, to pharmaceutical compositions comprising them, and to their use in the treatment of bacterial infections.

### BACKGROUND OF THE INVENTION

Infections caused by pathogenic bacteria are common worldwide, and thus antibacterial medicines to treat such infections are highly sought. Currently, β-lactam antibacterials (BLAs) are amongst the most widely used antibacterial treatments. However, the efficacy of BLAs is increasingly threatened by bacterial resistance, most importantly by the widespread dissemination of β-lactamases, which catalyse the hydrolysis and inactivation of BLA.

In combination with a suitable penicillin, Class A β-lactamase inhibitors (BLIs) have been components of highly successful medicines (e.g. as in Augmentin). However, the zinc ion dependent Class B metallo-β-lactamases (MBLs, or carbapenemases), are structurally and mechanistically distinct from Class A, C and D serine β-lactamases (SBLs). There is therefore a need for effective inhibitors of MBLs.

The increase in antibiotic resistance raises concerns that, at least in some regions, we are returning to a pre-antibiotic era, in particular in the case of Gram-negative infections. The increased prevalence of extended spectrum serine-β-lactamases (ESBL) and metallo-Q-lactamases (MBLs) means β-lactams are increasingly ineffective in treating Gram negative infections(1, 2). The advent of mcr-1 in 2015(3) and tetX3-5 in 2019(4) which mediate resistance to colistin and tigecycline, respectively, means all clinically vital antibiotics for serious Gram-negative infections are compromised. There are few novel anti Gram-negative drugs entering clinical trials; therefore, overcoming resistance to restore the activity of existing drugs, e.g. β-lactams, with an excellent safety record is of increasing importance(5).

The carbapenems, often ‘drugs of last resort’, manifest stability to ESBL, though are susceptible to SBL carbapenemases and all MBLs (6-8). Avibactam, relebactam, and vaborbactam are recently introduced SBL carbapenemase inhibitors(9-11), but excepting vaborbactam, which has a relatively limited activity spectrum(12, 13), these and classical SBL inhibitors (e.g. clavulanate) are increasingly susceptible to β-lactamase hydrolysis, including by MBLs which degrade all β-lactam classes(6, 14). Development of MBL inhibitors, in particular to protect carbapenems, is thus an unmet clinical need, especially in the developing world, where MBL producing bacteria are widely disseminated.

MBL inhibition is challenging because of structural diversity in their active sites(15, 16). By contrast with the SBLs, no clinically useful MBL inhibitors (MBLi) are available. The reported MBLi (17-19) lack the breadth of potency against the relevant MBL variants, required for widespread use(7, 20). (Note, that most MBLi inhibit by tight Zn(II) chelation, at the active site or in solution, the latter a property that may make it difficult to achieve selectivity compared to human metallo enzymes(6). Aspergillomarasmine A, a Zn(II) chelator and preclinical candidate ANT-2681 a Zn(II) binder, with MBL inhibition activity in an vivo mouse model, shows limited coverage of MBLs, as does a bicyclic boronate (VNRX-5133, taniborbactam) currently in Phase 3 trials).

WO2017093727 describes broad spectrum inhibitors of MBL. Such compounds display favourable activity but require further optimisation.

Thus, there remains a need for new treatments to combat MBL mediated antibacterial resistance.

The present invention was devised with the foregoing in mind.

### SUMMARY OF THE INVENTION

In one aspect, the present invention provides a compound as defined herein, or a pharmaceutically acceptable salt or solvate thereof.

In another aspect, the present invention provides a compound as defined herein, or a pharmaceutically acceptable salt or solvate thereof, for use as a medicament.

In another aspect, the present invention provides a compound as defined herein, or a pharmaceutically acceptable salt or solvate thereof, for use in the treatment of a bacterial infection.

In another aspect, the present invention provides a compound as defined herein, or a pharmaceutically acceptable salt or solvate thereof, in combination with a suitable antibacterial agent, for use in the treatment of a bacterial infection.

In another aspect, the present invention provides a pharmaceutical composition as defined herein which comprises a compound as defined herein, or a pharmaceutically acceptable salt or solvate thereof, and one or more pharmaceutically acceptable excipients.

In another aspect, the present invention provides a compound as defined herein, or a pharmaceutically acceptable salt or solvate thereof, or a pharmaceutical composition as defined herein, for use in the treatment of bacterial infections.

In another aspect, the present invention provides a compound as defined herein, or a pharmaceutically acceptable salt or solvate thereof, or a pharmaceutical composition as defined herein, for use in the production of a metallo-beta-lactamase inhibitory effect.

In another aspect, the present invention provides a method of inhibiting a bacterial metallo-beta-lactamase in vitro or in vivo, said method comprising contacting a cell with an effective amount of a compound as defined herein, or a pharmaceutically acceptable salt or solvate thereof.

In another aspect, the present invention provides a method of treating a bacterial infection in a patient in need of such treatment, said method comprising administering to said patient a therapeutically effective amount of a compound as defined herein, or a pharmaceutically acceptable salt or solvate thereof, or a pharmaceutical composition as defined herein, in combination with a suitable antibacterial agent.

In another aspect, the present invention provides the use of a compound, as defined herein, in combination with a suitable antibacterial agent, for the treatment of a bacterial infection.

In another aspect, the present invention provides the use of a compound, as defined herein, for the inhibition of a metallo-beta-lactamase

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups and analogues thereof. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkylene” means a (3-8C)cycloalkyl group covalently attached to a (1-6C)alkylene group, both of which are defined herein.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

Suitably, a nitrogen atom in a heterocyclic ring system may be in the form of alkylammonium salt, for example a dimethylpiperidin-1-ium salt:

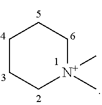

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members.

The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from: a benzene ring fused to a 5- or 6-membered ring containing 1, 2 or 3 ring heteroatoms; a pyridine ring fused to a 5- or 6-membered ring containing 1, 2 or 3 ring heteroatoms; a pyrimidine ring fused to a 5- or 6-membered ring containing 1 or 2 ring heteroatoms; a pyrrole ring fused to a 5- or 6-membered ring containing 1, 2 or 3 ring heteroatoms; a pyrazole ring fused to a 5- or 6-membered ring containing 1 or 2 ring heteroatoms; a pyrazine ring fused to a 5- or 6-membered ring containing 1 or 2 ring heteroatoms; an imidazole ring fused to a 5- or 6-membered ring containing 1 or 2 ring heteroatoms; an oxazole ring fused to a 5- or 6-membered ring containing 1 or 2 ring heteroatoms; an isoxazole ring fused to a 5- or 6-membered ring containing 1 or 2 ring heteroatoms; a thiazole ring fused to a 5- or 6-membered ring containing 1 or 2 ring heteroatoms; an isothiazole ring fused to a 5- or 6-membered ring containing 1 or 2 ring heteroatoms; a thiophene ring fused to a 5- or 6-membered ring containing 1, 2 or 3 ring heteroatoms; a furan ring fused to a 5- or 6-membered ring containing 1, 2 or 3 ring heteroatoms; a cyclohexyl ring fused to a 5- or 6-membered heteroaromatic ring containing 1, 2 or 3 ring heteroatoms; and a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like.

This specification also makes use of several composite terms to describe groups comprising more than one functionality. Such terms will be understood by a person skilled in the art. For example heterocyclyl(m-nC)alkyl comprises (m-nC)alkyl substituted by heterocyclyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In one aspect, the present invention relates to a compound of formula II or a pharmaceutically acceptable salt or solvate thereof, as shown below:

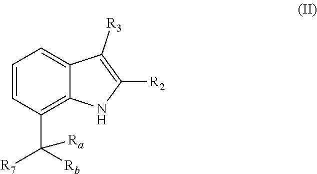

wherein
R₂ is selected from:
   i. —C(O)OH;
   ii. —C(O)OR_{2A}, wherein R_{2A} is selected from (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
   iii. —C(O)NR_{2B}R_{2C}; wherein R_{2B} and R_{2C} are each independently selected from hydrogen, (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
   iv. —C(O)NR_{2D}NR_{2B}R_{2E}; wherein R_{2D} is selected from hydrogen or (1-6C)alkyl and R_{2B} and R_{2C} are as defined above;
   v. tetrazolyl;
   vi. triazolyl;
   vii. —B(OR_{2F})(OR_{2G}), wherein R_{2F} and R_{2G} are each independently selected from hydrogen, (1-6C)alkyl or R_{2F} and R_{2G} are linked such that, together with the B and O atoms, they form a 5 or 6-membered heterocyclic ring, which is optionally substituted by (1-2C)alkyl;
   viii. trifluoromethylketone;
and wherein R^{A} is selected from halo, cyano, nitro or a group of the formula:

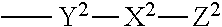

wherein
Y² is absent or a linker group of the formula —[CR^{A1}R^{A2}]ₘ— in which m is an integer selected from 1, 2, 3 or 4, and R^{A1} and R^{A2} are each independently selected from hydrogen or (1-2C)alkyl;
X² is absent or —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —S—, —SO—, —SO₂—, —S(O)₂N(R^{A3})—, or —N(R^{A3})SO₂— wherein R^{A3} is selected from hydrogen or methyl; and
Z² is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z² is optionally further substituted by one or more substituent groups independently selected from oxo, halo, cyano, nitro, hydroxy, carboxy, NR^{A4}R^{A5}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, heterocyclyl, heterocyclyloxy, heterocyclyl-(1-2C)alkyl, heteroaryl, heteroaryloxy, heteroaryl-(1-2C)alkyl, C(O)NR^{A4}R^{A5}, NR^{A4}C(O)R^{A5}, NR^{A4}S(O)₂R^{A5} and S(O)₂NR^{A4}R^{A5}; wherein R^{A4} and R^{A5} are each independently selected from hydrogen, (1-4C)alkyl or (3-6C)cycloalkyl or (3-6C)cycloalkyl(1-2C)alkyl; or R^{A4} and R^{A5} can be linked such that, together with the nitrogen atom to which they are attached, they form a 4-6 membered heterocyclic ring;
and wherein any alkyl, aryl, heterocyclyl or heteroaryl group present in a substituent group on Z² is optionally further substituted by halo, cyano, nitro, hydroxy, caboxy, NR^{A6}SR^{A7}, (1-2C)alkoxy, or (1-2C)alkyl; wherein R^{A6} and R^{A7} are selected from hydrogen or (1-2C)alkyl;
R₃ is selected from halo, aryl, (4-6C)cycloalkyl, 5- to 12-membered heteroaryl, 5- to 12-membered heterocyclyl, wherein said aryl, (4-6C)cycloalkyl, 5- to 12-membered heteroaryl, 5- to 12-membered heterocyclyl, ring system is optionally substituted by one or more R^{3A}
wherein each R^{3A} is independently halo, oxo, cyano, nitro, hydroxy or a group:

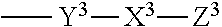

wherein
   Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
   X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})SO₂— wherein R^{B3} and R^{B4} are each independently selected from hydrogen or methyl; and
   Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
      and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
      and wherein any alkyl, aryl, heterocyclyl or heteroaryl group present in a substituent group on Z³ is optionally further substituted by halo, cyano, nitro, hydroxy, caboxy, NR^{B7}R^{B8}, (1-2C)alkoxy, or (1-2C)alkyl; wherein R^{B7} and R^{B8} are selected from hydrogen or (1-2C)alkyl;
   or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
   R₇ is a group:

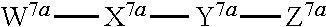

wherein:
   W^{7a} is absent or a linker group of the formula —[CR^{7A}R^{7B}]_{q}— in which q is an integer selected from 1, 2, 3 or 4, and each occurrence of R^{7A} and R^{7B} is each independently selected from hydrogen or (1-2C)alkyl, and
   X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —S—, —SO—, —SO₂—, —S(O)₂N(R^{7C})—, —N(R^{7D})SO₂—, —N(R^{7D})SO₂N(R^{7C})—, —C(NR^{7E})N(R^{7C})—, or —N(R^{7D})C(NR^{7E})N(R^{7C})—, wherein R^{7C}, R^{7D} and R^{7E} are each independently selected from hydrogen or (1-2C)alkyl, wherein any (1-2C)alkyl) is optionally substituted by oxo, halo, cyano, nitro, hydroxy, carboxy, amino, (1-2C)alkoxy; and
   Y^{7a} is absent or a linker group of the formula —[CR^{7F}R^{7G}]ᵣ— in which r is an integer selected from 1, 2, 3 or 4, and each occurrence of R^{7F} and R^{7G} is each independently selected from hydrogen or (1-2C)alkyl, and
   Z^{7a} is either:
   (i) (1-6C)alkyl optionally substituted by oxo, halo, cyano, nitro, hydroxy, carboxy, NR^{7H}R^{7I}, (1-4C)alkoxy; wherein R^{7H} and R^{7I} are each independently selected from hydrogen, (1-4C)alkyl or (3-6C)cycloalkyl or (3-6C)cycloalkyl(1-2C)alkyl
   with the proviso that if Z^{7a} is (1-6C)alkyl, then X^{7a} is not absent;
      or
   ii) (3-8C)cycloalkyl, aryl, 5- to 12-membered heterocyclyl or 5- to 12-membered heteroaryl; wherein each of (3-8C)cycloalkyl, aryl, 5 to 12-membered heterocyclyl, 5- to 12-membered heteroaryl is optionally substituted by:
   a) one or more of a substituent group independently selected from oxo, halo, cyano, nitro, hydroxy, carboxy, NR^{7H}R^{7I}, (1-4C)alkoxy, (1-4C)alkyl, (1-4C)alkylamino, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{7H}R^{7I}, NR^{7H}C(O)R^{7I}, NR^{7H}S(O)₂R^{7I} and S(O)₂NR^{7H}R^{7I}; wherein R^{7H} and R^{7I} are each independently selected from hydrogen, (1-4C)alkyl or (3-6C)cycloalkyl or (3-6C)cycloalkyl(1-2C)alkyl; or R^{7H} and R^{7I} can be linked such that, together with the nitrogen atom to which they are attached, they form a 4-7 membered heterocyclic ring;
      wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, C(O)NR^{7J}R^{7K}, NR^{7J}C(O)R^{7K}, NR^{7J}S(O)₂R^{7K} and S(O)₂NR^{7J}R^{7K}; wherein R^{7J} and R^{7K} are each independently selected from hydrogen or (1-2C)alkyl; and/or
   b) one or more of a group R^{Z}; wherein R^{Z} is a group of the formula:

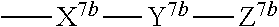

wherein:
   X^{7b} is absent or a linker group of the formula —[CR^{7L}R^{7M}]ₓ— in which x is an integer selected from 1, 2, 3 or 4, and each occurrence of R^{7L} and R^{7M} is each independently selected from hydrogen or (1-2C)alkyl, and
   Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N})—, —C(NR^{7P})N(R^{7N})—, or —N(R^{7P})C(═NR⁷1)N(R^{7N})—, wherein R^{7N}, R^{7P} and R^{7Q} are each independently selected from hydrogen or methyl; and Z^{7b} is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z^{7b} is optionally further substituted by one or more substituent groups independently selected from oxo, halo, cyano, nitro, hydroxy, carboxy, NR^{7R}R^{7S}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{7R}R^{7S}, NR^{7R}C(O)R^{7S}, NR^{7R}S(O)₂R^{7S} and S(O)₂NR^{7R}R^{7S}; wherein R^{7R} and R^{7S} are each independently selected from hydrogen, (1-4C)alkyl or (3-6C)cycloalkyl or (3-6C)cycloalkyl(1-2C)alkyl; or R^{7R} and R^{7S} can be linked such that, together with the nitrogen atom to which they are attached, they form a 4-7 membered heterocyclic ring;
   and wherein any alkyl, aryl, heterocyclyl or heteroaryl group present in a substituent group on Z^{7b} is optionally further substituted by halo, cyano, nitro, hydroxy, caboxy, NR^{7T}R^{7U}, (1-2C)alkoxy, or (1-2C)alkyl; wherein R^{7T} and R^{7U} are selected from hydrogen or (1-2C)alkyl;
   or R^{7T} and Z^{7U} can be linked such that, together with the nitrogen atom to which they are attached, they form a 4-7 membered heterocyclic ring, which is optionally substituted by oxo, halo, cyano, nitro, hydroxy, carboxy, NR^{7V}R^{7W}, (1-4C)alkoxy, (1-4C)alkyl, (1-4C)aminoalkyl, (3-8C)cycloalkyl, (3-8C)cycloalkyl-(1-3C)alkyl, (1-4C)alkanoyl, (1-4C)alkylsulphonyl, or C(O)NR^{7V}R^{7W}, NR^{7V}C(O)R^{7W}, NR^{7V}S(O)₂R^{7W} and S(O)₂NR^{7V}R^{7W}; wherein R^{7V} and R^{7W} are each independently selected from from hydrogen or (1-2C)alkyl; and
Rₐ is hydrogen or (1-4C)alkyl; and
R_{b} is selected from hydrogen or (1-4C)alkyl;
with the proviso that only one of Rₐ and R_{b} can be hydrogen.

In one aspect, the present invention relates to a compound of formula I or a pharmaceutically acceptable salt or solvate thereof, as shown below:

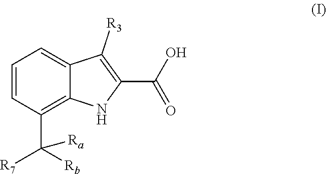

wherein
R₃ is selected from halo, aryl, (4-6C)cycloalkyl, 5- to 12-membered heteroaryl, 5- to 12-membered heterocyclyl, wherein said aryl, (4-6C)cycloalkyl, 5- to 12-membered heteroaryl, 5- to 12-membered heterocyclyl, ring system is optionally substituted by one or more R^{3A}.
wherein each R^{3A} is independently halo, oxo, cyano, nitro, hydroxy or a group:

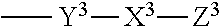

wherein
   Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
   X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, —S(O)(═NR^{B4})N(R^{B3})—, —C(═NR^{B4})N(R^{B3})—, —C(═O)N(R^{B3})—, —C(═NR^{B4}—, —S(O)(═NR^{B4})N(R^{B3})—, —S(O)(═NR^{B4})— or —N(R^{B4})SO₂— wherein R^{B3} and R^{B4} are each independently selected from hydrogen or methyl; and
   Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
      and wherein Z³ is optionally further substituted by one or more substituent groups independently selected from oxo, halo, cyano, nitro, hydroxy, carboxy, NR^{B5}R^{B6}, (1-4C)alkoxy, (1-40)alkyl, (3-80)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
      and wherein any alkyl, aryl, heterocyclyl or heteroaryl group present in a substituent group on Z³ is optionally further substituted by halo, cyano, nitro, hydroxy, caboxy, NR^{B7}R^{B8}, (1-20)alkoxy, or (1-2C)alkyl; wherein R^{B7} and R^{B8} are selected from hydrogen or (1-2C)alkyl;
   or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
R₇ is a group:

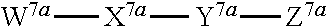

wherein:
W^{7a} is absent or a linker group of the formula —[CR^{7A}R^{7B}]q in which q is an integer selected from 1, 2, 3 or 4, and each occurrence of R^{7A} and R^{7B} is each independently selected from hydrogen or (1-2C)alkyl, and
X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —S—, —SO—, —SO₂—, —S(O)₂N(R^{7C})—, —N(R^{7D})SO₂—, —N(R^{7D})SO₂N(R^{7C})—, —C(NR^{7E})N(R^{7C})—, or —N(R^{7D})C(NR^{7E})N(R^{7C})—, wherein R^{7C}, R^{7D} and R^{7E} are each independently selected from hydrogen or (1-2C)alkyl, wherein any (1-2C)alkyl) is optionally substituted by oxo, halo, cyano, nitro, hydroxy, carboxy, amino, (1-2C)alkoxy; and
Y^{7a} is absent or a linker group of the formula —[CR^{7F}R^{7G}]ᵣ— in which r is an integer selected from 1, 2, 3 or 4, and each occurrence of R^{7F} and R^{7G} is each independently selected from hydrogen or (1-2C)alkyl, and
Z^{7a} is either:
   (i) (1-6C)alkyl optionally substituted by oxo, halo, cyano, nitro, hydroxy, carboxy, NR^{7H}R^{7I}, (1-4C)alkoxy or N⁺R^{7H}R^{7I}R^{7H′}; wherein R^{7H}, R^{7I} and R^{H′} are each independently selected from hydrogen, (1-4C)alkyl or (3-6C)cycloalkyl or (3-6C)cycloalkyl(1-2C)alkyl
   with the proviso that if Z^{7a} is (1-6C)alkyl, then X^{7a} is not absent;
      or
   ii) (3-8C)cycloalkyl, aryl, heterocyclyl or heteroaryl; each of which is optionally substituted by
   a) one or more of a substituent group independently selected from oxo, halo, cyano, nitro, hydroxy, carboxy, NR^{7H}R^{7I}, NR^{7H}R^{7I}R^{7H′}, (1-4C)alkoxy, (1-4C)alkyl, (1-4C)alkylamino, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{7H}R^{7I}, NR^{7H}C(O)R^{7I}, NR^{7H} S(O)₂R^{7I} and S(O)₂NR^{7H}R^{7I}; wherein R^{7H}, R^{7I} and R^{7H′} are each independently selected from hydrogen, (1-4C)alkyl or (3-6C)cycloalkyl or (3-6C)cycloalkyl(1-2C)alkyl; or R^{7H} and R^{7I} can be linked such that, together with the nitrogen atom to which they are attached, they form a 4-7 membered heterocyclic ring;
      wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, N⁺R^{7J}R^{7K}R^{7J′}, C(O)NR^{7J}R^{7K}, NR^{7J}C(O)R^{7K}, NR^{7J}S(O)₂R^{7K} and S(O)₂NR^{7J}R^{7K}; wherein R^{7J}, R^{7K} and R^{7J′} are each independently selected from hydrogen or (1-2C)alkyl; and/or
   b) one or more of a group R^{Z}; wherein R^{Z} is a group of the formula:

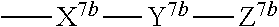

wherein:
   X^{7b} is absent or a linker group of the formula —[CR^{7L}R^{7M}]ₓ— in which x is an integer selected from 1, 2, 3 or 4, and each occurrence of R^{7L} and R^{7M} is each independently selected from hydrogen or (1-2C)alkyl, and
   Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N})—, —C(NR^{7P})N(R^{7N})—, or —N(R^{7P})C(═NR^{7Q})N(R^{7N})—, wherein R^{7N}, R^{7P} and R^{7Q} are each independently selected from hydrogen or methyl; and
   Z^{7b} is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z^{7b} is optionally further substituted by one or more substituent groups independently selected from oxo, halo, cyano, nitro, hydroxy, carboxy, NR^{7R}R^{7S}, N⁺R^{7R}R⁷SR^{7R′}R, (1-40)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{7R}R^{7S}, NR^{7R}C(O)R^{7S}, NR^{7R}S(O)₂R^{7S} and S(O)₂NR^{7R}R^{7S}; wherein R^{7R}, R^{7S} and R^{7R′} are each independently selected from hydrogen, (1-4C)alkyl or (3-6C)cycloalkyl or (3-6C)cycloalkyl(1-2C)alkyl; or R^{7R} and R^{7S} can be linked such that, together with the nitrogen atom to which they are attached, they form a 4-7 membered heterocyclic ring;
   and wherein any alkyl, aryl, heterocyclyl or heteroaryl group present in a substituent group on Z^{7b} is optionally further substituted by halo, cyano, nitro, hydroxy, caboxy, NR^{7T}R^{7U}, N⁺R^{7T}R^{7U}R^{7T′}, (1-2C)alkoxy, or (1-2C)alkyl; wherein R^{7T}, R^{7U} and R^{7T′} are each independently selected from hydrogen or (1-2C)alkyl;
   or R^{7T} and Z^{7U} can be linked such that, together with the nitrogen atom to which they are attached, they form a 4-7 membered heterocyclic ring, which is optionally substituted by oxo, halo, cyano, nitro, hydroxy, carboxy, NR^{7V}R^{7W}, NR^{7V}R^{7W}R^{7V′} (1-4C)alkoxy, (1-4C)alkyl, (1-4C)aminoalkyl, (3-8C)cycloalkyl, (3-8C)cycloalkyl-(1-3C)alkyl, (1-4C)alkanoyl, (1-4C)alkylsulphonyl, or C(O)NR^{7V}R^{7W}, NR^{7V}C(O)R^{7W}, NR^{7V}S(O)₂R^{7W} and S(O)₂NR^{7V}R^{7W}; wherein R^{7V}, R^{7W} and R^{7V′} are each independently selected from hydrogen or (1-2C)alkyl;
Rₐ is hydrogen or (1-4C)alkyl; and
R_{b} is selected from hydrogen or (1-4C)alkyl;
with the proviso that only one of Rₐ and R_{b} can be hydrogen.

Particular compounds of the invention include, for example, compounds of Formula I and Formula IIl, or any sub-formula thereof, or pharmaceutically acceptable salts and/or solvates thereof, wherein, unless otherwise stated, each of R₂, R₃, R₇, Rₐ and R_{b}, and any associated substituent groups has any of the meanings defined hereinbefore or in any of paragraphs (1) to (37) hereinafter:
(1) R₃ is selected from halo, aryl, (4-6C)cycloalkyl, 5- to 12-membered heteroaryl, 5- to 12-membered heterocyclyl, wherein said aryl, (4-6C)cycloalkyl, 5- to 12-membered heteroaryl, 5- to 12-membered heterocyclyl, ring system is optionally substituted by one or more R^{3A};
   wherein each R^{3A} is independently halo, oxo, cyano, nitro, hydroxy or a group:

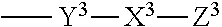

wherein
   Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
   X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, —C(═NR^{B4})N(R^{B3})—, —C(═O)N(R^{B3})—, —C(═NR^{B4}—, S(O)(═NR^{B4})N(R^{B3})—, —S(O)(═NR^{B4})— or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-80)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
   and wherein any alkyl, aryl, heterocyclyl or heteroaryl group present in a substituent group on Z³ is optionally further substituted by halo, cyano, nitro, hydroxy, caboxy, NR^{B7}R^{B8}, (1-20)alkoxy, or (1-2C)alkyl; wherein R^{B7} and R^{B8} are selected from hydrogen or (1-2C)alkyl;
   or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
(2) R₃ is selected from halo, phenyl, 5- or 6-membered heteroaryl or 5- or 6-membered heterocyclyl, fused heteroaryl or fused heterocyclyl; wherein said phenyl, 5- or 6-membered heteroaryl, 5- or 6-membered heterocyclyl, fused heteroaryl or fused heterocyclyl is optionally substituted by one or more R^{3A};
   wherein R^{3A} is halo, oxo, cyano, nitro, oxo, hydroxy or a group:

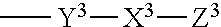

wherein
   Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
   X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})SO₂— wherein R^{B3} and R^{B4} are each independently selected from hydrogen or methyl; and
   Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
      and wherein Z³ is optionally further substituted by one or more substituent groups independently selected from oxo, halo, cyano, nitro, hydroxy, carboxy, NR^{B5}R^{B6}, (1-40)alkoxy, (1-4C)alkyl, (3-80)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
      and wherein any alkyl, aryl, heterocyclyl or heteroaryl group present in a substituent group on Z³ is optionally further substituted by halo, cyano, nitro, hydroxy, caboxy, NR^{B7}R^{B8}, (1-2C)alkoxy, or (1-20)alkyl; wherein R^{B7} and R^{B8} are selected from hydrogen or (1-2C)alkyl;
   or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
(3) R₃ is selected from halo, phenyl, 5- or 6-membered heteroaryl or 5- or 6-membered heterocyclyl, 8-10 membered fused heteroaryl or 8-10 membered fused heterocyclyl; wherein said phenyl, 5- or 6-membered heteroaryl or 5- or 6-membered heterocyclyl, 8-10 membered fused heteroaryl or 8-10 membered fused heterocyclyl is optionally substituted by one or more R^{3A};
   wherein each R^{3A} is independently selected from halo, oxo, cyano, nitro, oxo, hydroxy or a group:

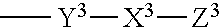

wherein
   Y³ is absent or a linker group of the formula —[CR^{B1}R^{B2}]ₙ— in which n is an integer selected from 1 or 2, and R^{B1} and R^{B2} are each independently selected from hydrogen or (1-2C)alkyl;
   X³ is selected from —SO—, —SO₂—, —S(O)₂N(R^{B3})—, —S(O)(NR^{B4})N(R^{B3})— or —N(R^{B4})SO₂— wherein R^{B3} and R^{B4} are each independently selected from hydrogen or methyl; and
   Z³ is hydrogen, (1-6C)alkyl or heterocyclyl;
      and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryloxy, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
(4) R₃ is selected from:

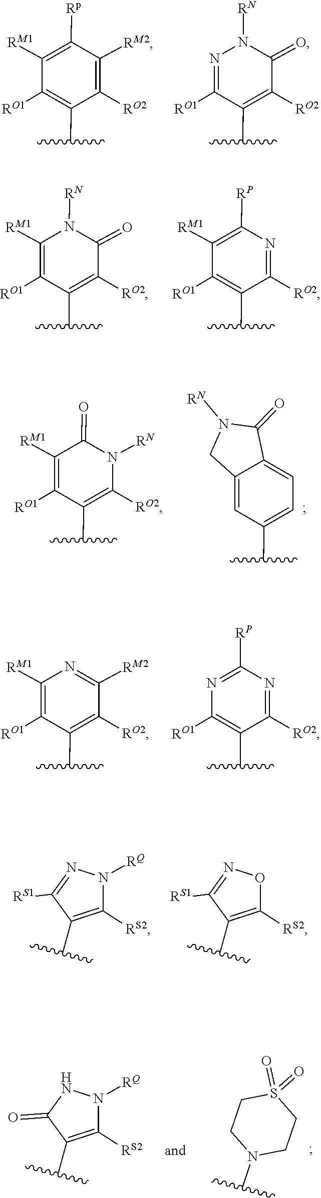

wherein:
R^{N} and R^{Q} are either hydrogen or methyl;
R^{O1} and R^{O2} are each independently selected from hydrogen or fluoro;
R^{M1} and R^{M2} are each independently selected from hydrogen, halo, cyano, hydroxy, (1-2C)alkyl, (1-2C)alkoxy; (1-2C)haloalkyl, (1-2C)haloalkoxy; and
R^{P} is a group:

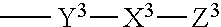

wherein
   Y³ is absent or a linker group of the formula —[CR^{B1}R^{B2}]ₙ— in which n is an integer selected from 1 or 2, and R^{B1} and R^{B2} are each independently selected from hydrogen or methyl;
   X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})—, or —N(R^{B4})SO₂—, wherein R^{B3} and R^{B4} are each independently selected from hydrogen or methyl; and
   Z³ is hydrogen, (1-6C)alkyl, (3-6C)cycloalkyl, (3-6C)cycloalkenyl, heteroaryl or heterocyclyl;
R^{S1} and R^{S2} are each independently selected from methyl, hydroxy or fluoro.
(5) R₃ is a group selected from:

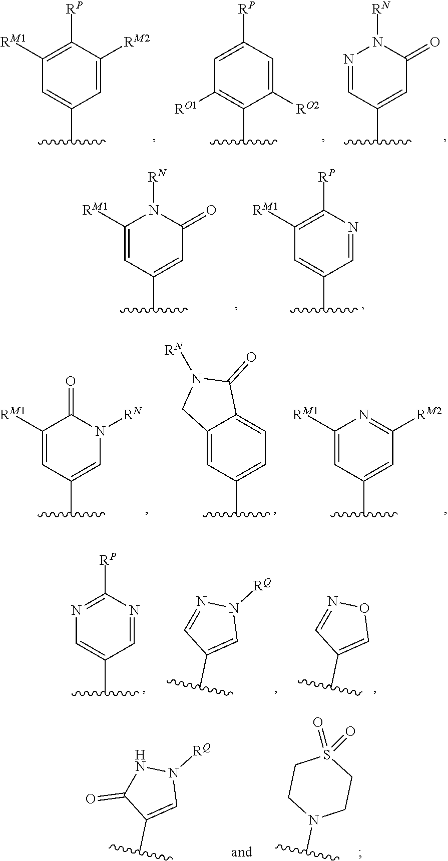

wherein:
R^{Q} and R^{N} are either hydrogen or methyl;
R^{O1} and R^{O2} are each independently selected from hydrogen or fluoro;
R^{M1} and R^{M2} are each independently selected from hydrogen, halo, cyano, hydroxy, (1-2C)alkyl, (1-2C)alkoxy; (1-2C)haloalkyl, (1-2C)haloalkoxy; and
R^{P} is a group:

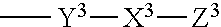

wherein
   Y³ is absent or a linker group of the formula —[CR^{B1}R^{B2}]ₙ— in which n is an integer selected from 1 or 2, and R^{B1} and R^{B2} are each independently selected from hydrogen or methyl;
   X³ is selected from or —N(R^{B3})—, —N(R^{B4})—C(O)—, —N(R^{B4})—C(O)O—, —C(O)—N(R^{B3})—, —N(R^{B4})C(O)N(R^{B3})—, —S—, —S(O)—, —S(O)₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})S(O)₂— wherein R^{B3} and R⁸⁴ are each independently selected from hydrogen or methyl; and
   Z³ is hydrogen, (1-2C)alkyl, (3-6C)cycloalkyl, or heterocyclyl.
(6) R₃ is a group selected from:

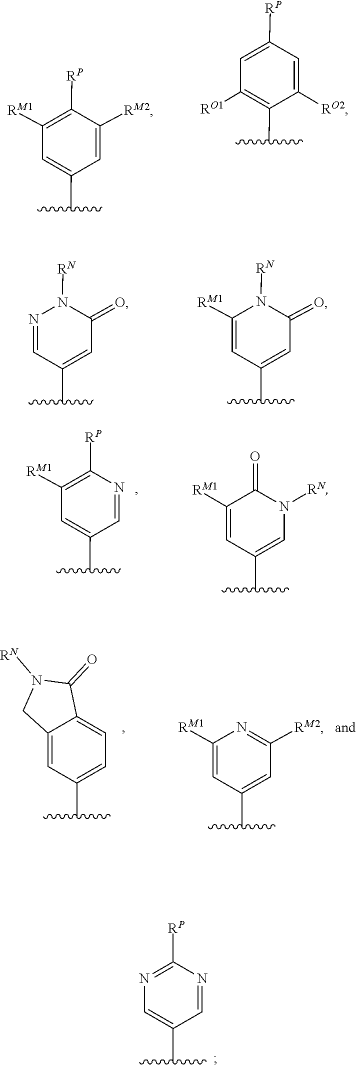

wherein:
R^{Q} and R^{N} are either hydrogen or methyl;
R^{O1} and R^{O2} are each independently selected from hydrogen or fluoro;
R^{M1} and R^{M2} are each independently selected from hydrogen, halo, cyano, hydroxy, (1-2C)alkyl, (1-2C)alkoxy; (1-2C)haloalkyl, (1-2C)haloalkoxy; and
R^{P} is a group:

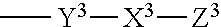

wherein
   Y³ is absent or a linker group of the formula —[CR^{B1}R^{B2}]ₙ— in which n is an integer selected from 1 or 2, and R^{B1} and R^{B2} are each independently selected from hydrogen or methyl;
   X³ is selected from or —N(R^{B3})—, —N(R^{B4})—C(O)—, —N(R^{B4})—C(O)O—, —C(O)—N(R^{B3})—, —N(R^{B4})C(O)N(R^{B3})—, —S—, —S(O)—, —S(O)₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})S(O)₂— wherein R^{B3} and R^{B4} are each independently selected from hydrogen or methyl; and
   Z³ is hydrogen, (1-2C)alkyl, (3-6C)cycloalkyl, or heterocyclyl.
(7) R₃ is a group:

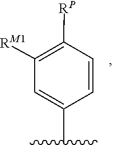

wherein
R^{P} is a group:

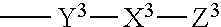

wherein
   Y³ is absent or a linker group of the formula —[CR^{B1}R^{B2}]ₙ— in which n is an integer selected from 1 or 2, and R^{B1} and R^{B2} are each independently selected from hydrogen or methyl;
   X³ is selected from or —N(R^{B3})—, —N(R^{B4})—C(O)—, —N(R^{B4})—C(O)O—, —C(O)—N(R^{B3})—, —N(R^{B4})C(O)N(R^{B3})—, —S—, —S(O), —S(O)₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})S(O)₂— wherein R^{B3} and R^{B4} are each independently selected from hydrogen or methyl; and
   Z³ is (1-2C)alkyl or heterocyclyl; and
R^{M1} is selected from halo, cyano, methoxy or hydroxy.
(8) R₃ is a group:

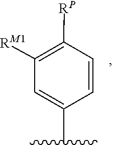

wherein
R^{P} is a group:

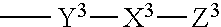

wherein
   Y³ is absent or a linker group of the formula —[CR^{B1}R^{B2}]—, wherein R^{B1} and R^{B2} are each independently selected from hydrogen or methyl;
   X³ is selected from or —N(R^{B3})—, —N(R^{B4})—C(O)—, —N(R^{B4})—C(O)O—, —C(O)—N(R^{B3})—, —N(R^{B4})C(O)N(R^{B3})—, —S—, —S(O)—, —S(O)₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})S(O)₂— wherein R^{B3} and R^{B4} are each independently selected from hydrogen or methyl; and
   Z³ is (1-2C)alkyl or 4- to 6-membered heterocyclyl;
R^{M1} is selected from halo, cyano, methoxy or hydroxy.
(9) R₃ is a group:

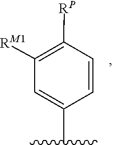

wherein
R^{P} is a group:

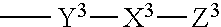

wherein
   Y³ is absent or a linker group of the formula —[CR^{B1}R^{B2}]—, wherein R^{B1} and R^{B2} are each independently selected from hydrogen or methyl;
   X³ is selected from or —S(O)₂—, or —N(R^{B4})S(O)₂—, wherein R^{B3} and R^{B4} are each independently selected from hydrogen or methyl; and
   Z³ is (1-2C)alkyl or 4- to 6-membered heterocyclyl;
R^{M1} is selected from halo or cyano, methoxy or hydroxy.
(10) R₃ is a group selected from:

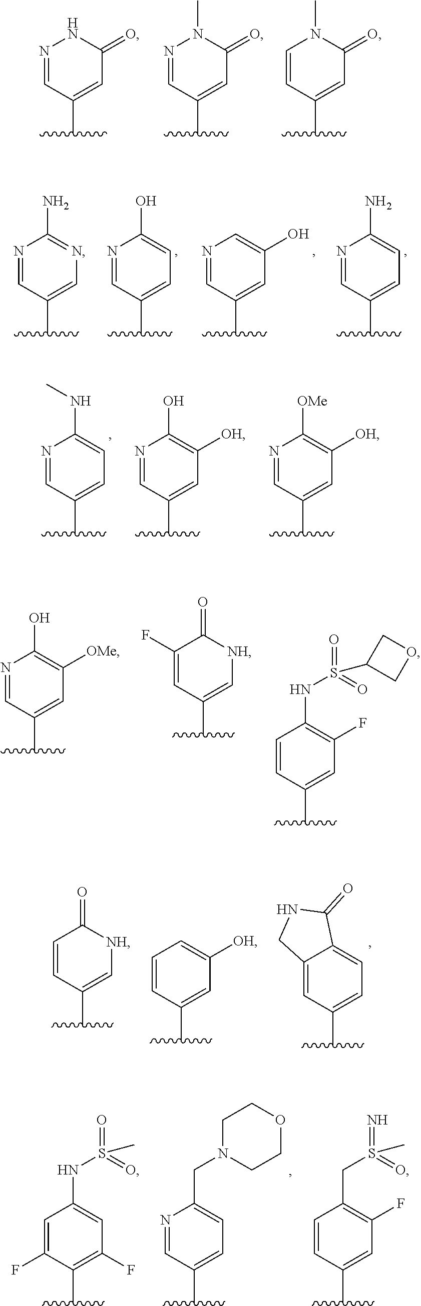

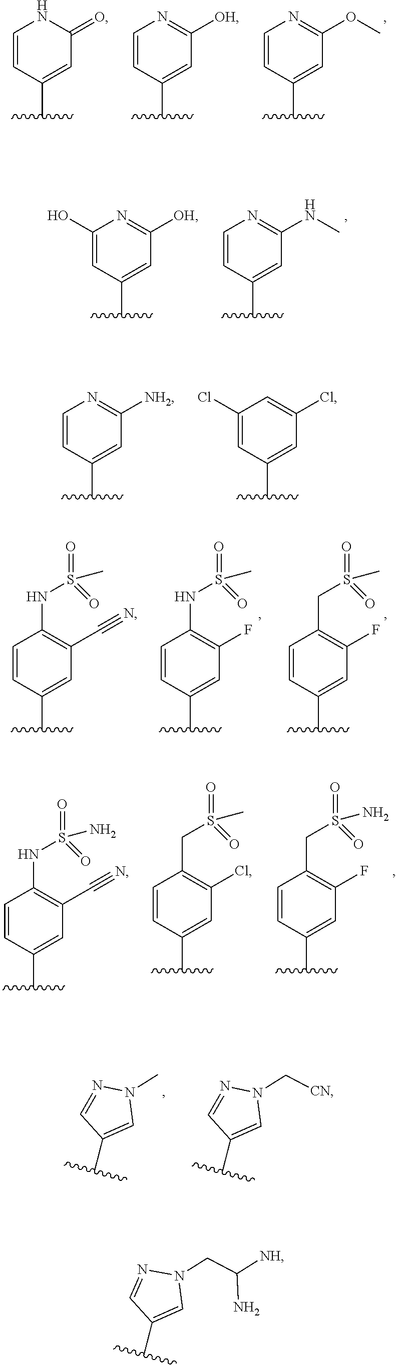

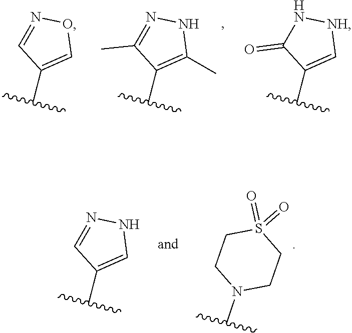

(11) R₃ is a group selected from:

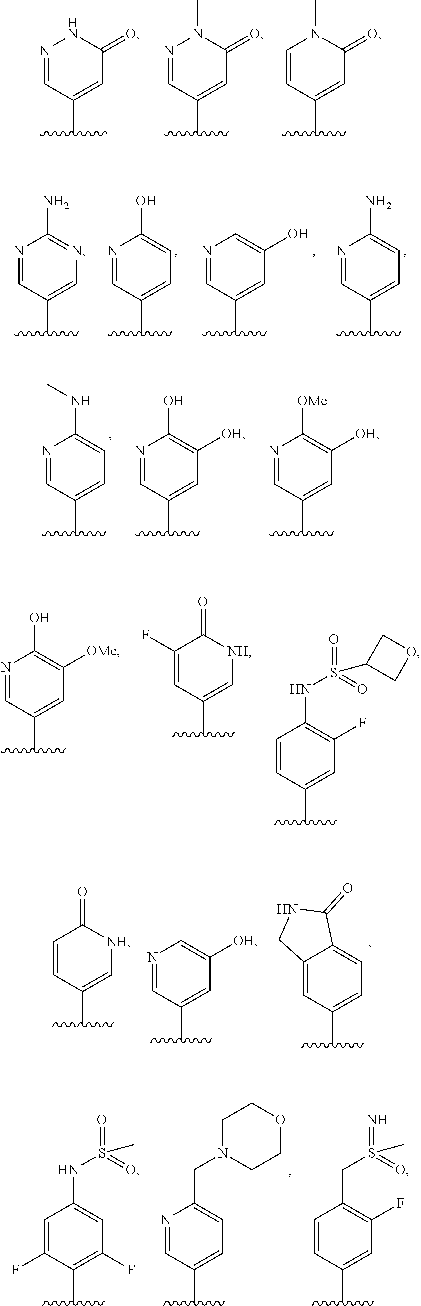

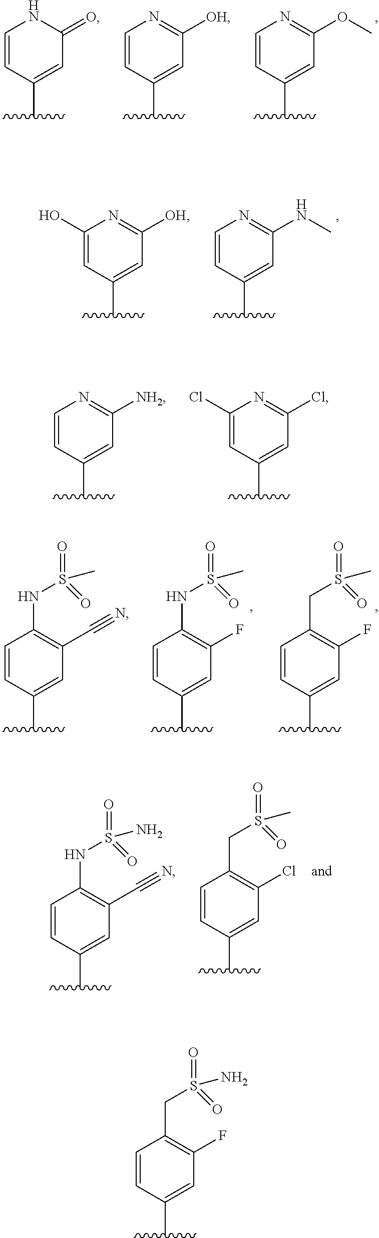

(12) R₃ is a group selected from:

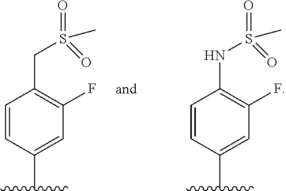 [SMILES: *c1ccc(CS(C)(=O)=O)c(F)c1 ; *c1ccc(NS(C)(=O)=O)c(F)c1]

(13) R₇ is a group:

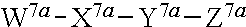

wherein:
W^{7a} is absent or a linker group of the formula —[CR^{7A}R^{7B}]q in which q is an integer selected from 1 or 2, and each occurrence of R^{7A} and R^{7B} is each independently selected from hydrogen or methyl, and
X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —N(R^{7D})SO₂—, —S—C(NR^{7E})N(R^{7C})—, or —N(R^{7D})C(NR^{7E})N(R^{7C})—, wherein R^{7C}, R^{7D} and R^{7E} are each independently selected from hydrogen or methyl; and
Y^{7a} is absent or a linker group of the formula —[CR^{7F}R^{7G}]ᵣ in which r is an integer selected from 1 or 2, and each occurrence of R^{7F} and R^{7G} is each independently selected from hydrogen or methyl, and
Z^{7a} is (3-8C)cycloalkyl, phenyl, 5- or 6-membered heterocyclyl, a fused 8-12 membered heterocyclic or heteroaryl ring system, 5- or 6-membered heteroaryl, a spirocyclic 8-10 membered heterocyclic ring system, a bridged (3-8C)cycloalkyl, or a bridged heterocyclic ring system, each of which is optionally substituted by:
   a) one or more of a substituent group independently selected from oxo, halo, cyano, nitro, hydroxy, carboxy, NR^{7H}R^{7I}, (1-40)alkoxy, (1-4C)alkyl, (1-4C)alkylamino, heterocyclyl-(1-2C)alkyl, heteroaryl, or heteroaryl-(1-2C)alkyl;
      wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, C(O) NR^{7J}R^{7K}, NR^{7J}C(O)R^{7K}, NR^{7J}S(O)₂R^{7K} and S(O)₂NR^{7J}R^{7K}; wherein R^{7J} and R^{7K} are each independently selected from hydrogen or (1-2C)alkyl; and/or
   b) one or more of a group R^{Z}; wherein R^{Z} is a group of the formula:

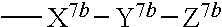

wherein:
   X^{7b} is absent or a linker group of the formula —[CR^{7L}R^{7M}]ₓ in which x is an integer selected from 1, 2 or 3, and each occurrence of R^{7L} and R^{7M} is each independently selected from hydrogen or (1-2C)alkyl, and
   Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N}—, —C(NR^{7P})N(R^{7N})—, or —N(R^{7P})C(═NR^{7Q})N(R^{7N})—, wherein R^{7N}, R^{7P} and R^{7Q} are each independently selected from hydrogen or methyl; and
   Z^{7b} is hydrogen, (1-6C)alkyl, aryl, (3-6C)cycloalkyl, heteroaryl or heterocyclyl;
   and wherein Z^{7b} is optionally further substituted by one or more substituent groups independently selected from oxo, halo, cyano, nitro, hydroxy, carboxy, NR^{7R}R^{7S}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl;
wherein R^{7R} and R^{7S} are each independently selected from hydrogen or (1-4C)alkyl;
and wherein any alkyl, aryl, heterocyclyl or heteroaryl group present in a substituent group on Z^{7b} is optionally further substituted by halo, cyano, nitro, hydroxy, caboxy, NR^{7T}R^{7U}, (1-2C)alkoxy, or (1-2C)alkyl; wherein R^{7T} and R^{7U} are selected from hydrogen or (1-2C)alkyl.
(14) R₇ is a group:

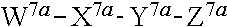

wherein:
W^{7a} is absent or a linker group of the formula —[CR^{7A}R^{7B}]—, wherein R^{7A} and R^{7B} is each independently selected from hydrogen or methyl; and
X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —N(R^{7D})SO₂—, —S—C(NR^{7E})N(R^{7C})—, or —N(R^{7D})C(═NR^{7E})N(R^{7C})—, wherein R^{7C}, R^{7D} and R^{7E} are each independently selected from hydrogen or methyl; and
Y^{7a} is absent or a linker group of the formula —[CR^{7F}R^{7G}]ᵣ— in which r is an integer selected from 1 or 2, and each occurrence of R^{7F} and R^{7G} is each independently selected from hydrogen or methyl, and
Z^{7a} is (3-8C)cycloalkyl, phenyl, 5- or 6-membered heterocyclyl, a fused heterocyclic ring system, 5- or 6-membered heteroaryl or a spirocyclic heterocyclic ring system, each of which is optionally substituted by:
   a) one or more of a substituent group independently selected from oxo, halo, cyano, nitro, hydroxy, carboxy, NR^{7H}R^{7I}, (1-40)alkoxy, (1-4C)alkyl, (1-4C)alkylamino, heterocyclyl-(1-2C)alkyl, heteroaryl, or heteroaryl-(1-2C)alkyl;
      wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, C(O) NR^{7J}R^{7K}, NR^{7J}C(O)R^{7K}, NR^{7J}S(O)₂R^{7K} and S(O)₂NR^{7J}R^{7K}; wherein R^{7J} and R^{7K} are each independently selected from hydrogen or (1-2C)alkyl; and/or
   b) one or more of a group R^{Z}; wherein R^{Z} is a group of the formula:

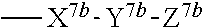

wherein:
   X^{7b} is absent or a linker group of the formula —[CR^{7L}R^{7M}]ₓ— in which x is an integer selected from 1, 2 or 3, and each occurrence of R^{7L} and R^{7M} is each independently selected from hydrogen or (1-2C)alkyl, and
   Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N})—, —C(NR^{7P})N(R^{7N})—, or —N(R^{7P})C(═NR^{7I})N(R^{7N})—, wherein R^{7N}, R^{7P} and R^{7Q} are each independently selected from hydrogen or methyl; and
   Z^{7b} is hydrogen, (1-6C)alkyl, aryl, (3-6C)cycloalkyl, 5- or 6 membered heteroaryl or 3- to 6-membered heterocyclyl;
   and wherein Z^{7b} is optionally further substituted by one or more substituent groups independently selected from oxo, halo, cyano, nitro, hydroxy, carboxy, NR^{7R}R^{7S}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl;
   wherein R^{7R} and R^{7S} are each independently selected from hydrogen or (1-4C)alkyl.
(15) R₇ is a group:

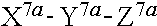

wherein:
   X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —N(R^{7D})SO₂—, —S—C(NR^{7E})N(R^{7C})—, or —N(R^{7D})C(═NR^{7E})N(R^{7C})—, wherein R^{7C}, R^{7D} and R^{7E} are each independently selected from hydrogen or methyl; and
   Y^{7a} is absent or a linker group of the formula —[CR^{7F}R^{7G}]—, wherein R^{7F} and R^{7G} are each independently selected from hydrogen or methyl, and
   Z^{7a} is (3-8C)cycloalkyl, phenyl, 5-membered heterocyclyl, a fused heterocyclic ring system, 5- or 6-membered heteroaryl or a spirocyclic heterocyclic ring system, each of which is optionally substituted by:
      a) one or more of a substituent group independently selected from oxo, halo, cyano, nitro, hydroxy, carboxy, NR^{7H}R^{7I}, (1-40)alkoxy, (1-4C)alkyl, (1-4C)alkylamino, heterocyclyl-(1-2C)alkyl, heteroaryl, or heteroaryl-(1-2C)alkyl;
         wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, C(O) NR^{7J}R^{7K}, NR^{7J}C(O)R^{7K}, NR^{7J}S(O)₂R^{7K} and S(O)₂NR^{7J}R^{7K}; wherein R^{7J} and R^{7K} are each independently selected from hydrogen or (1-2C)alkyl; and/or
      b) one or more of a group R^{Z}; wherein R^{Z} is a group of the formula:

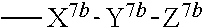

wherein:
   X^{7b} is absent or a linker group of the formula —[CR^{7L}R^{7M}]ₓ— in which x is an integer selected from 1, 2 or 3, and each occurrence of R^{7L} and R^{7M} is each independently selected from hydrogen or (1-2C)alkyl, and
   Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N})—, —C(NR^{7P})N(R^{7N})—, or —N(R^{7P})C(═NR^{7Q})N(R^{7N})—, wherein R^{7N}, R^{7P} and R^{7Q} are each independently selected from hydrogen or methyl; and
   Z^{7b} is hydrogen, (1-6C)alkyl, aryl, (3-6C)cycloalkyl, 5- or 6 membered heteroaryl or 3- to 6-membered heterocyclyl;
   and wherein Z^{7b} is optionally further substituted by one or more substituent groups independently selected from oxo, halo, cyano, nitro, hydroxy, carboxy, NR^{7R}R^{7S}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl;
wherein R^{7R} and R^{7S} are each independently selected from hydrogen or (1-4C)alkyl.
(16) R₇ is a group:

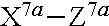

wherein
X^{7a} is absent or —O—, —C(O)—, —C(O)O—, —OC(O)—, —OC(O)N(R^{7C})—, —CH(OR^{7C})—, —N(R^{7C}—, —N(R^{7D})—C(O)—, —N(R^{7D})—C(O)O—, —C(O)—N(R^{7C})—, —N(R^{7D})C(O)N(R^{7C})—, —N(R^{7D})SO₂—, —S—C(NR^{7E})N(R^{7C})—, or —N(R^{7D})C(NR^{7E})N(R^{7C})—, wherein R^{7C}, R^{7D} and R^{7E} are each independently selected from hydrogen or methyl; and Z^{7a} is (3-8C)cycloalkyl, phenyl, 5- or 6-membered heterocyclyl, a fused heterocyclic ring system, 5- or 6-membered heteroaryl or a spirocyclic heterocyclic ring system, each of which is optionally substituted by:
   a) one or more of a substituent group independently selected from oxo, halo, cyano, nitro, hydroxy, carboxy, NR^{7H}R^{7I}, (1-4C)alkoxy, (1-4C)alkyl, (1-4C)alkylamino, heterocyclyl-(1-2C)alkyl, heteroaryl, or heteroaryl-(1-2C)alkyl;
      wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, C(O) NR^{7J}R^{7K}, NR^{7J}C(O)R^{7K}, NR^{7J}S(O)₂R^{7K} and S(O)₂NR^{7J}R^{7K}; wherein R^{7J} and R^{7K} are each independently selected from hydrogen or (1-2C)alkyl; and/or
   b) a group R^{Z}, having the formula:

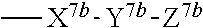

wherein:
   X^{7b} is absent or a linker group of the formula —[CR^{7L}R^{7M}]ₓ— in which x is an integer selected from 1, 2 or 3, and each occurrence of R^{7L} and R^{7M} is each independently selected from hydrogen or (1-2C)alkyl, and
   Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N}—, —C(NR^{7P})N(R^{7N})—, or —N(R^{7P})C(═NR^{7Q})N(R^{7N})—, wherein R^{7N}, R^{7P} and R^{7D} are each independently selected from hydrogen or methyl; and
   Z^{7b} is hydrogen, (1-6C)alkyl, (2-6C)alkenyl, (2-6C)alkynyl, aryl, (3-6C)cycloalkyl, (3-6C)cycloalkenyl, 5- or 6 membered heteroaryl or 4- to 6-membered heterocyclyl; and
   and wherein Z^{7b} is optionally further substituted by one or more substituent groups independently selected from oxo, halo, cyano, nitro, hydroxy, carboxy, NR^{7R}R^{7S}, (1-4C)alkoxy, (1-4C)alkyl; wherein R^{7R} and R^{7S} are each independently selected from hydrogen or (1-2C)alkyl.
(17) R₇ is a group:

—Z^{7a}
wherein
Z^{7a} is 5-membered heterocyclyl, 5- or 6-membered heteroaryl or a spirocyclic heterocyclic ring system,
each of which is optionally substituted by one or more of a substituent group independently selected from oxo or NR^{7H}R^{7I}, wherein R^{7H} and R^{7I} are independently selected from hydrogen or methyl;
and/or
Z^{7a} is optionally substituted by one or more R^{Z}, wherein R^{Z} has the formula:

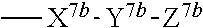

wherein:
   X^{7b} is absent or a linker group of the formula —[CR^{7L}R^{7M}]ₓ— in which x is an integer selected from 1, 2 or 3, and each occurrence of R^{7L} and R^{7M} is each independently selected from hydrogen or methyl, and
   Y^{7b} is absent or —N(R^{7N})—, —N(R^{7P})—C(O)—, —N(R^{7P})—C(O)O—, —C(O)—N(R^{7N})—, —N(R^{7P})C(O)N(R^{7N})—, —N(R^{7P})SO₂N(R^{7N})—, —C(NR^{7P})N(R^{7N})—, or —N(R^{7P})C(═NR⁷¹)N(R^{7N})—, wherein R^{7N}, R^{7P} and R^{7Q} are each independently selected from hydrogen or methyl; and
   Z^{7b} is hydrogen, (1-4C)alkyl, (3-6C)cycloalkyl, or 4 to 6 membered heterocyclyl; and and wherein Z^{7b} is optionally further substituted by one or more substituent groups independently selected from oxo, halo, cyano, NR^{7R}R^{7S}, (1-2C)alkoxy, (1-2C)alkyl; wherein R^{7R} and R^{7S} are each independently selected from hydrogen or methyl;
(18) R₇ is a group:

—Z^{7a}
wherein
Z^{7a} is 5- or 6-membered heterocyclyl, 5- or 6-membered heteroaryl or a spirocyclic heterocyclic ring system;
each of which is optionally substituted by one or more of a substituent group independently selected from oxo or NH₂;
and/or
Z^{7a} is optionally substituted by one or more R^{Z}, wherein R^{Z} has the formula:

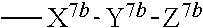

wherein:
   X^{7b} is absent or a linker group of the formula —[CR^{7L}R^{7M}]ₓ— in which x is an integer selected from 1 or 2, and each occurrence of R^{7L} and R^{7M} is each independently selected from hydrogen or methyl, and
   Y^{7b} is absent or —N(R^{7N})—, —N(R^{7P})—C(O)O—, —N(R^{7P})SO₂N(R^{7N})—, —C(NR^{7P})N(R^{7N})—, or —N(R^{7P})C(═NR^{7Q})N(R^{7N})—, wherein R^{7N}, R^{7P} and R^{7D} are each independently selected from hydrogen or methyl; and
   Z^{7b} is hydrogen, (1-4C)alkyl, (3-6C)cycloalkyl, or 4 to 6 membered heterocyclyl; and
   and wherein Z^{7b} is optionally further substituted by one or more substituent groups independently selected from oxo, halo, cyano, NR^{7R}R^{7S}, methoxy, methyl; wherein R^{7R} and R^{7S} are each independently selected from hydrogen or methyl;
(19) R₇ is a group:

—Z^{7a}
wherein
Z^{7a} is 5-membered heterocyclyl or a spirocyclic heterocyclic ring system; each of which is optionally substituted by one or more of a substituent group independently selected from oxo or NH₂;
and/or
Z^{7a} is optionally substituted by one or more R^{Z}, wherein R^{Z} has the formula:

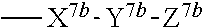

wherein:
   X^{7b} is absent or a linker group of the formula —[CR^{7L}R^{7M}]ₓ— in which x is an integer selected from 1 or 2, and each occurrence of R^{7L} and R^{7M} is each independently selected from hydrogen or methyl, and Y^{7b} is absent or —N(R^{7N})—, wherein R^{7N}, R^{7P} and R^{7Q} are each independently selected from hydrogen or methyl; and
   Z^{7b} is hydrogen, (1-4C)alkyl or 4 to 6 membered heterocyclyl; and and wherein Z^{7b} is optionally further substituted by one or more substituent groups independently selected from oxo, halo, cyano, NR^{7R}R^{7S}, methoxy, methyl; wherein R^{7R} and R^{7S} are each independently selected from hydrogen or methyl;
(20) R₇ is a group:

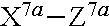

wherein
X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —N(R^{7D})SO₂—, —S—C(NR^{7E})N(R^{7C})—, or —N(R^{7D})C(NR^{7E})N(R^{7C})—, wherein R^{7C}, R^{7D} and R^{7E} are each independently selected from hydrogen or methyl; and
   Z^{7a} is a group selected from a group of the formula:

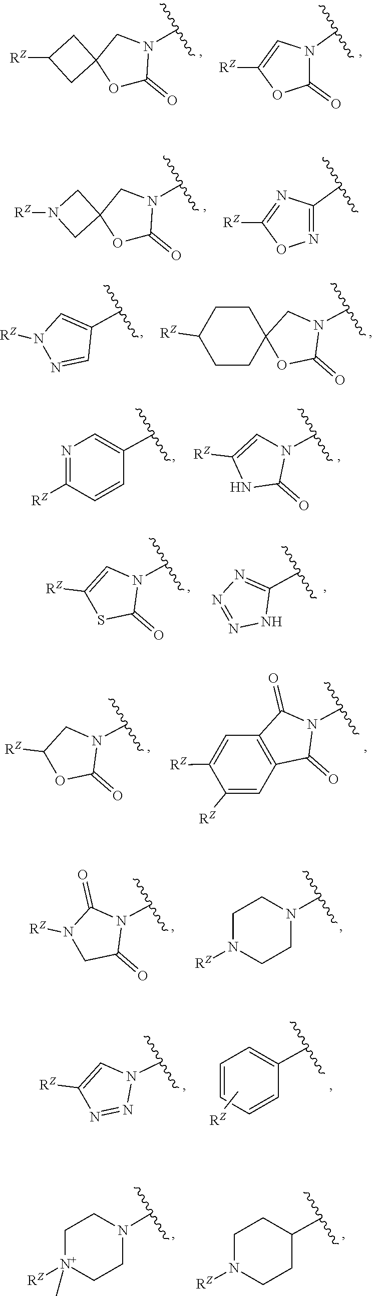

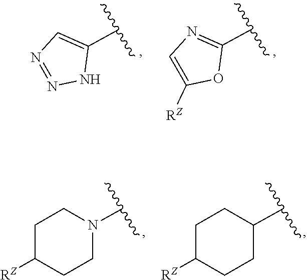

wherein each occurrence of R^{Z} independently Has the formula:

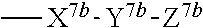

wherein:
   X^{7b} is absent or a linker group of the formula —[CR^{7L}R^{7M}]ₓ— in which x is an integer selected from 1 or 2, and each occurrence of R^{L} and R^{7M} is each independently selected from hydrogen or methyl, and
   Y^{7b} is absent or —N(R^{7N})—, —N(R^{7P})—C(O)O—, —N(R^{7P})SO₂N(R^{7N})—, —C(NR^{7P})N(R^{7N})—, or —N(R^{7P})C(═NR^{7Q})N(R^{7N})—, wherein R^{7N}, R^{7P} and R^{7D} are each independently selected from hydrogen or methyl; and
   Z^{7b} is hydrogen, (1-4C)alkyl, (3-6C)cycloalkyl, or 4 to 6 membered heterocyclyl; and
   and wherein Z^{7b} is optionally further substituted by one or more substituent groups independently selected from oxo, NR^{7R}R^{7S}, methyl; wherein R^{7R} and R^{7S} are each independently selected from hydrogen or methyl.
(21) R₇ is a group:

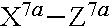

wherein
X^{7a} is absent or —O—, —C(O)—, —C(O)O—, —OC(O)—, —OC(O)N(R^{7C})—, —CH(OR^{7C})—, —N(R^{7C}—, —N(R^{7D})—C(O)—, —N(R^{7D})—C(O)O—, —C(O)—N(R^{7C})—, —N(R^{7D})C(O)N(R^{7C})—, —N(R^{7D})SO₂—, —S—C(NR^{7E})N(R^{7C})—, or —N(R^{7D})C(NR^{7E})N(R^{7C})—, wherein R^{7C}, R^{7D} and R^{7E} are each independently selected from hydrogen or methyl; and
Z^{7a} is a group selected from a group of the formula:

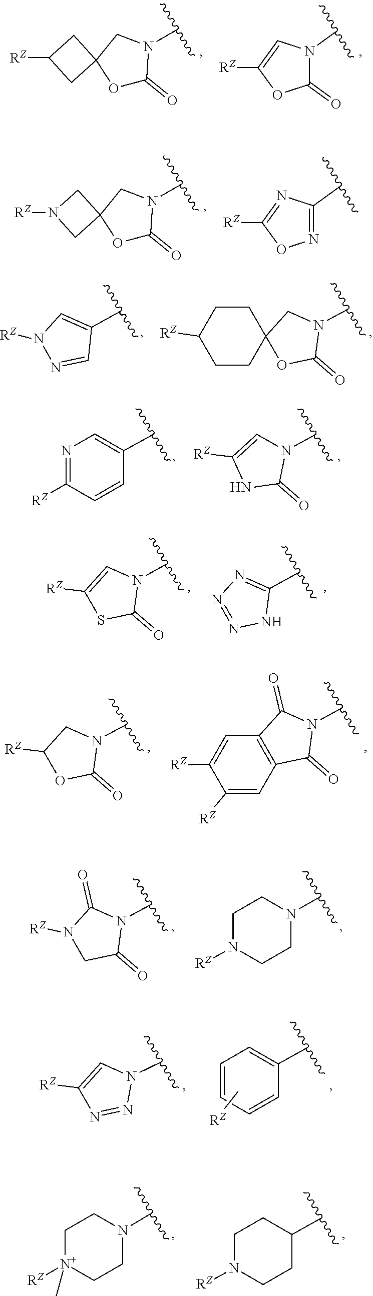

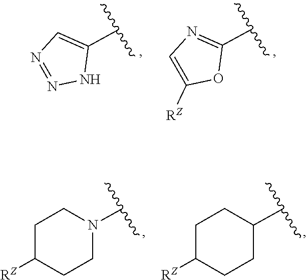

wherein each occurrence of R^{Z} is independently as defined herein.
(22) R₇ is a selected from a group of the formula:

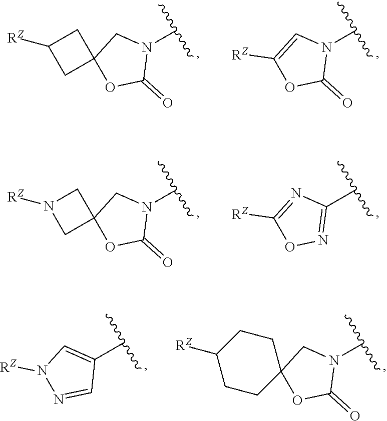

wherein R^{Z} has the formula:

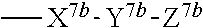

wherein:
   X^{7b} is absent or a linker group of the formula —[CR^{7L}R^{7M}]—, in which x is an integer selected from 1 or 2, and each occurrence of R^{7L} and R^{7M} is each independently selected from hydrogen or methyl; and
   Y^{7b} is absent or —N(R^{7N})—, —N(R^{7P})—C(O)O—, —N(R^{7P})SO₂N(R^{7N})—, —C(NR^{7P})N(R^{7N})—, or —N(R^{7P})C(═NR^{7Q})N(R^{7N})—, wherein R^{7N}, R^{7P} and R^{7D} are each independently selected from hydrogen or methyl; and
   Z^{7b} is hydrogen, (1-4C)alkyl, (3-6C)cycloalkyl, or 4 to 6 membered heterocyclyl; and
   and wherein Z^{7b} is optionally further substituted by one or more substituent groups independently selected from oxo, NR^{7R}R^{7S} and methyl; wherein R^{7R} and R^{7S} are each independently selected from hydrogen or methyl.
(23) R₇ is a selected from a group of the formula:

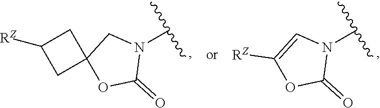

wherein
R^{Z} has the formula:

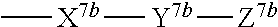

wherein:
   X^{7b} is absent or a linker group of the formula —[CR^{7L}R^{7M}]—, in which x is an integer selected from 1 or 2, and each occurrence of R^{7L} and R^{7M} is each independently selected from hydrogen or methyl; and
   Z^{7b} is (1-4C)alkyl, or 4 to 6 membered heterocyclyl; and
   and wherein Z^{7b} is optionally further substituted by one or more substituent groups independently selected from NR^{7R}R^{7S} and methyl; wherein R^{7R} and R^{7S} are each independently selected from hydrogen or methyl.
(24) R₇ is a selected from a group of the formula:

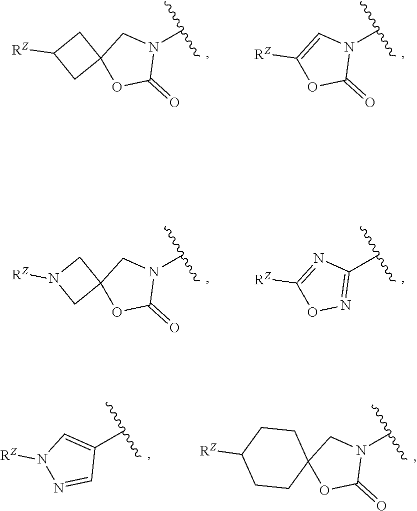

wherein R^{Z} is as defined herein;
(25) R₇ is a selected from a group of the formula:

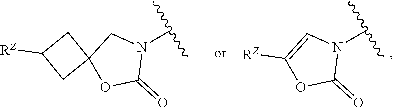

wherein R^{Z} is as defined herein.
(26) R₇ is a group:

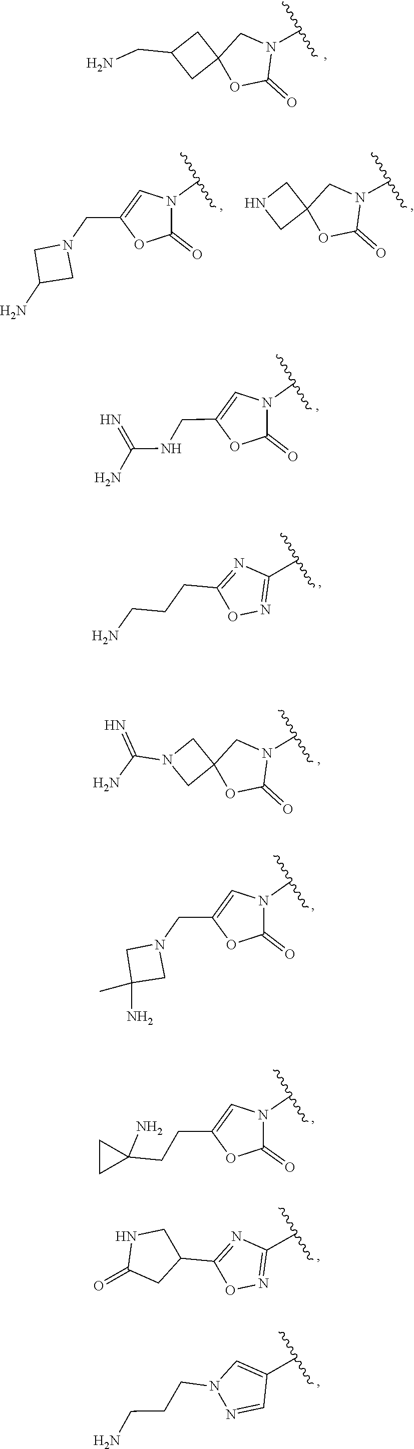

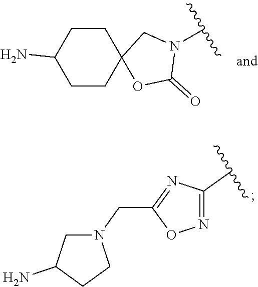 [SMILES: *N1CC2(CCC(N)CC2)OC1=O ; *c1noc(CN2CCC(N)C2)n1]

(27) R₇ is a group:

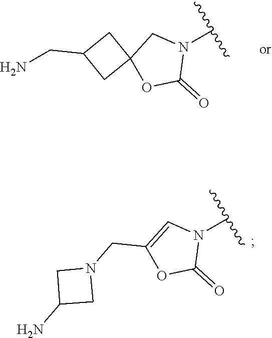 [SMILES: *N1CC2(CC(CN)C2)OC1=O ; *n1cc(CN2CC(N)C2)oc1=O]

(28) R₇ is a group:

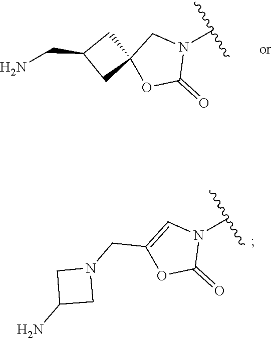 [SMILES: *N1C[C@]2(C[C@@H](CN)C2)OC1=O ; *n1cc(CN2CC(N)C2)oc1=O]

(29) Rₐ is C₁₋₄ alkyl:
(30) Rₐ is methyl or ethyl:
(31) Rₐ is methyl:
(32) R_{b} is hydrogen, methyl or ethyl:
(33) R_{b} is hydrogen
(34) R₂ is selected from:
   (i) —C(O)OH;
   (ii) —C(O)NR_{2B}R_{2C}; wherein R_{2B} and R_{2C} are each independently selected from hydrogen, (1-6C)alkyl, aryl or heteroaryl, each of which is optionally substituted by one or more substituent groups R^{A};
   (iii) —C(O)NR_{2D}NR_{2B}R_{2C}; wherein R_{2D} is selected from hydrogen or methyl and R_{2B} and R_{2C} are as defined above;
   (iv) tetrazolyl;
   and wherein R^{A} is selected from halo, cyano, or a group of the formula:

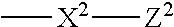

wherein
   X² is absent or —C(O)—, —SO₂—; and
   Z² is hydrogen, (1-4C)alkyl, phenyl, or a 5- or 6-membered heteroaryl;
      and wherein Z² is optionally further substituted by one or more substituent groups independently selected from halo, hydroxyl or (1-4C)alkyl;
(35) R₂ is selected from:
   (i) —C(O)OH;
   (ii) —C(O)NR_{2B}R_{2C}; wherein R_{2B} and R_{2C} are each independently selected from hydrogen, (1-6C)alkyl, aryl or heteroaryl, each of which is optionally substituted by one or more substituent groups R^{A};
   (iii) —C(O)NR_{2C} NR_{2B}R_{2C}; wherein R_{2D} is selected from hydrogen or methyl and R_{2B} and R_{2C} are as defined above;
   (iv) tetrazolyl;
   and wherein R^{A} is selected from halo, cyano or SO₂CH₃;
(36) R₂ is selected from —C(O)OH and tetrazolyl;
(37) R₂ is tetrazolyl.

In preferred embodiments of the invention, the compound is a compound according to Formula I.

Suitably, a heteroaryl or heterocyclyl group as defined herein is a monocyclic heteroaryl or heterocyclyl group comprising one, two or three heteroatoms selected from N, O or S.

Suitably, a heteroaryl is a 5- or 6-membered heteroaryl ring comprising one, two or three heteroatoms selected from N, O or S.

Suitably, a heterocyclyl group is a 4-, 5-, 6- or 7-membered heterocyclyl ring comprising one, two or three heteroatoms selected from N, O or S; or is a 8, 9, or 10-membered spiro-fused heterocyclylic ring system comprising one, two or three heteroatoms selected from N, O or S. Most suitably, a heterocyclyl group is a 5-, 6- or 7-membered ring comprising one, two or three heteroatoms selected from N, O or S [e.g. morpholinyl (e.g. 4-morpholinyl), pyridinyl, piperazinyl, homopiperazinyl or pyrrolidinonyl]. Suitably, a heterocyclyl group is a 8, 9, or 10-membered spiro-fused heterocyclylic ring system comprising one, two or three heteroatoms selected from N, O or S.

Suitably, R₃ is as defined in any one of paragraphs (1) to (12) above. More suitably, R₃ is as defined in any one of paragraphs (3) to (12). Even more suitably, R₃ is as defined in any one of paragraphs (6) to (12). Most suitably, R₃ is as defined in paragraph (9), (10), (11) or (12).

Suitably, R₇ is as defined in any one of paragraphs (13) to (28) above. More suitably, R₇ is as defined in any one of paragraphs (15) to (18) or (19) to (28) above. Most suitably, R₇ is as defined in paragraph (25), (26), (27) or (28).

Suitably, R₇ is as defined in paragraph (24) or (25), and R^{Z} is as defined in any one of paragraphs (12) to (23). More suitably, R₇ is as defined in paragraph (24) or (25), and R^{Z} is as defined in any one of paragraphs (16) to (23). More suitably, R₇ is as defined in any one of paragraphs (18), (19) or (22) to (28) above. Most suitably, R₇ is as defined in paragraph (24) or (25), and R^{Z} is as defined in paragraph (23).

Suitably, Rₐ is as defined in any one of paragraphs (29) to (31) above. More suitably, Rₐ is as defined in paragraph (30) or (31). Most suitably, Rₐ is as defined in paragraph (31), i.e. Rₐ is methyl.

Suitably, R_{b} is as defined in any one of paragraph (32) or (33). Most suitably, R_{b} is as defined in paragraph (33), i.e. R_{b} is hydrogen.

In certain embodiments, R³ is a phenyl ring comprising one or more substituent groups as defined herein. Modification of the phenyl ring through the presence of substituent groups can result in improved metabolic stability, for example by the inclusion of one or more fluorine substituents (e.g. 1 or 2) in available positions.

In a particular group of compounds of the invention, the compound has a structure according to Formula Ia or Ib (sub definitions of Formula I):

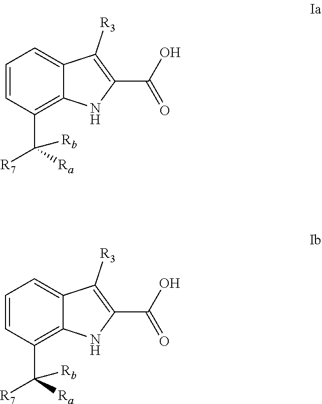

wherein R₃, R₇, Rₐ and R_{b} have any one of the meanings defined herein; or a pharmaceutically acceptable salt, hydrate and/or solvate thereof.

In an embodiment of the compounds of formula Ia or formula Ib:
R₃ is as defined in any one of paragraphs (1) to (12) above;
R₇ is as defined in any one of paragraphs (13) to (28) above;
Rₐ is as defined in any one of paragraphs (29) to (31) above; and
R_{b} is as defined in paragraph (32) or (33).

In an embodiment of the compounds of formula Ia or formula Ib:
R₃ is as defined in any one of paragraphs (6) to (12);
R₇ is as defined in any one of paragraphs (15) to (18) or (19) to (28) above;
Rₐ is as defined in paragraph (30) or (31); and
R_{b} is as defined in paragraph (33), i.e. R_{b} is hydrogen.

In an embodiment of the compounds of formula Ia or formula Ib:
R₃ is as defined in paragraph (11) or (12);
R₇ is as defined in paragraph (25), (26), (27) or (28);
Rₐ is as defined in paragraph (31), i.e. Rₐ is methyl; and
R_{b} is as defined in paragraph (33), i.e. R_{b} is hydrogen.

In a particular group of compounds of the invention, the compound has a structure according to Formula Ia, as defined herein.

In a particular group of compounds of the invention, R_{b} is hydrogen and the compound has a structure according to Formula Ic, Id or Ie (sub definitions of Formula I):

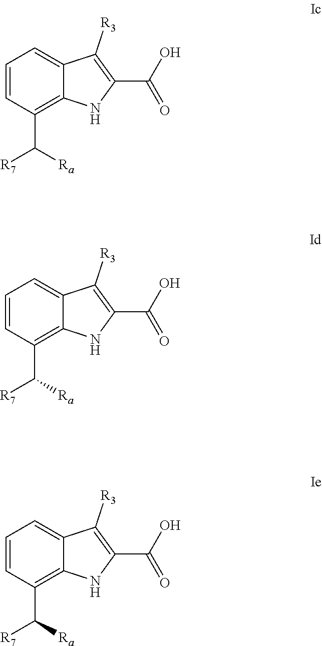

wherein R₃, R₇ and Rₐ have any one of the meanings defined herein; or a pharmaceutically acceptable salt, hydrate and/or solvate thereof.

In an embodiment of the compounds of Formula Ic, Id or Ie:
R₃ is as defined in any one of paragraphs (1) to (12) above;
R₇ is as defined in any one of paragraphs (13) to (28) above; and
Rₐ is as defined in any one of paragraphs (29) to (31) above.

In an embodiment of the compounds of Formula Ic, Id or Ie:
R₃ is as defined in any one of paragraphs (6) to (12);
R₇ is as defined in any one of paragraphs (15) to (18) or (19) to (28) above; and
Rₐ is as defined in paragraph (30) or (31).

In an embodiment of the compounds of Formula Ic, Id or Ie:
R₃ is as defined in paragraph (11) or (12);
R₇ is as defined in paragraph (25), (26), (27) or (28); and
Rₐ is as defined in paragraph (31), i.e. Rₐ is methyl.

In a particular group of compounds of the invention, the compound has a structure according to Formula Ic.

In a particular group of compounds of the invention, Rₐ is methyl, R_{b} is hydrogen and the compound has a structure according to Formula If, Ig or Ih (sub definitions of Formula I):

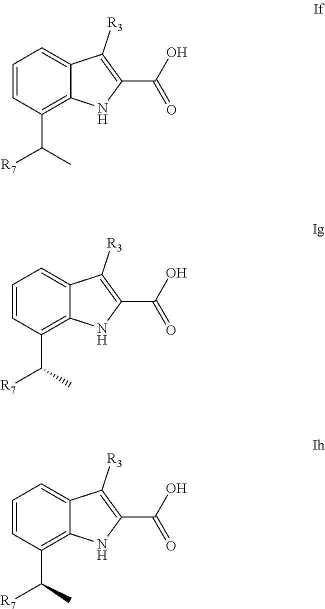

wherein R₃ and R₇ have any one of the meanings defined herein; or a pharmaceutically acceptable salt, hydrate and/or solvate thereof.

In an embodiment of the compounds of Formula If, Ig or Ih
R₃ is as defined in any one of paragraphs (1) to (12) above;
R₇ is as defined in any one of paragraphs (13) to (28) above;

In an embodiment of the compounds of Formula If, Ig or Ih
R₃ is as defined in any one of paragraphs (6) to (12) above;
R₇ is as defined in any one of paragraphs (15) to (18) or (19) to (28) above;

In an embodiment of the compounds of Formula If, Ig or Ih
R₃ is as defined in paragraph (11) or (12); and
R₇ is as defined in paragraph (25), (26), (27) or (28)

In a particular group of compounds of the invention, R₇ is as defined in paragraph (25), Rₐ is methyl, R_{b} is hydrogen and the compound has a structure according to Formula II or Ij (sub definitions of Formula I):

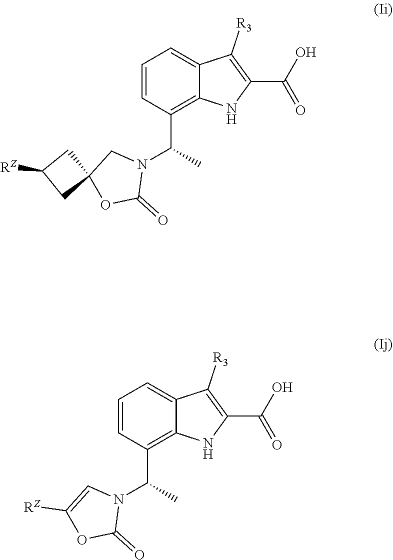

wherein R₃ and R^{Z} have any one of the meanings defined herein; or a pharmaceutically acceptable salt, hydrate and/or solvate thereof.

In an embodiment of the compounds of Formula Ii or Ij:
R₃ is as defined in any one of paragraphs (1) to (12) above;
R^{Z} is as defined in any one of paragraphs (12) to (23) above;

In an embodiment of the compounds of Formula Ii or Ij:
R₃ is as defined in any one of paragraphs (6) to (12) above;
R^{Z} is as defined in any one of paragraphs (15) to (23) above;

In an embodiment of the compounds of Formula Ii or Ij:
R₃ is as defined in paragraph (9), (10), (11) or (12) above; and
R^{Z} is as defined in paragraph (23) above;

In a particular group of compounds of the invention, R₃ is as defined in paragraph (7), Rₐ is methyl, R_{b} is hydrogen and the compound has a structure according to Formula Ik, Im or In (a sub definition of Formula I):

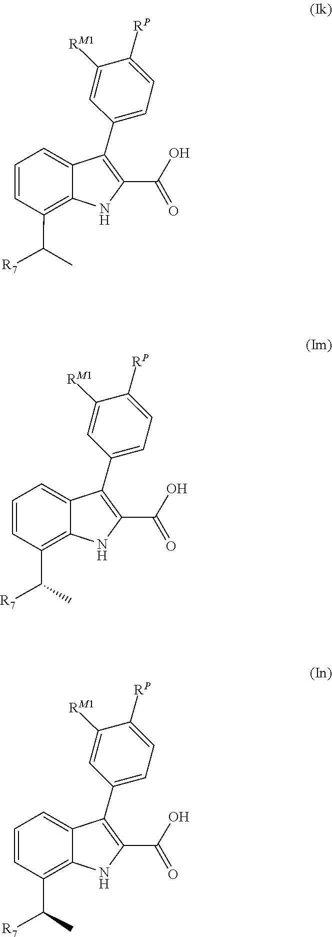

wheren R^{M1}, R^{P} and R₇ have any one of the meanings defined herein; or a pharmaceutically acceptable salt, hydrate and/or solvate thereof.

In an embodiment of the compounds of Formula Ik, Im or In:
R^{M1} is as defined in any one of paragraphs (4) to (9) above;
R^{P} is as defined in any one of paragraphs (4) to (9) above; and
R₇ is as defined in any one of paragraphs (13) to (28) above;
In an embodiment of the compounds of Formula Ik, Im or In:
R^{M1} is as defined in any one of paragraphs (6) to (9) above;
R^{P} is as defined in any one of paragraphs (6) to (9) above; and
R₇ is as defined in any one of paragraphs (15) to (18) or (19) to (28) above;
In an embodiment of the compounds of Formula Ik, Im or In:
R^{M1} is as defined in paragraph (8) or (9) above;
R^{P} is as defined in paragraph (8) or (9) above; and
R₇ is as defined in any one of paragraphs (18), (19) or (22) to (28) above;

In an embodiment of the compounds of Formula Ik, Im or In:
R^{M1} is as defined in paragraph (9) above;
R^{P} is as defined in paragraph (9) above; and
R₇ is as defined paragraph (25), (26), (27) or (28) above;
In a particular group of compounds of the invention, R₃ is as defined in paragraph (7), R₇ is as defined in paragraph (25), Rₐ is methyl, R_{b} is hydrogen and the compound has a structure according to Formula Io or Ip (a sub definition of Formula I):

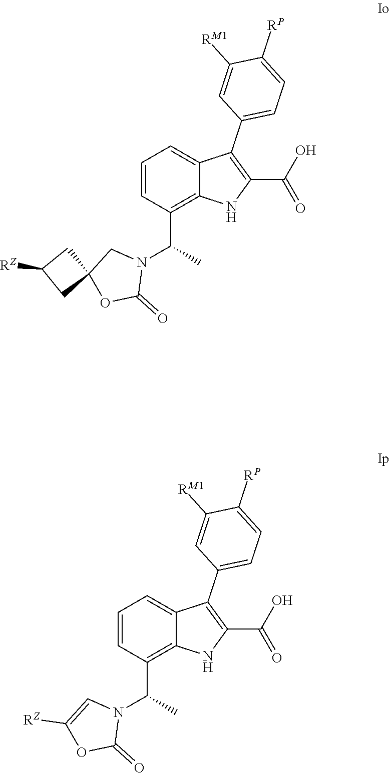

wheren R^{M1}, R^{P} and R^{Z} have any one of the meanings defined herein; or a pharmaceutically acceptable salt, hydrate and/or solvate thereof.

In an embodiment of the compounds of Formula Io or Ip:
R^{M1} is as defined in any one of paragraphs (4) to (9) above;
R^{P} is as defined in any one of paragraphs (4) to (9) above; and
R^{Z} is as defined in any one of paragraphs (12) to (23) above;

In an embodiment of the compounds of Formula Io or Ip:
R^{M1} is as defined in any one of paragraphs (6) to (9) above;
R^{P} is as defined in any one of paragraphs (6) to (9) above; and
R^{Z} is as defined in any one of paragraphs (16) to (23) above;

In an embodiment of the compounds of Formula Io or Ip:
R^{M1} is as defined in paragraph (8) or (9) above;
R^{P} is as defined in paragraph (8) or (9) above; and
R^{Z} is as defined in paragraph (23) above;

Particular compounds of the present invention include any of the compounds exemplified in the present application, or a pharmaceutically acceptable salt or solvate thereof, and, in particular, any of the following:
IC-1: 3-(3,5-Dichlorophenyl)-7-(1-(phenylsulfonamido)ethyl)-1H-indole-2-carboxylic acid
IC-2: 3-(3,5-Dichlorophenyl)-7-(1-(methylsulfonamido)ethyl)-1H-indole-2-carboxylic acid
IC-3: 3-(3-Fluoro-4-((methylsulfonyl)methyl)phenyl)-7-(1-(1-methyl-1H-pyrazol-4-yl)ethyl)-1H-indole-2-carboxylic acid
IC-4: 3-(3-Fluoro-4-((methylsulfonyl)methyl)phenyl)-7-(1-(thiophen-2-yl)ethyl)-1H-indole-2-carboxylic acid
IC-5: 7-(1-(1-(3-Aminopropyl)-1H-pyrazol-4-yl)ethyl)-3-(3-fluoro-4-((methylsulfonyl) methyl)phenyl)-1H-indole-2-carboxylic acid
IC-6: 3-(3-Fluoro-4-((methylsulfonyl)methyl)phenyl)-7-(1-(2-oxo-2H-pyran-3-yl)ethyl)-1H-indole-2-carboxylic acid
IC-7: 7-(1-(2-Chlorothiazol-5-yl)ethyl)-3-(3-fluoro-4-((methylsulfonyl)methyl)phenyl)-1H-indole-2-carboxylic acid
IC-8: 3-(3-Fluoro-4-((methylsulfonyl)methyl)phenyl)-7-(1-(4-((sulfamoylamino)methyl)-1H-1,2,3-triazol-1-yl)ethyl)-1H-indole-2-carboxylic acid
IC-9: 7-(1-(2H-Tetrazol-5-yl)ethyl)-3-(3-fluoro-4-((methylsulfonyl)methyl)phenyl)-1H-indole-2-carboxylic acid
IC-10: 7-[1-[4-(3-Aminopropyl)triazol-1-yl]ethyl]-3-[3-chloro-4-(methylsulfonylmethyl)phenyl]-1H-indole-2-carboxylic acid
IC-11: 7-(1-((4-(Aminomethyl)benzoyl)amino)ethyl)-3-(6-(morpholin-4-ylmethyl)pyridin-3-yl)-1H-indole-2-carboxylic acid
IC-12: 3-(3-Fluoro-4-((methylsulfonyl)methyl)phenyl)-7-(1-(piperazin-1-yl)ethyl)-1H-indole-2-carboxylic acid
IC-13: 7-[1-[3-(3-Aminopropyl)-2,5-dioxo-imidazolidin-1-yl]ethyl]-3-[3-fluoro-4-(methylsulfonylmethyl)phenyl]-1H-indole-2-carboxylic acid
IC-14: 7-((S)-1-((2S,4r)-2-(Aminomethyl)-6-oxo-5-oxa-7-azaspiro[3.4]octan-7-yl)ethyl)-3-(3-fluoro-4-(methylsulfonamido)phenyl)-1H-indole-2-carboxylic acid
IC-15: 7-((S)-1-((2S,4r)-2-(aminomethyl)-6-oxo-5-oxa-7-azaspiro[3.4]octan-7-yl)ethyl)-3-(6-oxo-1,6-dihydropyridin-3-yl)-1H-indole-2-carboxylic acid
IC-16: 7-((1S)-1-(2-(aminomethyl)-6-oxo-5-oxa-7-azaspiro[3.4]octan-7-yl)ethyl)-3-(3-cyano-4-(methylsulfonamido)phenyl)-1H-indole-2-carboxylic acid
IC-17: 7-((1S)-1-(2-(aminomethyl)-6-oxo-5-oxa-7-azaspiro[3.4]octan-7-yl)ethyl)-3-(2-methoxypyridin-4-yl)-1H-indole-2-carboxylic acid
IC-18: 7-((S)-1-((2S,4r)-2-(aminomethyl)-6-oxo-5-oxa-7-azaspiro[3.4]octan-7-yl)ethyl)-3-(2-hydroxypyridin-4-yl)-1H-indole-2-carboxylic acid
IC-19: 7-((1S)-1-(2-(aminomethyl)-6-oxo-5-oxa-7-azaspiro[3.4]octan-7-yl)ethyl)-3-(3-fluoro-4-((S-methylsulfonimidoyl)methyl)phenyl)-1H-indole-2-carboxylic acid
IC-20: 7-((1S)-1-(2-(aminomethyl)-6-oxo-5-oxa-7-azaspiro[3.4]octan-7-yl)ethyl)-3-(2-methoxypyridin-4-yl)-1H-indole-2-carboxylic acid
IC-21: 7-((S)-1-((2S,4r)-2-(aminomethyl)-6-oxo-5-oxa-7-azaspiro[3.4]octan-7-yl)ethyl)-3-(2,6-difluoro-4-(methylsulfonamido)phenyl)-1H-indole-2-carboxylic acid
IC-22: 7-((S)-1-((2R,4s)-2-(aminomethyl)-6-oxo-5-oxa-7-azaspiro[3.4]octan-7-yl)ethyl)-3-(1-oxoisoindolin-5-yl)-1H-indole-2-carboxylic acid
IC-23: 7-((S)-1-((2S,4r)-2-(aminomethyl)-6-oxo-5-oxa-7-azaspiro[3.4]octan-7-yl)ethyl)-3-(5-hydroxypyridin-3-yl)-1H-indole-2-carboxylic acid
IC-24: 7-((S)-1-((2S,4r)-2-(aminomethyl)-6-oxo-5-oxa-7-azaspiro[3.4]octan-7-yl)ethyl)-3-(3-fluoro-4-(oxetane-3-sulfonamido)phenyl)-1H-indole-2-carboxylic acid
IC-25: 7-((S)-1-((2S,4r)-2-(aminomethyl)-6-oxo-5-oxa-7-azaspiro[3.4]octan-7-yl)ethyl)-3-(6-hydroxy-5-methoxypyridin-3-yl)-1H-indole-2-carboxylic acid
IC-26: 7-((1S)-1-(2-(aminomethyl)-6-oxo-5-oxa-7-azaspiro[3.4]octan-7-yl)ethyl)-3-(5-fluoro-6-oxo-1,6-dihydropyridin-3-yl)-1H-indole-2-carboxylic acid
IC-27: (S)-7-(1-(2-(aminomethyl)-6-oxo-5-oxa-7-azaspiro[3.4]octan-7-yl)ethyl)-3-(5-hydroxy-6-methoxypyridin-3-yl)-1H-indole-2-carboxylic acid
IC-28: 7-((S)-1-((2S,4r)-2-(aminomethyl)-6-oxo-5-oxa-7-azaspiro[3.4]octan-7-yl)ethyl)-3-(1-methyl-6-oxo-1,6-dihydropyridazin-4-yl)-1H-indole-2-carboxylic acid
IC-29: 7-((S)-1-((2S,4r)-2-(aminomethyl)-6-oxo-5-oxa-7-azaspiro[3.4]octan-7-yl)ethyl)-3-(5,6-dihydroxypyridin-3-yl)-1H-indole-2-carboxylic acid
IC-31: 3-(2,6-dihydroxypyridin-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
IC-32: 3-(1-methyl-2-oxo-1,2-dihydropyridin-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
IC-33: 3-(6-oxo-1,6-dihydropyridazin-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
IC-34: 3-(2-Aminopyridin-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
IC-35: 3-[2-(methylamino)pyridin-4-yl]-7-[(1S)-1-[(2r,4r)-2-(aminomethyl)-6-oxo-5-oxa-7-azaspiro[3.4]octan-7-yl]ethyl]-1H-indole-2-carboxylic acid
IC-36: 3-[6-(methylamino)pyridin-3-yl]-7-[(1S)-1-[(2r,4r)-2-(aminomethyl)-6-oxo-5-oxa-7-azaspiro[3.4]octan-7-yl]ethyl]-1H-indole-2-carboxylic acid
IC-37: 3-(6-aminopyridin-3-yl)-7-[(1S)-1-[(2r,4r)-2-(aminomethyl)-6-oxo-5-oxa-7-azaspiro[3.4]octan-7-yl]ethyl]-1H-indole-2-carboxylic acid
IC-38: 3-(pyridin-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
IC-39: 3-(pyridin-3-yl)-7-[(1S)-1-[(2r,4r)-2-(aminomethyl)-6-oxo-5-oxa-7-azaspiro[3.4]octan-7-yl]ethyl]-1H-indole-2-carboxylic acid
IC-40: 7-((S)-1-((2S,4r)-2-(aminomethyl)-6-oxo-5-oxa-7-azaspiro[3.4]octan-7-yl)ethyl)-3-(1,1-dioxidothiomorpholino)-1H-indole-2-carboxylic acid
IC-41: 7-((S)-1-((2S,4r)-2-(aminomethyl)-6-oxo-5-oxa-7-azaspiro[3.4]octan-7-yl)ethyl)-3-(2-oxoindolin-5-yl)-1H-indole-2-carboxylic acid
IC-42: 7-((S)-1-((2S,4r)-2-(aminomethyl)-6-oxo-5-oxa-7-azaspiro[3.4]octan-7-yl)ethyl)-3-(2-aminopyrimidin-5-yl)-1H-indole-2-carboxylic acid
IC-43: 7-((S)-1-((2S,4r)-2-(aminomethyl)-6-oxo-5-oxa-7-azaspiro[3.4]octan-7-yl)ethyl)-3-(1H-pyrazol-4-yl)-1H-indole-2-carboxylic acid
IC-44: 3-(1-methyl-1H-pyrazol-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
IC-45: 7-((S)-1-((2S,4r)-2-(aminomethyl)-6-oxo-5-oxa-7-azaspiro[3.4]octan-7-yl)ethyl)-3-(1-(cyanomethyl)-1H-pyrazol-4-yl)-1H-indole-2-carboxylic acid
IC-46: 3-(1-(2-amino-2-iminoethyl)-1H-pyrazol-4-yl)-7-((S)-1-((2S,4r)-2-(aminomethyl)-6-oxo-5-oxa-7-azaspiro[3.4]octan-7-yl)ethyl)-1H-indole-2-carboxylic acid
IC-47: 3-(1-(2-amino-2-oxoethyl)-1H-pyrazol-4-yl)-7-((S)-1-((2S,4r)-2-(aminomethyl)-6-oxo-5-oxa-7-azaspiro[3.4]octan-7-yl)ethyl)-1H-indole-2-carboxylic acid
IC-48: 7-((S)-1-((2S,4r)-2-(aminomethyl)-6-oxo-5-oxa-7-azaspiro[3.4]octan-7-yl)ethyl)-3-(isoxazol-4-yl)-1H-indole-2-carboxylic acid
IC-49: 7-((S)-1-((2S,4r)-2-(aminomethyl)-6-oxo-5-oxa-7-azaspiro[3.4]octan-7-yl)ethyl)-3-(3,5-dimethyl-1H-pyrazol-4-yl)-1H-indole-2-carboxylic acid
IC-50: 7-((S)-1-((2S,4r)-2-(aminomethyl)-6-oxo-5-oxa-7-azaspiro[3.4]octan-7-yl)ethyl)-3-(3-oxo-2,3-dihydro-1H-pyrazol-4-yl)-1H-indole-2-carboxylic acid
IC-51: 7-[(1S)-1-{5-[(3-Aminoazetidin-1-yl)methyl]-2-oxo-2,3-dihydro-1,3-oxazol-3-yl}ethyl]-3-(2,6-difluoro-4-methanesulfonamidophenyl)-1H-indole-2-carboxylic acid
IC-52: 7-[(1S)-1-{5-[(3-aminoazetidin-1-yl)methyl]-2-oxo-2,3-dihydro-1,3-oxazol-3-yl}ethyl]-3-{6-[(morpholin-4-yl)methyl]pyridin-3-yl}-1H-indole-2-carboxylic acid
IC-53: 7-[(1S)-1-{5-[(3-aminoazetidin-1-yl)methyl]-2-oxo-2,3-dihydro-1,3-oxazol-3-yl}ethyl]-3-(3-fluoro-4-{[imino(methyl)oxo-λ⁶-sulfanyl]methyl}phenyl)-1H-indole-2-carboxylic acid
IC-54: 7-[(1S)-1-{5-[(3-Aminoazetidin-1-yl)methyl]-2-oxo-2,3-dihydro-1,3-oxazol-3-yl}ethyl]-3-(2-oxo-1,2-dihydropyridin-4-yl)-1H-indole-2-carboxylic acid
IC-55: 7-[(1S)-1-{5-[(3-aminoazetidin-1-yl)methyl]-2-oxo-2,3-dihydro-1,3-oxazol-3-yl}ethyl]-3-(3-cyano-4-methanesulfonamidophenyl)-1H-indole-2-carboxylic acid
IC-56: 7-[(1S)-1-{5-[(3-aminoazetidin-1-yl)methyl]-2-oxo-2,3-dihydro-1,3-oxazol-3-yl}ethyl]-3-(2-methoxypyridin-4-yl)-1H-indole-2-carboxylic acid
IC-57 7-[(1S)-1-{5-[(3-Aminoazetidin-1-yl)methyl]-2-oxo-2,3-dihydro-1,3-oxazol-3-yl}ethyl]-3-(3-fluoro-4-methanesulfonamidophenyl)-1H-indole-2-carboxylic acid
IC-58: (S)-3-(3-fluoro-4-((methylsulfonyl)methyl)phenyl)-7-(1-(5-(guanidinomethyl)-2-oxooxazol-3(2H)-yl)ethyl)-1H-indole-2-carboxylic acid
IC-59: 7-((1S)-1-((4-(aminomethyl)benzoyl)amino)ethyl)-3-(6-(morpholin-4-ylmethyl)pyridin-3-yl)-1H-indole-2-carboxylic acid
IC-60: 7-((1S)-1-(2S,4r)-(2-(aminomethyl)-6-oxo-5-oxa-7-azaspiro[3.4]oct-7-yl)ethyl)-3-(3-fluoro-4-((methylsulfonyl)methyl)phenyl)-1H-indole-2-carboxylic acid
IC-61: 7-((1S)-1-(2R,4s)-(2-(aminomethyl)-6-oxo-5-oxa-7-azaspiro[3.4]oct-7-yl)ethyl)-3-(3-fluoro-4-((methylsulfonyl)methyl)phenyl)-1H-indole-2-carboxylic acid
IC-62: 7-[1-[5-(3-Aminopropyl)-1,2,4-oxadiazol-3-yl]ethyl]-3-[3-fluoro-4-(methylsulfonyl methyl)phenyl]-1H-indole-2-carboxylic acid
IC-63: 3-[3-Fluoro-4-(methylsulfonylmethyl)phenyl]-7-[1-[5-(5-oxopyrrolidin-3-yl)-1,2,4-oxadiazol-3-yl]ethyl]-1H-indole-2-carboxylic acid
IC-64: 7-[1-[5-(3-Aminopropyl)-2-oxo-1H-imidazol-3-yl]ethyl]-3-[3-fluoro-4-(methylsulfonylmethyl)phenyl]-1H-indole-2-carboxylic acid
IC-65: (S)-3-(3-fluoro-4-((methylsulfonyl)methyl)phenyl)-7-(1-(6-oxo-5-oxa-2,7-diazaspiro[3.4]octan-7-yl)ethyl)-1H-indole-2-carboxylic acid
IC-66: (S)-7-(1-(2-carbamimidoyl-6-oxo-5-oxa-2,7-diazaspiro[3.4]octan-7-yl)ethyl)-3-(3-fluoro-4-((methylsulfonyl)methyl)phenyl)-1H-indole-2-carboxylic acid
IC-67: (S)-7-(1-(5-((3-aminoazetidin-1-yl)methyl)-2-oxooxazol-3(2H)-yl)ethyl)-3-(3-fluoro-4-((methylsulfonyl)methyl)phenyl)-1H-indole-2-carboxylic acid
IC-68: (S)-7-(1-(5-((3-amino-3-methylazetidin-1-yl)methyl)-2-oxooxazol-3(2H)-yl)ethyl)-3-(3-fluoro-4-((methylsulfonyl)methyl)phenyl)-1H-indole-2-carboxylic acid
IC-69: (S)-7-(1-(5-(2-(1-Aminocyclopropyl)ethyl)-2-oxooxazol-3(2H)-yl)ethyl)-3-(3-fluoro-4-((methylsulfonyl)methyl)phenyl)-1H-indole-2-carboxylic acid
IC-70: (S)-7-(1-(5-(3-aminopropyl)-2-oxooxazol-3(2H)-yl)ethyl)-3-(3-fluoro-4-((methylsulfonyl)methyl)phenyl)-1H-indole-2-carboxylic acid
IC-71: (S)-7-(1-(8-amino-2-oxo-1-oxa-3-azaspiro[4.5]decan-3-yl)ethyl)-3-(3-fluoro-4-((methylsulfonyl)methyl)phenyl)-1H-indole-2-carboxylic acid
IC-72: 7-(1-((5-(3-aminopropyl)oxazol-2-yl)oxy)ethyl)-3-(3-fluoro-4-((methylsulfonyl)methyl)phenyl)-1H-indole-2-carboxylic acid
IC-73: 3-(3-Fluoro-4-((methylsulfonyl)methyl)phenyl)-7-(1-(5-(3-guanidinopropyl)-2-oxooxazol-3(2H)-yl)ethyl)-1H-indole-2-carboxylic acid
IC-74: 7-(1-(5-(2-(1-aminocyclopropyl)ethyl)-2-oxooxazol-3(2H)-yl)ethyl)-3-(3-fluoro-4-((methylsulfonyl)methyl)phenyl)-1H-indole-2-carboxylic acid
IC-75: 7-(1-(5-(3-(Dimethylamino)propyl)-2-oxooxazol-3(2H)-yl)ethyl)-3-(3-fluoro-4-((methylsulfonyl)methyl)phenyl)-1H-indole-2-carboxylic acid
IC-76: 3-(3-(1-(2-carboxy-3-(3-fluoro-4-((methylsulfonyl)methyl)phenyl)-1H-indol-7-yl)ethyl)-2-oxo-2,3-dihydrooxazol-5-yl)-N, N, N-trimethylpropan-1-aminium IC-77: 7-(1-((5-(2-aminoethyl)oxazol-2-yl)oxy)ethyl)-3-(3-fluoro-4-((methylsulfonyl)methyl)phenyl)-1H-indole-2-carboxylic acid
IC-78: 3-(3-Fluoro-4-((methylsulfonyl)methyl)phenyl)-7-(1-(5-(hydroxymethyl)-2-oxooxazol-3(2H)-yl)ethyl)-1H-indole-2-carboxylic acid
IC-79: 7-(1-(5-(Aminomethyl)-2-oxooxazol-3(2H)-yl)ethyl)-3-(3-fluoro-4-((methylsulfonyl)methyl)phenyl)-1H-indole-2-carboxylic acid
IC-80: 7-(1-(5-((2-Aminoacetamido)methyl)-2-oxooxazol-3(2H)-yl)ethyl)-3-(3-fluoro-4-((methylsulfonyl)methyl)phenyl)-1H-indole-2-carboxylic acid
IC-81: 7-[1-[5-[[(3S)-3-Aminopyrrolidin-1-yl]methyl]-2-oxo-oxazol-3-yl]ethyl]-3-[3-fluoro-4-(methylsulfonylmethyl)phenyl]-1H-indole-2-carboxylic acid
IC-82: 7-[1-[5-[[(3R)-3-Aminopyrrolidin-1-yl]methyl]-2-oxo-oxazol-3-yl]ethyl]-3-[3-fluoro-4-(methylsulfonylmethyl)phenyl]-1H-indole-2-carboxylic acid
IC-83: 7-(1-(5-(3-Aminopropyl)-2-oxooxazolidin-3-yl)ethyl)-3-(3-fluoro-4-((methylsulfonyl)methyl)phenyl)-1H-indole-2-carboxylic acid
IC-84: 7-(1-(5-(3-Aminopropyl)-2-oxooxazolidin-3-yl)ethyl)-3-(3-fluoro-4-((methylsulfonyl)methyl)phenyl)-1H-indole-2-carboxylic acid
IC-86: 7-(1-((4-(Aminomethyl)benzyl)oxy)ethyl)-3-(3-cyano-4-(methylsulfonamido)phenyl)-1H-indole-2-carboxylic acid
IC-87: (S)-7-(1-((4-(Aminomethyl)benzyl)oxy)ethyl)-3-(3-chloro-4-((methylsulfonyl)methyl)phenyl)-1H-indole-2-carboxylic acid
IC-88: (R)-7-(1-((4-(Aminomethyl)benzyl)oxy)ethyl)-3-(3-chloro-4-((methylsulfonyl)methyl)phenyl)-1H-indole-2-carboxylic acid
IC-89: 3-(3-Fluoro-4-((methylsulfonyl)methyl)phenyl)-7-(1-(piperidin-4-ylmethoxy)ethyl)-1H-indole-2-carboxylic acid
IC-90: 7-(1-((1,1-Dimethylpiperidin-1-ium-4-yl)methoxy)ethyl)-3-(3-fluoro-4-((methylsulfonyl)methyl)phenyl)-1H-indole-2-carboxylate IC-91: 7-(1-((1H-1,2,3-triazol-5-yl)methoxy)ethyl)-3-(3-fluoro-4-((methylsulfonyl)methyl)phenyl)-1H-indole-2-carboxylic acid
IC-92: 7-(1-(4-(Ammoniomethyl)phenoxy)ethyl)-3-(3-fluoro-4-((methylsulfonyl)methyl)phenyl)-1H-indole-2-carboxylate IC-93: 7-(1-((5-(3-Aminopropyl)oxazol-2-yl)amino)ethyl)-3-(3-fluoro-4-((methylsulfonyl)methyl)phenyl)-1H-indole-2-carboxylic acid
IC-94: 7-(1-((5-(2-aminoethyl)oxazol-2-yl)amino)ethyl)-3-(3-fluoro-4-((methylsulfonyl)methyl)phenyl)-1H-indole-2-carboxylic acid
IC-95: 7-(1-((4-(Aminomethyl)piperidine-1-carbonyl)oxy)ethyl)-3-(3-fluoro-4-((methylsulfonyl)methyl)phenyl)-1H-indole-2-carboxylic acid
IC-96: 7-(1-(((4-(Aminomethyl)phenyl)carbamoyl)oxy)ethyl)-3-(3-fluoro-4-((methylsulfonyl)methyl)phenyl)-1H-indole-2-carboxylic acid
IC-97: 7-(1-(4-(Aminomethyl)-N-methylbenzamido)ethyl)-3-(3-fluoro-4-((methylsulfonyl)methyl)phenyl)-1H-indole-2-carboxylic acid
IC-98: 7-[1-(5,6-Dihydroxy-1,3-dioxo-2,3-dihydro-1H-isoindol-2-yl)ethyl]-3-[3-fluoro-4-(methanesulfonylmethyl)phenyl]-1H-indole-2-carboxylic acid
IC-99: 7-[1-[[4-(Aminomethyl)cyclohexanecarbonyl]amino]ethyl]-3-[3-fluoro-4-(methylsulfonylmethyl) phenyl]-1H-indole-2-carboxylic acid
IC-100: 7-[1-[[2-(4-Aminocyclohexyl)acetyl]amino]ethyl]-3-[3-fluoro-4-(methylsulfonylmethyl)phenyl]-1H-indole-2-carboxylic acid
IC-101: 7-(1-(((4-(aminomethyl)phenoxy)carbonyl)amino)ethyl)-3-(3-fluoro-4-((methylsulfonyl)methyl)phenyl)-1H-indole-2-carboxylic acid
IC-102: (4-(3-(1-(2-Carboxy-3-(3-fluoro-4-((methylsulfonyl)methyl)phenyl)-1H-indol-7-yl)ethyl)ureido)phenyl)methanaminium chloride
IC-104: 3-[3-Fluoro-4-(methanesulfonylmethyl)phenyl]-7-(1-{[2-(1H-imidazol-4-yl)ethyl]carbamoyl}propan-2-yl)-1H-indole-2-carboxylic acid
IC-107: (2S,4r)-2-(Aminomethyl)-7-((S)-1-(3-(3-fluoro-4-((methylsulfonyl)methyl)phenyl)-2-(1H-tetrazole-5-yl)-1H-indol-7-yl)ethyl)-5-oxa-7-azaspiro[3.4]octan-6-one

Further compounds of the invention, or a pharmaceutically acceptable salt or solvate thereof, include:
7-((S)-1-((2S,4r)-2-(aminomethyl)-6-oxo-5-oxa-7-azaspiro[3.4]octan-7-yl)ethyl)-3-(1-methyl-1H-pyrazol-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(1-(cyanomethyl)-1H-pyrazol-4-yl)-1H-indole-2-carboxylic acid
3-(1-(2-amino-2-iminoethyl)-1H-pyrazol-4-yl)-7-((S)-1-((2S,4r)-2-(aminomethyl)-6-oxo-5-oxa-7-azaspiro[3.4]octan-7-yl)ethyl)-1H-indole-2-carboxylic acid
3-(1-(2-amino-2-oxoethyl)-1H-pyrazol-4-yl)-7-((S)-1-((2S,4r)-2-(aminomethyl)-6-oxo-5-oxa-7-azaspiro[3.4]octan-7-yl)ethyl)-1H-indole-2-carboxylic acid
7-((S)-1-((2S,4r)-2-(aminomethyl)-6-oxo-5-oxa-7-azaspiro[3.4]octan-7-yl)ethyl)-3-(isoxazol-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(3,5-dimethyl-1H-pyrazol-4-yl)-1H-indole-2-carboxylic acid
7. N07-7-((S)-1-((2S,4r)-2-(aminomethyl)-6-oxo-5-oxa-7-azaspiro[3.4]octan-7-yl)ethyl)-3-(3-oxo-2,3-dihydro-1H-pyrazol-4-yl)-1H-indole-2-carboxylic acid

The various functional groups and substituents making up the compounds of the formula I or formula II are typically chosen such that the molecular weight of the compound of the formula I or formula II does not exceed 1000. More usually, the molecular weight of the compound will be less than 900, for example less than 800, or less than 700, or less than 650, or less than 600. More preferably, the molecular weight is less than 550 and, for example, is 500 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn-Ingold-Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶O and ¹⁸O; and the like.

It is also to be understood that certain compounds of the formula I or formula II may exist in solvated as well as unsolvated forms such as, for example, hydrated forms. It is to be understood that the invention encompasses all such solvated forms that possess antiproliferative activity.

It is also to be understood that certain compounds of the formula I or formula II may exhibit polymorphism, and that the invention encompasses all such forms that possess antiproliferative activity.

Compounds of the formula I or formula II may exist in a number of different tautomeric forms and references to compounds of the formula I or formula II include all such forms. For the avoidance of doubt, where a compound can exist in one of several tautomeric forms, and only one is specifically described or shown, all others are nevertheless embraced by formula I or formula II. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

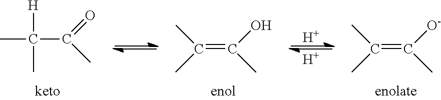

Compounds of the formula I or formula II containing an amine function may also form N-oxides. A reference herein to a compound of the formula I or formula II that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula I or formula II may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula I or formula II and in-vivo cleavable amide derivatives that may be formed at a carboxy group or an amino group in a compound of the formula I or formula II.

Accordingly, the present invention includes those compounds of the formula I or formula II as defined hereinbefore when made available by organic synthesis and when made available within the human or animal body by way of cleavage of a pro-drug thereof. Accordingly, the present invention includes those compounds of the formula I or formula II that are produced by organic synthetic means and also such compounds that are produced in the human or animal body by way of metabolism of a precursor compound, that is a compound of the formula I or formula II may be a synthetically-produced compound or a metabolically-produced compound.

A suitable pharmaceutically acceptable pro-drug of a compound of the formula I or formula II is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula I or formula II that possesses a carboxy group is, for example, an in vivo cleavable ester thereof. An in vivo cleavable ester of a compound of the formula I or formula II containing a carboxy group is, for example, a pharmaceutically acceptable ester which is cleaved in the human or animal body to produce the parent acid. Suitable pharmaceutically acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the formula I or formula II that possesses a hydroxy group is, for example, an in vivo cleavable ester or ether thereof. An in vivo cleavable ester or ether of a compound of the formula I or formula II containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N-(C₁0.6)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula I or formula II that possesses a carboxy group is, for example, an in vivo cleavable amide thereof, for example an amide formed with an amine such as ammonia, a C₁₋₄alkylamine such as methylamine, a (C₁₋₄alkyl)₂amine such as dimethylamine, N-ethyl-N-methylamine or diethylamine, a C₁₋₄alkoxy-C₂₋₄alkylamine such as 2-methoxyethylamine, a phenyl-C₁₋₄ alkylamine such as benzylamine and amino acids such as glycine or an ester thereof.

A suitable pharmaceutically acceptable pro-drug of a compound of the formula I or formula II that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula I or formula II may be exerted in part by one or more metabolites that are formed within the human or animal body after administration of a compound of the formula I or formula II. As stated hereinbefore, the in vivo effects of a compound of the formula I or formula II may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of formula I or formula II will vary depending on the nature of R₂, R₃, R₇, Rₐ and R_{b} and any substituent groups associated therewith. Suitable processes for their preparation are described further in the accompanying Examples.

Once a compound of formula I or formula II has been synthesised by any one of the processes defined herein, the processes may then further comprise the additional steps of:
(i) removing any protecting groups present;
(ii) converting the compound formula I formula II into another compound of formula I or formula II;
(iii) forming a pharmaceutically acceptable salt, hydrate or solvate thereof; and/or
(iv) forming a prodrug thereof.

An example of (ii) above is when a compound of formula I or formula II is synthesised and then one or more of the groups R₂, R₃, R₇, Rₐ and R_{b} may be further reacted to change the nature of the group and provide an alternative compound of formula I.

The resultant compounds of formula I or formula II can be isolated and purified using techniques well known in the art.

### Biological Activity

The enzyme and in-vitro cell-based assays described in accompanying Example section, or elsewhere in the literature, may be used to measure the pharmacological effects of the compounds of the present invention.

Although the pharmacological properties of the compounds of formula I or formula II vary with structural change, as expected, the compounds of the invention were found to be active in these enzyme assays.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier. For example, solid oral forms may contain, together with the active compound, diluents, such as, for example, lactose, dextrose, saccharose, cellulose, corn starch or potato starch; lubricants, such as, for example, silica, talc, stearic acid, magnesium or calcium stearate, and/or polyethylene glycols; binding agents; such as, for example, starches, arabic gums, gelatin, methylcellulose, carboxymethylcellulose or polyvinyl pyrrolidone; disaggregating agents, such as, for example, starch, alginic acid, alginates or sodium starch glycolate; effervescing mixtures; dyestuffs; sweeteners; wetting agents, such as, for example, lecithin, polysorbates, laurylsulphates; and, in general, non toxic and pharmacologically inactive substances used in pharmaceutical formulations. Such pharmaceutical compositions may be manufactured in by conventional methods known in the art, such as, for example, by mixing, granulating, tableting, sugar coating, or film coating processes.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing). Suitably, oral or parenteral administration is preferred. Most suitably, oral administration is preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I or formula II will naturally vary according to the nature and severity of the condition, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The compounds of the present invention are inhibitors of metallo-beta-lactamases (MBLs). Many bacteria have developed resistance to β-lactam antibacterials (BLAs) and one of the main resistance mechanisms is the hydrolysis of BLAs by MBLs. Thus, the inhibition of bacterial MBLs by the compounds of the present invention can significantly enhance the activity of BLAs, when administered with a compound of the present invention.

The present invention provides compounds that function as inhibitors of metallo-beta-lactamases.

The present invention therefore provides a method of inhibiting bacterial metallo-beta-lactamase activity in vitro or in vivo, said method comprising contacting a cell with an effective amount of a compound, or a pharmaceutically acceptable salt, hydrate or solvate thereof, or a pharmaceutical composition as defined herein.

The present invention also provides a method for the prevention or treatment of bacterial infection in a patient in need of such treatment, said method comprising administering to said patient a therapeutically effective amount of a compound, or a pharmaceutically acceptable salt, hydrate or solvate thereof, or a pharmaceutical composition as defined herein, in combination with a suitable antibacterial agent.

In a preferred embodiment, the antibacterial agent is a β-lactam antibacterial agent, or analogue thereof. Non limiting examples of suitable R-lactam antibacterial agents include carbapenems (e.g. meropenem, faropenem, imipenem, ertapenem, doripenem, panipenem/betamipron and biapenem as well as razupenem, tebipenem, lenapenem and tomopenem), ureidopenicillins (e.g. piperacillin), carbacephems (e.g. loracarbef) and cephalosporins (e.g. cefpodoxime, ceftazidime, cefotaxime, ceftriaxone, ceftobiprole, and ceftaroline). Specific examples of suitable β-lactam antibacterial agents include, for example, temocillin, piperacillin, cefpodoxime, ceftazidime, cefotaxime, ceftriaxone, meropenem, faropenem, imipenem, loracarbef, ceftobiprole and ceftaroline.

The present invention also provides a method of inhibiting bacterial infection, in vitro or in vivo, said method comprising contacting a cell with an effective amount of a compound, or a pharmaceutically acceptable salt, hydrate or solvate thereof, as defined herein, in combination with a suitable antibacterial agent.

The present invention also provides a compound, or a pharmaceutically acceptable salt, hydrate or solvate thereof, or a pharmaceutical composition as defined herein for use in therapy.

The present invention also provides a compound, or a pharmaceutically acceptable salt, hydrate or solvate thereof, or a pharmaceutical composition as defined herein for use in the treatment of a bacterial infection. In one embodiment, the treatment may be prophylactic (i.e. intended to prevent disease).

The present invention provides a compound, or a pharmaceutically acceptable salt, hydrate or solvate thereof, as defined herein for use in the inhibition of metallo-beta-lactamase activity.

Furthermore, the present invention provides a compound, or a pharmaceutically acceptable salt, hydrate or solvate thereof, as defined herein for use in the treatment of a disease or disorder in which metallo-beta-lactamase activity is implicated.

The present invention also provides a kit of parts comprising a compound, or a pharmaceutically acceptable salt, hydrate or solvate thereof, or a pharmaceutical composition as defined herein, and a BLA and/or a BLA linked to a formula (I) or formula (II) compound.

The present invention also provides a conjugate comprising a compound of formula (I) or formula (II) linked to an antibacterial agent via a suitable linker. The antibacterial agent may be any of the beta-lactam antibiotics described herein.

The compounds of the present invention may be linked to the antibacterial agent by means of one or more covalent bonds or linker groups formed between the antibacterial agent and any suitable position on the compound of Formula (I) or Formula (II).

Any suitable linking group that is capable of connecting the compound of Formula I or Formula (II) to the antibacterial agent be used. Suitable linking groups are well known in the art. The compounds of the invention may also be associated with the antibacterial agent by means of one or more ionic or covalent interactions.

Examples of suitable linking groups are known in the art and include, but are not limited to, alkyl and aryl groups, including substituted alkyl and aryl groups and heteroalkyl (particularly oxo groups) and heteroaryl groups, including alkylamine groups.

In particular embodiments, the linker is cleavable in vivo.

The term “bacterial infection” will be understood to refer to the invasion of bodily tissue by any pathogenic microorganisms that proliferate, resulting in tissue injury that can progress to disease. Suitably, the pathogenic microorganism is a bacteria.

The bacterial infection may be caused by Gram-negative or Gram-positive bacteria.

For example, the bacterial infection may be caused by bacteria from one or more of the following families; Clostridium, Pseudomonas, Escherichia, Klebsiella, Enterococcus, Enterobacter, Serratia, Stenotrophomonas, Aeromonas, Morganella, Yersinia, Salmonella, Proteus, Pasteurella, Haemophilus, Citrobacter, Burkholderia, Brucella, Moraxella, Mycobacterium, Streptococcus or Staphylococcus. Particular examples include Clostridium, Pseudomonas, Escherichia, Klebsiella, Enterococcus, Enterobacter, Streptococcus and Staphylococcus. The bacterial infection may, for example, be caused by one or more bacteria selected from Moraxella catarrhalis, Brucella abortus, Burkholderia cepacia, Citrobacter species, Escherichia coli, Haemophilus Pneumonia, Kiebsiella Pneumonia, Pasteurella multocida, Proteus mirabilis, Salmonella typhimurium, Clostridium difficile, Yersinia enterocolitica Mycobacterium tuberculosis, Staphylococcus aureus, group B streptococci, Streptococcus Pneumonia, and Streptococcus pyogenes, e.g. from E. coli and K. pneumoniae.

It will be understood by a person skilled in the art that the patient in need thereof is suitably a human, but may also include, but is not limited to, primates (e.g. monkeys), commercially farmed animals (e.g. horses, cows, sheep or pigs) and domestic pets (e.g. dogs, cats, guinea pigs, rabbits, hamsters or gerbils). Thus the patient in need thereof may be any mammal that is capble of being infected by a bacterium.

### Routes of Administration

The compounds of the present invention, or pharmaceutical compositions comprising these compounds, may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Diagnostic Uses

The compounds of the present invention, or pharmaceutical compositions comprising these compounds in combination with a suitable antibacterial agent, may also be used in methods for the detection of metallo-beta-lactamases. It will be appreciated that the compounds of formula (I) or formula (II) may be modified to enable various types of assays known is the literature, such as those using spectroscopic such as fluorescence or luminescence based methods. Thus, in one variation a sample containing bacteria which is suspected of expressing MBLs can be cultured (a) in the presence of a beta-lactam antibiotic agent; and (b) in the presence of the antibiotic combination of the invention. If the bacteria are seen to grow under conditions (a), this suggests that a beta-lactamase, able to hydrolyse the antibiotic agent, is causing resistance of the bacteria to the antibiotic agent. However, if the bacteria do not grow under condition (b), i.e. in the presence of compound of the present invention and a suitable antibacterial agent, then the beta-lactamases present have been inhibited. Such a result suggests that the beta-lactamases are metallo-beta-lactamases. The method can be used to determine whether bacteria express metallo-beta-lactamase enzymes.

### EXAMPLES

Carbapenems are vital medicines, but their efficacy is increasingly compromised by metallo-β-lactamases (MBLs). A high-throughput screen for NDM-1 inhibitors identified indole-2-carboxylates (InCs) as potential β-lactamase stable β-lactam mimics. Subsequent SAR studies reveal InCs as a new class of potent MBL inhibitor, active against all MBL classes of major clinical relevance. Crystallographic studies reveal the InC binding mode to MBLs mimics that of intact β-lactams, including with respect to maintenance of a Zn(II) bound hydrolytic water. InCs restore carbapenem activity against multiple drug resistant Gram-negative bacteria and have a low frequency of resistance. InCs have a good in-vivo safety profile and combined with meropenem show strong in-vivo efficacy in peritonitis and thigh mouse infection models. The results highlight the potential for clinical utilisation of InC-carbapenem combinations and of mechanism-guided approaches to combatting globally disseminated resistant mechanisms.

Imitation of the initial substrate binding mode has been successfully employed for SBL inhibition (e.g. as occurs with clavulanate) and that β-lactam antibiotics are mimics of the substrates of their transpeptidase targets(9, 21-24) The inventors have identified and optimised indole carboxylates (InCs) as new broad spectrum MBLi. InCs protect carbapenems from MBL activity in MDR and XDR (extensively drug-resistant) Gram-negative pathogens in vitro and in vivo mouse infection models.

### 1 Synthetic Chemistry

### 1.1 General Information

Reagents were from commercial suppliers and used as supplied. Solvents (including dried solvents) for chemical transformations, work-up and chromatography were from Sigma-Aldrich (HPLC grade) or Acros Organics, and were used without purification. Silica gel 60 F254 analytical thin layer chromatography (TLC) plates were from Merck (Darmstadt, Germany) and visualized under UV light and/or with potassium permanganate stain. Chromatographic purifications were performed using prepacked SNAP columns and a Biotage Isolera Purification system. Deuterated solvents were from Sigma-Aldrich. All ¹H and ¹³C NMR spectra were recorded using a BrukerAVIII HD 400, AVII 500 or AVIII 600 spectrometers (400, 500 or 600 MHz for ¹H NMR and 101, 126 or 151 MHz for ¹³C NMR). Chemical shifts (δ_{H}) are in parts per million (ppm) and are referenced to the residual solvent peak, and coupling constants (J) are reported in Hertz (Hz) and are reported to the nearest 0.5 Hz. Low resolution mass spectra (m/z) were recorded using a Waters LCT Premier spectrometer using electrospray ionisation (ESI). High resolution mass spectra were recorded using a Bruker μTOF (ESI) spectrometer by the internal service at the Department of Chemistry, University of Oxford. The m/z values are reported in Daltons. Compound names are those generated by MestReNova v14.1 software following IUPAC nomenclature.

Note on stereochemistry: Unless explicitly drawn with wedged bonds compounds were prepared as racemic mixtures.

### 1.2 Synthesis and Characterization of Indole Carboxylates and Intermediates

### 1.2.1 C7 Modification

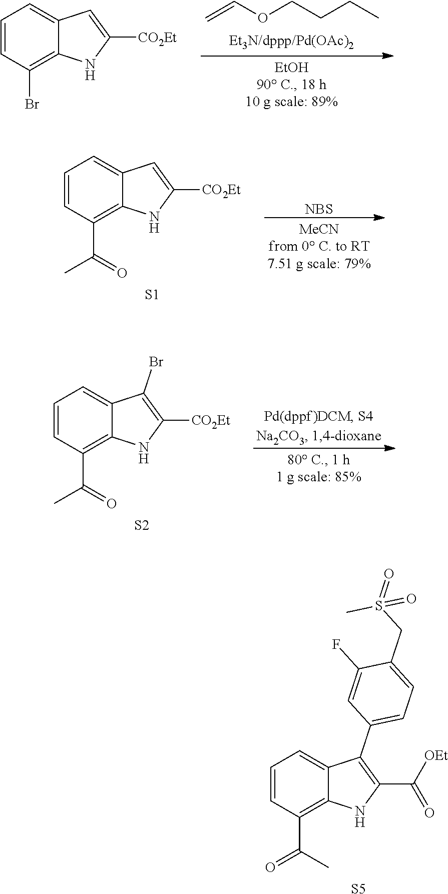

### Ethyl 7-acetyl-1H-indole-2-carboxylate (S1)

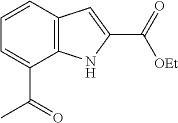 [SMILES: CCOC(=O)c1cc2cccc(C(C)=O)c2[nH]1]

To ethyl 7-bromo-1H-indole-2-carboxylate (10.0 g, 37.3 mmol, 1 eq), Pd(OAc)₂ (251 mg, 1.12 mmol, 0.03 eq) and 1,3-bis(diphenylphosphino)propane (DPPP, 923 mg, 2.24 mmol, 0.06 eq) was added degassed EtOH (75 ml) at room temperature under Ar atmosphere, followed by triethylamine (15.6 mL, 112 mmol, 3.0 eq) and butyl vinyl ether (15.1 mL, 112 mmol, 3.0 eq). The reaction mixture was then heated to 80° C. for 18 h and then the volatiles removed in vacuo and the residue was redissolved in 2:1 THF: CH₂Cl₂ (110 mL) and treated with 4 M HCl (40 mL) and stirred for 1 h. The yellow reaction mixture was then diluted with EtOAc (500 mL) and the organic layer washed with H₂O (200 mL), sat. NaHCO₃ (200 mL), brine (200 mL) and dried over Na₂SO₄, filtered and evaporated to dryness. The yellow oil was washed with hexane (100 mL) and the yellow precipitate was filtered off and dried in vacuo to afford the desired compound S1 in 89% yield as an off-white solid (7.70 g).

¹H NMR (400 MHz, CDC₃) δ ppm 10.80 (1H, br. s, NH), 7.93 (1H, dt, J=8.0, 0.9 Hz, Ar—H), 7.89 (1H, dd, J=7.5, 1.0 Hz, Ar—H), 7.26 (1H, d, J=2.4 Hz, Ar—H), 7.21 (1H, t, J=7.7 Hz, Ar—H), 4.43 (2H, q, J=7.1 Hz, OCH₂CH₃), 2.71 (3H, s, CH₃), 1.43 (3H, t, J=7.1 Hz, OCH₂CH₃); ¹³C NMR (101 MHz, CDC₃) δ ppm 199.8, 161.4, 135.3, 129.4, 129.1, 128.9, 128.2, 121.1, 120.0, 108.4, 61.2, 26.6, 14.5; LRMS [M+H]*232.1.

### Ethyl 7-acetyl-3-bromo-1H-indole-2-carboxylate (S2)

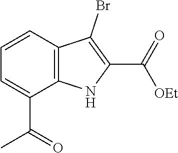 [SMILES: CCOC(=O)c1[nH]c2c(C(C)=O)cccc2c1Br]

To a solution of indole S1 (7.51 g, 32.5 mmol) in MeCN (105 mL) at 0° C. was added N-bromosuccinimide (6.07 g, 34.1 mmol, 1.05 eq) and the reaction mixture was warmed to room temperature and stirred for 1 h. The reaction mixture was then diluted with EtOAc (300 mL) and the organic layer was washed sequentially with 10% Na₂S₂O₃ (2×100 mL), H₂O (4×100 mL) and brine (200 mL), dried over Na₂SO₄ and filtered through a pad of silica gel and concentrated in vacuo to give the product as a yellow solid which was purified by recrystallization (from hot 50 mL heptane and 7 mL EtOAc) to afford the desired compound S2 in 79% yield as yellow plates (7.99 g).

¹H NMR (300 MHz, CDC₃) δ ppm 10.87 (1H, s, N—H), 7.98-7.91 (2H, m, Ar—H), 7.30 (1H, dd, J=8.1, 7.4 Hz, Ar—H), 4.47 (2H, q, J=7.1 Hz, OCH₂CH₃), 2.72 (3H, s, CH₃), 1.46 (3H, t, J=7.1 Hz, OCH₂CH₃); ¹³C NMR (101 MHz, CDC₃) δ ppm 199.7, 160.2, 133.9, 129.3, 129.2, 127.7, 125.7, 121.1, 120.6, 98.5, 61.6, 26.6, 14.45; LCMS [M+H]⁺ 310.0.

### 4-Bromo-2-fluoro-1-((methylsulfonyl)methyl)benzene (S3)

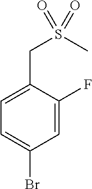

Degassed EtOH (22 mL) was added to a mixture of 4-bromo-1-(bromomethyl)-2-fluorobenzene (2.95 g, 11.0 mmol) and sodium methanesulfinate (1.46 g, 14.3 mmol, 1.3 eq) at rt. The resultant reaction mixture stirred at 80° C. for 1 h before being concentrated in vacuo. The residue was partitioned between EtOAc and H₂O and the organic layer was washed additionally with H₂O (×3), brine, dried over MgSO₄ and concentrated in vacuo. The crude mixture was washed on a P3 frit (eluent hexane/Et₂O, 1:1) and concentrated in vacuo to give the desired product S3 in 92% yield as a colorless solid (2.71 g).

¹H NMR (300 MHz, CDC₃) δ ppm 7.42-7.33 (3H, m, Ar—H), 4.27 (2H, s, CH₂SO₂CH₃), 2.83 (3H, br q, J=0.9 Hz, CH₃); LRMS [M-SO₂Me]⁺ 186.9.

### 2-(3-Fluoro-4-((methylsulfonyl)methyl)phenyl)-4,4,5,5-tetramethyl-1,3,2-dioxaborolane (S4)

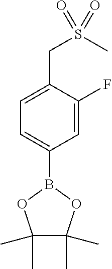

Argn was bubbled through a mixture of arylbromide S3 (2.55 g, 9.55 mmol), bis(pinacolato)diboron (2.67 g, 10.5 mmol, 1.1 eq), potassium acetate (2.81 g, 28.6 mmol) and Pd(dppf)Cl₂·DCM (234 mg, 0.286 mmol, 0.03 eq) in 1,4-dioxane (38 mL) at rt for 10 min. The resultant reaction mixture was left to stir at 100° C. for 2 h then cooled to rt, poured into Et₂O (100 mL) and stirred at rt for 30 min. The solution was then filtered through a silica plug (eluent Et₂O) and concentrated in vacuo. The residue was purified by flash column chromatography (eluent hexane/EtOAc, 2:1 to 1:1) followed by recrystallization from a minimum amount of toluene. The crystals were filtered off, washed with cold toluene (×2) and hexane. The desired product S4 was obtained in 88% yield as large beige prisms (2.40 g).

¹H NMR (300 MHz, CDCl₃) δ ppm 7.63 (1H, dd, J=7.4, 1.1 Hz, Ar—H), 7.55 (1H, dd, J=10.1, 1.1 Hz, Ar—H), 7.49 (1H, t, J=7.4 Hz, Ar—H), 4.33 (2H, s, CH₂SO₂CH₃), 2.79 (3H, br q, J=0.9 Hz, CH₃), 1.34 (12H, s, 4×CH₃); LCMS [M+H]⁺ 315.0.

### Ethyl 7-acetyl-3-(3-fluoro-4-((methylsulfonyl)methyl)phenyl)-1H-indole-2-carboxylate (S5)

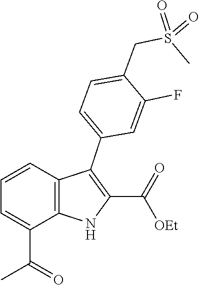 [SMILES: CCOC(=O)c1[nH]c2c(C(C)=O)cccc2c1-c1ccc(CS(C)(=O)=O)c(F)c1]

A suspension of bromoindole S2 (1.00 g, 3.22 mmol, 1.0 eq), the pinacol ester S4 (1.11 g, 3.55 mmol, 1.1 eq) and Pd(dppf)Cl₂-DCM (132 mg, 0.16 mmol, 0.05 eq) in dioxane (26 mL) was degassed by bubbling Ar for 10 min at RT and then a solution of 2 M aq Na₂CO₃ (7 mL) was added. The reaction mixture was stirred for 1 h at 80° C. and then cooled to RT and was quenched by the addition of ammonium pyrrolidinedithiocarbamate (80 mg, 0.48 mmol, 0.15 eq) in H₂O (1 mL) and stirred for 30 min; then partitioned between CH₂Cl₂ and H₂O, filtered through Celite®, and the organic layer was washed with H₂O, brine, dried (MgSO₄) and the volatiles removed in vacuo. Purification by FCC (CH₂Cl₂: EtOAc 20:1) followed by washing of the product with cold EtOAc (−78° C.) afforded the desired compound S5 in 85% yield as a colorless solid (1.14 g).

¹H NMR (300 MHz, CDCl₃) δ ppm 10.98 (1H, s, N—H), 7.97 (1H, dd, J=7.5, 1.0 Hz, Ar—H), 7.88 (1H, d, J=8.1, 0.9 Hz, Ar—H), 7.59 (1H, t, J=8.0 Hz, Ar—H), 7.46-7.34 (2H, m, Ar—H), 7.27 (1H, t, J=7.3 Hz, Ar-H), 4.40 (2H, s, CH₂SO₂CH₃), 4.35 (2H, q, J=7.1 Hz, OCH₂CH₃), 2.90 (3H, d, J=7.3 Hz, CH₃), 2.75 (3H, s, CH₂SO₂CH₃), 1.31 (3H, t, J=7.2 Hz, OCH₂CH₃); ¹³C NMR (101 MHz, CDCl₃) δ ppm 199.9 (C═O), 160.9 (COOEt), 160.5 (d, J_{FC}=247.8 Hz, ArCF), 136.9 (d, J_{FC}=9.0 Hz), 134.2, 132.3 (d, J_{FC}=3.4 Hz), 128.9, 128.9, 127.8, 127.5 (d, J_{FC}=3.3 Hz), 124.8, 121.7 (d, J_{FC}=2.1 Hz), 121.1, 120.7, 118.2 (d, J_{FC}=22.4 Hz) 114.8 (d, J_{FC}=14.8 Hz), 61.4 (OCH₂CH₃), 54.3 (d, J_{FC}=2.8 Hz S(═O)₂CH₂), 39.6 (d, J_{FC}=2.4 Hz, S(═O)₂CH₃), 26.7 (C(═O)CH₃), 14.3 (OCH₂CH₃); LCMS [M+H]⁺ 418.03; HRMS (TOF, ESI⁺) m/z: [M+Na]⁺ Calcd for C₂₁H₂₀FNO₅SNa 440.0938; Found 440.0951.

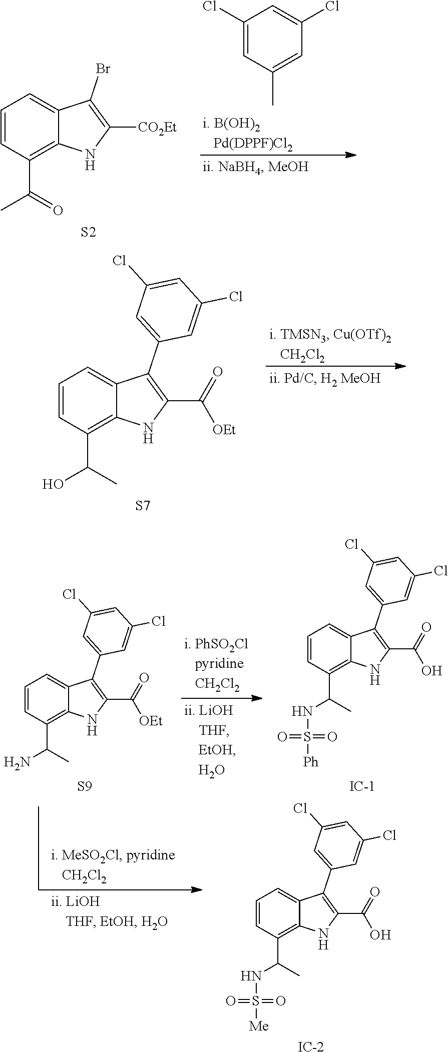

A suspension of bromoindole S2 (99 mg, 0.320 mmol), 3,5-dichlorophenylboronic acid (73 mg, 0.38 mmol) and Pd(dppf)Cl₂·DCM (12 mg, 0.016 mmol, 0.05 eq) in dioxane (3 mL) was degassed by bubbling Ar for 10 min at RT and then a solution of 2 M aq Na₂CO₃ (0.75 mL) was added. The reaction mixture was heated in the microwave for 1 hour at 100° C. and then cooled to RT, filtered through Celite® (washing with EtOAc). The reaction mixture was diluted with 1 M aq HCl, and the aq layer was extracted with EtOAc; the combined organics were washed (brine), dried (MgSO₄) and the volatiles removed in vacuo. Purification by FCC (KP-Sil 25 g, 2% to 8% EtOAc in cyclohexane) afforded the desired product S6 in 72% yield (87 mg).

To the ketone S6 in MeOH at −10° C. was added NaBH₄ and the reaction was monitored by TLC. Upon complete consumption of starting material (SM), the reaction was quenched by the addition of sat NaHCO₃ and CH₂Cl₂ and stirring was maintained for 5 minutes. The layers were separated and the aqueous layer was extracted with CH₂Cl₂; the combined organic phases were dried (MgSO₄), filtered and concentrated in vacuo. Purification of the crude mixture by FCC (KP-Sil 25 g, cyclohexane: EtOAc 5-30%) afforded the alcohol S7 as a colourless solid in 82% yield (332 mg).

To the alcohol S7 (378 mg, 1.00 mmol) in CH₂Cl₂ (5 ml) was added TMSN₃ (0.160 mL, 1.20 mmol) followed by Cu(OTf)₂ (18.1 mg, 0.050 mmol) and the reaction was stirred at rt for 1 h whereupon it was quenched by the addition of H₂O. The aqueous layer was extracted with CH₂Cl₂, the combined organics were washed with brine, dried (MgSO₄) and concentrated. Purification of the crude mixture by FCC (KP-Sil 25 g, cyclohexane: EtOAc 0-20%) afforded the azide S8 as a colourless solid in 82% yield (332 mg).

¹H NMR (400 MHz, CDCl₃) δ 9.48 (s, 1H), 7.54 (dt, J=8.1, 1.0 Hz, 1H), 7.45-7.34 (m, 7H), 7.29-7.21 (m, 8H), 7.15 (dd, J=8.1, 7.2 Hz, 1H), 4.98 (q, J=6.8 Hz, 1H), 4.31 (q, J=7.1 Hz, 2H), 1.71 (d, J=6.9 Hz, 3H), 1.27 (t, J=7.1 Hz, 3H); ¹³C NMR (101 MHz, CDCl₃) δ 161.7, 136.6, 134.3, 113.0, 129.3, 128.5, 127.4, 124.7, 123.8, 123.5, 121.4, 121.3, 121.1, 61.5, 59.9, 20.5, 14.1; LRMS (TOF, ESI⁻) m/z: [M−H]⁻ Calcd for C₁₉H₁₅Cl₂N₄O₂ 401.1; Found 401.0.

To the azido indole S8 (418 mg, 1.04 mmol) in MeOH (15 ml) was added Pd/C (110 mg) and the reaction was placed under a hydrogen atmosphere (balloon pressure) and was stirred at RT until completion (by TLC analysis). The reaction mixture was filtered through a plug of silica, eluting with CH₂Cl₂ and the volatiles were removed in vacuo. Purification of the crude mixture by FCC (KP-Sil 25 g, CH₂Cl₂: MeOH 0-7%) afforded the amine S9 as a colourless foam in 76% yield (297 mg).

¹H NMR (400 MHz, CDCl3) δ 10.81 (s, 1H), 7.47 (dd, J=8.0, 1.2 Hz, 1H), 7.45 (d, J=1.9 Hz, 2H), 7.36 (t, J=1.9 Hz, 1H), 7.18-7.06 (m, 2H), 4.58 (q, J=6.5 Hz, 1H), 4.31 (q, J=7.2 Hz, 2H), 1.54 (d, J=6.6 Hz, 3H), 1.28 (t, J=7.2 Hz, 3H); ¹³C NMR (101 MHz, CDCl₃) δ 161.9, 137.3, 134.4, 134.2, 130.1, 129.3, 128.1, 127.0, 123.1, 122.9, 121.3, 120.5, 119.6, 61.2, 51.3, 25.2, 14.2; LRMS (TOF, ESI⁻) m/z: [M−H]⁻ Calcd for C₁₉H₁₇Cl₂N₂O₂ 375.1; Found 375.0.

### IC-1: 3-(3,5-Dichlorophenyl)-7-(1-(phenylsulfonamido)ethyl)-1H-indole-2-carboxylic acid

To the amine amine S9 (80 mg, 0.21 mmol) in CH₂Cl₂ (3 mL) at 0° C. was added pyridine (21 μL, 0.25 mmol) followed by phenylsulfonyl chloride (30 μL, 0.23 mmol). The reaction mixture was warmed to RT and upon completion, as judged by TLC analysis, the reaction was quenched by the addition of MeOH, filtered through a plug of silica, eluting with EtOAc, and the volatiles were removed under reduced pressure. Purification of the crude mixture by FCC (KP-Sil 10 g, CH₂Cl₂: MeOH 0-5%) afforded the desired product as a colourless foam in 50% yield (54 mg).

¹H NMR (400 MHz, CDCl₃) δ 10.37 (s, 1H), 7.90-7.80 (m, 2H), 7.59-7.49 (m, 2H), 7.47-7.40 (m, 4H), 7.38 (t, J=1.9 Hz, 1H), 7.18 (dt, J=7.2, 1.0 Hz, 1H), 7.09 (dd, J=8.1, 7.2 Hz, 1H), 4.99 (p, J=7.1 Hz, 1H), 4.83 (d, J=8.1 Hz, 1H), 4.34 (qd, J=7.2, 1.2 Hz, 2H), 1.42 (d, J=6.8 Hz, 3H), 1.33 (t, J=7.1 Hz, 3H).

To the ethyl ester (47 mg, 0.091 mmol) in 3:1:1 THF: MeOH: H₂O (2.5 mL) was added LiOH·H₂O (19 mg, 0.45 mmol, 5 eq). Upon completion of the reaction, as judged by TLC analysis the mixture was acidified with 1 M KHSO₄ and the aqueous layer was extracted with Et₂O; the combined organics were washed with brine, dried (MgSO₄) and concentrated. Purification of the crude reaction mixture by FCC (KP-Sil 10 g, cyclohexane: EtOAc 10-30%, 3% AcOH) afforded the desired indole carboxylate IC-1 as a colourless solid in 83% yield (37 mg).

¹H NMR (400 MHz, MeOD) δ 7.63 (dt, J=8.2, 1.1 Hz, 2H), 7.46-7.36 (m, 4H), 7.31-7.18 (m, 4H), 7.03 (t, J=7.7 Hz, 1H), 5.05 (q, J=6.9 Hz, 1H), 1.48 (d, J=6.8 Hz, 3H).

### IC-2: 3-(3,5-Dichlorophenyl)-7-(1-(methylsulfonamido)ethyl)-1H-indole-2-carboxylic acid

To the amine amine S9 (80 mg, 0.21 mmol) in CH₂Cl₂ (3 mL) at 0° C. was added pyridine (21 μL, 0.25 mmol) followed by methanesulfonyl chloride (18 μL, 0.23 mmol). The reaction mixture was warmed to RT and upon completion, as judged by TLC analysis, the reaction was quenched by the addition of MeOH, filtered through a plug of silica, eluting with EtOAc, and the volatiles were removed under reduced pressure. Purification of the crude mixture by FCC (KP-Sil 10 g, CH₂Cl₂: MeOH 0-5%) afforded the sulfonamide as a pale-yellow foam in 51% yield (49 mg).

¹H NMR (400 MHz, CDCl₃) δ 10.28 (s, 1H), 7.57 (dd, J=8.2, 0.9 Hz, 1H), 7.45-7.36 (m, 2H), 7.31 (dt, J=7.3, 0.9 Hz, 1H), 7.17 (dd, J=8.1, 7.2 Hz, 1H), 5.13 (p, J=7.1 Hz, 1H), 4.79 (d, J=7.8 Hz, 1H), 4.31 (qd, J=7.2, 0.9 Hz, 2H), 2.94 (s, 2H), 1.79 (d, J=6.9 Hz, 3H), 1.28 (t, J=7.5 Hz, 3H).

To the ethyl ester (41 mg, 0.090 mmol) in 3:1:1 THF: MeOH: H₂O (2.5 mL) was added LiOH·H₂O (19 mg, 0.45 mmol, 5 eq). Upon completion of the reaction, as judged by TLC analysis the mixture was acidified with 1 M KHSO₄ and the aqueous layer was extracted with Et₂O; the combined organics were washed with brine, dried (MgSO₄) and concentrated. Purification of the crude reaction mixture by FCC (KP-Sil 10 g, cyclohexane: EtOAc 20-40%, 3% AcOH) afforded the desired indole carboxylate IC-2 as a colourless solid in 63% yield (39 mg).

¹H NMR (400 MHz, MeOD) δ 7.49-7.34 (m, 4H), 7.17 (dd, J=8.1, 7.2 Hz, 1H), 5.19 (q, J=6.9 Hz, 1H), 2.78 (s, 3H), 1.66 (d, J=7.0 Hz, 3H).

### 1.2.2 Reductive Hydrazone Coupling

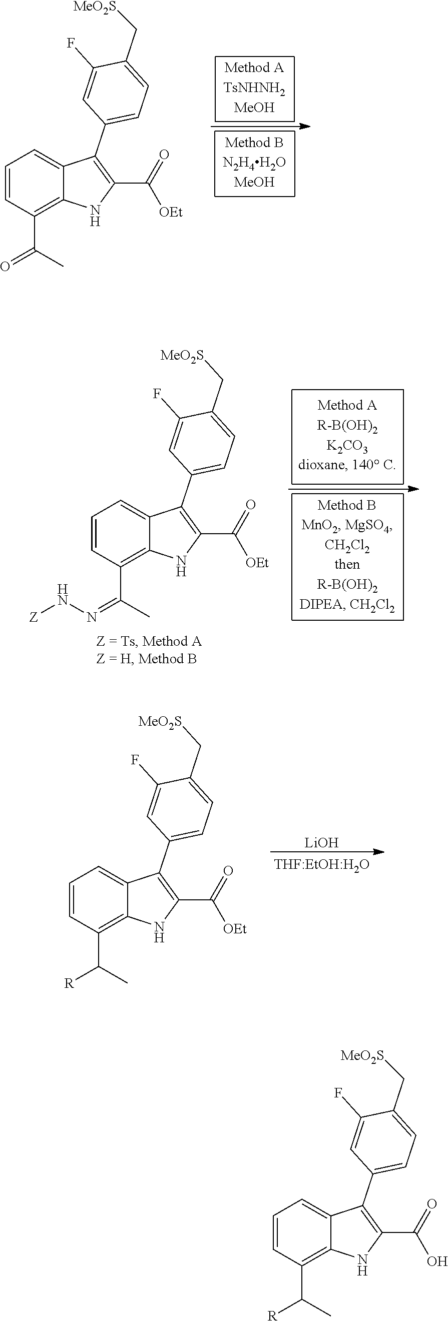

### Ethyl-3-(3-fluoro-4-((methylsulfonyl)methyl)phenyl)-7-(1-(2-tosylhydrazine ylidene)ethyl)-1H-indole-2-carboxylate

To the ketone S5 (458 mg, 1.10 mmol) in MeOH (5.5 mL) and CH₂Cl₂ (1 mL) was added TsNHNH₂ (225 mg, 1.21 mmol, 1.10 eq) at RT; no conversion was observed after 4 hours and then pTSA (50 mg) was added and the reaction was warmed to 40° C. for 2 hours. TLC analysis at this stage showed complete consumption of SM (3:2 cyclohexane (CyH): EtOAc); the volatiles were removed in vacuo and the reaction mixture was triturated with minimal MeOH to afford the desired compound S10 as an off-white solid in 81% yield (520 mg).

¹H NMR (400 MHz, CDCl₃) δ 11.27 (s, 1H), 8.14-7.99 (m, 2H), 7.77 (s, 1H), 7.72-7.64 (m, 1H), 7.58 (t, J=7.8 Hz, 1H), 7.52 (dd, J=7.5, 0.9 Hz, 1H), 7.47-7.41 (m, 2H), 7.33-7.28 (m, 1H), 7.17 (dd, J=8.1, 7.5 Hz, 1H), 4.44 (q, J=7.1 Hz, 2H), 4.40 (s, 2H), 2.90 (d, J=0.8 Hz, 3H), 2.38 (s, 3H), 2.30 (s, 3H), 1.40 (t, J=7.1 Hz, 3H).

### Method a for Reductive Tosyl Hydrazone Coupling

To a sealable vial, was added the tosyl hydrazone (1 eq), the boronic acid (3.0 eq) and K₂CO₃ (3.0 eq), then 1,4-dioxane (0.2 M); the vial was sealed and the reaction was heated at 140° C. and stirred for 16 h. The reaction mixture was cooled, diluted with CH₂Cl₂ and sat aq NaHCO₃, then extracted with CH₂Cl₂, dried (Na₂SO₄) and the volatiles were removed in vacuo and the crude mixture purified.

### IC-3: 3-(3-Fluoro-4-((methylsulfonyl)methyl)phenyl)-7-(1-(1-methyl-1H-pyrazol-4-yl)ethyl)-1H-indole-2-carboxylic acid

According to Method A, to a sealed vial was added the tosylhydrazone S10 (29 mg, 0.050 mmol, 1.0 eq), (1-methyl-1H-pyrazol-4-yl)boronic acid (19 mg, 0.15 mmol, 3.0 eq) and K₂CO₃ (21 mg, 0.15 mmol, 3.0 eq) then 1,4-dioxane (0.25 mL). Purification by FCC (CH₂Cl₂: EtOAc, 0-80%) afforded the coupled product in 83% yield (20 mg).

¹H NMR (400 MHz, CDCl₃) δ 8.85 (s, 1H), 7.55 (t, J=7.9 Hz, 1H), 7.48 (dt, J=8.1, 0.9 Hz, 1H), 7.42-7.33 (m, 3H), 7.28-7.24 (m, 1H), 7.19-7.11 (m, 2H), 4.42 (dq, J=1775.4, 7.1 Hz, 1H), 4.39 (s, 2H), 4.27 (q, J=7.1 Hz, 2H), 3.86 (s, 3H), 2.88 (d, J=0.9 Hz, 3H), 1.73 (d, J=7.2 Hz, 3H), 1.25 (t, J=7.0 Hz, 3H); ¹³C NMR (101 MHz, CDC₃) δ162.7 (d, J_{FC}=246.0 Hz), 161.5, 137.9, 137.4 (d, J_{FC}=8.9 Hz), 134.0, 132.0 (d, J_{FC}=3.5 Hz), 129.9, 128.2, 127.9, 127.3 (d, J_{FC}=2.9 Hz), 125.5, 123.7, 123.0, 121.9, 121.7, 119.5, 118.0 (d, J_{FC}=22.1 Hz), 114.4 (d, J_{FC}=15.1 Hz), 61.2, 54.3 (d, J_{FC}=2.8 Hz), 39.4 (d, J_{FC}=2.6 Hz), 38.9, 31.9, 21.5, 14.0; LRMS (TOF, ESI+) m/z: [M+H]⁺ Calcd for C₂₅H₂₇FN₃O₄S 484.2; Found 484.2.

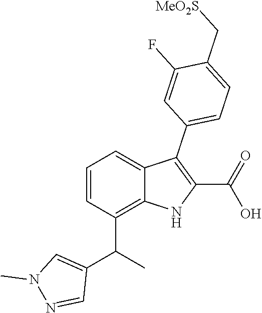 [SMILES: CC(c1cnn(C)c1)c1cccc2c(-c3ccc(CS(C)(=O)=O)c(F)c3)c(C(=O)O)[nH]c12]

To the ester (20 mg, 0.041 mmol) was added LiOH (9 mg, 0.21 mmol, 5 eq), H₂O (0.10 mL), EtOH (0.10 mL) and THF (0.20 mL) and the reaction mixture was stirred at RT overnight. The reaction mixture was neutralised (1 M HCl) and then concentrated in vacuo and then purified by FCC (Snap-Ultra, 10 g, CH₂Cl₂:EtOAc 0-100%) to afford the indole carboxylate IC-3 in 89% yield (16 mg).

¹H NMR (400 MHz, DMSO) δ 11.68 (1H, s), 7.52 (1H, t, J=8.1 Hz), 7.49 (1H, s), 7.41-7.34 (2H, m), 7.34-7.27 (2H, m), 7.15-6.99 (2H, m), 4.92 (1H, q, J=7.0 Hz), 4.60 (2H, s), 3.76 (3H, s), 3.06 (3H, s), 1.55 (3H, d, J=7.0 Hz); ¹³C NMR (101 MHz, DMSO) δ 162.7, 159.1, 137.2, 137.1, 134.1, 132.3, 132.2, 128.4, 127.1, 126.5, 125.7, 122.1, 121.0, 120.4, 117.8, 117.5, 117.3, 114.2, 53.1, 39.9, 38.4, 28.7, 22.3; LRMS (TOF, ESI⁺) m/z: [M+H]⁺ Calcd for C₂₃H₂₃FN₃O4S 456.1; Found 456.2.

### IC-4: 3-(3-Fluoro-4-((methylsulfonyl)methyl)phenyl)-7-(1-(thiophen-2-yl)ethyl)-1H-indole-2-carboxylic acid

According to Method A, to a sealed vial was added the tosylhydrazone S10 (59 mg, 0.10 mmol, 1.0 eq), 2-thiophene boronic acid (38 mg, 0.30 mmol, 3.0 eq) and K₂CO₃ (42 mg, 0.30 mmol, 3.0 eq) then 1,4-dioxane (0.50 mL). Purification by FCC (cyclohexane: EtOAc, 0-80%) afforded the desired compound in 59% yield (29 mg).

¹H NMR (400 MHz, CDCl₃) δ 8.80 (1H, s), 7.60-7.48 (2H, m), 7.44-7.30 (3H, m), 7.24-7.15 (2H, m), 7.03-6.94 (2H, m), 4.72 (1H, q, J=7.1 Hz), 4.38 (2H, s), 4.25 (2H, q, J=7.1 Hz), 2.88 (3H, s), 1.87 (3H, d, J=7.1 Hz), 1.22 (3H, t, J=7.1 Hz); ¹³C NMR (101 MHz, CDCl₃) δ 161.4, 160.3 (d, J_{FC}=247.8 Hz), 149.0, 137.3 (d, J_{FC}=8.8 Hz), 134.0, 132.0 (d, J_{FC}=3.3 Hz), 129.4, 128.0, 127.3 (d, J_{FC}=3.2 Hz), 126.9, 124.4, 124.2, 123.8, 123.0, 121.8, 121.6, 119.9, 118.0 (d, J_{FC}=22.3 Hz), 114.4 (d, J_{FC}=14.4 Hz), 61.1, 54.2 (d, J_{FC}=2.7 Hz), 39.4 (d, J_{FC}=2.5 Hz), 37.2, 22.1, 14.0; LRMS (TOF, ESI⁺) m/z: [M+Na]⁺ Calcd for C₂₅H₂₄FNNaO₄S₂ 508.1; Found 508.1.

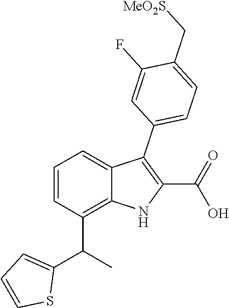 [SMILES: CC(c1cccs1)c1cccc2c(-c3ccc(CS(C)(=O)=O)c(F)c3)c(C(=O)O)[nH]c12]

To the ester (12 mg, 0.024 mmol) was added LiOH (5 mg, 0.12 mmol, 5 eq), H₂O (0.10 mL), EtOH (0.10 mL) and THF (0.20 mL) and the reaction mixture was stirred at RT overnight. The reaction mixture was neutralised (1 M HCl) and then concentrated in vacuo and then purified by FCC (Snap-Ultra, 10 g, CH₂Cl₂:EtOAc 0-100%) to afford the desired compound IC-4 in 27% yield (3 mg).

¹H NMR (500 MHz, MeOD) δ 7.55 (1H, t, J=7.9 Hz), 7.47-7.33 (3H, m), 7.25-7.20 (2H, m), 7.13-7.06 (1H, m), 6.98 (1H, dt, J=3.5, 1.1 Hz), 6.95 (1H, dd, J=5.1, 3.5 Hz), 5.04 (1H, q, J=7.1 Hz), 4.55 (2H, s), 2.98 (3H, s), 1.80 (3H, d, J=7.1 Hz); ¹³C NMR (126 MHz, MeOD) δ 163.3, 160.8 (d, J_{FC}=247.1 Hz), 149.5, 137.8 (d, J_{FC}=8.9 Hz), 134.3, 131.9 (d, J_{FC}=3.5 Hz), 130.5, 127.7, 126.7 (d, J_{FC}=3.3 Hz), 126.1, 124.0, 123.7, 123.3, 122.6, 121.5, 120.9, 118.6, 117.5 (d, J_{FC}=22.5 Hz), 114.2 (d, J_{FC}=15.2 Hz), 53.2 (d, J_{FC}=2.4 Hz), 38.6, 35.1, 21.6; HRMS (TOF, ESI⁺) m/z: [M+H]⁺ Calcd for C₂₃FH₂₁NO₄S₂ 458.08905; Found 458.08905.

### IC-5: 7-(1-(1-(3-Aminopropyl)-1H-pyrazol-4-yl)ethyl)-3-(3-fluoro-4-((methylsulfonyl) methyl)phenyl)-1H-indole-2-carboxylic acid

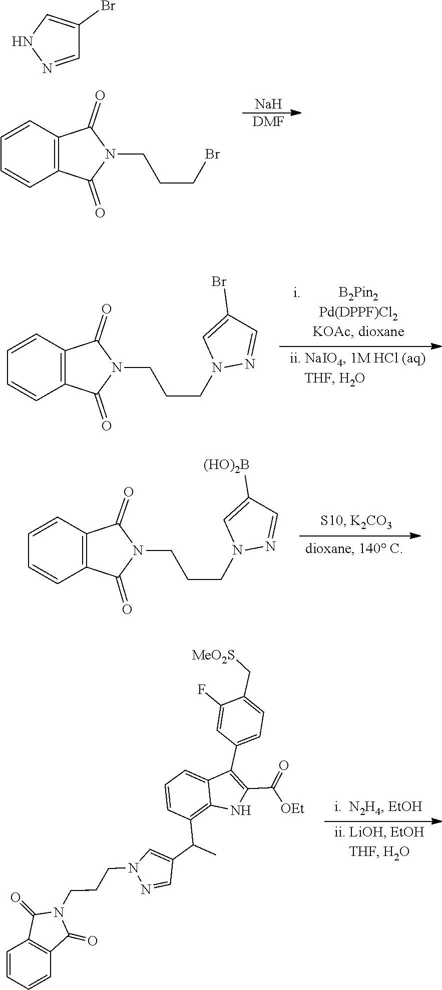

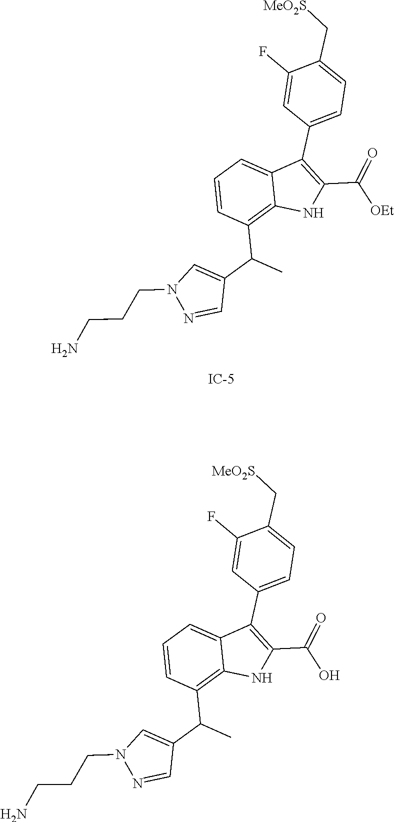 [SMILES: CCOC(=O)c1[nH]c2c(C(C)c3cnn(CCCN)c3)cccc2c1-c1ccc(CS(C)(=O)=O)c(F)c1 ; "IC-5" ; CC(c1cnn(CCCN)c1)c1cccc2c(-c3ccc(CS(C)(=O)=O)c(F)c3)c(C(=O)O)[nH]c12]

To a solution of bromo pyrazole (1.47 g, 10 mmol) in DMF (33 mL) at 0° C. was added NaH (0.44 g, 11 mmol, 60% in mineral oil) portionwise. The reaction was stirred for 30 minutes and then N-(3-bromopropyl)phthalimide (2.95 g, 11 mmol) was added; then quenched after 2 hours by the addition of sat NH₄Cl and Et₂O. The aqueous layer was extracted (Et₂O), the combined organics were washed with brine, dried (Na₂SO₄) and then concentrated in vacuo. Purification by FCC (CyH:EtOAc 0-50%) afforded the bromopyrazole in 61% yield (2.04 g). ¹H NMR (400 MHz, CDCl₃) δ 7.85 (2H, dd, J=5.4, 3.1 Hz), 7.73 (2H, dd, J=5.4, 3.0 Hz), 7.55 (1H, d, J=0.7 Hz), 7.41 (1H, d, J=0.7 Hz), 4.15 (2H, t, J=6.7 Hz), 3.74 (2H, t, J=6.5 Hz), 2.27 (2H, p, J=6.7 Hz).

To the bromopyrazole (833 mg, 2.5 mmol) in a microwavable vial was added B₂Pin₂ (953 mg, 3.75 mmol, 1.5 eq), PdDPPFCl₂ (92 mg, 0.125 mmol, 0.05 eq), KOAc (735 mg, 7.5 mmol, 3 eq) and dioxane (10 ml). The reaction mixture was sparged with N₂ and then subjected to microwave irradiation at 110° C. for 2 h. The reaction was cooled, diluted with H₂O and extracted with EtOAc; the combined organics were washed (brine), dried (Na₂SO₄), concentrated in vacuo and the crude material was purified by FCC (CH₂Cl₂:EtOAc 0-100%) to afford the pinacol ester as an off-white solid in 34% yield (326 mg).

¹H NMR (400 MHz, CDCl₃) δ 7.88-7.81 (2H, m), 7.76 (1H, d, J=0.6 Hz), 7.75 (1H, d, J=0.6 Hz), 7.72 (2H, dd, J=5.5, 3.0 Hz), 4.19 (2H, t, J=7.0 Hz), 3.74 (2H, t, J=6.7 Hz), 2.35-2.24 (2H, m), 1.31 (12H, s).

To the pinacol ester (100 mg, 0.262 mmol) in THF (1.5 mL) and H₂O (0.52 mL) was added NaIO₄ (170 mg, 0.787 mmol, 3 eq) at RT and the reaction was stirred for 30 min and then 1 M aq. HCl (0.18 mL, 0.18 mmol, 0.7 eq) was added. After 2 hours, the reaction mixture was diluted with EtOAc, and the aqueous layer was extracted with EtOAc. The combined organics were washed with brine, dried (Na₂SO₄) and concentrated in vacuo to afford the crude boronic acid in quantitative yield (78 mg).

¹H NMR (400 MHz, CDCl₃) δ 7.85 (2H, dd, J=5.4, 3.0 Hz), 7.72 (2H, dd, J=5.5, 3.0 Hz), 7.49 (1H, dd, J=9.4, 2.0 Hz), 6.22 (1H, t, J=2.1 Hz), 4.20 (2H, t, J=6.7 Hz), 3.73 (2H, t, J=6.7 Hz), 2.29 (2H, p, J=6.7 Hz); LRMS (TOF, ESI⁺) m/z: [M+H]⁺ Calcd for C₁₄H₁₅BN₃O₄ 300.1; Found 300.1.

According to Method A, to the boronic acid (46 mg, 0.15 mmol, 3 eq) was added tosylhydrazone S10 (29 mg, 0.050 mmol, 1.0 eq) and K₂CO₃ (21 mg, 0.15 mmol, 3 eq) then 1,4-dioxane (0.40 mL). Purification by FCC (cyclohexane: EtOAc, 0-80%) afforded the desired compound in 76% yield (25 mg).

¹H NMR (400 MHz, CDCl₃) δ 8.78 (1H, s), 7.88-7.79 (2H, m), 7.75-7.69 (2H, m), 7.61-7.46 (2H, m), 7.44-7.31 (4H, m), 7.25-7.12 (2H, m), 4.46-4.34 (3H, m), 4.21 (2H, q, J=7.1 Hz), 4.13 (2H, t, J=6.9 Hz), 3.71 (2H, td, J=6.7, 3.6 Hz), 2.88 (3H, d, J=0.9 Hz), 2.33-2.23 (2H, m), 1.73 (3H, d, J=7.2 Hz), 1.19 (3H, t, J=7.1 Hz); ¹³C NMR (101 MHz, CDCl₃) δ 168.3, 161.4, 138.3, 137.6, 134.1, 133.9, 132.0, 132.0, 129.9, 128.8, 127.9, 127.8, 127.3, 125.0, 123.8, 123.3, 123.0, 121.7, 119.5, 118.1, 117.9, 116.6, 61.1, 54.3, 49.7, 39.4, 39.4, 35.3, 32.1, 29.4, 21.4, 14.0; LRMS (TOF, ESI⁺) m/z: [M+H]⁺ Calcd for C₃₅H₃₄FN₄O₅S 657.2; Found 657.2.

To the N-protected amine (20 mg, 0.030 mmol) in EtOH (0.3 mL) at RT was added hydrazine monohydrate (2 μL, 0.036 mmol, 1.2 eq) and the reaction mixture was stirred for 60 h, whereupon it was concentrated and purified by FCC (CH₂Cl₂:MeOH). The combined product fractions were dried and subjected to lithium hydroxide hydrolysis [6.3 mg LiOH, H₂O (0.10 mL), EtOH (0.10 mL) and THF (0.20 mL)]. Purification by R^{P} Biotage FCC afforded the desired compound IC-5 in 47% yield (6.6 mg) over two steps.

¹H NMR (500 MHz, MeOD) δ 7.59-7.53 (2H, m), 7.46-7.33 (3H, m), 7.22 (1H, d, J=7.1 Hz), 7.12 (2H, t, J=7.6 Hz), 4.74 (1H, q, J=7.1 Hz), 4.59 (2H, s), 4.26 (2H, t, J=6.6 Hz), 3.02 (3H, s), 2.98-2.89 (2H, m), 2.27-2.11 (2H, m), 1.72 (3H, d, J=7.1 Hz); ¹³C NMR (126 MHz, MeOD) δ 163.2, 160.8 (d, J_{FC}=247.2 Hz), 138.2, 137.7, 134.4, 132.0, 130.8, 128.4, 127.7, 126.6, 126.6, 123.6, 122.5, 121.7, 121.0, 118.4, 117.5 (d, J_{FC}=22.3 Hz), 114.3 (d, J_{FC}=15.1 Hz), 53.2, 48.3, 38.9, 37.0, 30.1, 28.1, 20.8; HRMS (TOF, ESI⁺) m/z: [M+H]⁺ Calcd for C₂₅H₂₈FN₄O₄S 499.18098; Found 499.18066.

### IC-6: 3-(3-Fluoro-4-((methylsulfonyl)methyl)phenyl)-7-(1-(2-oxo-2H-pyran-3-yl)ethyl)-1H-indole-2-carboxylic acid

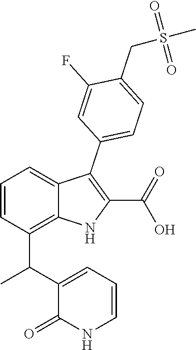 [SMILES: CC(c1ccc[nH]c1=O)c1cccc2c(-c3ccc(CS(C)(=O)=O)c(F)c3)c(C(=O)O)[nH]c12]

According to Method A, to a sealed vial was added the tosylhydrazone S10 (29 mg, 0.050 mmol, 1.0 eq), (2-oxo-1,2-dihydropyridin-3-yl)boronic acid (10 mg, 0.07 mmol), K₂CO₃ (10 mg, 0.07 mmol) then 1,4-dioxane (0.25 mL). Purification by FCC (DCM:EtOAc, 0.1% formic acid; gradient: 5-−80%) afforded the pyridone in 28% yield (7 mg, 0.01 mmol).

¹H NMR (400 MHz, CDCl₃) δ ppm 11.15 (s, 1H), 7.61 (d, J=8.1 Hz, 1H), 7.54 (t, J=7.9 Hz, 1H), 7.51-7.48 (m, 1H), 7.42-7.39 (m, 1H), 7.38-7.36 (m, 1H), 7.37-7.34 (m, 1H), 7.33-7.27 (m, 1H), 7.22-7.16 (m, 1H), 6.96 (q, J=7.2 Hz, 1H), 6.79-6.67 (m, 1H), 6.28-6.15 (m, 1H), 4.37 (s, 2H), 4.36-4.29 (m, 2H), 2.87 (d, J=0.9 Hz, 3H), 1.90 (d, J=7.2 Hz, 3H), 1.35 (t, J=7.1 Hz, 3H); ¹³C NMR (101 MHz, CDCl₃) δ ppm 163.3, 161.7, 161.2, 139.4, 134.8, 133.7, 132.2, 132.1, 127.7, 127.4, 124.7, 124.2, 121.7, 121.6, 121.0, 120.9, 118.2, 118.0, 114.4, 108.1, 61.2, 54.4, 47.7, 39.6, 18.2, 14.3. LRMS [M+Na]⁺ 519.2; HRMS (TOF, ESI⁺) m/z: [M+Na]⁺ Calcd for C₂₆H₂₄FNNaO₆S 519.13604; Found 519.13641.

The ester (7 mg, 0.01 mmol) was dissolved in 1:1:1 THF: MeOH:LiOH (2 M, 125 μL) in a sealed vial. The reaction mixture was heated up in a sand bath to 100° C., and stirred for 4 h. The reaction was quenched by the addition of 1 M HCl (aq.), extracted with EtOAc (3×10 mL) and DCM (2×10 mL). The combined organics were dried over MgSO₄ and concentrated under reduced pressure. The crude product was purified by reversed phase FCC (H₂O:ACN, 0.1% formic acid; gradient: 5→80%) to obtain the desired indole carboxylate IC-6 in 11% yield (0.7 mg, 0.002 mmol) as a pale white solid.

¹H NMR (400 MHz, DMSO) δ 11.37 (s, 1H), 7.94-7.85 (m, 1H), 7.54-7.47 (m, 3H), 7.47-7.42 (m, 1H), 7.41-7.34 (m, 2H), 7.17 (t, J=7.7 Hz, 1H), 6.80-6.72 (m, 1H), 6.56 (d, J=9.1 Hz, 1H), 6.35 (t, J=6.7 Hz, 1H), 4.59 (s, 2H), 3.06 (s, 3H), 1.85 (d, J=7.2 Hz, 3H). ¹³C NMR (126 MHz, DMSO) δ ppm 167.0, 162.2, 162.0, 159.4, 140.0, 135.2, 133.9, 132.5, 131.7, 128.7, 127.1, 126.5, 125.7, 124.3, 121.7, 121.0, 119.6, 117.5, 114.5, 107.2, 53.0, 47.8, 38.1, 18.2. LRMS [M+H]⁺ 469.3; HRMS (TOF, ESI⁺) m/z: [M+H]⁺ Calcd for C₂₄H₂₁FN₂O₅S 469.12389; Found 469.12388.

### IC-7: 7-(1-(2-Chlorothiazol-5-yl)ethyl)-3-(3-fluoro-4-((methylsulfonyl)methyl)phenyl)-1H-indole-2-carboxylic acid

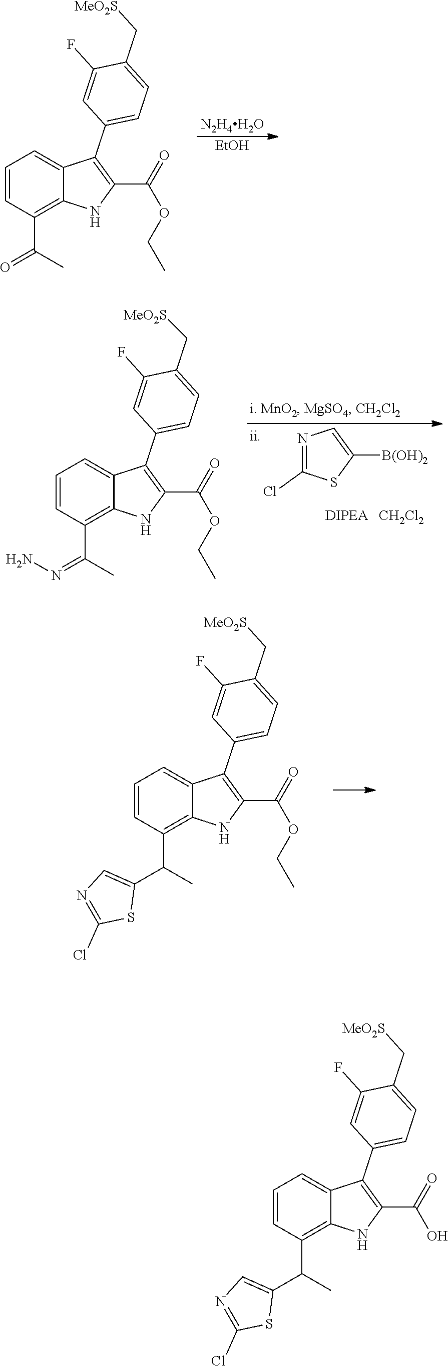

To a suspension of ketone S5 (182 mg, 0.44 mmol) in EtOH (1 mL) and CH₂Cl₂ (1 mL) was added hydrazine monohydrate (23 μL, 0.48 mmol, 1.1 eq) and the reaction mixture was heated to 70° C. for 12 hours. After 3 hours a second equivalent of hydrazine was added and then the volatiles were removed in vacuo and the crude mixture was purified by FCC (cyclohexane: EtOAc 0-40%) to give the hydrazone product in 55% yield (100 mg).

¹H NMR (400 MHz, CDCl₃) δ 11.24 (s, 1H), 7.63-7.53 (m, 3H), 7.51 (dd, J=7.5, 1.0 Hz, 1H), 7.45-7.35 (m, 3H), 7.18 (dd, J=8.1, 7.4 Hz, 1H), 5.56 (s, 2H), 4.39 (s, 2H), 4.34 (q, J=7.2 Hz, 2H), 2.88 (s, 3H), 2.75 (s, 1H), 2.30 (s, 3H), 1.27 (t, J=7.2 Hz, 3H); LRMS (TOF, ESI⁺) m/z: [M+Na]⁺ Calcd for C₂₁H₂₃FN₃O4S 432.1; Found 432.2.

To a solution of the hydrazone (22 mg, 0.05 mmol) in CH₂Cl₂ (1 mL) was added MgSO₄ (9 mg, 0.075 mmol, 1.5 eq), the reaction mixture was cooled to 0° C. and MnO₂ (17 mg, 0.20 mmol, 4 eq) was added and the reaction was slowly warmed to RT over 2 h. The reaction mixture was filtered through cotton wool into a new vial (with 1-2 mL CH₂Cl₂ for transfer). To a separate vial was added the thiazole boronic acid (12 mg, 0.075 mmol, 1.5 eq) in CH₂Cl₂ (1 mL) and DIPEA (26 μL, 0.15 mmol, 3 eq). To the boronic acid solution was added the crude diazo solution dropwise and the reaction mixture was stirred for 1 h, whereupon the solvents were removed in vacuo and the crude reaction mixture was purified by FCC (CH₂Cl₂: EtOAc 0-80%) to afford the thiazole product in 46% yield (12 mg).

¹H NMR (400 MHz, CDCl₃) δ 8.87 (1H, s), 7.57-7.47 (2H, m), 7.40-7.30 (3H, m), 7.26-7.05 (2H, m), 4.64 (1H, q, J=7.1 Hz), 4.36 (2H, s), 4.28-4.22 (2H, m), 2.85 (3H, s), 1.82 (3H, d, J=7.1 Hz), 1.22 (3H, d, J=7.0 Hz); LRMS (TOF, ESI⁺) m/z: [M+Na]⁺ Calcd for C₂₄H₂₂ClFN₂NaO₄S₂ 543.0; Found 543.0.

To the ester (12 mg, 0.023 mmol) was added LiOH (6 mg), H₂O (0.10 mL), EtOH (0.10 mL) and THF (0.20 mL) and the reaction mixture was stirred at RT overnight. The reaction mixture was neutralised (1 M HCl) and then concentrated in vacuo and then purified by FCC (Snap-Ultra, 10 g, CH₂Cl₂:EtOAc 0-100%) to afford the desired indole carboxylate IC-7 in 12% yield (1.3 mg).

¹H NMR (400 MHz, MeOD-d4) δ 7.56 (1H, td, J=7.7, 2.0 Hz), 7.47 (1H, dd, J=8.1, 1.1 Hz), 7.45-7.34 (3H, m), 7.25 (1H, d, J=7.2 Hz), 7.18-7.10 (1H, m), 5.08 (1H, q, J=7.1 Hz), 4.55 (2H, s), 2.99 (3H, s), 1.81 (3H, d, J=7.0 Hz); LRMS (TOF, ESI⁺) m/z: [M+Na]⁺ Calcd for C₂₂H₁₈ClFN₂NaO₄S₂ 515.0; Found 515.0.

### 1.2.3 Racemic C7 Amine Synthesis

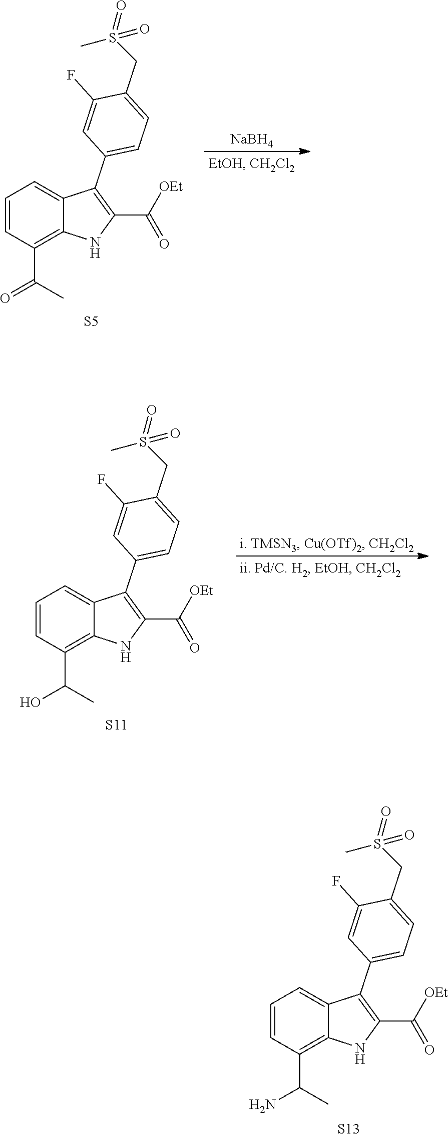

To the ketone S5 (400 mg, 0.958 mmol) in 1:2 DCM-EtOH (15 mL) cooled in an ice bath was added NaBH₄ (72.5 mg, 1.92 mmol) and reaction mixture was stirred at rt for 3 h and then concentrated in vacuo. To the crude residue was added sat. aq. NaHCO₃, and then extracted with EtOAc. The combined EtOAc extracts were dried over Na₂SO₄, filtered and concentrated in vacuo. Purification by FCC (KP-Sil 25 g, DCM:EtOAc gradient) afforded the alcohol S11 as a white solid in 98% yield (392 mg).

¹H NMR (500 MHz, CDCl₃) δ=9.82 (s, 1H), 7.63-7.50 (m, 2H), 7.46-7.36 (m, 2H), 7.19-7.06 (m, 2H), 5.36-5.30 (m, 1H), 4.39 (s, 2H), 4.32 (q, J=7.1, 2H), 2.89 (s, 3H), 2.22 (d, J=3.8, 1H), 1.71 (d, J=6.5, 3H), 1.27 (t, J=7.1, 3H); HRMS (TOF, ESI⁺) m/z: [M+Na]⁺ Calcd for C₂₁H₂₂FNNaO₅S 442.10949; Found 442.10915.

To the indole alcohol S11 (300 mg, 0.715 mmol) in DCM (1.5 mL) at rt was added TMSN₃ (0.142 mL, 1.07 mmol) and Cu(OTf)₂ (13 mgm 0.0358 mmol) and the reaction mixture was stirred at rt for 30 min. The resultant mixture was diluted with water and extracted with DCM; the combined organic extracts were dried over Na₂SO₄, filtered and concentrated in vacuo to obtain the azide S12 as a white fluffy solid which was used without further purification in quantitative yield (318 mg).

¹H NMR (500 MHz, CDCl₃) δ=9.42 (s, 1H), 7.57 (dt, J=7.9, 3.8, 2H), 7.44-7.33 (m, 2H), 7.24 (d, J=1.0, 1H), 7.16 (dd, J=8.1, 7.2, 1H), 4.99 (q, J=6.8, 1H), 4.40 (s, 2H), 4.33 (q, J=7.1, 2H), 3.00-2.85 (m, 3H), 1.74 (d, J=6.8, 3H), 1.27 (t, J=7.1, 3H); LRMS [M−H]⁻ 443.0.

To the azido indole S12 (320 mg, 0.720 mmol) in EtOH (3.6 mL) and DCM (2.9 mL) was added Pd/C (7.7 mg, 0.072 mmol). The solution was bubbled through with H₂, and a further 2 mL of DCM was added to the reaction mixture and the reaction mixture was stirred overnight with a H₂ balloon. The resultant mixture was filtered through Celite®, rinsing with DCM and the filtrate was concentrated in vacuo to afford the crude amine S13 which was used without further purification in 96% yield (290 mg).

¹H NMR (400 MHz, MeOD) δ=7.57 (t, J=7.8, 1H), 7.44 (dd, J=8.2, 1.1, 1H), 7.41-7.30 (m, 3H), 7.22-7.10 (m, 1H), 4.74 (q, J=6.6, 1H), 4.57 (s, 2H), 4.29 (q, J=7.1, 2H), 3.00 (s, 3H), 1.55 (d, J=6.7, 3H), 1.24 (t, J=7.1, 3H); LRMS [M-H]⁻ 417.0.

### IC-8: 3-(3-Fluoro-4-((methylsulfonyl)methyl)phenyl)-7-(1-(4-((sulfamoylamino)methyl)-1H-1,2,3-triazol-1-yl)ethyl)-1H-indole-2-carboxylic acid

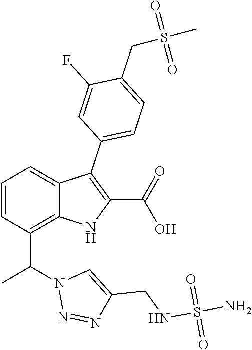 [SMILES: CC(c1cccc2c(-c3ccc(CS(C)(=O)=O)c(F)c3)c(C(=O)O)[nH]c12)n1cc(CNS(N)(=O)=O)nn1]

Azide S12 (50 mg, 0.11 mmol) and 3-(sulfamoylamino)prop-1-yne (18 mg, 0.11 mmol) were dissolved in 3:1 H₂O:THF in a microwavable reaction vessel. To this mixture a solution of CuSO₄ (0.4 mL, 10 mol %, aq.) followed by a solution of ascorbic acid (0.8 mL, 20 mol %, aq.) were added. The reaction mixture was heated to 100° C. using microwave irradiation for 30 minutes. The mixture was diluted with CH₂Cl₂, the phases were separated and the aqueous phase was extracted with CH₂Cl₂ (3×10 mL). The combined organic phases were washed with brine (1×10 mL), dried over MgSO₄ and concentrated under reduced pressure. The crude product was purified by automated FCC (CH/EtOAc gradient: 2→40%) to afford the triazole in 79% yield as a pale white solid (51 mg, 0.09 mmol).

LRMS [M+Na]⁺ 601.2; HRMS (TOF, ESI⁺) m/z: [M+Na]⁺ Calcd for C₂₄H₂₇FN₆NaOSS₂ 579.14958; Found 579.14902.

The ethyl ester (30 mg, 0.05 mmol) was dissolved in 1:1:1 THF: MeOH: 2 M LiOH H₂O (1.5 mL) in a sealed vial. The reaction mixture was heated up in a sand bath to 100° C., and stirred for 4 h. The reaction was quenched by the addition of 1 M HCl (aq.), extracted with EtOAc (3×10 mL) and DCM (2×10 mL). The combined organics were dried over MgSO₄ and concentrated under reduced pressure. The crude product was purified by FCC (DCM:MeOH, 0.1% formic acid; gradient: 2→10%) to obtain the desired indole carboxylate IC-8 in 61% yield (17 mg, 0.03 mmol) as a pale white solid.

¹H NMR (400 MHz, DMSO) δ ppm 12.04 (s, 1H), 8.16 (s, 1H), 7.53 (t, J=7.8 Hz, 1H), 7.48-7.43 (m, 1H), 7.42-7.33 (m, 2H), 7.19 (d, J=7.1 Hz, 1H), 7.11 (t, J=7.7 Hz, 1H), 6.87-6.79 (m, 1H), 4.61 (s, 2H), 4.14 (s, 2H), 3.06 (s, 3H), 1.96 (d, J=6.9 Hz, 3H); ¹³C NMR (101 MHz, DMSO) δ ppm 162.5, 144.5, 136.5, 136.5, 133.2, 132.4, 127.6, 126.6, 125.9, 125.0, 121.0, 120.6, 120.2, 117.7, 117.6, 117.4, 114.5, 114.4, 54.2, 53.0, 38.3, 20.9. LRMS [M+H]⁺ 551.2.

### IC-9: 7-(1-(2H-Tetrazol-5-yl)ethyl)-3-(3-fluoro-4-((methylsulfonyl)methyl)phenyl)-1H-indole-2-carboxylic acid

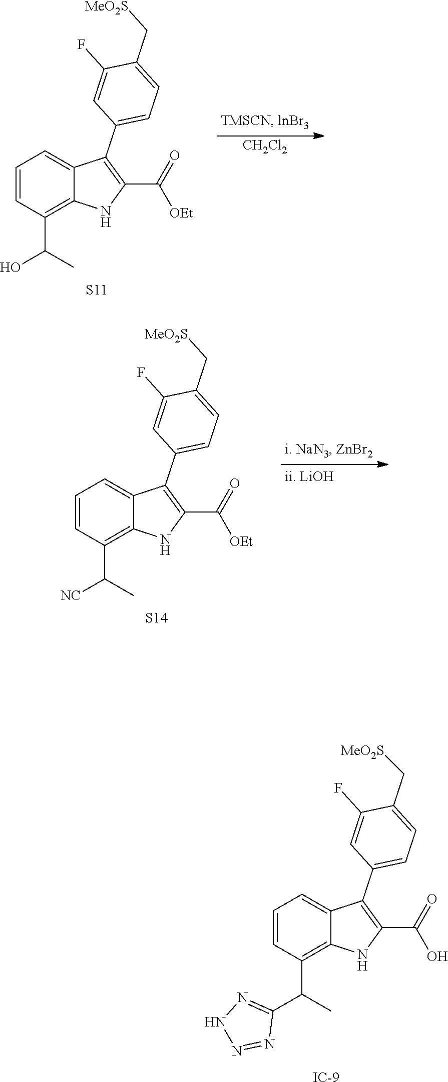

To a suspension of InBr₃ (26.8 mg, 0.0758 mmol) and TMSCN (0.190 mg, 1.52 mmol) in CH₂Cl₂ (2.5 mL) was added a solution of the alcohol S11 (318 mg, 0.758 mmol) in CH₂Cl₂ (2.5 mL) dropwise at RT and the reaction was stirred at RT overnight. The reaction mixture was diluted with CH₂Cl₂, washed with H₂O and the organic layer was dried (Na₂SO₄) and concentrated in vacuo. Purification by FCC (SNAP-Ultra 10 g, cyclohexane to EtOAc 0-100%) afforded the nitrile S14 in 79% yield (255 mg).

To a sealed vial was added S14 (43 mg, 0.10 mmol), sodium azide (7 mg, 0.11 mmol, 1.1 eq) and ZnBr₂ (23 mg, 0.10 mmol, 1.0 eq) then water (0.3 ml) and THF (0.05 ml). The suspension was heated to reflux for 24 h, whereupon the reaction mixture was cooled to RT, 1 M aq HCl was added and then extracted with EtOAc (×3). The combined organics were washed with 0.25 M NaOH then brine, dried (Na₂SO₄). LCMS analysis showed approximately 50% conversion to the tetrazole. Attempted purification by FCC (CH₂Cl₂:MeOH; 0-20%) afforded a mixture of SM and tetrazole (33 mg) which was taken through to the hydrolysis step. To the above mixture (33 mg) was added H₂O (0.1 ml), EtOH (0.1 mL), THE (0.2 ml) and LiOH (15 mg, 0.35 mmol) at RT and stirring was maintained for 24 h. The reaction mixture was neutralised with 1 M HCl, dried and the crude was dissolved in DMSO/H₂O, filtered and purified by semi-preparative HPLC. The desired indole carboxylate IC-9 was obtained as a colourless solid in 9% yield (3.77 mg) over two steps.

¹H NMR (500 MHz, MeOD) δ 7.59 (1H, t, J=7.8 Hz), 7.51 (1H, dd, J=8.2, 1.1 Hz), 7.45-7.36 (2H, m), 7.17-7.11 (1H, m), 7.06 (1H, d, J=7.2 Hz), 5.22 (1H, q, J=7.2 Hz), 4.58 (2H, s), 3.02 (3H, s), 1.91 (3H, d, J=7.2 Hz); ¹³C NMR (126 MHz, MeOD) δ 162.9, 160.8 (d, J_{FC}=247.2 Hz), 159.4, 137.5 (d, J_{FC}=8.9 Hz), 134.0, 132.0 (d, J_{FC}=3.4 Hz), 128.2, 126.7 (d, J_{FC}=3.0 Hz), 125.8, 124.4, 122.8, 121.8, 121.1, 119.7, 117.5 (d, J_{FC}=22.6 Hz), 114.4 (d, J_{FC}=15.2 Hz), 53.2 (d, J_{FC}=2.4 Hz), 38.6, 30.7, 18.4.

### IC-10: 7-[1-[4-(3-Aminopropyl)triazol-1-yl]ethyl]-3-[3-chloro-4-(methylsulfonylmethyl)phenyl]-1H-indole-2-carboxylic acid

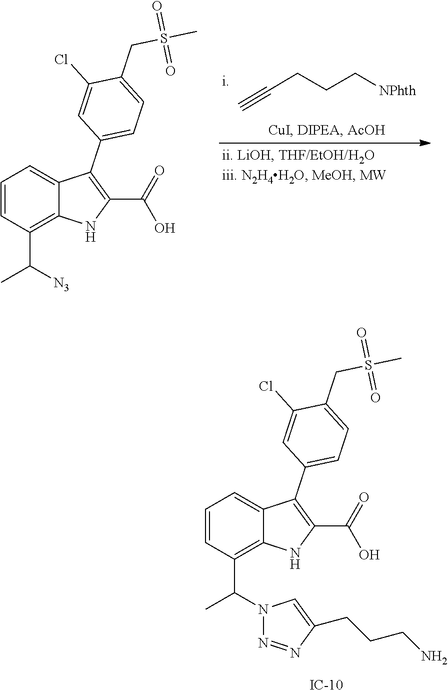

To a stirred solution of ethyl 7-(1-azidoethyl)-3-[3-chloro-4-(methylsulfonylmethyl)phenyl]-1H-indole-2-carboxylate (100 mg, 0.217 mmol) in dry THF (8 mL/mmol) at RT were sequentially added 2-pent-4-ynylisoindoline-1,3-dione (1.2 eq.), CuI (1 eq.), DIPEA (1.2 eq.) and AcOH (1.2 eq.). The solution was stirred for 3 days at RT. The solution was diluted with EtOAc, washed with aq. sat. NH4Cl and brine. The aqueous phase was extracted with EtOAc (×3).

The organic phase was dried over Na₂SO₄ anh., filtered and evaporated. The residue was purified by flash chromatography DCM/EtOAc (85/15) and afforded the iodosubsituted triazole in 55% yield (96 mg) and the desired triazole in 25% yield (36 mg).

Iodo triazole: ¹H NMR (400 MHz, CDCl₃) δ 10.16 (s, 1H), 7.81 (dd, J=5.5, 3.0 Hz, 2H), 7.69 (dd, J=5.4, 3.1 Hz, 2H), 7.64 (dd, J=5.9, 4.9 Hz, 2H), 7.58 (d, J=8.1 Hz, 1H), 7.50 (dd, J=8.0, 1.7 Hz, 1H), 7.38 (d, J=6.8 Hz, 1H), 7.14 (dd, J=8.2, 7.2 Hz, 1H), 5.95 (q, J=7.2 Hz, 1H), 4.53 (s, 2H), 4.31 (q, J=7.1 Hz, 2H), 3.76 (t, J=7.2 Hz, 2H), 2.87 (s, 3H), 2.74-2.67 (m, 2H), 2.23 (d, J=7.2 Hz, 3H), 2.14-2.05 (m, 2H), 1.31 (t, J=7.1 Hz, 3H); ¹³C NMR (101 MHz, CDC₃) δ 168.4, 161.0, 151.1, 136.4, 134.0, 133.8, 132.8, 132.4, 132.2, 132.1, 130.0, 129.0, 125.3, 125.3, 124.1, 123.3, 123.1, 122.1, 121.4, 121.1, 79.6, 61.3, 60.8, 58.0, 39.9, 37.7, 27.7, 23.9, 20.2, 14.3.

Triazole: ¹H NMR (400 MHz, CDCl₃) δ 9.95 (s, 1H), 7.80 (dd, J=5.5, 3.0 Hz, 2H), 7.69 (dd, J=5.4, 3.1 Hz, 2H), 7.65-7.61 (m, 2H), 7.58 (d, J=8.1 Hz, 1H), 7.49 (dd, J=8.0, 1.7 Hz, 1H), 7.42 (s, 1H), 7.40 (d, J=7.2 Hz, 1H), 7.18 (dd, J=8.1, 7.3 Hz, 1H), 6.18 (q, J=7.1 Hz, 1H), 4.53 (s, 2H), 4.30 (tt, J=7.2, 3.6 Hz, 2H), 3.70 (t, J=7.0 Hz, 2H), 2.87 (s, 3H), 2.73 (t, J=7.6 Hz, 2H), 2.14 (d, J=7.2 Hz, 3H), 2.08-1.98 (m, 2H), 1.29 (t, J=7.1 Hz, 3H); ¹³C NMR (101 MHz, CDCl₃) δ 168.5, 160.9, 147.7, 136.4, 134.1, 133.8, 133.6, 132.5, 132.1, 132.1, 129.9, 128.5, 125.3, 124.2, 123.6, 123.3, 123.0, 122.1, 121.5, 121.2, 120.3, 61.3, 57.9, 57.5, 39.9, 37.4, 28.1, 23.2, 19.5, 14.3; LC-MS [M□H]^{□} 672.0; HRMS (TOF, ESI^{□}) m/z: [M□H]^{□} Calcd for C₃₄H₃₃O₆N₅³⁵Cl³²S 674.18346; Found 674.18365.

To the triazole (35 mg, 0.051 mmol) in THF/EtOH/H₂O (8 mL/4 mL/4 mL/mmol) was added LiOH·H₂O (10 eq.) and the reaction was stirred at room temperature overnight. The reaction mixture was acidified to pH=2 with 2 M HCl and extracted with EtOAc (×6). The organic phase was dried over Na₂SO₄ anh., filtered and the solvent was removed in vacuo and the crude residue was used in the next step without further purification (33 mg, >99% yield).

¹H NMR (400 MHz, MeOD) δ 8.51 (s, 1H), 7.95 (dd, J=7.7, 0.9 Hz, 1H), 7.64-7.56 (m, 3H), 7.52 (dd, J=7.3, 5.7 Hz, 2H), 7.45 (dd, J=12.2, 4.3 Hz, 2H), 7.38 (d, J=7.2 Hz, 1H), 7.17 (t, J=7.7 Hz, 1H), 6.82 (q, J=6.4 Hz, 1H), 4.68 (s, 2H), 3.40 (t, J=6.1 Hz, 2H), 3.05-2.94 (m, 5H), 2.16 (d, J=6.8 Hz, 3H), 2.06-1.95 (m, 2H); ¹³C NMR (101 MHz, MeOD) δ 173.2, 169.2, 164.0, 145.9, 139.7, 137.7, 135.5, 134.9, 133.7, 133.3, 132.9, 131.3, 130.7, 130.5, 129.7, 128.8, 126.6, 126.5, 126.3, 124.1, 123.8, 122.8, 122.8, 122.5, 79.5, 60.0, 58.2, 40.6, 39.5, 28.9, 21.9, 20.5.

To the carboxylic acid (33 mg, 0.051 mmol) in dry MeOH (70 mL/mmol) was added hydrazine monohydrate (60 eq.) and the reaction mixture was heated at 85° C. during 3 h under microwave irradiation. The mixture was concentrated in vacuo and the residue was purified by reverse phase chromatography [gradient from water (0.5% formic acid) 100% to acetonitrile (0.5% formic acid) 100%] to afford the desired indole carboxylate IC-10 in 85% yield as a white solid (22 mg).

¹H NMR (400 MHz, MeOD) δ 7.86 (s, 1H), 7.64 (dd, J=4.8, 3.1 Hz, 2H), 7.55-7.47 (m, 2H), 7.31 (d, J=7.2 Hz, 1H), 7.20-7.12 (m, 1H), 6.54 (q, J=7.0 Hz, 1H), 4.70 (s, 2H), 3.00 (s, 3H), 2.99-2.91 (m, 2H), 2.79 (t, J=7.5 Hz, 2H), 2.11 (d, J=7.1 Hz, 3H), 1.98 (dt, J=15.1, 7.6 Hz, 2H); ¹³C NMR (101 MHz, MeOD) δ 164.0, 147.4, 137.9, 135.5, 134.8, 133.7, 132.9, 130.7, 129.7, 126.7, 125.9, 125.9, 123.7, 122.7, 122.7, 122.3, 122.2, 58.2, 57.7, 40.5, 40.1, 28.2, 23.2, 20.6; LC-MS [M□H]^{□} 516.2; HRMS (TOF, ESI^{□}) m/z: [M□H]^{□} Calcd for C₂₄H₂₇O₄N₅³⁵Cl³²S 516.14668; Found 516.14722.

### IC-11: 7-(1-((4-(Aminomethyl)benzoyl)amino)ethyl)-3-(6-(morpholin-4-ylmethyl)pyridin-3-yl)-1H-indole-2-carboxylic acid

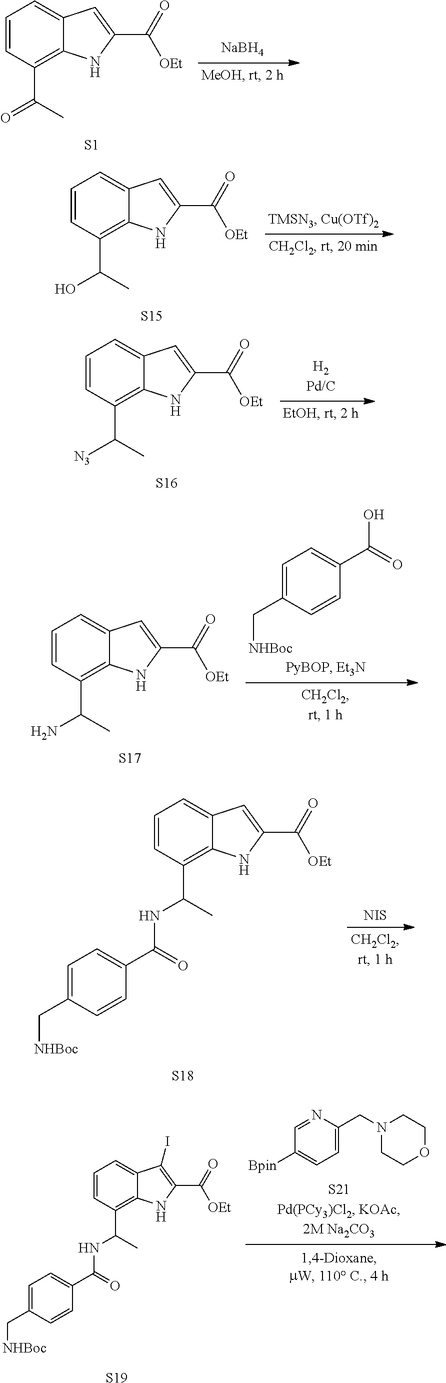

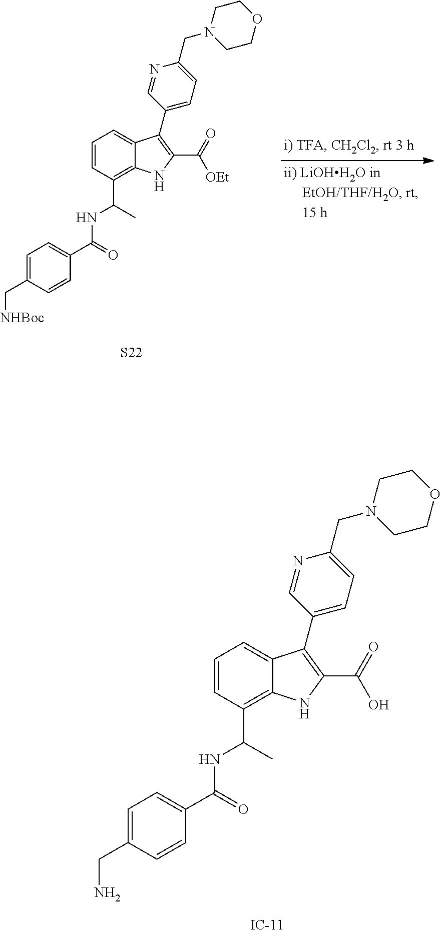 [SMILES: CCOC(=O)c1[nH]c2c(C(C)NC(=O)c3ccc(CNC(=O)OC(C)(C)C)cc3)cccc2c1-c1ccc(CN2CCOCC2)nc1 ; "S22" ; CC(NC(=O)c1ccc(CN)cc1)c1cccc2c(-c3ccc(CN4CCOCC4)nc3)c(C(=O)O)[nH]c12 ; "IC-11"]

### Ethyl 7-(1-hydroxyethyl)-1H-indole-2-carboxylate (alcohol S15)

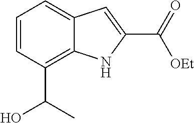 [SMILES: CCOC(=O)c1cc2cccc(C(C)O)c2[nH]1]

To indole S1 (500 mg, 2.16 mmol, 1.0 eq) in EtOH (44 mL), cooled in an ice bath to 0° C. was added NaBH₄ (164 mg, 4.32 mmol, 2.0 eq); the mixture was stirred at room temperature for 2 h. Upon completion, the solvents were removed in vacuo and the crude residue was treated with sat. aq NaHCO₃ (10 mL), then extracted with EtOAc (2×10 mL). The combined EtOAc extracts were dried over Na₂SO₄, filtered and concentrated in vacuo to obtain the desired alcohol S15 in 90% isolated yield as a white solid (506 mg, 99%).

¹H NMR (600 MHz, CDCl₃) δ ppm 9.64 (1H, br. s, NH), 7.60 (1H, dt, J=8.0, 1.0 Hz, Ar—H), 7.23 (1H, d, J=2.0 Hz, Ar—H), 7.13-7.01 (2H, m, Ar—H), 5.26 (1H, qd, J=6.5, 3.5 Hz, CH₃CHOH), 4.40 (2H, q, J=7.0 Hz, OCH₂CH₃), 2.28 (1H, d, J=3.5 Hz, OH), 1.66 (3H, d, J=6.5 Hz, CH₃CHOH), 1.41 (3H, t, J=7.0 Hz, OCH₂CH₃); ¹³C NMR (151 MHz, CDCl₃) δ ppm 162.1 (CO₂Et), 134.6, 128.9, 128.5, 127.8 (Ar—C), 121.9, 121.6, 120.5, 108.7 (Ar—CH), 70.5 (CH₃CHOH), 61.1 (OCH₂CH₃), 24.1 (CH₃CHOH), 14.6 (OCH₂CH₃); LRMS [M−H]⁻ 232.1; HRMS (TOF, ESI*) m/z: [M+H]⁺ Calcd for C₁₃H₁₆O₃N 234.11247; Found 234.11275.

### 3-(1-(2-(Ethoxycarbonyl)-1H-indol-7-yl)ethyl)triaza-1,2-dien-2-ium-1-ide (S16)

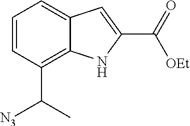 [SMILES: CCOC(=O)c1cc2cccc(C(C)N=[N+]=[N-])c2[nH]1]

To ethyl 7-(1-hydroxyethyl)-1H-indole-2-carboxylate (alcohol S15) (94 mg, 0.403 mmol, 1.0 eq) in CH₂Cl₂ (0.8 mL) was added TMSN₃ (0.08 mL, 0.604 mmol, 1.5 eq) and Cu(OTf)₂ (7.3 mg, 0.02 mmol, 0.05 eq) and the reaction mixture stirred for 20 min. Upon completion, the reaction mixture was diluted with water (5 mL), then extracted with CH₂Cl₂ (3×5 mL). The combined organic extracts were dried over Na₂SO₄, filtered and concentrated in vacuo to obtain the desired compound S16 as a white solid which was used without further purification.

¹H NMR (400 MHz, CDCl₃) δ ppm 9.24 (1H, s, NH), 7.65 (1H, d, J=8.0 Hz, Ar—H), 7.25 (1H, d, J=2.0 Hz, Ar—H), 7.19 (1H, ddd, J=7.0, 1.0, 0.5 Hz, Ar—H), 7.12 (1H, dd, J=8.0, 7.0 Hz, Ar—H), 4.94 (1H, q, J=7.0 Hz, CH₃CHN₃), 4.43 (2H, q, J=7.0 Hz, OCH₂CH₃), 1.70 (3H, d, J=7.0 Hz, CH₃CHN₃), 1.43 (3H, t, J=7.0 Hz, OCH₂CH₃); ¹³C NMR (101 MHz, CDCl₃) δ ppm 162.0 (CO₂Et), 134.2, 128.7, 128.1, 124.5 (Ar—C), 122.9, 122.8, 120.7, 109.0 (Ar—CH), 61.3 (OCH₂CH₃), 60.0 (CH₃CHN₃), 20.5 (CH₃CHN₃), 14.6 (OCH₂CH₃); LRMS [M+H]⁺ 259.0; HRMS (TOF, ESI⁺) m/z: [M+H]⁺ Calcd for C₁₃H₁₃O₂N₄ 257.10440; Found 257.10443.

### Ethyl 7-(1-aminoethyl)-1H-indole-2-carboxylate (S17)

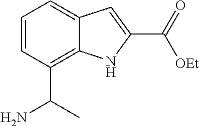 [SMILES: CCOC(=O)c1cc2cccc(C(C)N)c2[nH]1]

To azide S16 (138 mg, 0.534 mmol, 1.0 eq) in EtOH (2.7 mL) was added 10% Pd/C (56.9 mg, 0.0534 mmol, 0.1 eq); and the resultant mixture sparged with H₂. The reaction mixture was then left to stir for 2 h with a H₂ balloon attachment (with the needle reaching the solvent) until completion. The reaction mixture was filtered through a plug of Decalite®, rinsing with CH₂Cl₂. The filtrate was concentrated in vacuo to obtain the crude product S17, which was used in the next step without further purification.

¹H NMR (600 MHz, CDCl₃) δ ppm 10.52 (1H, s, NH), 7.57 (1H, d, J=8.0 Hz, Ar—H), 7.21 (1H, d, J=1.5 Hz, Ar—H), 7.10 (1H, d, J=7.0 Hz, Ar—H), 7.06 (1H, t, J=7.5 Hz, Ar—H), 4.54 (1H, q, J=6.5 Hz, CH₃CHNH₂), 4.41 (2H, q, J=7.0 Hz, OCH₂CH₃), 1.51 (3H, d, J=6.5 Hz, CH₃CHNH₂), 1.42 (3H, t, J=7.0 Hz, OCH₂CH₃); ¹³C NMR (151 MHz, CDCl₃) δ ppm 162.4 (CO₂Et), 135.8, 130.0, 128.4, 127.5 (Ar—C), 122.2, 121.3, 120.7, 108.5 (Ar—CH), 61.1 (OCH₂CH₃), 51.5 (CH₃CHNH₂), 25.3 (CH₃CHNH₂), 14.7 (OCH₂CH₃); LRMS [M−NH₂]⁺ 216.0; HRMS (TOF, ESI⁺) m/z: [M−NH₂]⁺ Calcd for C₁₃H₁₄O₂N 216.10191; Found 216.10210.

### Ethyl 7-(1-((4-(((tert-butoxycarbonyl)amino)methyl)benzoyl)amino)ethyl)-1H-indole-2-carboxylate (S18)

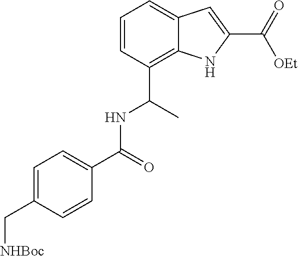 [SMILES: CCOC(=O)c1cc2cccc(C(C)NC(=O)c3ccc(CNC(=O)OC(C)(C)C)cc3)c2[nH]1]

To a solution of 4-[(tert-butoxycarbonylamino)methyl]benzoic acid (143 mg, 0.568 mmol, 1.2 eq) in CH₂Cl₂ (1.4 mL) was added amine S17 (110 mg, 0.474 mmol, 1.0 eq), benzotriazole-1-yl-oxy-tris-pyrrolidino-phosphonium hexafluoro-phosphate (296 mg, 0.568 mmol, 1.2 eq) and Et₃N (0.198 mL, 1.42 mmol, 3.0 eq). The resultant mixture was stirred for 1 h. Upon completion, the reaction mixture was diluted with CH₂Cl₂ (5 mL) and washed with 1 M HCl (2 mL). The organic solution was dried over Na₂SO₄, filtered and concentrated in vacuo. The product was purified using a Biotage KP-Sil column, with gradient elution from 100% CH₂Cl₂ to 20% EtOAc in CH₂Cl₂. The desired product S18 was obtained in 99% isolated yield over three steps as a white solid (220 mg).

¹H NMR (600 MHz, CDCl₃) δ ppm 10.63 (1H, s, NH), 7.72 (2H, d, J=8.0 Hz, Ar—H), 7.63 (1H, d, J=8.0 Hz, Ar—H), 7.30 (3H, dd, J=13.0, 7.5 Hz, Ar—H), 7.22 (1H, d, J=2.0 Hz, Ar—H), 7.11 (1H, t, J=7.5 Hz, Ar—H), 6.34 (1H, d, J=9.5 Hz, NH), 5.99-5.85 (1H, m, CH₃CH), 4.88 (1H, s, NH), 4.53-4.34 (2H, m, OCH₂CH₃), 4.31 (2H, d, J=6.0 Hz, CH₂), 1.83 (3H, d, J=7.0 Hz, CH₃CH), 1.52-1.33 (12H, m, OCH₂CH₃+C(CH₃)₃); ¹³C NMR (151 MHz, CDC₃) δ ppm 167.5, 161.8, 156.0 (C═O), 143.3, 136.0, 132.9, 128.3, 126.5 (Ar—C), 127.9, 127.6, 122.4, 120.6, 120.3, 108.6 (Ar—CH), 79.9 (C(CH₃)₃), 61.0 (OCH₂CH₃), 44.4 (CH₂), 44.0 (CH₃CH), 28.5 (C(CH₃)₃), 19.0 (CH₃CH), 14.6 (OCH₂CH₃); HRMS (TOF, ESI⁺) m/z: [M+Na]⁺ Calcd for C₂₆H₃₁O₅N₃Na 488.21564; Found 488.21559.

### Ethyl 7-(1-((4-(((tert-butoxycarbonyl)amino)methyl)benzoyl)amino)ethyl)-3-iodo-1H-indole-2-carboxylate (S19)

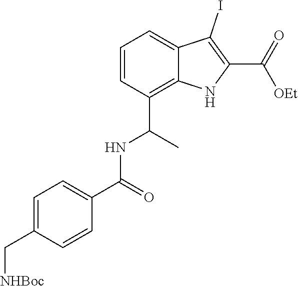 [SMILES: CCOC(=O)c1[nH]c2c(C(C)NC(=O)c3ccc(CNC(=O)OC(C)(C)C)cc3)cccc2c1I]

To a solution of ethyl indole S18 (220 mg, 0.473 mmol, 1.0 eq) in CH₂Cl₂/DMF (4.4 mL, 10:1) was added N-iodosuccinimide (117 mg, 0.52 mmol, 1.1 eq) and the reaction mixture stirred for 2 h. Upon completion, the reaction mixture was partitioned between CH₂Cl₂ (5 mL) and saturated aqueous NaHCO₃ solution (5 mL); the organic extracts were dried over Na₂SO₄, filtered and concentrated in vacuo and purification by FCC (BIOTAGE KP-Sil 25 g column, with gradient elution from 100% CH₂Cl₂ to 20% EtOAc in CH₂Cl₂) gave the desired product S19 in 87% yield as a pale yellow solid (243 mg).

¹H NMR (500 MHz, DMSO-d₆) δ ppm 11.97 (1H, s, NH), 9.00 (1H, d, J=8.0 Hz, NH), 7.84 (2H, d, J=8.0 Hz, Ar—H), 7.44 (1H, t, J=6.0 Hz, NH), 7.39 (1H, d, J=7.0 Hz, Ar—H), 7.32 (3H, dd, J=11.5, 8.0 Hz, Ar—H), 7.17 (1H, dd, J=8.0, 7.0 Hz, Ar—H), 5.97-5.51 (1H, m, CH₃CH), 4.49-4.30 (2H, m, OCH₂CH₃), 4.16 (2H, d, J=6.0 Hz, CH₂), 1.59 (3H, d, J=7.0 Hz, CH₃CH), 1.41 (3H, t, J=7.0 Hz, OCH₂CH₃), 1.39 (9H, s, C(CH₃)₃); ¹³C NMR (126 MHz, DMSO-d₆) δ ppm 165.9, 160.3, 155.8 (C═O), 143.7, 134.5, 132.6, 131.0, 130.0, 127.2 (Ar—C), 127.4, 126.6, 122.3, 121.3, 121.2 (Ar—CH), 77.9 (C(CH₃)₃), 66.8 (Cl), 60.8 (OCH₂CH₃), 44.4 (CH₃CH), 43.1 (CH₂), 28.2 (C(CH₃)₃), 20.6 (CH₃CH), 14.2 (OCH₂CH₃); HRMS (TOF, ESI⁻) m/z: [M−H]⁻ Calcd for C₂₆H₂₉O₅N₃I 590.11574; Found 590.11667.

### Synthesis of C-3 Side Chain

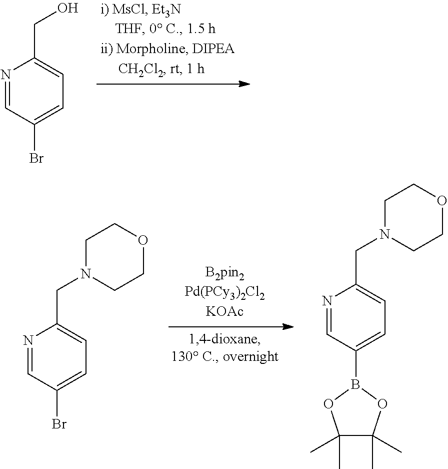 [SMILES: OCc1ccc(Br)cn1 ; Brc1ccc(CN2CCOCC2)nc1 ; CC1(C)OB(c2ccc(CN3CCOCC3)nc2)OC1(C)C]

### 4-((5-Bromopyridin-2-yl)methyl)morpholine (S20)

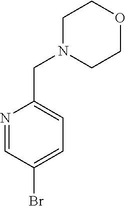 [SMILES: Brc1ccc(CN2CCOCC2)nc1]

To a solution of (5-bromopyridin-2-yl)methanol (2 g, 10.6 mmol, 1.0 eq) in THF (32 mL) at 0° C. were added Et₃N (4.43 mL, 31.9 mmol, 1.2 eq) and methanesulfonyl chloride (1.65 mL, 21.3 mmol, 2.0 eq); the resultant mixture was stirred at 0° C. for 1.5 h. The reaction mixture was partitioned between EtOAc (30 mL) and water (10 mL); the organics were dried over Na₂SO₄, filtered and concentrated in vacuo. The crude residue obtained was then dissolved in CH₂Cl₂ (32 mL) and this solution morpholine (1.02 mL, 11.7 mmol, 2.0 eq) and N,N-diisopropylethylamine (2.22 mL, 12.8 mmol, 1.2 eq) were added and the reaction mixture stirred at room temperature for 1 h by which point no limiting reagent was observed. The organic solution was washed with water (20 mL), dried over Na₂SO₄, filtered and concentrated in vacuo. The product was purified using a BIOTAGE KP-Sil 25 g column, with gradient elution from 100% cyclohexane to 100% EtOAc. The desired product S20 was obtained in 93% isolated yield as a yellow oil which solidified upon standing (2.55 g).

¹H NMR (400 MHz, CDC₃) δ ppm 8.61 (1H, dd, J=2.5, 0.5 Hz, Ar—H), 7.77 (1H, dd, J=8.5, 2.5 Hz, Ar—H), 7.33 (1H, d, J=8.5 Hz, Ar—H), 3.76-3.68 (4H, m, morpholino-CH₂), 3.60 (2H, s, CH₂), 2.52-2.46 (4H, m, morpholino-CH₂); ¹³C NMR (101 MHz, CDCl₃) δ ppm 156.9, 119.2 (Ar—C), 150.5, 139.1, 124.7 (Ar—CH), 67.0 (morpholino-CH₂), 64.3 (CH₂), 53.8 (morpholino-CH₂); HRMS (TOF, ESI⁺) m/z: [M+H]⁺ Calcd for C₁₀H₁₄O₁N₂⁷⁹Br 257.02840; Found 257.02840.

### 4-((5-(4,4,5,5-Tetramethyl-1,3,2-dioxaborolan-2-yl)pyridin-2-yl)methyl)morpholine (S21)

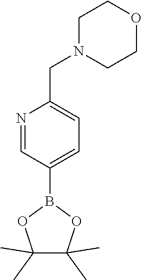 [SMILES: CC1(C)OB(c2ccc(CN3CCOCC3)nc2)OC1(C)C]

To pyridyl bromide S20 (490 mg, 1.91 mmol, 1 eq), bis(pinacolato)diboron (581 mg, 2.29 mmol, 1.2 eq), potassium acetate (281 mg, 2.86 mmol, 1.5 eq) in 1,4-dioxane (7.8 mL) in a microwavable vial was added Pd(PCy₃)₂Cl₂ (70 mg, 0.095 mmol, 0.05 eq). The reaction vial was sealed and the resultant mixture was degassed with argon, then heated at 130° C. for 15 h. Upon completion, the resultant mixture was cooled, then filtered through Celite®, washing with EtOAc. The filtrate was concentrated in vacuo to obtain the desired compound S21, which was used for the subsequent Suzuki-Miyaura cross-coupling reaction without further purification. [M+H]⁺ 305.2.

### Ethyl 7-(1-((4-(((tert-butoxycarbonyl)amino)methyl)benzoyl)amino)ethyl)-3-(6-(morpholin-4-ylmethyl)pyridin-3-yl)-1H-indole-2-carboxylate (S22)

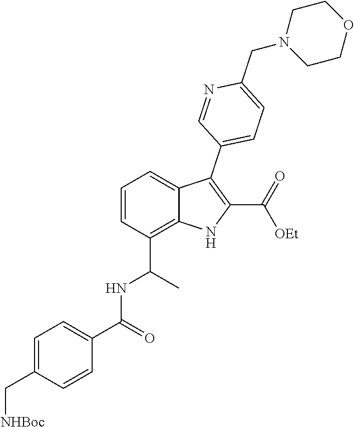 [SMILES: CCOC(=O)c1[nH]c2c(C(C)NC(=O)c3ccc(CNC(=O)OC(C)(C)C)cc3)cccc2c1-c1ccc(CN2CCOCC2)nc1]

To the crude pinacol ester S21 (148 mg, 0.487 mmol, 1.2 eq) in 1,4-dioxane (10 mL) in a microwavable vial, was added iodoindole S19 (240 mg, 0.406 mmol, 1.0 eq), Pd(PCy₃)₂Cl₂ (15 mg, 0.02 mmol, 0.05 eq), 2 M Na₂CO₃ (0.81 mL, 1.62 mmol, 4 eq). The reaction mixture was purged with argon and subjected to microwave irradiation at 110° C. for 5 h. The reaction mixture was filtered through Celite®, eluting with EtOAc (20 mL) and water (10 mL). The organics were separated from the filtrate and the aqueous layer further extracted with EtOAc (2×10 mL). The combined organic extracts were dried over Na₂SO₄, filtered, then concentrated in vacuo. The crude material was purified using a BIOTAGE KP-Sil 25 g column [100% CH₂Cl₂ to 10% MeOH in CH₂Cl₂] to afford the desired product S22 in 73% yield as a yellow solid (191 mg).

¹H NMR (500 MHz, CDCl₃) δ ppm 10.99 (1H, s, NH), 8.80-8.65 (1H, m, Ar—H), 7.86 (1H, dd, J=8.0, 2.0 Hz, Ar—H), 7.78-7.69 (2H, m, Ar—H), 7.54 (1H, d, J=8.0 Hz, Ar—H), 7.47 (1H, d, J=8.0 Hz, Ar—H), 7.36 (1H, d, J=7.5 Hz, Ar—H), 7.28 (2H, d, J=8.0 Hz, Ar—H), 7.12 (1H, dd, J=8.0, 7.0 Hz, Ar—H), 6.54 (1H, d, J=9.5 Hz, NH), 5.94 (1H, dt, J=9.0, 7.0 Hz, CH₃CH), 4.96 (1H, s, NH), 4.47-4.19 (4H, m, OCH₂CH₃+CH₂), 3.75 (4H, t, J=4.5 Hz, morpholino-CH₂), 3.71 (2H, s, CH₂), 2.56 (4H, t, J=4.5 Hz, morpholino-CH₂), 1.85 (3H, d, J=7.0 Hz, CH₃CH), 1.44 (9H, s, C(CH₃)₃), 1.33 (3H, t, J=7.0 Hz, OCH₂CH₃); ¹³C NMR (126 MHz, CDC₃) δ ppm 167.6, 161.5, 156.5 (C═O), 150.6, 138.6, 135.0, 132.7, 128.4, 127.9, 127.6, 127.5, 126.7, 124.0, 122.5, 121.4, 120.9, 120.9, 120.1 (Ar-C+Ar-CH), 75.1 (C(CH₃)₃), 67.1 (morpholino-CH₂), 65.0 (CH₂), 61.0 (OCH₂CH₃), 54.0 (morpholino-CH₂), 44.3 (CH₂), 43.8 (CH₃CH), 28.5 (C(CH₃)₃), 18.9 (CH₃CH), 14.3 (OCH₂CH₃); HRMS (TOF, ESI⁻) m/z: [M−H]⁻ Calcd for C₃₆H₄₄O₆N₅ 642.32861; Found 642.32885.

### IC-11: 7-(1-((4-(Aminomethyl)benzoyl)amino)ethyl)-3-(6-(morpholin-4-ylmethyl)pyridin-3-yl)-1H-indole-2-carboxylic acid

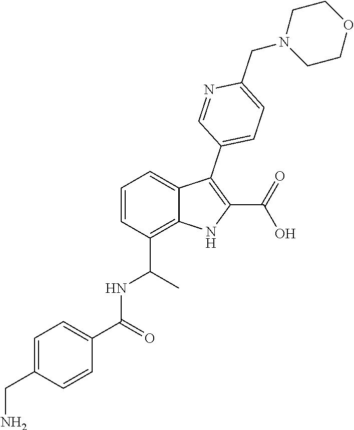 [SMILES: CC(NC(=O)c1ccc(CN)cc1)c1cccc2c(-c3ccc(CN4CCOCC4)nc3)c(C(=O)O)[nH]c12]

To a solution of N-Boc protected amine S22 (191 mg, 0.297 mmol, 1.0 eq) dissolved in CH₂Cl₂ (3.7 mL), cooled to 0° C., was added trifluoroacetic acid (TFA, 0.23 mL, 2.97 mmol, 10.0 eq) dropwise. The reaction mixture was allowed to warm to room temperature and stirred overnight. The resultant mixture was concentrated in vacuo co-evaporating with toluene. The crude residue was dissolved in THF/EtOH/H₂O (4 mL, 2:1:1) and treated with LiOH·H₂O (63 mg, 1.49 mmol, 5.0 eq). The resultant solution was stirred at room temperature overnight.

Upon completion, the resultant mixture was acidified to pH 1 with 2 M HCl, then concentrated in vacuo. The crude residue was purified using a BIOTAGE C18 SNAP-ultra column, with gradient elution using MeOH—H₂O. The solvents used for column purification were prepared by adding 1 mL of 2 M HCl to 1 L each of MeCN and of H₂O. The fractions containing the product were concentrated in vacuo and the resultant residue was re-suspended in 1:1 MeCN:H₂O, then lyophilized to obtain the desired indole carboxylate IC-11 in >99% yield as a HCl salt as a yellow powder (199 mg).

¹H NMR (500 MHz, DMSO-d₆) δ ppm 11.80 (1H, s, CO₂H), 9.16 (1H, d, J=8.0 Hz, NH), 8.77 (1H, d, J=2.0 Hz, Ar—H), 8.47 (2H, br. s, NH₂), 8.05 (1H, dd, J=8.0, 2.0 Hz, Ar—H), 7.94 (2H, d, J=8.5 Hz, Ar—H), 7.71 (1H, d, J=8.0 Hz, Ar—H), 7.59 (2H, d, J=8.0 Hz, Ar—H), 7.37 (2H, t, J=7.0 Hz, Ar—H), 7.12 (1H, t, J=7.5 Hz, Ar—H), 5.80 (1H, app p, J=7.0 Hz, CH₃CH), 4.57 (2H, s, CH₂), 4.08 (2H, app q, J=6.0 Hz, CH₂), 3.90 (4H, t, J=4.5 Hz, morpholino-CH₂), 3.36-3.29 (4H, m, morpholino-CH₂, obscured by water), 1.63 (3H, d, J=7.0 Hz, CH₃CH); ¹³C NMR (126 MHz, DMSO-d₆) δ ppm 165.5, 162.5 (C═O), 150.5, 138.9, 128.7, 127.6, 124.4, 121.8, 121.0, 118.6 (Ar—CH), 148.0, 137.2, 134.2, 133.5, 130.3, 129.9, 127.4, 124.8, 117.9 (Ar—C), 63.1 (morpholino-CH₂), 59.6 (CH₂), 51.5 (morpholino-CH₂), 45.1 (CH₃CH), 41.8 (CH₂), 20.8 (CH₃CH); HRMS (TOF, ESI⁺) m/z: [M+H]⁺ Calcd for C₂₉H₃₂O₄N₅ 514.24488; Found 514.24474.

### IC-12: 3-(3-Fluoro-4-((methylsulfonyl)methyl)phenyl)-7-(1-(piperazin-1-yl)ethyl)-1H-indole-2-carboxylic acid

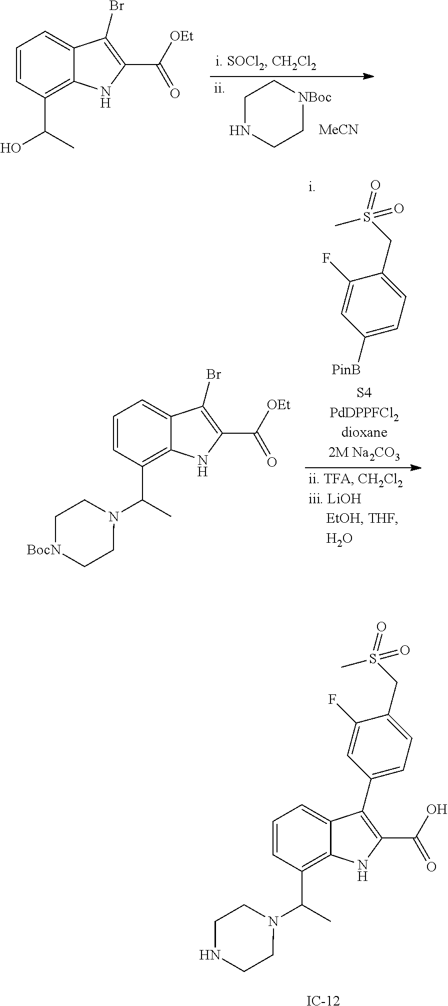

To ethyl 3-bromo-7-(1-hydroxyethyl)-1H-indole-2-carboxylate (50 mg, 0.16 mmol) was added CH₂Cl₂ (3 mL) then SOC₂ (18 μL, 0.240 mmol) at rt. Stirring was maintained for 2 h and then a 2^{nd} portion of SOCl₂ was added and the reaction mixture stirred overnight whereupon the volatiles were removed in vacuo. The crude reaction mixture was dissolved in MeCN (4 mL) and then N-Boc piperazine was added (45 mg, 0.24 mmol) and then stirred for 20 h. The reaction was quenched by the addition of EtOAc and brine; the phases were separated, the organic layer dried (Na₂SO₄) and concentrated. Purification by FCC (hexane EtOAc mixtures) afforded the intermediate with a contaminant (50 mg); the mixture was taken through to the next step. To the mixture (50 mg, 0.10 mmol) was added pinacol ester S4 (36 mg, 0.114 mmol) and PdDPPFCl₂ (5 mg, 0.007 mmol) in a microwavable vial. Degassed 1,4-dioxane (3 ml) was added followed by 2 M Na₂CO₃ (0.15 mL) and the reaction was heated under MW at 100° C. for 80 mins. The reaction mixture was cooled, filtered through Celite®, concentrated and purified by FCC (0-100% EtOAc in hexanes) to afford the cross-coupled product in 73% yield over 3 steps (45 mg).

To the N-Boc protected piperazine (45 mg) was added CH₂Cl₂ (2 mL) followed by TFA (0.1 mL) and the reaction was stirred for 18 h at RT, whereupon the reaction mixture was concentrated, dissolved in CH₂Cl₂ and washed with water. The organic layer was dried, concentrated in vacuo and the crude product was dissolved in THF (2 mL) and EtOH (1 mL) and then 2 M aq NaOH (0.5 mL) was added and the reaction was stirred for 24 h. The reaction mixture was diluted with CH₂Cl₂ and water; and the aqueous layer was extracted with CH₂Cl₂ and then 2-MeTHF, the combined organics were washed (brine), dried (Na₂SO₄) and concentrated. Purification by FCC (CH₂Cl₂ MeOH 0-20%) afforded the desired indole carboxylate IC-12 as a colourless solid in 67% yield (25 mg).

¹H NMR (400 MHz, MeOD) δ=7.54-7.42 (m, 4H), 7.11-7.05 (m, 1H), 7.02 (dd, J=8.0, 7.1 Hz, 1H), 4.51 (s, 2H), 3.73 (q, J=6.7 Hz, 1H), 2.96 (s, 3H), 2.92 (s, 3H), 2.80-2.57 (m, 3H), 2.46 (br s, 2H), 1.50 (d, J=6.7 Hz, 3H); ¹³C NMR (101 MHz, MeOD) δ 169.5, 163.4, 160.9, 140.9, 140.8, 133.8, 133.0, 133.0, 131.8, 129.4, 129.2, 128.1, 128.1, 123.0, 121.3, 119.8, 118.9, 118.7, 117.9, 114.5, 114.4, 65.4, 54.8, 54.8, 52.4, 46.7, 39.8, 18.3; HRMS (TOF, ESI⁺) m/z: [M+H]⁺ Calcd for C₂₃H₂₇FN₃O4S 460.1701; Found 460.1706.

### IC-13: 7-[1-[3-(3-Aminopropyl)-2,5-dioxo-imidazolidin-1-yl]ethyl]-3-[3-fluoro-4-(methylsulfonylmethyl)phenyl]-1H-indole-2-carboxylic acid

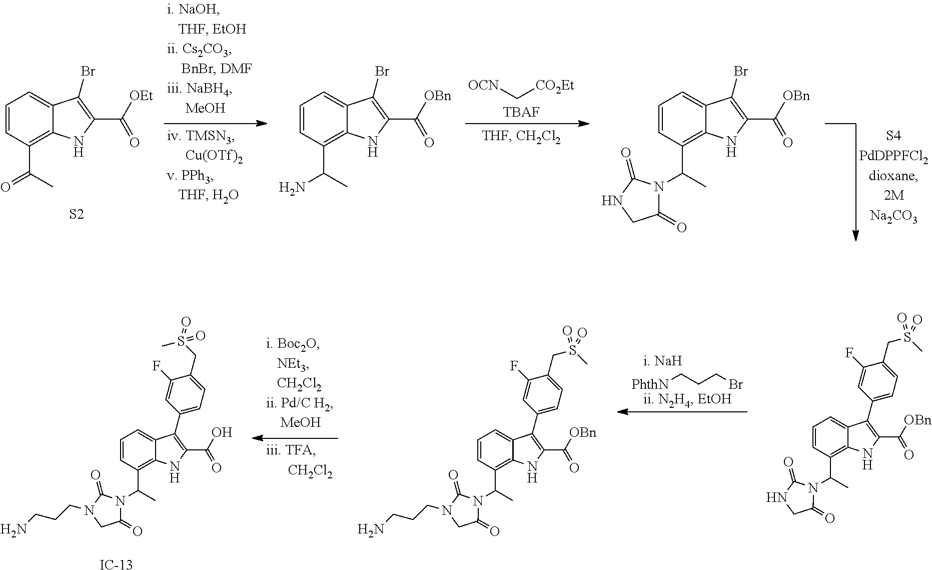

To a stirred suspension of 7-acetyl-3-bromo-1H-indole-2-carboxylic acid (1.07 g, 3.79 mmol) in MeOH/water (120 ml; 9:1) was added cesium carbonate (800 mg, 2.46 mmol) in small portions over 5 minutes and stirring was maintained over 30 mins and then the volatiles were concentrated in vacuo, azeotroping excess water with toluene (20 mL). The resulting beige amorphous solid was suspended in DMF (20 ml) at 0° C. and a solution of benzyl bromide (650 mg, 3.79 mmol) in DMF (20 ml) was added dropwise over 5 minutes. The mixture was stirred at 0° C. for 1 h, then 2 h at RT; the reaction mixture was quenched by the addition of water (40 ml) and ethyl acetate (200 ml). The organic phase was washed with brine (50 ml); dried (Na₂SO₄) and concentrated under reduced pressure to afford an amorphous yellow solid. The yellow solid was purified on silica (0→25% EtOAc in hexane) to afford the desired product as a beige powder in 63% yield (893 mg).

To a stirred solution of ketone (886 mg, 2.38 mmol) in THF (30 ml) was added NaBH₄ (181 mg, 9.52 mmol) at room temperature and the reaction was stirred for 4 hours, and then a second portion of NaBH₄ was added and then stirred overnight. The reaction was quenched with water (10 ml) and diluted with ethyl acetate (150 ml); the organic phase was washed with sat. NH₄Cl (30 ml), dried (Na₂SO₄) and concentrated under reduced pressure to afford a yellow oil. Purification by FCC (0→30% EtOAc in hexanes) gave the desired product as a white powder in 92% yield (817 mg).

To a stirred solution of the alcohol (810 mg, 2.16 mmol) in CH₂Cl₂ (20 ml) at room temperature was added azidotrimethylsilane (0.45 ml, 3.25 mmol) and copper triflate (39 mg. 0.108 mmol, 0.05 eq). The reaction was stirred for 30 minutes, then quenched with water (25 ml) and diluted with CH₂Cl₂ (50 ml). The organic phase was washed with brine (25 ml), dried (Na₂SO₄) and concentrated under reduced pressure to afford the azide as a brown powder in 98% yield (845 mg) which was used without further purification.

To a stirred solution of the azide (831 mg, 2.08 mmol) in THF (20 ml) was added water (1 ml) and triphenylphosphine (600 mg, 2.29 mmol) and the reaction was heated at reflux for 3 hours before cooling to room temperature and concentrating under reduced pressure to afford a yellow oil. Purification by FCC (CHCl₃ MeOH 0-2%) afforded the desired product as a 3:1 mixture with PPh₃O (712 mg, estimated yield 69%) and the mixture was used in the next step.

A solution of ethyl 2-isocyanatoacetate (30 mg, 2.12 mmol) in CH₂Cl₂ (1 ml) was added to a stirred solution of the amine (704 mg; 75% purity) in CH₂Cl₂ (15 ml) and the reaction was stirred for 1 h and then concentrated. The residue was dissolved in THF (3.5 ml) and then TBAF (1 M soln in THF; 0.35 ml) was added the reaction mixture heated to 60° C. for 1.5 hours and then quenched by the addition of water (25 mL) and diluted with EtOAc. The organic layer was washed with brine (10 ml), dried (Na₂SO₄) and concentrated under reduced pressure to afford a pale oil which solidified on standing in air. Purification by trituration (CH₂Cl₂) then FCC (EtOAc in hexanes 0-65%) afforded the desired product as a white powder in 62% yield (399 mg).

To the bromoindole (390 mg, 0.86 mmol), pinacol ester S4 (322 mg, 1.03 mmol), PdDPPFCl₂ (31 mg, 0.043 mmol) was added 1,4-dioxane (6.4 mL) and 2 M aq Na₂CO₃ (1.3 mL); the reaction mixture was degassed and then heated in the microwave at 100° C. for 1.5 h. The reaction mixture was cooled, filtered through Celite® (eluting EtOAc) and concentrated in vacuo. Purification by FCC (0-65% EtOAc in hexanes) afforded the cross-coupled product in 75% yield (361 mg).

To a stirred solution of the imidazolidine-2,4-dione (130 mg, 0.23 mmol) in DMF (2.5 mL) at 0° C. was added NaH (60% in mineral oil, 15 mg, 0.345 mmol) and the reaction was stirred for 45 min and then 2-(4-bromobutyl)isoindoline-1,3-dione (130 mg, 0.46 mmol) in DMF (2.5 ml) was added dropwise over 1 minute and the reaction mixture was allowed to warm to rt over 15 h whereupon the reaction mixture was quenched with brine (5 mL), diluted with EtOAc (100 mL). The organic phase was dried (Na₂SO₄) and concentrated in vacuo and purification by FCC (0-65% EtOAc in hexanes) afforded the product as a beige powder in 75% yield (132 mg).

To a solution of the N-phthlamide protected amine (129 mg, 0.169 mmol) in EtOH (15 mL) was added N₂H₄H₂O (0.15 ml, 3.37 mmol) and the reaction mixture was heated to 60° C. for 2 h; the reaction mixture was cooled to RT, diluted with CH₂Cl₂ and filtered and concentrated to afford the primary amine. The residue was dissolved in CH₂Cl₂ (4 mL), NEt₃ (37 uL, 0.253 mmol) and Boc₂O (55.2 mg, 0.253 mmol) and the reaction mixture was stirred at RT overnight. The reaction was concentrated in vacuo then purified by FCC (0-70% EtOAc in hexanes) to give the N-Boc amine in 76% yield (22 mg).

A solution of the benzyl ester (20 mg, 0.022 mmol) in MeOH (2 ml) and THF (0.5 ml) was degassed with argon for 15 minutes before adding Pd/C (10%; 3 mg) and then placed under a hydrogen atmosphere (balloon pressure) at RT. After 30 minutes at room temperature a second portion of Pd/C was added and after 2 h TLC analysis showed complete consumption of SM. The reaction mixture was filtered through Celite®, eluting with MeOH; the volatiles removed under reduced pressure and the crude mixture was purified by FCC (CH₂Cl₂: MeOH 9:1) to afford the product as a pale yellow oil in 86% yield (15 mg).

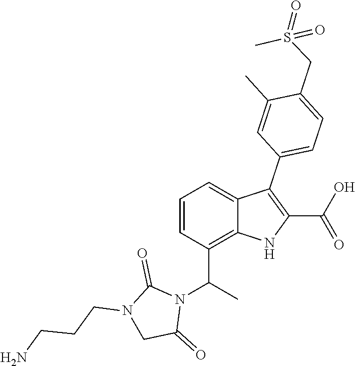 [SMILES: Cc1cc(-c2c(C(=O)O)[nH]c3c(C(C)N4C(=O)CN(CCCN)C4=O)cccc23)ccc1CS(C)(=O)=O]

To the N-Boc protected amine (14 mg, 0.022 mmol) was added CH₂Cl₂ (2 mL) followed by TFA (0.1 mL) at 0° C. and the reaction was stirred for 2 h whereupon it was concentrated to afford a yellow oil. Trituration with Et₂O (20 mL) and filtration afforded the desired indole carboxylate IC-13 as an amorphous colourless solid in 47% yield (5.5 mg).

¹H NMR (400 MHz, MeOD) δ=7.69-7.48 (m, 3H), 7.43-7.28 (m, 2H), 7.13 (ddd, J=8.2, 7.3, 1.4, 1H), 5.75 (q, J=7.4, 1H), 4.55 (s, 2H), 4.20-3.87 (m, 2H), 3.50 (td, J=6.7, 3.7, 2H), 3.31 (p, J=1.6, 4H), 2.99 (s, 3H), 2.97-2.89 (m, 2H), 2.01 (d, J=7.3, 3H), 1.95-1.84 (m, 2H); HRMS (TOF, ESI⁺) m/z: [M+H]⁺ Calcd for C₂₅H₂₈FN₄O₆S 531.1708; Found 531.1706.

### 1.2.4 Spirocyclic C7 Building Block Synthesis

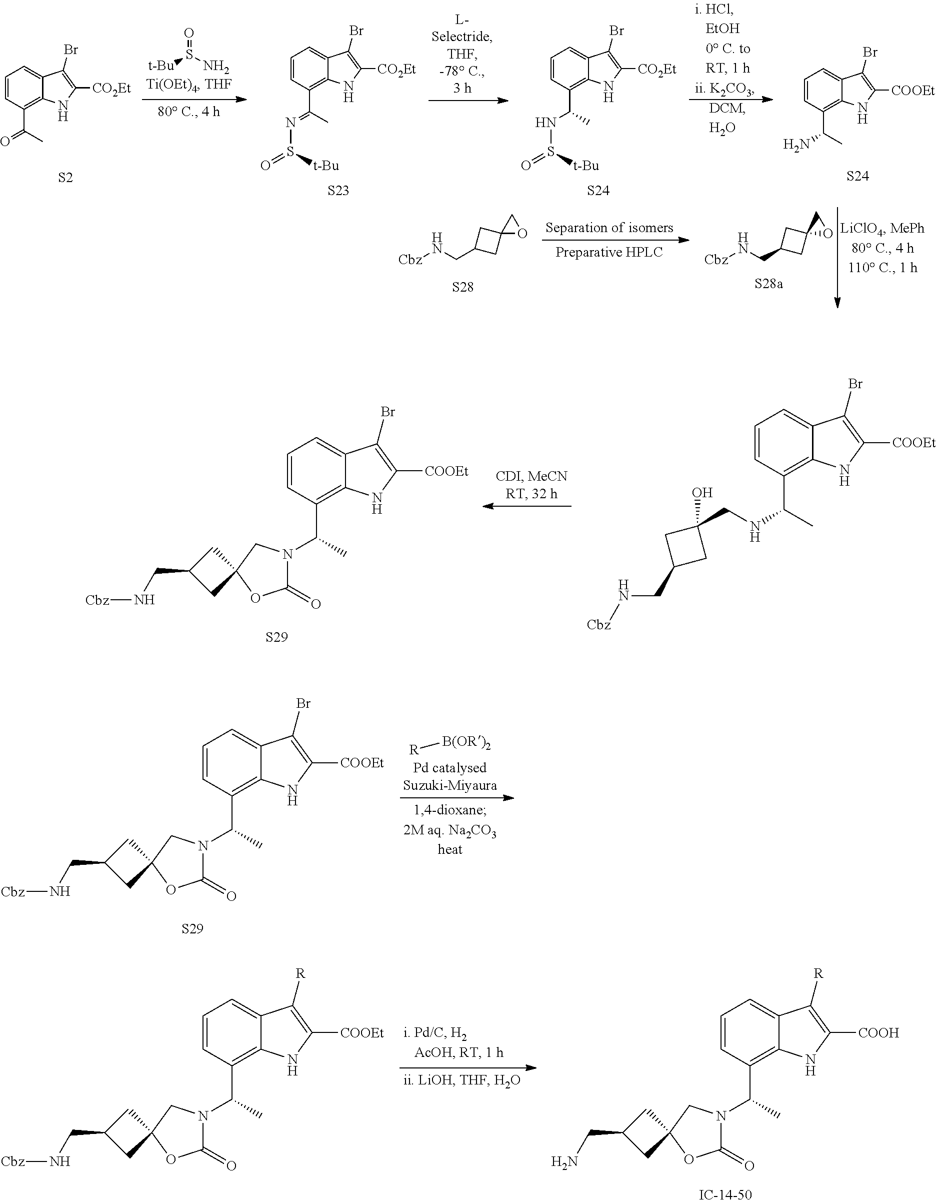

### (((1E)-1-(3-Bromo-2-(ethoxycarbonyl)-1H-indol-7-yl)ethylidene)amino)((R)-tert-butyl)sulfaniumolate (S23)

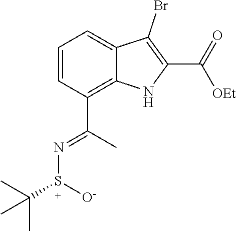

To a solution of ketone S2 (4.00 g, 12.9 mmol, 1.00 eq) in anhydrous THF (32 mL) under Ar atmosphere was added (R)-t-Bu sulfinimide (1.76 g, 14.2 mmol, 1.10 eq) and titanium ethoxide (4.92 mL, 23.2 mmol, 1.80 eq) at room temperature and the resulting yellow solution was heated at 75° C. for 24 h in a sealed tube. The resulting brown solution was cooled to rt, diluted with EtOAc, poured into a beaker with brine with stirring for 15 min; the resulting greenish yellow slurry was filtrated through a pad of Celite®, the filter cake washed with EtOAc, filtrate washed with brine, dried (Na₂SO₄) and concentrated in vacuo. The crude material was purified by FCC [100% CH₂Cl₂ to 2% MeOH in CH₂Cl₂] to afford the desired product S23 in 82% yield as a yellow solid (4.39 g).

¹H NMR (400 MHz, CDCl₃) δ ppm 11.16 (1H, br. s, NH), 7.87 (1H, dd, J=5.3, 0.9 Hz, Ar—H), 7.85 (1H, dd, J=4.9, 0.9 Hz, Ar—H), 7.29 (1H, t, J=7.8 Hz, Ar—H), 4.45 (2H, qd, J=7.1, 2.0 Hz, OCH₂CH₃), 2.91 (3H, s, CH₃), 1.43 (3H, t, J=7.1 Hz, OCH₂CH₃), 1.41 (9H, s, C(CH₃)₃); ¹³C NMR (101 MHz, CDC₃) δ ppm 178.6, 160.3, 133.6, 129.2, 128.1, 126.7, 125.2, 121.5, 120.9, 98.9, 61.5, 56.8, 22.7, 19.9, 14.6; LRMS [M+H]⁺413.1. (((1 S)-1-(3-Bromo-2-(ethoxycarbonyl)-1H-indol-7-yl)ethyl)amino)((R)-tert-butyl)sulfaniumolate (S24)

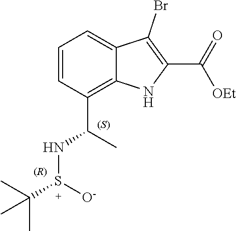

o a solution of the imine S23 (4.38 g, 10.6 mmol) in anhydrous THF (44 mL) at −76° C. was added 1 M L-Selectride in THF (11.7 mL, 11.7 mmol, 1.10 eq) dropwise and the resulting yellow suspension was stirred for 1 h between −76 and −70° C. before warming to rt. The reaction mixture was quenched by the addition of sat. NH₄Cl; the aqueous layer was extracted with EtOAc (×2), the organic layers were combined, washed with water (×2), then brine, dried (Na₂SO₄) and concentrated in vacuo. Purification of the crude material by FCC [CH₂Cl₂ to CH₂Cl₂:MeOH 50:1] and then repurification (×2) of mixed fractions by FCC [petroleum ether: EtOAc 2:1 to EtOAc] afforded the desired compound S24 as a yellow foam in 69% yield (3.02 g).

[α]_{D}²⁰=−76.0 (c 0.5, CHCl₃); ¹H NMR (400 MHz, CDCl₃) δ ppm 9.97 (1H, br. s, NH), 7.62 (1H, dt, J=8.1, 1.0 Hz, Ar—H), 7.27 (1H, ddd, J=7.2, 1.2, 0.6 Hz, Ar—H), 7.17 (1H, dd, J=8.1, 7.2 Hz, Ar—H), 4.91 (1H, qd, J=6.7, 2.7 Hz, CHCH₃), 4.51-4.34 (2H, m, OCH₂CH₃), 3.74 (1H, d, J=2.8 Hz, NHCH), 1.68 (3H, d, J=6.8 Hz, CHCH₃), 1.42 (3H, t, J=7.1 Hz, OCH₂CH₃), 1.24 (9H, s, C(CH₃)₃); ¹³C NMR (101 MHz, CDCl₃) δ ppm 160.9, 133.1, 128.9, 126.3, 125.0, 124.3, 121.5, 121.2, 98.6, 61.5, 55.8, 52.7, 22.9, 22.8, 14.4; LRMS [M+H]⁺ 415.1.

To a solution of the N-protected amine S24 (14.2 g, 34.1 mmol) in EtOH (68 mL) was added 4 M HCl (17 mL, 68 mmol) at 0° C. and the reaction mixture was warmed to RT and stirred for 1 hour and the volatiles removed in vacuo. The crude reaction mixture was slurried in refluxing Et₂O, cooled to RT, filtered and washed with Et₂O and dried to constant mass to afford the amine S25·HCl as a white powder in 92% yield (10.9 g).

[α]_{D}²⁰=+17 (c 1.0, EtOH); ¹H NMR

The amine S25·HCl (1.80 g, 5.18 mmol) was dissolved in water (180 mL) and CH₂Cl₂ (180 mL) and K₂CO₃ (2.15 g, 15.5 mmol) was added; the resulting emulsion was stirred for 15 min. The layers were separated and the aqueous layer was extracted with CH₂Cl₂ (2×100 mL); the combined organics were washed with brine (50 mL) and dried over Na₂SO₄, filtered and evaporated (30 mL of heptane was added and coevaporated) to afford the amine S25 as a white solid in 99% yield (1.61 g).

### (3-Methylidenecyclobutyl)methanamine (S26)

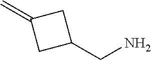

To a stirred solution of 3-methylene-1-cyano-cyclobutane (5.00 mL, 46.0 mmol) in Et₂O (70 ml) at 0° C. was slowly added 2.4 M LiAlH₄ solution in THF (32.6 mL, 78.2 mmol, 1.70 eq) and the reaction was allowed to warm to room temperature over one hour. The reaction flask was then placed in a cool water bath (10° C.) and the reaction mixture was then quenched by the addition of H₂O (3 mL, with stirring for 5 min), 15% aq. NaOH (3 mL, with stirring for 5 min) then H₂O (9 mL, with stirring for 15 min); and then the reaction was warmed to RT and stirred for 15 min and filtered and washed with Et₂O. The organic layer was dried (Na₂SO₄), filtered and concentrated (note: product is low boiling: 132° C., 744 torr) to afford the desired compound S26 as a pale yellow oil in 99% yield (4.47 g) which was used without further purification.

¹H NMR (300 MHz, CDCl₃) δ ppm 4.75 (2H, p, J=2.5 Hz, CH₂), 2.82-2.68 (4H, m, NH₂CH₂, (CH_{A}CH_{B})₂), 2.39-2.24 (3H, m, CH, (CH_{A}CH_{B})₂); ¹³C NMR (101 MHz, CDCl₃) δ ppm 146.9, 106.3, 47.7, 35.3, 33.1.

### Benzyl ((3-methylidenecyclobutyl)methyl)carbamate (S27)

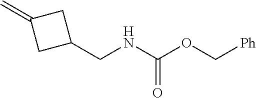 [SMILES: C=C1CC(CNC(=O)OCc2ccccc2)C1]

To a stirred solution of amine S26 (4.47 g, 45.5 mmol) in CH₂Cl₂ (100 mL) at 0° C. was slowly added Et₃N (7.62 mL, 54.7 mmol, 1.2 eq) and benzyl chloroformate (8.55 g, 50.1 mmol, 1.1 eq). The resultant reaction mixture was allowed to warm to rt and left to stir overnight. H₂O was then added, and the layers were separated. The aqueous layer was extracted with EtOAc. The combined organic extracts were dried over Na₂SO₄, filtered and concentrated in vacuo. The residue was purified by column chromatography (eluent MTBE/hexane, 20:80 to 50:50) and the desired product S27 was obtained in 74% yield as a colorless oil (8.0 g).

¹H NMR (300 MHz, CDCl₃) δ ppm 7.41-7.27 (5H, m, Ar—H), 5.10 (2H, s, CH₂Ph), 4.77 (2H, p, J=2.3 Hz, CH₂), 3.29 (2H, t, J=6.5 Hz, NH₂CH₂), 2.85-2.69 (2H, m, (CH_{A}CH_{B})₂), 2.47 (1H, h, J=6.7, CH), 2.41-2.26 (2H, m, (CH_{A}CH_{B})₂); ¹³C NMR (101 MHz, CDCl₃) δ ppm 156.6, 145.9, 136.6, 128.6, 128.6, 128.2, 106.9, 66.8, 46.0, 35.3, 30.0; LRMS 232.1.

### Benzyl (1-oxaspiro[2.3]hex-5-ylmethyl)carbamate (S28)

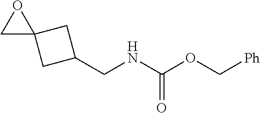 [SMILES: O=C(NCC1CC2(CO2)C1)OCc1ccccc1]

To a solution of alkene S27 (8.00 g, 33.6 mmol) in CH₂Cl₂ (70 mL) was added m-CPBA (9.93 g, 40.3 mmol, 1.2 eq) at 0° C. The mixture was allowed to warm to 22° C. and stirred for 4 h. An aqueous solution of Na₂SO₃ was then added and the reaction mixture was stirred for 20 min. The organic and aqueous layers were separated, and the latter was extracted with CH₂Cl₂ (2×250 mL). The combined organic extracts were washed with sat. aq. NaHCO₃, dried over Na₂SO₄, filtered and concentrated in vacuo and afforded the epoxide S28 as a 1:1 mixture of diasteromers in 99% yield (8.49 g).

¹H NMR (300 MHz, CDCl₃) δ ppm 7.40-7.30 (5H, m, Ar—H), 5.10 (2H, s, CH₂Ph), 4.83 (1H, s, N—H), 3.42-3.24 (2H, m, NH₂CH₂), 2.75-2.03 (7H, m, (CH₂)₂, OCH₂, CH); ¹³C NMR (101 MHz, CDC₃) δ ppm 156.7, 156.6, 136.6, 128.7, 128.3, 128.3, 66.9, 58.6, 56.9, 52.6, 52.5, 46.3, 45.8, 34.7, 33.9, 27.0, 26.4; LRMS [M+H]⁺ 248.0.

### Separation of Diastereomers

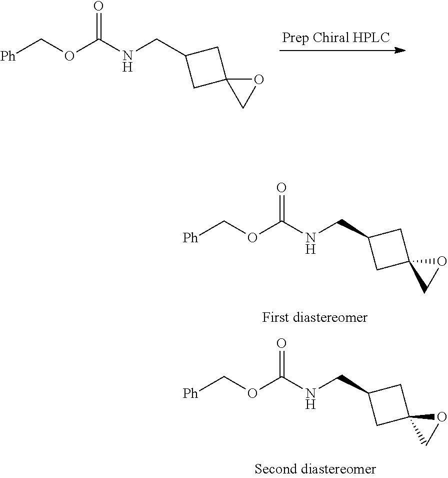 [SMILES: O=C(NCC1CC2(CO2)C1)OCc1ccccc1 ; O=C(NC[C@H]1C[C@@]2(CO2)C1)OCc1ccccc1 ; O=C(NC[C@H]1C[C@]2(CO2)C1)OCc1ccccc1]

The diastereomers S28a and S28b were separated by preparative chiral HPLC [chiralpak ID column, 250×30 mm, 5 μm, eluent 90:10 heptane:ⁱPrOH to 85:15 over 20 min, 600-800 mg per injection] to afford colourless oils that solidified upon standing in the fridge.

A solution of the amine S25 (10.1 g, 32.4 mmol), epoxide S28a (6.68 g, 27.0 mmol) and LiClO₄ (2.73 g, 25.6 mmol, 0.95 eq) in anh toluene (123 mL) was heated at 80° C. for 4 h and then 1 h at 110° C. The resulting cloudy suspension was cooled to RT, diluted with CH₂Cl₂ (100 mL) and sat. aq. NaHCO₃ was added; the layers were separated, the aqueous layer extracted with CH₂Cl₂ (100 mL×2), organic layers combined, washed with brine, dried over Na₂SO₄ and concentrated in vacuo to afford cyclobutyl alcohol as a beige foam (75% purity, ca 23% starting amine, 6% of bis-) which was taken through to the next step.

To a yellow solution of aminoalcohol (15.1 g, 20.3 mmol, 75% purity) in MeCN (230 mL) was added CDI (9.87 g, 60.8 mmol) and the reaction was stirred at RT for 16 h. UPLC analysis at this stage showed 57% conversion and a second portion of CDI was added (5 g, 1.5 equiv) and stirring was maintained for a further 16 h, at which point UPLC analysis indicated full conversion. The reaction mixture was concentrated in vacuo, diluted with EtOAc (250 mL) and aq. 1 M HCl (100 mL); the aqueous layer was extracted with EtOAc (100 mL×2); the combined organic layers were washed with sat. aq. NaHCO₃ (50 mL), brine (50 ml) and dried over Na₂SO₄ and concentrated in vacuo. Purification by FCC (CH₂Cl₂: acetone 10:1) afforded the bromoindole building block S29 in 94% yield as a white solid (11.4 g).

### 1.2.5 Spirocyclic Series

### IC-14: 7-((S)-1-((2S,4r)-2-(Aminomethyl)-6-oxo-5-oxa-7-azaspiro[3.4]octan-7-yl)ethyl)-3-(3-fluoro-4-(methylsulfonamido)phenyl)-1H-indole-2-carboxylic acid

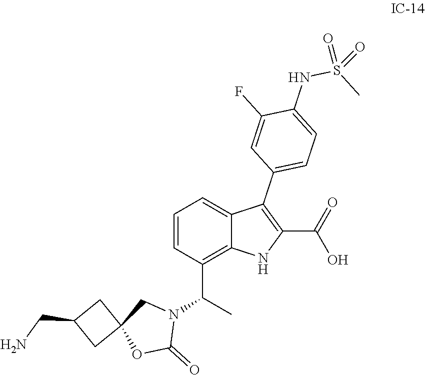 [SMILES: C[C@@H](c1cccc2c(-c3ccc(NS(C)(=O)=O)c(F)c3)c(C(=O)O)[nH]c12)N1C[C@]2(C[C@@H](CN)C2)OC1=O]

To a flask was charged bromoindole S29 (534 mg, 0.914 mmol), N-[2-fluoro-4-(4,4,5,5-tetramethyl-1,3,2-dioxaborolan-2-yl)phenyl]methanesulfonamide (374 mg, 1.19 mmol), Pd(dppf)Cl₂ (37 mg, 0.045 mmol), then 1,4-dioxane (8 mL) and 2 M Na₂CO₃ (2 mL) and the reaction mixture was degassed and then heated at 80° C. for 4 h. The reaction mixture was cooled to RT and quenched at RT with a solution of ammonium pyrrolidine-1-carbodithioate (23 mg, 0.14 mmolin 1 mL H₂O) and stirred for 1 h. The reaction mixture was diluted with EtOAc and brine, and the aqueous layer was extracted with EtOAc; the combined organics were dried (Na₂SO₄) and concentrated in vacuo. Purification by FCC (DCM then DCM:MeOH 50:1) and then by FCC (Petroleum ether: EtOAc 3:2 to 1:2) afforded the cross-coupled product in 80% yield (512 mg).

To the N-Cbz amine (452 mg, 0.65 mmol) in acetic acid (22 mL) was added 10% Pd/C (208 mg, 0.196 mmol); the reaction mixture was placed under a hydrogen atmosphere (5 bar) at RT for 20 h. The reaction mixture was filtered through a pad of Celite®, and the filter cake was washed with water (5×10 mL). The volatiles were removed in vacuo and dissolved in DCM, washed with sat. NaHCO₃; the aqueous layer was extracted with DCM then EtOAc; the organic layers were combined, washed with brine, dried (Na₂SO₄) and concentrated in vacuo to afford the amine in 93% yield (337 mg).

To the ethyl ester (337 mg, 0.603 mmol) in THF (8 mL) at 0° C. was added a solution of LiOH·H₂O (127 mg, 0.302 mmol) in 2 mL H₂O dropwise and the reaction mixture was warmed to RT and stirred for 22 h. The reaction was quenched at RT by the addition of KHSO₄ (423 mg, 0.302 mmol) and the mixture was concentrated to approx. half volume and then stirred overnight at RT. Purification by RP-FCC (Biotage KP-C18; eluent 0.05% AcOH water:MeCN 95:5 to 5:95) afforded the indole carboxylate IC-14 in 48% yield (155 mg).

¹H NMR (400 MHz, DMSO-d6) 10.42-10.23 (br s, 1H), 9.48-8.26 (br s, 4H), 7.54-7.44 (m, 2H), 7.39-7.31 (m, 2H), 7.16 (d, J=7.2 Hz, 1H), 7.02 (t, J=7.7 Hz, 1H), 5.44 (q, J=7.0 Hz, 1H), 3.67 (d, J=9.7 Hz, 1H), 3.11 (d, J=9.7 Hz, 1H), 3.06 (s, 3H), 2.90-2.74 (m, 2H), 2.61-2.54 (m, 1H, overlapped with DMSO signal), 2.47-2.42 (m, 1H, overlapped with DMSO signal), 2.40-2.30 (m, 1H), 2.17 (dd, J=12.0, 5.8 Hz, 1H), 2.03-1.94 (m, 1H), 1.61 (d, J=7.0 Hz, 3H); LRMS [M+H]⁺ 531.

### IC-15: 7-((S)-1-((2S,4r)-2-(aminomethyl)-6-oxo-5-oxa-7-azaspiro[3.4]octan-7-yl)ethyl)-3-(6-oxo-1,6-dihydropyridin-3-yl)-1H-indole-2-carboxylic acid_

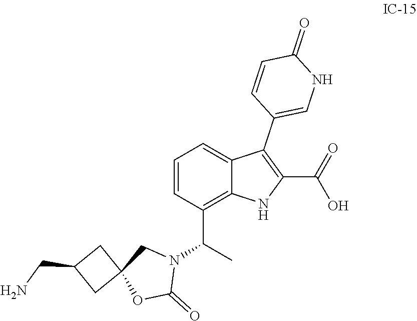 [SMILES: C[C@@H](c1cccc2c(-c3ccc(=O)[nH]c3)c(C(=O)O)[nH]c12)N1C[C@]2(C[C@@H](CN)C2)OC1=O]

To a flask was charged bromoindole S29 (1.50 g, 2.57 mmol), 2-benzyloxy-5-(4,4,5,5-tetramethyl-1,3,2-dioxaborolan-2-yl)pyridine (1.12 g, 3.59 mmol), Pd(dppf)Cl₂ (105 mg, 0.128 mmol), then 1,4-dioxane (16 mL) and 2 M Na₂CO₃ (4 mL) and the reaction mixture was degassed and then heated at 95° C. for 1 h. The reaction mixture was cooled to RT and quenched at RT with a solution of ammonium pyrrolidine-1-carbodithioate (63 mg, 0.39 mmolin 1 mL H₂O) and stirred for 1 h. The reaction mixture was diluted with EtOAc and brine, and the aqueous layer was extracted with EtOAc (3×50 mL); the combined organics were dried (Na₂SO₄) and concentrated in vacuo. Purification by FCC (KP-Sil 50 g, hexanes: EtOH, 0-50%) afforded the cross-coupled product in 96% yield (1.70 g).

To the N-Cbz amine (2.07 g, 3.52 mmol) in acetic acid (84 mL) was added 10% Pd/C (1.12 g, 1.1 mmol) and the reaction mixture was placed under a hydrogen atmosphere (1 bar) at RT for 2 h. The reaction mixture was filtered through a pad of Celite®, and the filter cake was washed with water (5×10 mL). The volatiles were removed in vacuo and the crude reaction mixture was purified by FCC (Biotage KP-C18-HS 30 g; eluent 0.05% AcOH water 50-0%; MeCN 0-50%) and afforded the amine in 70% yield (1.31 g).

To the ethyl ester (1.31 g, 2.49 mmol) in H₂O (8 mL) at 0° C. was added a solution of LiOH·H₂O (1.05 g, 24.9 mmol) in 7 mL H₂O dropwise and the reaction mixture was warmed to RT and stirred for 24 h. The reaction was quenched at RT by the addition of AcOH (1.43 mL, 24.9 mmol) and the mixture was concentrated to approx. 7 mL and then purified by FCC (Biotage KP-C18-HS 30 g; eluent 0.05% AcOH water 50-0%; MeCN 0-50%) to afford the indole carboxylate IC-15 in 50% yield (630 mg).

¹H NMR (400 MHz, DMSO-d6): 10.2-10.1 (1H, br s) 7.65 (1H, dd, J=9.4, 2.5 Hz) 7.52-7.48 (1H, m) 7.42-7.39 (1H, m) 7.13 (1H, d, J=7.2 Hz) 6.99 (1H, dd, J=7.2, 7.2 Hz) 6.31 (1H, d, J=9.4 Hz) 5.41 (1H, q, J=7.2 Hz) 3.65 (1H, d, J=9.2 Hz) 3.07 (1H, d, J=9.2 Hz) 2.86-2.75 (2H, m) 2.61-2.51 (1H, m) 2.47-2.42 (1H, m) 2.38-2.29 (1H, m) 2.20-2.12 (1H, m) 2.00-1.93 (1H, m) 1.59 (3H, d, J=7.0 Hz); LRMS [M+H]⁺ 437.

### Suzuki-Miyaura Coupling

The bromoindole S29, pinacol ester (1-3 eq), Pd catalyst (5-10 mol %) in 4:1 1,4-dioxane and 2 M aq Na₂CO₃ were degassed and heated in an oil bath or under microwave irradiation until completion. Aqueous work up then purification on silica gel afforded the cross-coupled product.

### Removal of Protecting Groups

The N-Cbz amine in AcOH was hydrogenated with 10% Pd/C under a hydrogen atmosphere (1-5 bar) for 1-5 h. Following consumption of startin material, the reaction mixture was filtered through a pad of Celite®, and the filter cake was washed with water and volatiles removed in vacuo and used crude in the next step or purified by FCC as appropriate.

### Ester Hydrolysis

To the ethyl ester in THF:EtOH:H₂O mixtures was added LiOH (5 eq) as a solid or as a solution in H₂O until consumption of starting materials. The reaction was neutralised with acid (HCl, KHSO₄ or AcOH) and then concentrated in vacuo. The indole carboxylates were purified by RP-FCC, preparative HPLC and/or crystallisation as appropriate.

The following indole carboxylates were prepared according via Suzuki-Miyaura coupling, N-CBz cleavage and ester hydrolysis:

| | IUPAC | |
|---|---|---|
| Structure/Identifier | Name | Data |
| 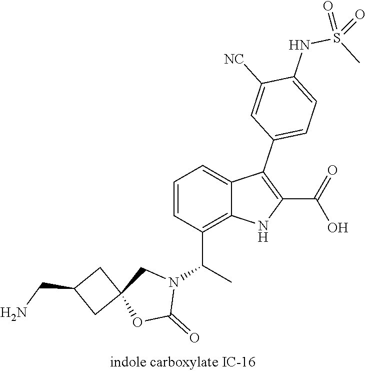 [SMILES: C[C@@H](c1cccc2c(-c3ccc(NS(C)(=O)=O)c(C#N)c3)c(C(=O)O)[nH]c12)N1C[C@]2(C[C@@H](CN)C2)OC1=O] | 7-((1S)-1- (2-(amino- methyl)- 6-oxo- 5-oxa-7- azaspiro[3. 4]octan-7- yl)ethyl)-3- (3-cyano- 4- (methylsul- fonamido) phenyl)- 1H-indole- 2- carboxylic acid | ¹H NMR (300 MHz, DMSO-d6) 10.77- 10.62 (br s, 1H), 8.47-7.83 (br s, 2H), 7.60 (s, 1H), 7.56-7.43 (m, 2H), 7.38 (d, J = 8.7 Hz, 1H), 7.26 (d, J = 7.2 Hz, 1H), 7.09 (t, J = 7.7 Hz, 1H), 5.52 (q, J = 6.9 Hz, 1H), 3.71 (d, J = 9.7 Hz, 1H), 3.28-3.20 (m, 2H, overlapped with water signal), 2.89-2.81 (m, 4H), 2.47-2.33 (m, 2H, overlapped with DMSO signal), 2.24-2.11 (m, 1H), 2.10-1.98 (m, 1H), 1.62 (d, J = 7.0 Hz, 3H); LRMS [M + H]⁺ 538. |
| 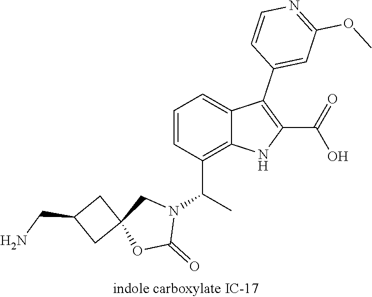 [SMILES: COc1cc(-c2c(C(=O)O)[nH]c3c([C@H](C)N4C[C@]5(C[C@@H](CN)C5)OC4=O)cccc23)ccn1] | 7-((1S)-1- (2-(amino- methyl)- 6-oxo- 5-oxa-7- azaspiro[3. 4]octan-7- yl)ethyl)-3- (2- methoxy- pyridin-4- yl)-1H- indole-2- carboxylic acid | ¹H NMR (400 MHz, DMSO-d6) 10.63- 10.22 (br s, 1H), 9.40-8.39 (br s, 2H), 8.09 (d, J = 5.3Hz, 1H), 7.46 (d, J = 8.1 Hz, 1H), 7.19-7.12 (m, 2H), 7.06-6.99 (m, 1H), 6.99-6.95 (m, 1H), 5.44 (q, J = 7.0 Hz, 1H), 3.86 (s, 3H), 3.67 (d, J = 9.6 Hz, 1H, overlapped with water signal), 3.11 (d, J = 9.6 Hz, 1H, overlapped with water signal), 2.88-2.75 (m, 2H), 2.60-2.53 (m, 1H, overlapped with DMSO signal), 2.49-2.43 (m, 1H, overlapped with DMSO signal), 2.39-2.31 (m, 1H), 2.17 (dd, J = 12.1, 5.7 Hz, 1H), 1.98 (dd, J = 12.1, 5.7 Hz, 1H), 1.59 (d, J = 7.0 Hz, 3H); LRMS [M + H]⁺ 451. |
| 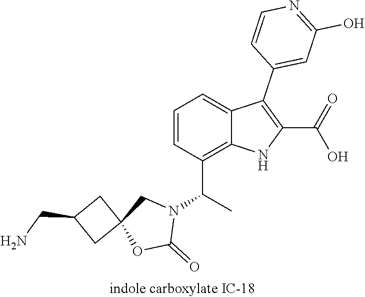 [SMILES: C[C@@H](c1cccc2c(-c3ccnc(O)c3)c(C(=O)O)[nH]c12)N1C[C@]2(C[C@@H](CN)C2)OC1=O] | 7-((S)-1- ((2S,4r)-2- (amino- methyl)- 6-oxo- 5-oxa-7- azaspiro[3. .4]octan-7- yl)ethyl)-3- (2- hydroxy- pyridin-4- yl)-1H- indole-2- carboxylic acid | ¹H NMR (400 MHz, AcOH-d4): 7.70 (dd, J = 6.8, 0.5 Hz, 1H), 7.69 (dd, J = 8.2, 0.5 Hz, 1H), 7.43 (d, J = 7.4 Hz, 1H), 7.24 (dd, J = 8.2, 7.4 Hz, 1H), 7.02 (dd, J = 1.6, 0.5 Hz, 1H), 6.86 (dd, J = 6.8, 1.6 Hz, 1H), 5.64 (q, J = 7.0 Hz, 1H), 3.74 (d, J = 9.6 Hz, 1H), 3.18 (d, J = 9.6 Hz, 1H), 3.16-3.06 (m, 2H), 2.76-2.64 (m, 2H), 2.55-2.45 (m , 1H), 2.28-2.21 (m, 1H), 2.01-1.95 (m, 1H), 1.76 (d, J = 7.0 Hz, 3H), —NH₂, —CO₂H and —Ar—OH signals not observed; LRMS [M + H]⁺ 437. |
| 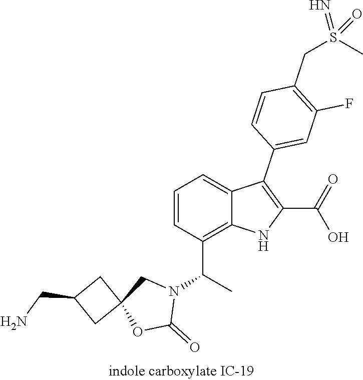 [SMILES: C[C@@H](c1cccc2c(-c3ccc(CS(C)(=N)=O)c(F)c3)c(C(=O)O)[nH]c12)N1C[C@]2(C[C@@H](CN)C2)OC1=O] | 7-((1S)-1- (2-(amino- methyl)- 6-oxo- 5-oxa-7- azaspiro[3. 4]octan-7- yl)ethyl)-3- (3-fluoro- 4-((S- methylsul- fonimidoyl) methyl) phenyl)- 1H-indole- 2- carboxylic acid | ¹H NMR (400 MHz, DMSO-d6) 10.54- 10.03 (br s, 1H), 8.62-7.68 (br s, 2H), 7.52- 7.42 (m, 3H), 7.39 (dd, J = 7.9, 1.6 Hz, 1H), 7.15 (d, J = 7.2 Hz, 1H), 7.01 (t, J = 7.6 Hz, 1H), 5.43 (q, J = 7.0 Hz , 1H), 4.51- 4.39 (m, 2H), 3.90-3.68 (br s, 1H), 3.66 (d, J = 9.7 Hz, 1H), 3.08 (d, J = 9.7 Hz, 1H), 2.89 (s, 3H), 2.85-2.74 (m, 2H), 2.60- 2.53 (m, 1H, overlapped with DMSO signal), 2.49-2.42 (m, 1H, overlapped with DMSO signal), 2.39-2.29 (m, 1H), 2.17 (dd, J = 12.5, 6.1 Hz, 1H), 1.96 (dd, J = 12.5, 6.1 Hz, 1H), 1.60 (d, J = 7.0 Hz, 3H). Signal of —CO₂H not observed; LRMS [M + H]⁺ 529 |
| 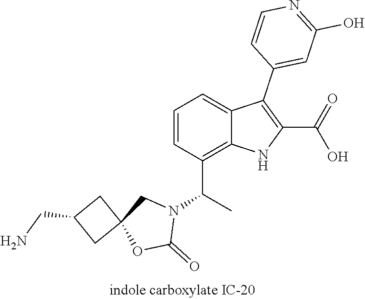 | 7-((1S)-1- (2-(amino- methyl)- 6-oxo- 5-oxa-7- azaspiro[3. 4]octan-7- yl)ethyl)-3- (2- methoxy- pyridin-4- yl)-1H- indole-2- carboxylic acid | 1H NMR (400 MHz, DMSO-d6) 10.63- 10.22 (br s, 1H), 9.40-8.39 (br s, 2H), 8.09 (d, J = 5.3 Hz, 1H), 7.46 (d, J = 8.1 Hz, 1H), 7.19-7.12 (m, 2H), 7.06-6.99 (m, 1H), 6.99-6.95 (m, 1H), 5.44 (q, J = 7.0 Hz, 1H), 3.86 (s, 3H), 3.67 (d, J = 9.6 Hz, 1H, overlapped with water signal), 3.11 (d, J = 9.6 Hz, 1H, overlapped with water signal), 2.88-2.75 (m, 2H), 2.60-2.53 (m, 1H, overlapped with DMSO signal), 2.49-2.43 (m, 1H, overlapped with DMSO signal), 2.39-2.31 (m, 1H), 2.17 (dd, J = 12.1, 5.7 Hz, 1H), 1.98 (dd, J = 12.1, 5.7 Hz, 1H), 1.59 (d, J = 7.0 Hz, 3H); LRMS [M + H]⁺ 451. |
| 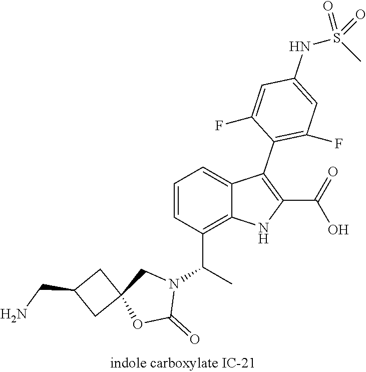 [SMILES: C[C@@H](c1cccc2c(-c3c(F)cc(NS(C)(=O)=O)cc3F)c(C(=O)O)[nH]c12)N1C[C@]2(C[C@@H](CN)C2)OC1=O] | 7-((S)-1- ((2S,4r)-2- (amino- methyl)- 6-oxo- 5-oxa-7- azaspiro[3. 4]octan-7- yl)ethyl)-3- (2,6- difluoro-4- (methylsul- fonamido) phenyl)- 1H-indole- 2- carboxylic acid | ¹H NMR (400 MHz, DMSO-d6) 10.66- 10.35 (br s, 1H), 9.22-8.14 (br s, 4H), 7.16 (d, J = 7.5 Hz, 2H), 7.01 (t, J = 7.6 Hz, 1H), 6.91-6.82 (m, 2H), 5.48 (q, J = 7.0 Hz, 1H), 3.70 (d, J = 9.7 Hz, 1H), 3.19 (d, J = 9.7 Hz, 1H, overlapped with water signal), 3.13 (s, 3H), 2.89-2.76 (m, 2H), 2.48- 2.42(m, 1H, overlapped with DMSO signal) 2.42-2.34 (m, 1H), 2.23-2.13 (m, 1H), 2.05-1.97 (m, 1H), 1.61 (d, J = 7.0 Hz, 3H), LRMS [M + H]⁺ 549. |
| 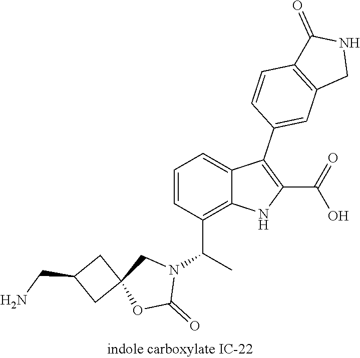 [SMILES: C[C@@H](c1cccc2c(-c3ccc4c(c3)CNC4=O)c(C(=O)O)[nH]c12)N1C[C@]2(C[C@@H](CN)C2)OC1=O] | 7-((S)-1- ((2R,4s)- 2-(amino- methyl)- 6-oxo- 5-oxa-7- azaspiro[3. 4]octan-7- yl)ethyl)-3- (1- oxoisoindo- lin-5-yl)- 1H-indole- 2- carboxylic acid | ¹H NMR (300 MHz, DMSO-d6) 10.35 (br s, 1H), 8.47 (s, 1H), 7.68 (s, 1H), 7.63 (s, 2H), 7.43 (d, J = 8.0 Hz, 1H), 7.13 (d, J = 7.3 Hz, 1H), 6.98 (t, J = 7.6 Hz, 1H), 5.45 (q, J = 7.0 Hz, 1H), 4.37 (s, 2H), 3.67 (d, J = 9.6 Hz, 1H), 3.10 (d, J = 9.6 Hz, 1H), 2.90- 2.70 (m, 2H), 2.63-2.52 (m, 1H), 2.46- 2.40 (m, 1H), 2.39-2.25 (m, 1H), 2.24-2.11 (m, 1H), 2.03-1.92 (m, 1H), 1.58 (d, J = 7.0 Hz, 3H). Signals of COOH and NH2 not observed; LRMS [M + H]⁺ 475. |
| 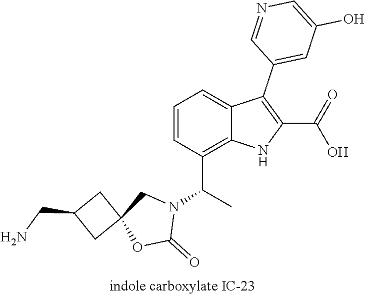 [SMILES: C[C@@H](c1cccc2c(-c3cncc(O)c3)c(C(=O)O)[nH]c12)N1C[C@]2(C[C@@H](CN)C2)OC1=O] | 7-((S)-1- ((2S,4r)-2- (amino- methyl)- 6-oxo- 5-oxa-7- azaspiro[3. 4]octan-7- yl)ethyl)-3- (5- hydroxypy- ridin-3-yl)- 1H-indole- 2- carboxylic acid | ¹H NMR (400 MHz, DMSO-d6): 10.35 (br s, 1H), 8.17 (d, J = 1.8 Hz, 1H), 8.00 (d, J = 2.7 Hz, 1H), 7.42 (dd, J = 8.0, 0.6 Hz, 1H), 7.37 (dd, J = 2.7, 1.8 Hz, 1H), 7.16 (d, J = 7.2 Hz, 1H), 7.03 (t, J = 7.6 Hz, 1H), 5.45 (d, J = 7.0 Hz, 1H), 3.68 (d, J = 9.7 Hz, 1H), 3.14 (d, J = 9.7 Hz, 1H), 2.89-2.75 (m, 2H), 2.48-2.35 (m, 3H), 2.22-2.12 (m, 1H), 2.03-1.95 (m, 1H), 1.91 (s, 3H), 1.61 (d, J = 7.0 Hz, 3H), five exchangeable proton signals —NH₂, —CO₂H, indole-N—H— not observed; LRMS [M + H]⁺ 437. |
| 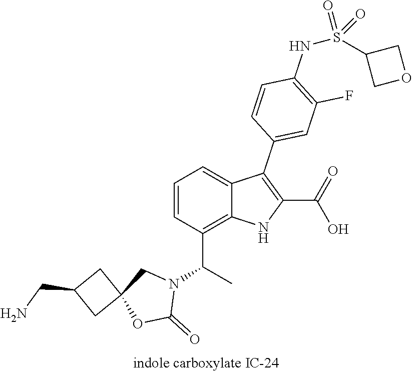 [SMILES: C[C@@H](c1cccc2c(-c3ccc(NS(=O)(=O)C4COC4)c(F)c3)c(C(=O)O)[nH]c12)N1C[C@]2(C[C@@H](CN)C2)OC1=O] | 7-((S)-1- ((2S,4r)-2- (amino- methyl)- 6-oxo- 5-oxa-7- azaspiro[3. 4]octan-7- yl)ethyl)-3- (3-fluoro- 4- (oxetane- 3-sulfon- amido) phenyl)- 1H- indole-2- carboxylic acid | ¹H NMR (400 MHz, DMSO-d6): 10.35 (1H, br s), 7.49-7.40 (2H, m), 7.33-7.27 (2H, m), 7.15 (1H, d, J = 7.2 Hz), 7.01 (1H, t, J = 7.6 Hz), 5.43 (1H, q, J = 7.0 Hz), 4.85-4.78 (2H, m), 4.74-4.68 (2H, m), 4.68-4.59 (1H, m), 3.67 (1H, d, J = 9.7 Hz), 3.10 (1H, d, J = 9.7 Hz), 2.88-2.76 (3H, m), 2.40-2.33 (1H, m), 2.21-2.13 (1H, m), 2.02-1.93 (1H, m), 1.60 (3H, d, J = 7.0 Hz), five exchangeable proton signals not observed; LRMS [M + H]⁺ 573. |
| 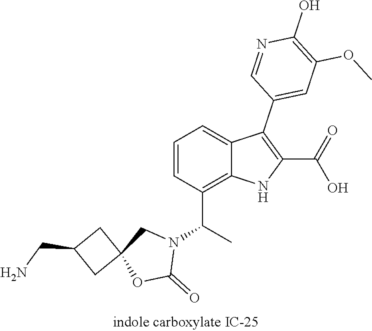 [SMILES: COc1cc(-c2c(C(=O)O)[nH]c3c([C@H](C)N4C[C@]5(C[C@@H](CN)C5)OC4=O)cccc23)cnc1O] | 7-((S)-1- ((2S,4r)-2- (amino- methyl)- 6-oxo- 5-oxa-7- azaspiro[3. 4]octan-7- yl)ethyl)-3- (6- hydroxy-5- methoxy- pyridin-3- yl)-1H- indole-2- carboxylic acid | 1H NMR (400 MHz, DMSO-d6): 10.16 (br s, 1H), 7.47 (d, J = 8.0, 1.0 Hz, 1H), 7.18-7.11 (m, 2H), 7.07 (d, J = 2.0 Hz, 1H), 7.02 (t, J = 7.6 Hz, 1H), 5.42 (q, J = 7.0 Hz, 1H), 3.67 (s, 3H), 3.66 (d, J = 9.7 Hz, 1H), 3.09 (d, J = 9.7 Hz, 1H), 2.86-2.75 (m, 2H), 2.48-2.34 (m, 3H), 2.20-2.11 (m, 1H), 2.02-1.93 (m, 1H), 1.90 (s, 3H), 1.61 (d, J = 7.0 Hz, 3H), five exchangeable proton signals —NH₃, —CO₂H, indole-N—H— not observed; LRMS [M + H]⁺ 467. |
| 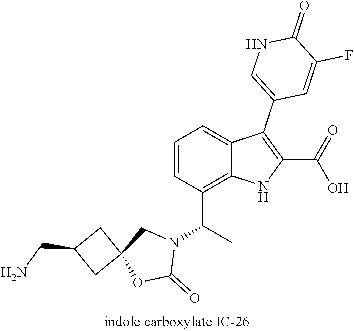 [SMILES: C[C@@H](c1cccc2c(-c3c[nH]c(=O)c(F)c3)c(C(=O)O)[nH]c12)N1C[C@]2(C[C@@H](CN)C2)OC1=O] | 7-((1S)-1- (2-(amino- methyl)- 6-oxo- 5-oxa-7- azaspiro[3. 4]octan-7- yl)ethyl)-3- (5-fluoro- 6-oxo-1,6- dihydropy- ridin-3-yl)- 1H-indole- 2- carboxylic acid | ¹H NMR (400 MHz, DMSO-d6) 10.3-10.1 (br s, 1H), 7.64 (dd, J = 12.5, 2.2 Hz, 1H), 7.44 (dd, J = 8.1, 1.0 Hz, 1H), 7.32 (d, J = 2.2 Hz, 1H), 7.14 (d, J = 7.2 Hz, 1H), 7.01 (dd, J = 7.6, 7.6 Hz, 1H), 5.42 (q, J = 7.0 Hz, 1H), 3.66 (d, J = 9.7 Hz, 1H, overlapped with water), 3.08 (d, J = 9.6 Hz, 1H, overlapped with water), 2.88-2.79 (m, 2H), 2.60-2.53 (m, 1H, overlapped with DMSO), 2.46-2.36 (m, 1H), 2.35-2.30 (m, 1H), 2.22-2.13 (m, 1H), 1.97 (dd, J = 12.9, 6.1 Hz, 1H), 1.59 (d, J = 7.0 Hz, 3H); LRMS [M + H]⁺ 455. |
| 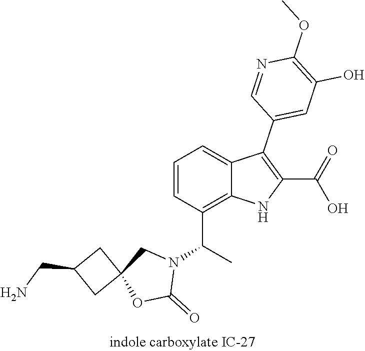 [SMILES: COc1ncc(-c2c(C(=O)O)[nH]c3c([C@H](C)N4C[C@]5(C[C@@H](CN)C5)OC4=O)cccc23)cc1O] | (S)-7-(1- (2-(amino- methyl)- 6-oxo- 5-oxa-7- azaspiro[3. 4]octan-7- yl)ethyl)-3- (5- hydroxy-6- methoxy- pyridin-3- yl)-1H- indole-2- carboxylic acid | ¹H NMR (400 MHz, DMSO-d6) 10.2-10.1 (1H, br s) 7.71 (1H, d, J = 1.9 Hz) 7.43-7.39 (1H, m) 7.33 (1H, d, J = 2.0 Hz) 7.15-7.11 (1H, m) 7.02-6.97 (1H, m) 5.42 (1H, q, J = 7.0 Hz) 3.89 (3H, s) 3.69-3.63 (1H, m) 3.12-3.07 (1H, m) 2.83-2.73 (2H, m) 2.51- 2.41(1H, m) 2.38-2.32 (1H, m) 2.18-2.11 (1H, m) 2.01-1.92 (1H, m) 1.60 (3H, d, J = 7.0 Hz); LRMS [M + H]⁺ 467. |
| 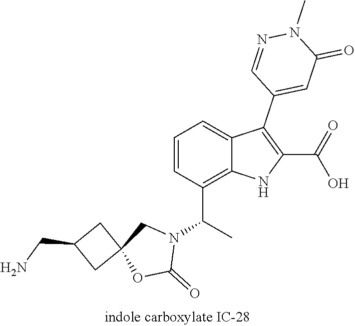 [SMILES: C[C@@H](c1cccc2c(-c3cnn(C)c(=O)c3)c(C(=O)O)[nH]c12)N1C[C@]2(C[C@@H](CN)C2)OC1=O] | 7-((S)-1- ((2S,4r)-2- (amino- methyl)- 6-oxo- 5-oxa-7- azaspiro[3. 4]octan-7- yl)ethyl)-3- (1-methyl- 6-oxo-1,6- dihydro- pyridazin- 4-yl)-1H- indole-2- carboxylic acid | ¹H NMR (400 MHz, DMSO) δ = 8.12 (d, J = 2.1, 1H), 7.50 (d, J = 8.0, 1H), 7.18 (d, J = 7.2, 1H), 7.07 (t, J = 7.6, 1H), 6.87 (d, J = 2.1, 1H), 5.45 (q, J = 7.0, 1H), 3.65 (s, 3H), 3.14 (d, J = 9.6, 1H), 2.82 (dd, J = 7.2, 3.4, 2H), 2.45 (dd, J = 8.9, 3.4, 1H), 2.35 (td, J = 10.8, 8.8, 3.8, 1H), 2.22-2.12 (m, 1H), 2.06-1.96 (m, 1H), 1.90 (s, 3H), 1.60 (d, J = 7.0, 4H). |
| 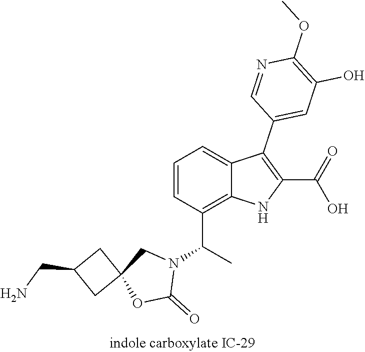 | 7-((S)-1- ((2S,4r)-2- (amino- methyl)- 6-oxo- 5-oxa-7- azaspiro[3 4]octan-7- yl)ethyl)-3- (5,6- dihydroxy pyridin-3- yl)-1H- indole-2- carboxylic acid | ¹H NMR (400 MHz, DMSO-d6): 10.21 (br s, 1H), 7.42 (d, J = 8.0, 1.0 Hz, 1H), 7.13 (d, J = 7.2 Hz, 1H), 7.04-6.95 (m, 3H), 5.41 (q, J = 7.0 Hz, 1H), 3.66 (d, J = 9.6 Hz, 1H), 3.09 (d, J = 9.6 Hz, 1H), 2.86-2.75 (m, 2H), 2.53-2.51 (m, 1H), 2.48-2.42 (m, 1H), 2.39- 2.33 (m, 1H), 2.21-2.12 (m, 1H), 2.01-1.93 (m, 1H), 1.90 (s, 3H), 1.59 (d, J = 7.0 Hz, 3H), six exchangeable proton signals not observed; LRMS [M + H]⁺ 453. |
| 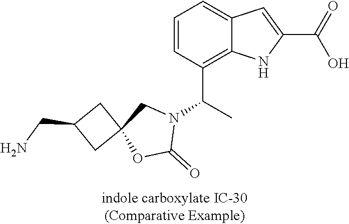 [SMILES: C[C@@H](c1cccc2cc(C(=O)O)[nH]c12)N1C[C@]2(C[C@@H](CN)C2)OC1=O] | 7-[(1S)-1- [(2r,4r)-2- (amino- methyl)- 6-oxo- 5-oxa-7- azaspiro[3. 4]octan-7- yllethyl]- 1H-indole- 2- carboxylic acid | 1H NMR (400 MHz, D₂O) δ 7.64-7.60 (1H, m), 7.27 (1H, dd, J = 7.3, 1.1 Hz), 7.18 (1H, s), 7.10 (1H, t, J = 7.7 Hz), 5.29 (1H, q, J = 7.0 Hz), 3.59 (1H, d, J = 9.9 Hz), 2.97 (1H, d, J = 9.9 Hz), 2.92-2.83 (2H, m), 2.59 (2H, dq, J = 16.2, 8.4 Hz), 2.36-2.25 (1H, m), 2.13-2.03 (1H, m), 1.79-1.68 (1H, m), 1.57 (3H, d, J = 7.0 Hz); LRMS (TOF, ESI⁺) m/z: [M + Na]⁺ Calcd for C₁₈H₂₁N₃NaO₄ 366.1; Found 366.2. |
| 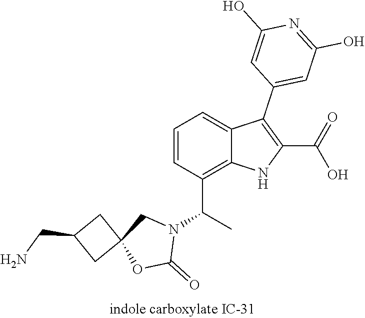 [SMILES: C[C@@H](c1cccc2c(-c3cc(O)nc(O)c3)c(C(=O)O)[nH]c12)N1C[C@]2(C[C@@H](CN)C2)OC1=O] | 3-(2,6- dihydroxy pyridin-4- yl)-7-[(1S)- 1-[(2r,4r)- 2-(amino- methyl)- 6-oxo- 5-oxa-7- azaspiro[3. 4]octan-7- ylethyl]- 1H-indole- 2- carboxylic acid | ¹H NMR (500 MHz, DMSO) δ = 11.21 (s, 1H), 7.61 (t, J = 8.4, 1H), 7.49 (d, J = 8.1, 1H), 7.32-7.01 (m, 3H), 2.85 (m, 4 H), 2.39 (dd, J = 13.2, 8.9, 2H), 2.20 (p, J = 6.5, 2H), 2.10-2.03 (m, 2H), 1.60 (m, 4H). |
| 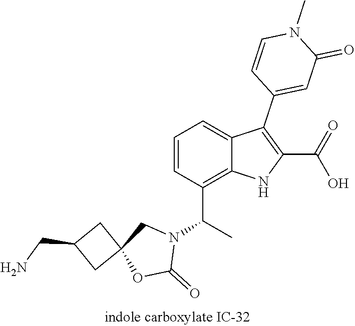 [SMILES: C[C@@H](c1cccc2c(-c3ccn(C)c(=O)c3)c(C(=O)O)[nH]c12)N1C[C@]2(C[C@@H](CN)C2)OC1=O] | 3-(1- methyl-2- oxo-1,2- dihydro- pyridin- 4-yl)- 7-[(1S)-1- [(2r,4r)-2- (amino- methyl)- 6-oxo- 5-oxa-7- azaspiro[3. 4]octan-7- yl]ethyl]- 1H-indole- 2- carboxylic acid | ¹H NMR (700 MHz, DMSO) δ ppm 7.56 (1H, d, J = 7.0 Hz, 1H), 7.48 (1H, d, J = 8.0 Hz), 7.17 (1H, d, J = 7.2 Hz), 7.05 (1H, t, J = 7.7 Hz), 6.49 (1H, d, J = 2.0 Hz), 6.44 (1H, dd, J = 7.0, 1.9 Hz), 5.45 (1H, q, J = 6.5 Hz), 3.69 (1H, d, J = 9.8 Hz), 3.16 (1H, d, J = 9.7 Hz), 2.90-2.81 (2H, m), 2.58- 2.45 (3H, m), 2.36 (1H, ddt, J = 12.6, 8.8, 5.2 Hz), 2.19 (1H, dd, J = 12.9, 6.3 Hz), 2.01 (1H, dd, J = 12.8, 6.3 Hz), 1.59 (3H, d, J = 6.8 Hz); HRMS (TOF, ESI⁺) m/z: [M + H]⁺ Calcd for C₂₄H₂₇N₄O₅ 451.1976; Found 451.1975. |
| 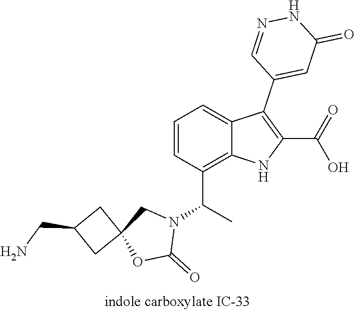 [SMILES: C[C@@H](c1cccc2c(-c3cn[nH]c(=O)c3)c(C(=O)O)[nH]c12)N1C[C@]2(C[C@@H](CN)C2)OC1=O] | 3-(6-oxo- 1,6- dihydro- pyridazin- 4-yl)-7- [(1S)-1- [(2r,4r)-2- (amino- methyl)- 6-oxo- 5-oxa-7- azaspiro[3. 4]octan-7- yl]ethyl]- 1H-indole- 2- carboxylic acid | ¹H NMR (500 MHz, DMSO) δ = 12.97 (s, 1H), 11.33 (s, 1H), 7.53 (d, J = 8.1, 1H), 7.31 (d, J = 7.2, 1H), 7.18 (t, J = 7.7, 1H), 6.88 (s, 1H), 5.56 (q, J = 7.0, 1H), 3.76 (d, J = 9.6, 1H), 2.86 (d, J = 7.5, 2H), 2.60-2.52 (m, 1H), 2.40 (ddd, J = 12.6, 8.6, 3.7, 1H), 2.29-2.19 (m, 1H), 2.16-2.08 (m, 1H), 1.60 (d, J = 7.0, 3H); HRMS (TOF, ESI⁺) m/z: [M + H]⁺ Calcd for C₂₂H₂₄N₅O₅ 438.1772; Found 438.1772. |
| 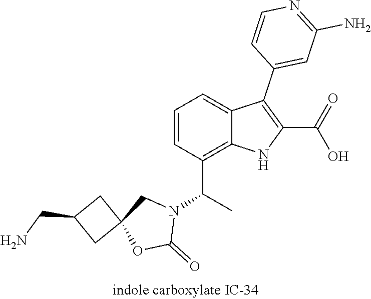 [SMILES: C[C@@H](c1cccc2c(-c3ccnc(N)c3)c(C(=O)O)[nH]c12)N1C[C@]2(C[C@@H](CN)C2)OC1=O] | 3-(2- Aminopyri- din-4-yl)- 7-[(1S)-1- [(2r,4r)-2- (amino- methyl)- 6-oxo- 5-oxa-7- azaspiro[3. 4]octan-7- yl]ethyl]- 1H-indole- 2- carboxylic acid | ¹H NMR (400 MHz, MeOH-d₄) δ ppm 7.81 (1H, d, J = 5.9 Hz), 7.64 (1H, dd, J = 8.1, 1.0 Hz), 7.31 (1H, d, J = 7.3 Hz), 7.19-7.13 (1H, m), 7.08-7.01 (2H, m), 5.53 (1H, q, J = 7.1 Hz), 3.66 (1H, d, J = 9.8 Hz), 3.05 (1H, d, J = 9.8 Hz), 3.01-2.88 (2H, m), 2.71-2.57 (2H, m), 2.52-2.41 (1H, m), 2.19 (1H, dd, J = 12.6, 6.7 Hz), 1.73 (3H, d, J = 7.1 Hz); m/z (ESI⁺) 436.2 ([M + H]⁺, 100%); HRMS (ESI⁺) m/z [M + H]⁺ Calcd for C₂₃H₂₆N₅O₄⁺ 436.1979, found 436.1978. |
| 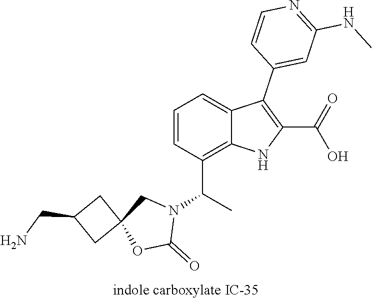 [SMILES: CNc1cc(-c2c(C(=O)O)[nH]c3c([C@H](C)N4C[C@]5(C[C@@H](CN)C5)OC4=O)cccc23)ccn1] | 3-[2- (methyl- amino) pyridin- 4-yl]-7- [(1S)-1- [(2r,4r)-2- (amino- methyl)- 6-oxo- 5-oxa-7- azaspiro[3. 4]octan-7- yl]ethyl]- 1H-indole- 2- carboxylic acid | ¹H NMR (400 MHz, MeOH-d₄) δ ppm 7.92 (1H, d, J = 5.7 Hz), 7.64 (1H, d, J = 8.0 Hz), 7.29 (1H, d, J = 7.3 Hz), 7.14 (1H, d, J = 7.7 Hz), 6.92-6.87 (2H, m), 5.53 (1H, q, J = 7.1 Hz), 3.64 (1H, d, J = 9.7 Hz), 3.05 (1H, d, J = 9.7 Hz), 2.99-2.86 (5H, m), 2.71-2.57 (2H, m), 2.53-2.44 (1H, m), 2.21-2.14 (1H, m), 1.98-1.90 (1H, m), 1.74 (3H, d, J = 7.1 Hz); m/z (ESI⁺) 450.2 ([M + H]⁺, 100%); HRMS (ESI⁺) m/z [M + H]⁺ Calcd for C₂₄H₂₈N₅O₄⁺ 450.2136, found 450.2126. |
| 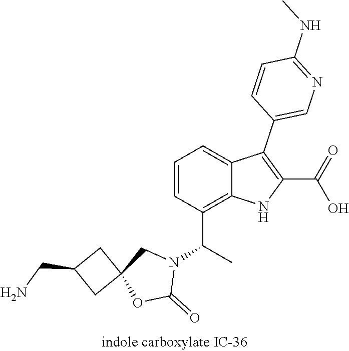 [SMILES: CNc1ccc(-c2c(C(=O)O)[nH]c3c([C@H](C)N4C[C@]5(C[C@@H](CN)C5)OC4=O)cccc23)cn1] | 3-[6- (methyl- amino) pyridin- 3-yl]-7- [(1S)-1- [(2r,4r)-2- (amino- methyl)- 6-oxo- 5-oxa-7- azaspiro[3. 4]octan-7- yl]ethyl]- 1H-indole- 2- carboxylic acid | ¹H NMR (400 MHz, MeOH-d₄) δ ppm 8.07 (1H, dd, J = 9.3, 2.0 Hz), 7.98 (1H, d, J = 1.9 Hz), 7.58 (1H, dd, J = 8.2, 0.9 Hz), 7.43 (1H, d, J = 7.3 Hz), 7.22 (1H, dd, J = 8.1, 7.2 Hz), 7.11 (1H, d, J = 9.2 Hz), 5.59 (1H, q, J = 7.1 Hz), 3.75 (1H, d, J = 9.9 Hz), 3.15-3.08 (4H, m), 3.01-2.94 (2H, m), 2.72-2.59 (2H, m), 2.52-2.41 (1H, m), 2.30-2.18 (1H, m), 2.05-1.93 (1H, m), 1.76 (3H, d, J = 7.1 Hz); m/z (ESI⁺) 450.2 ([M + H]⁺, 100%); HRMS (ESI⁺) m/z [M + H]⁺ Calcd for C₂₄H₂₈N₅O₄⁺ 450.2136, found 450.2133. |
| 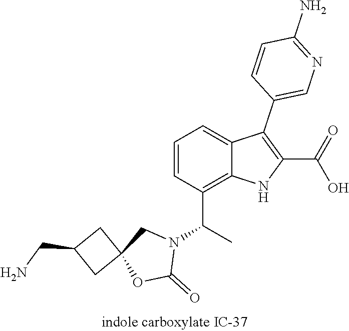 [SMILES: C[C@@H](c1cccc2c(-c3ccc(N)nc3)c(C(=O)O)[nH]c12)N1C[C@]2(C[C@@H](CN)C2)OC1=O] | 3-(6-amino- pyridin- 3-yl)- 7-[(1S)-1- [(2r,4r)-2- (amino- methyl)- 6-oxo- 5-oxa-7- azaspiro[3. 4]octan-7- yl]ethyl]- 1H-indole- 2- carboxylic acid | ¹H NMR (400 MHz, MeOH-d₄) δ ppm 8.08 (1H, m), 7.73 (1H, dd, J = 8.5, 2.3 Hz), 7.51 (1H, d, J = 8.0 Hz), 7.25 (1H, d, J = 7.3 Hz), 7.11-7.04 (1H, m), 6.67 (1H, dd, J = 8.4, 0.8 Hz), 5.51 (1H, q, J = 6.3 Hz), 3.60 (1H, d, J = 9.8 Hz), 2.99 (1H, d, J = 9.7 Hz), 2.95-2.82 (2H, m), 2.66-2.55 (2H, m), 2.53-2.43 (1H, m), 2.14 (1H, d, J = 6.3), 1.73 (3H, d, J = 7.2 Hz); m/z (ESI⁺) 436.2 ([M + H]⁺, 100%); HRMS (ESI⁺) m/z [M + H]⁺ Calcd for C₂₃H₂₆N₅O₄⁺ 436.1979, found 436.1974. |
| 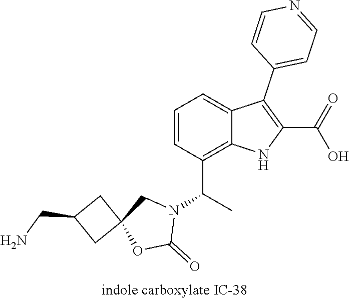 [SMILES: C[C@@H](c1cccc2c(-c3ccncc3)c(C(=O)O)[nH]c12)N1C[C@]2(C[C@@H](CN)C2)OC1=O] | 3-(pyridin- 4-yl)-7- [(1S)-1- [(2r,4r)-2- (amino- methyl)- 6-oxo- 5-oxa-7- azaspiro[3. 4]octan-7- yl]ethyl]- 1H-indole- 2- carboxylic acid | ¹H NMR (400 MHz, MeOD) δ 8.60 (2H, d, J = 5.1 Hz), 8.33 (1H, s), 7.76 (2H, d, J = 5.2 Hz), 7.62 (1H, d, J = 8.0 Hz), 7.39 (1H, d, J = 7.2 Hz), 7.21 (1H, t, J = 7.7 Hz), 5.57 (1H, q, J = 7.0 Hz), 3.71 (1H, d, J = 9.8 Hz), 3.09 (1H, d, J = 9.8 Hz), 3.02-2.94 (3H, m), 2.68 (1H, tt, J = 11.9, 5.8 Hz), 2.49 (1H, dd, J = 13.3, 6.4 Hz), 2.32-2.15 (1H, m), 1.95 (1H, dd, J = 13.3, 6.6 Hz), 1.76 (3H, d, J = 7.0 Hz); HRMS (TOF, ESI⁺) m/z: [M + H]⁺ Calcd for C₂₃H₂₅N₄O₄ 421.1870; Found 421.1870. |
| 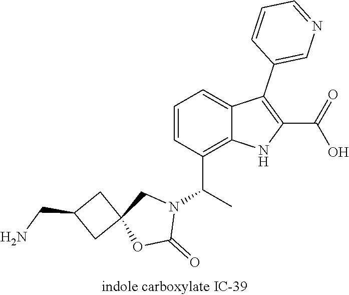 [SMILES: C[C@@H](c1cccc2c(-c3cccnc3)c(C(=O)O)[nH]c12)N1C[C@]2(C[C@@H](CN)C2)OC1=O] | 3-(pyridin- 3-yl)-7- [(1S)-1- [(2r,4r)-2- (amino- methyl)- 6-oxo- 5-oxa-7- azaspiro[3. 4]octan-7- yl]ethyl]- 1H-indole- 2- carboxylic acid | ¹H NMR (500 MHz, DMSO) δ ppm 9.17 (1H, d, J = 2.0 Hz), 8.93 (1H, dd, J = 5.7, 1.1 Hz), 8.80 (1H, dt, J = 8.1, 1.7 Hz), 8.26 (1H, s), 8.19 (1H, dd, J = 8.1, 5.8 Hz), 7.55 (1H, d, J = 8.1 Hz), 7.38 (1H, d, J = 7.2 Hz), 7.23 (1H, t, J = 7.7 Hz), 5.62 (1H, q, J = 7.0 Hz), 3.81 (1H, d, J = 9.7 Hz), 3.42 (1H, dd, J = 9.7, 3.9 Hz), 2.86 (2H, dd, J = 6.9, 3.4 Hz), 2.68-2.52 (2H, m), 2.46-2.36 (1H, m), 2.27 (1H, ddd, J = 13.6, 6.4, 2.3 Hz), 2.22-2.12 (1H, m), 1.62 (3H, d, J = 7.1 Hz); ¹³C NMR (126 MHz, DMSO) δ 163.4, 162.2, 157.0, 147.9, 142.7, 139.7, 133.9, 133.9, 127.3, 126.7, 126.0, 122.6, 122.1, 119.7, 115.4, 78.7, 52.5, 47.5, 43.1, 38.1, 37.9, 25.0, 17.8; HRMS (TOF, ESI⁺) m/z: [M + H]⁺ Calcd for C₂₃H₂₅N₄O₄ 421.1870; Found 421.1869. |
| 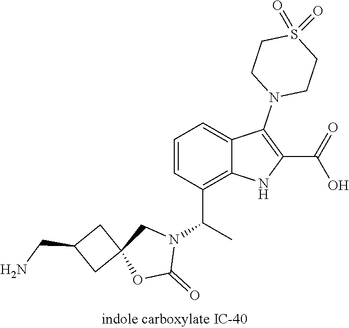 [SMILES: C[C@@H](c1cccc2c(N3CCS(=O)(=O)CC3)c(C(=O)O)[nH]c12)N1C[C@]2(C[C@@H](CN)C2)OC1=O] | 7-((S)-1- ((2S,4r)-2- (amino- methyl)- 6-oxo- 5-oxa-7- azaspiro[3. 4]octan-7- yl)ethyl)-3- (1,1- dioxidothio morpholino)- 1H- indole-2- carboxylic acid | Prepared by Buchwald-Hartwig coupling with Pd₂(dba)₃, SPhos, Cs₂CO₃, 1,4- dioxane ¹H NMR (300 MHz, AcOH-d4): 7.83 (d, J = 8.2 Hz, 1H), 7.37 (d, J = 7.2 Hz, 1H), 7.17 (t, J = 7.7 Hz, 1H), 5.56 (q, J = 6.9 Hz, 1H), 3.98-3.78 (m, 4H), 3.73 (d, J = 9.5 Hz, 1H), 3.50-3.30 (m, 4H), 3.24-3.03 (m, 3H), 2.85-2.59 (m, 2H), 2.57-2.41 (m, 1H), 2.31-2.16 (m, 1H), 2.00-1.89 (m, 1H, overlaps with AcOH-d4), 1.72 (d, J = 7.0 Hz, 3H). |
| 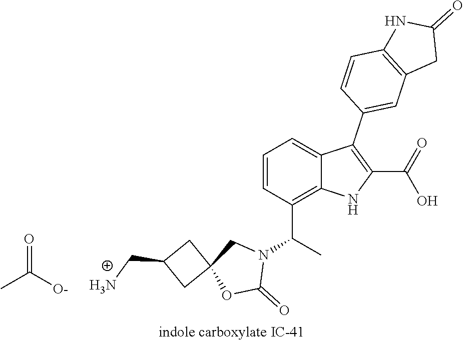 | ((2S,4r)-7- ((S)-1-(2- carboxy-3- (2- oxoindolin- 5-yl)-1H- indol-7- yl)ethyl)-6- oxo-5-oxa- 7- azaspiro[3. 4]octan-2- yl)methan aminium acetate | ¹H NMR (400 MHz, AcOH-d4): 7.61-7.57 (m, 1H), 7.52-7.49 (m, 1H), 7.49-7.44 (m, 1H), 7.38 (d, J = 7.1 Hz, 1H), 7.19- 7.10 (m, 2H), 5.65 (q, J = 7.0 Hz, 1H), 3.76- 3.69 (m, 3H), 3.17 (d, J = 9.7 Hz, 1H), 3.15-3.08 (m, 2H), 2.80-2.65 (m, 2H), 2.55-2.46 (m, 1H), 2.28-2.21 (m, 1H), 2.02-1.94 (m, 1H), 1.76 (d, J = 7.0 Hz, 3H); LRMS 475. |
| 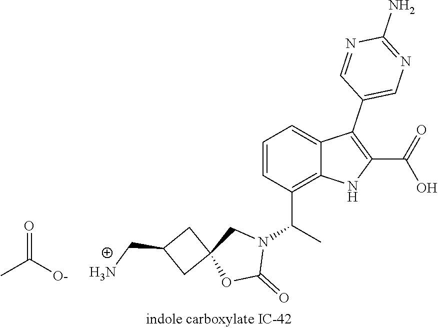 | ((2S,4r)-7- ((S)-1-(3- (2- aminopyri- midin-5- yl)-2- carboxy- 1H-indol- 7-yl)ethyl)- 6-oxo-5- oxa-7- azaspiro[3. 4]octan-2- yl)methan aminium acetate | ¹H NMR (400 MHz, AcOH-d4): 8.60 (s, 2H), 7.65 (d, J = 8.1 Hz, 1H), 7.43 (d, J = 7.2 Hz, 1H), 7.23 (t, J = 7.7 Hz, 1H), 5.64 (q, J = 7.0 Hz, 1H), 3.74 (d, J = 9.6 Hz, 1H), 3.20-3.07 (m, 3H), 2.80-2.64 (m, 2H), 2.56-2.46 (m, 1H), 2.29-2.20 (m, 1H), 2.01-1.95 (m, 1H, overlaps with AcOH- d4), 1.76 (d, J = 7.0 Hz, 3H); LRMS 437. |
| 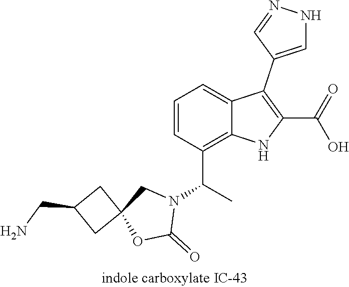 [SMILES: C[C@@H](c1cccc2c(-c3cn[nH]c3)c(C(=O)O)[nH]c12)N1C[C@]2(C[C@@H](CN)C2)OC1=O] | 7-((S)-1- ((2S,4r)-2- (amino- methyl)- 6-oxo- 5-oxa-7- azaspiro[3. 4]octan-7- yl)ethyl)-3- (1H- pyrazol-4- yl)-1H- indole-2- carboxylic acid | ¹H NMR (400 MHz, acetic acid-d4) 8.13 (s, 2H), 7.76 (d, J = 8.1 Hz, 1H), 7.38 (d, J = 7.2 Hz, 1H), 7.16 (t, J = 7.7 Hz, 1H), 5.63 (q, J = 7.0 Hz, 1H), 3.73 (d, J = 9.7 Hz, 1H), 3.25-3.03 (m, 3H), 2.88-2.61 (m, 2H), 2.57-2.41 (m, 1H), 2.33-2.23 (m, 1H), 2.02-1.92 (m, 1H, overlaps with AcOH- d4), 1.75 (d, J = 7.0 Hz, 3H); LRMS 410. |

| | |
|---|---|
| 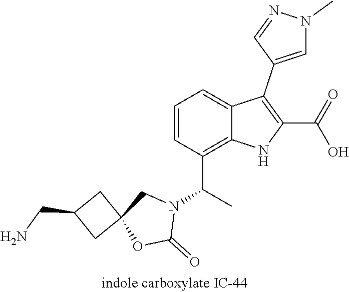 [SMILES: C[C@@H](c1cccc2c(-c3cnn(C)c3)c(C(=O)O)[nH]c12)N1C[C@]2(C[C@@H](CN)C2)OC1=O] | 3-(1-methyl-1H-pyrazol-4-yl)-7-[(1S)-1-[(2r,4r)-2- (aminomethyl)-6-oxo-5-oxa-7-azaspiro[3.4]octan-7- ylethyl]-1H-indole-2-carboxylic acid |
| 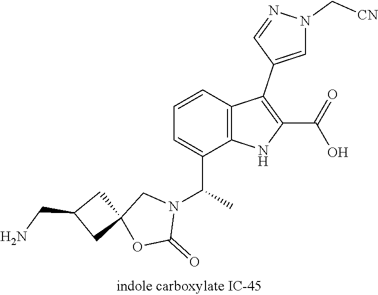 [SMILES: C[C@@H](c1cccc2c(-c3cnn(CC#N)c3)c(C(=O)O)[nH]c12)N1C[C@]2(C[C@@H](CN)C2)OC1=O] | 7-((S)-1-((2S,4r)-2-(aminomethyl)-6-oxo-5-oxa-7- azaspiro[3.4]octan-7-yl)ethyl)-3-(1-(cyanomethyl)-1H- pyrazol-4-yl)-1H-indole-2-carboxylic acid |
| 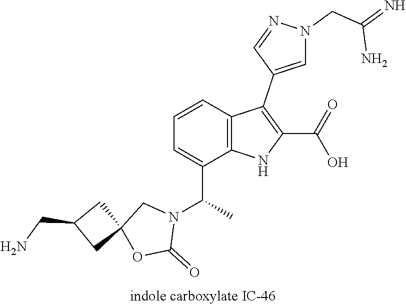 [SMILES: C[C@@H](c1cccc2c(-c3cnn(CC(=N)N)c3)c(C(=O)O)[nH]c12)N1C[C@]2(C[C@@H](CN)C2)OC1=O] | 3-(1-(2-amino-2-iminoethyl)-1H-pyrazol-4-yl)-7-((S)-1- ((2S,4r)-2-(aminomethyl)-6-oxo-5-oxa-7- azaspiro[3.4]octan-7-yl)ethyl)-1H-indole-2-carboxylic acid |
| 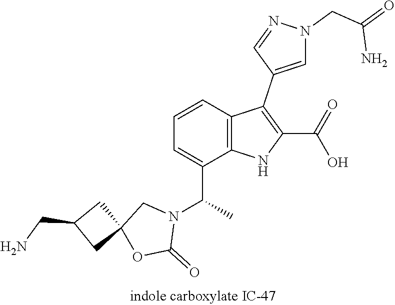 [SMILES: C[C@@H](c1cccc2c(-c3cnn(CC(N)=O)c3)c(C(=O)O)[nH]c12)N1C[C@]2(C[C@@H](CN)C2)OC1=O] | 3-(1-(2-amino-2-oxoethyl)-1H-pyrazol-4-yl)-7-((S)-1- ((2S,4r)-2-(aminomethyl)-6-oxo-5-oxa-7- azaspiro[3.4]octan-7-yl)ethyl)-1H-indole-2-carboxylic acid |
| 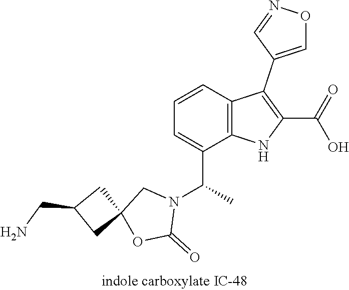 [SMILES: C[C@@H](c1cccc2c(-c3cnoc3)c(C(=O)O)[nH]c12)N1C[C@]2(C[C@@H](CN)C2)OC1=O] | 7-((S)-1-((2S,4r)-2-(aminomethyl)-6-oxo-5-oxa-7- azaspiro[3.4]octan-7-yl)ethyl)-3-(isoxazol-4-yl)-1H- indole-2-carboxylic acid |
| 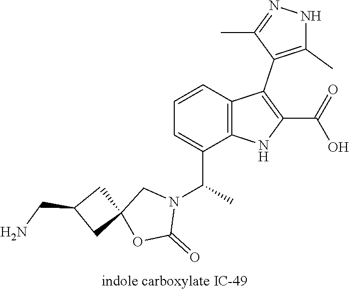 [SMILES: Cc1n[nH]c(C)c1-c1c(C(=O)O)[nH]c2c([C@H](C)N3C[C@]4(C[C@@H](CN)C4)OC3=O)cccc12] | 7-((S)-1-((2S,4r)-2-(aminomethyl)-6-oxo-5-oxa-7- azaspiro[3.4]octan-7-yl)ethyl)-3-(3,5-dimethyl-1H- pyrazol-4-yl)-1H-indole-2-carboxylic acid |
| 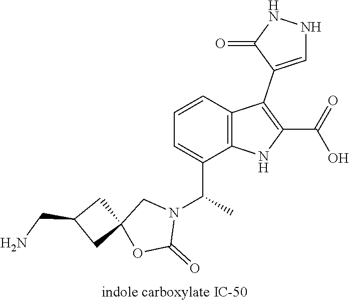 [SMILES: C[C@@H](c1cccc2c(-c3c[nH][nH]c3=O)c(C(=O)O)[nH]c12)N1C[C@]2(C[C@@H](CN)C2)OC1=O] | 7-((S)-1-((2S,4r)-2-(aminomethyl)-6-oxo-5-oxa-7- azaspiro[3.4]octan-7-yl)ethyl)-3-(3-oxo-2,3-dihydro-1H- pyrazol-4-yl)-1H-indole-2-carboxylic acid |

### Oxazolone Azetidine Building Block

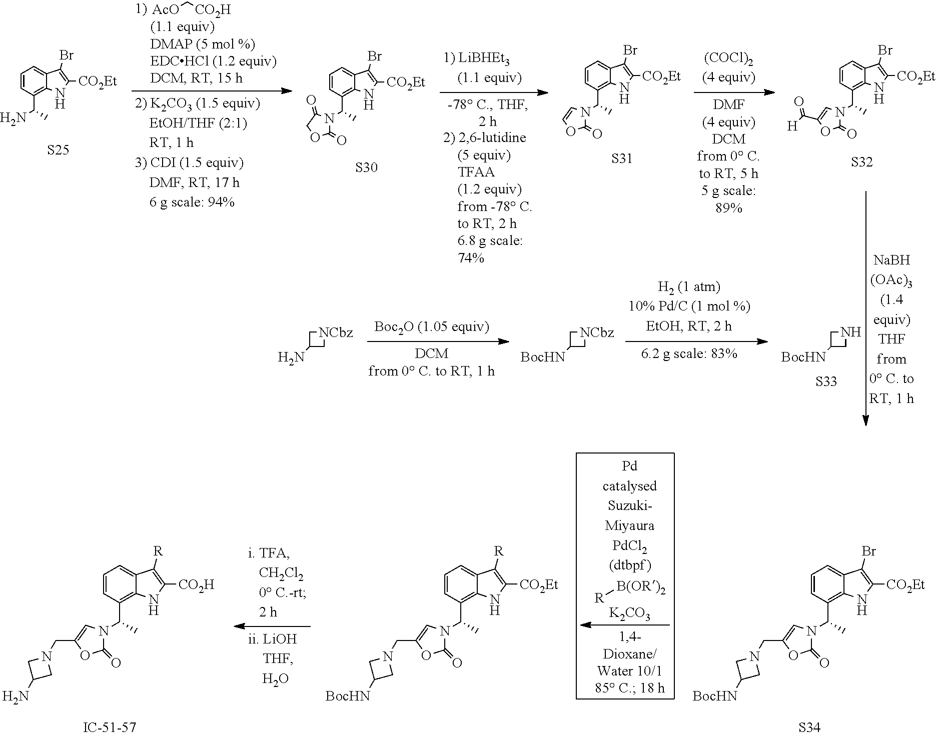

To a solution of the amine S25 (6.02 g, 19.4 mmol), 2-acetoxyacetic acid (2.51 g, 21.3 mmol, 1.10 eq) and DMAP (0.118 g, 0.97 mmol, 0.050 eq) in CH₂Cl₂ (60 mL) at 0° C. was added EDC·HCl (4.45 g, 23.2 mmol) in one portion and the reaction was stirred for 18 h at RT. The reaction mixture was diluted with CH₂Cl₂, washed sequentially with 1 M HCl, H₂O, brine, dried (Na₂SO₄) and the volatiles removed in vacuo to afford the intermediate as a yellowish solid (7.88 g). The crude acetoxyamide was dissolved in EtOH/THF (2:1, 60 mL) and treated with K₂CO₃ (2.96 g, 21.3 mmol, 1.10 eq) and the reaction was stirred for 1 h and then partitioned between EtOAc (50 mL) and H₂O (100 mL). The aqueous layer was extracted with EtOAc (2×50 mL), the combined organics were washed with H₂O (3×50 mL), sat. NH₄Cl, brine, dried (Na₂SO₄) and concentrated in vacuo to afford a yellowish solid (7.02 g). The crude hydroxyamide was dissolved in DMF (60 mL) under argon atmosphere at RT and then CDI (4.71 g, 29.0 mmol) was added in one portion and the reaction was stirred for 17 h and then the volatiles were removed in vacuo. The residue was dissolved in EtOAc, washed sequentially with 4 M HCl (×2), H₂O (×4), sat. NaHCO₃, brine, then dried (Na₂SO₄) and concentrated in vacuo (7.48 g). Purification by trituration in refluxing Et₂O (50 mL) afforded, after filtration and washing with cold Et₂O the desired compound S30 as a white solid in 94% yield over 3 steps (7.197 g).

To a solution of S30 (6.83 g, 17.3 mmol) in THF (80 mL) at −78° C. was added LiBHEt₃ (1.7 M in THF, 11.2 mL, 19.0 mmol, 1.10 eq) over 1 h and stirring was maintained at this temperature for a further hour. Lutidine (10.1 mL, 86.4 mmol, 5 eq) was then added dropwise over 10 min at −78° C. and the reaction stirred for a further 10 min and then trifluoroacetic anhydride (2.88 mL, 20.7 mmol, 1.20 eq) was added dropwise over 10 min and stirring maintained for 10 min. The cooling bath was removed and the reaction mixture was warmed to RT and stirred for 1 h and then quenched with sat. NH₄Cl (50 mL) and then stirred vigorously for 1 h under air. The reaction mixture was diluted with EtOAc; the organic layer was washed with 4 M HCl (×2), H₂O, sat NaHCO₃, brine, dried (Na₂SO₄), and then filtered through silica (6 cm diameter ×1.5 cm height) washing with EtOAc to afford 7.28 g of a yellow solid. Purification by FCC (200 g silica, hexane:EtOAc 2:1 to 1:1) then trituration in refluxing Et₂O (30 mL) afforded the oxazolone S31 in 75% yield (4.88 g) as a colourless solid.

A solution of oxalyl chloride (4.52 mL, 53.4 mmol, 4.0 eq) in CH₂Cl₂ (5 mL) was added dropwise to a solution of DMF (4.13 mL, 53.4 mmol, 4.0 eq) in CH₂Cl₂ (20 mL) at 0° C. over 30 min under Ar atmosphere and the reaction mixture was stirred at RT for 30 min and then cooled to 0° C. To this solution was added oxazolone S31 (5.06 g, 13.4 mmol, 1.0 eq) in CH₂Cl₂ (40 mL) via cannula and the reaction mixture was warmed to RT and stirred for 5 h. The obtained yellow solution was quenched with 3 M NaOAc (53 mL, 12 equiv) at 0° C. and stirred for 30 min at RT and diluted with CH₂Cl₂ (140 mL). The organic layer was washed sequentially with 4 M HCl (×2), H₂O (×4), sat. NaHCO₃, brine, then dried (Na₂SO₄) and concentrated in vacuo (5.33 g). Purification by trituration in refluxing EtOAc (15 mL) afforded the aldehyde S32 as a pale yellow solid in 89% yield (4.82 g)

[α]_{D}²⁰=+42 (c 0.5, CHCl₃).

To a solution of benzyl 3-aminoazetidine-1-carboxylate (6.19 g, 30.0 mmol) in CH₂Cl₂ (60 mL) at 0° C. was added Boc₂O (6.88 g, 31.5 mmol) and the reaction mixture was allowed to warm to RT over 1 h and then the volatiles were removed in vacuo. Purification by FCC (200 g silica, hexane: EtOAc 3:1 to 2:1) afforded the N-Boc protected amine (8.58 g) which was subsequently dissolved in EtOH (160 mL), and 10% Pd/C (0.32 g, 0.30 mmol) was added and the reaction mixture placed under an hydrogen atmosphere for 2 h at RT. The reaction mixture was flushed with argen, filtered through Celite®, then through a 0.2 μm RC filtration disc and finally transferred to a P3 frit and washed with cold Et₂O (×2) to afford the aminoazetidine S33 as a white free-flowing powder in 83% yield over two steps (4.30 g).

To a suspension of the aldehyde S32 (4.82 g, 11.8 mmol) in THF (56 mL) at 0° C. was added azetidine S33 (2.65 g, 15.4 mmol) and the resulting solution was stirred at this temperature for 10 min and then NaBH(OAc)₃ (3.51 g, 16.6 mmol) was added in one portion.

The reaction mixture was allowed to warm up to RT and stirred for 1 h and then quenched by the addition of sat. NaHCO₃ (30 mL) stirring for a further 30 min before being diluted with EtOAc. The milky aqueous layer was separated; the organic layer was washed sequentially with 1 M Na₂CO₃ (×2), H₂O, brine, dried (Na₂SO₄) and concentrated under reduced pressure (7.14 g crude). Purification by FCC (150 g silica, 6 cm diameter×10 cm height, loading in minimal toluene; EtOAc/aq.NH₃ 100:1 to EtOAc/EtOH/aq. NH₃ 100:5:1) afforded the bromoindole S34 in 96% yield as a white powder (6.39 g).

### IC-51: 7-[(1S)-1-{5-[(3-Aminoazetidin-1-yl)methyl]-2-oxo-2,3-dihydro-1,3-oxazol-3-yl}ethyl]-3-(2,6-difluoro-4-methanesulfonamidophenyl)-1H-indole-2-carboxylic acid

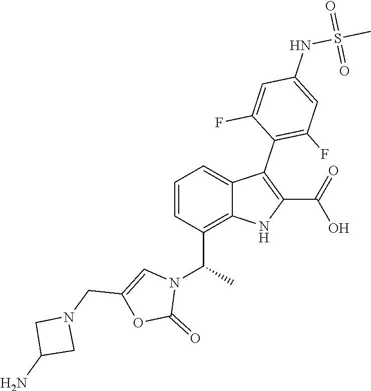 [SMILES: C[C@@H](c1cccc2c(-c3c(F)cc(NS(C)(=O)=O)cc3F)c(C(=O)O)[nH]c12)n1cc(CN2CC(N)C2)oc1=O]

A microwavable vial was charged with bromoindole S33 (400 mg, 0.71 mmol), N-[3,5-difluoro-4-(4,4,5,5-tetramethyl-1,3,2-dioxaborolan-2-yl)phenyl]methanesulfonamide (604 mg, 1.63 mmol, 90% purity), Pd(dtbpf)Cl₂ (69.4 mg, 0.107 mmol), K₂CO₃ (294 mg, 2.13 mmol) followed by 1,4-dioxane (8 mL) and H₂O (0.80 mL) and the reaction mixture was degassed and then heated at 85° C. for 18 h. The reaction mixture was cooled, diluted with EtOAc and brine, and the aqueous layer was extracted with EtOAc (3×50 mL); the combined organics were dried (Na₂SO₄) and concentrated in vacuo. Purification by FCC (KP-Sil 25 g, hexanes: EtOH, 0-30%) afforded the cross-coupled product in 75% yield (369 mg).

To the N-Boc aminoazetidine (360 mg, 0.522 mmol) in CH₂Cl₂ (10 mL) at 0° C. was added TFA (1.19 mL, 15.6 mmol) and the reaction mixture was warmed to RT and stirred for 1.5 h and then concentrated in vacuo. Purification by prep. HPLC [Atlantis column, 95/5 to 5/95 H₂O: MeCN, 0.05% TFA], evaporation of the organics and then partitioning between CH₂Cl₂ and 0.5 M Na₂CO₃ (50 mL) and extraction of the aqueous layer with CH₂Cl₂ (×6) then removal of the volatiles afforded the aminoazetidine in 71% yield (220 mg).

To aminoazetidine (220 mg, 0.373 mmol) in THF (2.0 mL) was added a solution of LiOH·H₂O (157 mg, 3.75 mmol in 2 mL H₂O) at 0° C. and the reaction mixture was gradually warmed to 19° C. over a period of 24 h. The reaction was quenched at 19° C. by the dropwise addition of KHSO₄ (526 mg, 3.75 mmol in 3 mL H₂O) and the precipate was filtered and washed with H₂O (5 mL). Purification by preparative HPLC twice [Atlantis column, 95/5 to 5/95 H₂O: MeCN, 0.05% AcOH] afforded the indole carboxylate IC-51 in 18% yield (41 mg).

[α]_{D}²⁰=+29.1 (c 0.14, DMSO); ¹H NMR (400 MHz, DMSO-d₆) 11.2-11.2 (1H, br s) 7.19-7.15 (2H, m) 7.15-7.11 (1H, m) 7.06-7.01 (1H, m) 6.92-6.86 (2H, m) 5.84 (1H, q, J=7.0 Hz) 3.60-3.53 (1H, m) 3.48-3.41 (3H, m) 3.14 (3H, s) 3.01-2.90 (2H, m) 2.54 (2H, s) 1.72 (3H, d, J=7.0 Hz)

### IC-52: 7-[(1S)-1-{5-[(3-aminoazetidin-1-yl)methyl]-2-oxo-2,3-dihydro-1,3-oxazol-3-yl}ethyl]-3-{6-[(morpholin-4-yl)methyl]pyridin-3-yl}-1H-indole-2-carboxylic acid

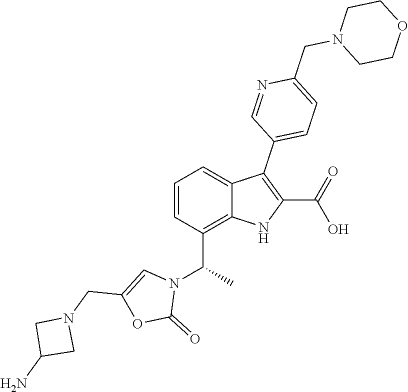 [SMILES: C[C@@H](c1cccc2c(-c3ccc(CN4CCOCC4)nc3)c(C(=O)O)[nH]c12)n1cc(CN2CC(N)C2)oc1=O]

To the bromoindole S33 (650 mg, 1.15 mmol), pinacol ester S21 (912 mg, 1.50 mmol), PdDPPFCl₂ (47 mg, 0.058 mmol) in a microwavable vial was added 1,4-dioxane (8 mL) and 2 M aq Na₂CO₃ (2 mL); the reaction mixture was degassed and then heated at 80° C. for 18 h. The reaction mixture was cooled, filtered through Celite® (eluting EtOAc) and concentrated in vacuo. Purification by FCC (0-50% EtOH in hexanes) afforded the cross-coupled product in 84% yield (638 mg).

To the N-Boc amine (628 mg, 0.950 mmol) in CH₂Cl₂ (10 mL) at 0° C. was added TFA (2.17 mL, 28.3 mmol) and the reaction was warmed to RT and stirred for 1.5 h and the volatiles were removed in vacuo. The residue was partitioned with shaking between CH₂Cl₂ and 1 M Na₂CO₃, and the aqueous layer was extracted with CH₂Cl₂ (50 mL×3); the combined organics were dried (Na₂SO₄) and concentrated. Purification by FCC (KP-Sil 50 g CH₂Cl₂:MeOH/aq NH₃ 75/15/1) afforded the amine in 52% yield (275 mg).

To the ethyl ester (275 mg, 0.491 mmol) in THF (1.55 mL) was added a solution of LiOH·H₂O (103 mg, 2.45 mmol in 1.55 mL H₂O) at 0° C. and the reaction mixture was gradually warmed to 19° C. over a period of 24 h. The reaction was quenched at 19° C. by the dropwise addition of KHSO₄ (344 mg, 2.45 mmol in 3 mL H₂O) and the volatiles removed in vacuo. Purification by preparative HPLC [Atlantis column, 95/5 to 5/95 H₂O: MeCN, 0.05% AcOH] afforded the indole carboxylate IC-52 in 28% yield (75.0 mg).

¹H NMR (400 MHz, DMSO-d6) 11.5-11.4 (1H, br s) 8.60 (1H, dd, J=2.2, 0.7 Hz) 7.88 (1H, dd, J=8.0, 2.2 Hz) 7.52-7.49 (1H, m) 7.42 (1H, dd, J=8.0, 0.7 Hz) 7.28 (1H, s) 7.20-7.16 (1H, m) 7.13-7.08 (1H, m), 5.90 (1H, q, J=7.0 Hz) 3.75-3.68 (1H, m) 3.65 (2H, s) 3.64-3.60 (4H, m) 3.50-3.44 (4H, m) 3.38 (2H, s) 3.14-3.08 (4H, m) 1.72 (3H, d, J=7.0 Hz); HRMS (TOF, ESI⁺) m/z: [M+H]⁺ Calcd for C₂₈H₃₃N₆O₅ 533.2507; Found 533.2523.

### IC-53: 7-[(1S)-1-{5-[(3-aminoazetidin-1-yl)methyl]-2-oxo-2,3-dihydro-1,3-oxazol-3-yl}ethyl]-3-(3-fluoro-4-{[imino(methyl)oxo-λ⁶-sulfanyl]methyl}phenyl)-1H-indole-2-carboxylic acid

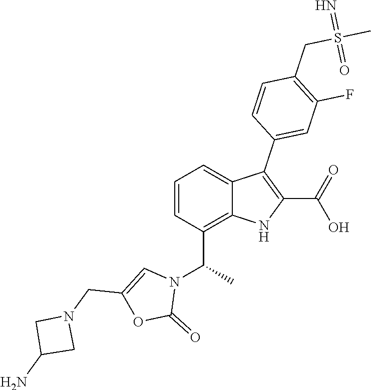 [SMILES: C[C@@H](c1cccc2c(-c3ccc(CS(C)(=N)=O)c(F)c3)c(C(=O)O)[nH]c12)n1cc(CN2CC(N)C2)oc1=O]

To the bromoindole S33 (700 mg, 1.24 mmol), [2-fluoro-4-(4,4,5,5-tetramethyl-1,3,2-dioxaborolan-2-yl)phenyl]methyl-imino-methyl-oxo-lambda6-sulfane (562 mg, 1.62 mmol), PdDPPFCl₂ (51 mg, 0.062 mmol) in a microwavable vial was added 1,4-dioxane (8 mL) and 2 M aq Na₂CO₃ (2 mL); the reaction mixture was degassed and then heated at 80° C. for 2 h. The reaction mixture was cooled and diluted with EtOAc and brine; and the aqueous layer was extracted with EtOAc, the combined organics were dried (Na₂SO₄) and concentrated in vacuo. Purification by FCC (0-30% EtOH in hexanes) afforded the cross-coupled product in 84% yield (700 mg).

To the N-Boc amine (688 mg, 1.03 mmol) in CH₂Cl₂ (10 mL) at 0° C. was added TFA (2.34 mL, 30.6 mmol) and the reaction was warmed to RT and stirred for 1.5 h and the volatiles were removed in vacuo. The residue was partitioned with shaking between CH₂Cl₂ and 1 M Na₂CO₃, and the aqueous layer was extracted with CH₂Cl₂ (50 mL×3); the combined organics were dried (Na₂SO₄) and concentrated to afford the amine in 93% yield (542 mg).

To the ethyl ester (542 mg, 0.95 mmol) in THF (3 mL) was added a solution of LiOH·H₂O (200 mg, 4.76 mmol in 3 mL H₂O) at 0° C. and the reaction mixture was gradually warmed to 19° C. over a period of 24 h. The reaction was quenched at 19° C. by the dropwise addition of KHSO₄ (451 mg, 3.21 mmol in 3 mL H₂O) and the formed precipitate was filtered. Purification by preparative HPLC [Atlantis column, 95/5 to 5/95 H₂O: MeCN, 0.05% AcOH] afforded the indole carboxylate IC-53 in 3% yield (15.0 mg).

¹H NMR (400 MHz, DMSO-d6) 11.0-10.9 (1H, br s) 7.53-7.48 (2H, m) 7.45 (1H, dd, J=11.3, 1.5 Hz) 7.39 (1H, dd, J=7.9, 1.5 Hz) 7.19-7.15 (2H, m) 7.10-7.04 (1H, m) 5.82 (1H, q, J=7.0 Hz) 4.46 (2H, s) 3.66-3.57 (1H, m) 3.47-3.40 (3H, m) 3.34 (2H, s) 3.05-2.97 (2H, m) 2.90 (3H, s) 1.73 (3H, d, J=7.0 Hz); HRMS (TOF, ESI⁺) m/z: [M+H]⁺ Calcd for C₂₆H₂₉FN₅O₅S 542.1868; Found 542.1875.

### IC-54: 7-[(1S)-1-{5-[(3-Aminoazetidin-1-yl)methyl]-2-oxo-2,3-dihydro-1,3-oxazol-3-yl}ethyl]-3-(2-oxo-1,2-dihydropyridin-4-yl)-1H-indole-2-carboxylic acid

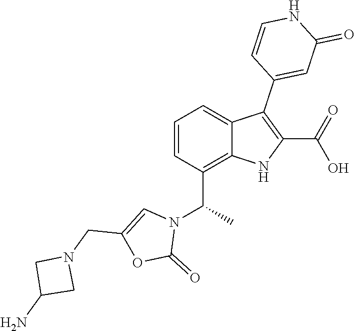 [SMILES: C[C@@H](c1cccc2c(-c3cc[nH]c(=O)c3)c(C(=O)O)[nH]c12)n1cc(CN2CC(N)C2)oc1=O]

4-Bromo-2-fluoro-pyridine (1.58 g, 9.00 mmol) was added to a predried (4A MS) solution of (4-methoxyphenyl)methanol (1.49 g, 10.8 mmol) in THF (9 mL) under argon atmosphere at 0° C. and then solid t-BuOK (1.21 g, 10.8 mmol) was added portionwise over 20 min. The reaction mixture was stirred at 0° C. for 30 min then 1 h at RT, then diluted into hexane (100 mL), stirred for 10 min, filtered through a pad of silica gel topped with Celite® eluting with hexane EtOAc (10:1) and the volatile organics removed in vacuo. Purification by FCC (hexane: EtOAc 15:1) afforded the aryl ether in in 89% yield (2.36 g).

To a flask was added the pyridyl bromide (3.15 g, 10.7 mmol), B₂Pin₂ (3.26 g, 12.8 mmol), PdDPPFCl₂ (437 mg, 0.535 mmol), KOAc (3.15 g, 32.1 mmol) and dioxane (21 mL) and the reaction mixture was degassed at RT for 20 min and then heated at 90° C. for 15 h. The dark-brown reaction mixture was cooled to RT, diluted with hexane/Et₂O (1:1), stirred for 10 min and filtered through silica covered with Celite® and then concentrated to afforded a brownish oil (4.37 g). The crude was redissolved in hexane/Et₂O (1:1), washed with 2% aqueous solution of ammonium pyrrolidinedithiocarbamate (5×5 mL), brine, dried (MgSO₄) and filtered through silica covered with tightly packed MgSO₄ and then concentrated (3.67 g). Recrystalisation from hexane (bp.→RT), then washing with cold hexane (×3) afforded the pinacol ester in 83% yield as large colourless prisms (3.04 g).

To the bromoindole S33 (676 mg, 1.20 mmol), the pinacol ester (492 mg, 1.44 mmol), PdDPPFCl₂ (49 mg, 0.060 mmol) was added 1,4-dioxane (9.6 mL) and 2 M aq Na₂CO₃ (2.4 mL); the reaction mixture was degassed and then heated at 80° C. for 1 h. The reaction mixture was cooled and quenched by the addition of ammonium pyrrolidinedithiocarbamate (30 mg, 0.18 mmol, 1 mL H₂O) and stirred for 1 h at RT. The reaction mixture was filtered through Celite® (eluting EtOAc) and the filtrate was washed with H₂O (3×), brine, dried (Na₂SO₄), filtered and concentrated. and concentrated in vacuo. Purification by FCC (EtOAc aq NH₃ 100:1) afforded the cross-coupled product in 93% yield (781 mg).

To the N-Boc amine (703 mg, 1.01 mmol) in CH₂Cl₂ (10 mL) at 0° C. was added TFA (2.32 mL, 30.2 mmol) and the reaction was warmed to RT and stirred for 2 h and the volatiles were removed in vacuo. The residue was suspended in Et₂O (20 mL) at RT, the yellow supernatant discarded and the residual solid triturated with Et₂O (2×10 mL) afforded the amine as the bistrifluoroacetate salt as a yellowish powder in 93% yield (835 mg).

The ethyl ester (670 mg, 0.950 mmol) was sonicated for 10 min in H₂O (7 mL) and then a solution of LiOH·H₂O (199 mg, 4.75 mmol in 1 mL H₂O) at RT and the reaction mixture was vigorously stirred for 14 h. The reaction was quenched by the dropwise addition of AcOH (0.217 mL, 3.80 mmol) and then filtered through a 0.45 μm RC disc. Purification by preparative HPLC [Reversed phase PrepHPLC (Atlantis, 5 μm, 30×100 mm, H₂O+0.5% AcOH/MeCN+0.5% AcOH 10→70% gradient] afforded the indole carboxylate IC-54 in 93% yield as a yellowish powder (398 mg).

¹H NMR (400 MHz, D₂O) 7.41 (d, J=6.7 Hz, 1H), 7.12 (d, J=8.1 Hz, 1H), 7.03 (d, J=7.3 Hz, 1H), 6.78 (t, J=7.7 Hz, 1H), 6.56 (d, J=1.1 Hz, 1H), 6.46 (s, 1H), 6.32 (dd, J=6.8, 1.6 Hz, 1H), 5.50 (q, J=7.0 Hz, 1H), 4.08 (p, J=7.2 Hz, 1H), 3.97-3.83 (m, 2H), 3.66-3.57 (m, 2H), 3.52 (d, J=14.5 Hz, 1H), 3.44 (d, J=14.5 Hz, 1H), 1.63 (d, J=7.0 Hz, 3H); HRMS (TOF, ESI⁺) m/z: [M+H]⁺ Calcd for C₂₃H₂₄N₅O₅ 450.1772; Found 450.1777.

### IC-55: 7-[(1S)-1-{5-[(3-aminoazetidin-1-yl)methyl]-2-oxo-2,3-dihydro-1,3-oxazol-3-yl}ethyl]-3-(3-cyano-4-methanesulfonamidophenyl)-1H-indole-2-carboxylic acid

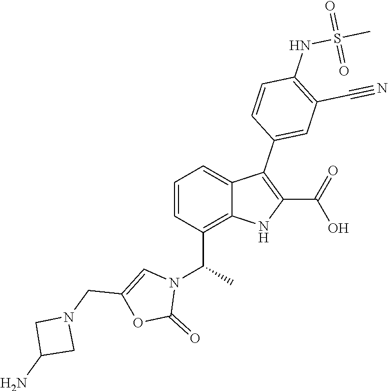 [SMILES: C[C@@H](c1cccc2c(-c3ccc(NS(C)(=O)=O)c(C#N)c3)c(C(=O)O)[nH]c12)n1cc(CN2CC(N)C2)oc1=O]

To the bromoindole S33 (800 mg, 1.42 mmol), N-[2-cyano-4-(4,4,5,5-tetramethyl-1,3,2-dioxaborolan-2-yl)phenyl] methane sulfonamide (610 mg, 1.70 mmol, 90% purity), PdDPPFCl₂ (58 mg, 0.071 mmol) in a microwavable vial was added 1,4-dioxane (9.5 mL) and 2 M aq Na₂CO₃ (3.5 mL); the reaction mixture was degassed and then heated at 90° C. for 1 h. The reaction mixture was cooled and quenched by the addition of ammonium pyrrolidinedithiocarbamate (35 mg, 0.21 mmol, 1 mL H₂O) and then diluted with EtOAc and the aqueous layer was extracted with EtOAc; the combined organics washed with brine, dried (Na₂SO₄) and concentrated in vacuo. Purification by FCC (0-11% MeOH in EtOAc) and then by FCC (EtOAc:MeOH: aq NH₃ 100:10:1) afforded the cross-coupled product in 65% yield (627 mg).

To the N-Boc amine (627 mg, 0.923 mmol) in CH₂Cl₂ (10 mL) at 0° C. was added TFA (2.11 mL, 27.5 mmol) and the reaction was warmed to RT and stirred for 1.5 h and the volatiles were removed in vacuo. The residue was partitioned with shaking between CH₂Cl₂ and 0.5 M Na₂CO₃, and the aqueous layer was extracted with CH₂Cl₂ (50 mL×3); the combined organics were dried (Na₂SO₄) and concentrated; as well as the aqueous layer. The residues were combined and purified by FCC (CH₂Cl₂:MeOH/aq NH₃ 300/60/2) afforded the amine in 67% yield (360 mg).

To the ethyl ester (372 mg, 0.643 mmol) in THF (3 mL) was added a solution of LiOH·H₂O (135 mg, 3.21 mmol in 3 mL H₂O) at 0° C. and the reaction mixture was gradually warmed to 19° C. over a period of 24 h. The reaction was quenched at 19° C. by the dropwise addition of KHSO₄ (451 mg, 3.21 mmol in 3 mL H₂O) and the volatiles removed in vacuo. Purification by preparative HPLC [Xbridge eluent 0.1% AcOH water 95-5%; MeCN 5-95%, and then Atlantis column, 95/5 to 5/95 H₂O: MeCN, 0.05% AcOH] afforded the indole carboxylate IC-55 in 21% yield (75.0 mg).

¹H NMR (400 MHz, DMSO-d6) 11.3-11.2 (1H, br s) 7.65-7.61 (1H, m) 7.53 (1H, dd, J=8.2, 1.5 Hz) 7.45 (1H, dd, J=8.2, 1.0 Hz) 7.42-7.39 (1H, m) 7.25 (1H, s) 7.19-7.16 (1H, m) 7.11-7.06 (1H, m) 5.88 (1H, q, J=7.0 Hz) 3.75-3.68 (1H, m) 3.48-3.40 (2H, m) 3.38 (2H, s) 3.15-3.09 (2H, m) 2.88 (3H, s) 1.72 (3H, d, J=7.0 Hz); HRMS (TOF, ESI⁺) m/z: [M+H]⁺ Calcd for C₂₆H₂₇N₆O₆S 551.1707; Found 551.1727.

### IC-56: 7-(1-(((4-(Aminomethyl)phenyl)carbamoyl)oxy)ethyl)-3-(3-fluoro-4-((methylsulfonyl)methyl)phenyl)-1H-indole-2-carboxylic acid

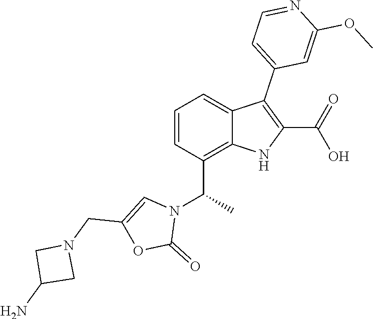 [SMILES: COc1cc(-c2c(C(=O)O)[nH]c3c([C@H](C)n4cc(CN5CC(N)C5)oc4=O)cccc23)ccn1]

To the bromoindole S33 (700 mg, 1.24 mmol), 2-methoxy-4-(4,4,5,5-tetramethyl-1,3,2-dioxaborolan-2-yl)pyridine (475 mg, 1.62 mmol, 80% purity), PdDPPFCl₂ (51 mg, 0.062 mmol) in a microwavable vial was added 1,4-dioxane (9.5 mL) and 2 M aq Na₂CO₃ (3.5 mL); the reaction mixture was degassed and then heated at 90° C. for 1 h. The reaction mixture was cooled and quenched by the addition of ammonium pyrrolidinedithiocarbamate (35 mg, 0.21 mmol, 1 mL H₂O) and then diluted with EtOAc and the aqueous layer was extracted with EtOAc; the combined organics washed with brine, dried (Na₂SO₄) and concentrated in vacuo. Purification by FCC (0-11% MeOH in EtOAc) afforded the cross-coupled product in 74% yield (541 mg).

To the N-Boc amine (535 mg, 0.904 mmol) in CH₂Cl₂ (8 mL) at 0° C. was added TFA (2.06 mL, 26.9 mmol) and the reaction was warmed to RT and stirred for 2 h and the volatiles were removed in vacuo. The residue was partitioned with shaking between CH₂Cl₂ and 1 M Na₂CO₃, and the aqueous layer was extracted with CH₂Cl₂ (50 mL×3); the combined organics were dried (Na₂SO₄) and concentrated. Purification by FCC (KP-Sil 50 g CH₂Cl₂:MeOH/aq NH₃ 150/15/1) afforded the amine in 86% yield (402 mg).

To the ethyl ester (400 mg, 0.814 mmol) in THF (3.5 mL) was added a solution of LiOH·H₂O (171 mg, 2.45 mmol in 3.5 mL H₂O) at 0° C. and the reaction mixture was gradually warmed to 19° C. over a period of 24 h. The reaction was quenched at 19° C. by the dropwise addition of KHSO₄ (1.00 g, 7.14 mmol in 3 mL H₂O) and the volatiles removed in vacuo. Purification by RP Biotage purification [KP-C18-HS (eluent H₂O+0.5% AcOH/MeCN 0-50%] then by preparative HPLC [XBridge column, 95/5 to 5/95 H₂O: MeCN, 0.1% AcOH] afforded the desired compound indole carboxylate IC-56 in 30% yield (120 mg).

¹H NMR (400 MHz, DMSO-d6) 11.1-10.9 (1H, brs) 8.14 (1H, d, J=5.2 Hz) 7.48-7.44 (1H, m) 7.19-7.12 (3H, m) 7.09-7.03 (1H, m) 6.95 (1H, s) 5.82 (1H, q, J=7.0 Hz) 3.88 (3H, s) 3.64-3.56 (1H, m) 3.48-3.30 (4H, m) 3.04-2.95 (2H, m) 1.72 (3H, d, J=7.0 Hz); HRMS (TOF, ESI⁺) m/z: [M+H]⁺ Calcd for C₂₄H₂₆N₅O₅ 464.1928; Found 464.1941.

### IC-57: 7-[(1S)-1-{5-[(3-Aminoazetidin-1-yl)methyl]-2-oxo-2,3-dihydro-1,3-oxazol-3-yl}ethyl]-3-(3-fluoro-4-methanesulfonamidophenyl)-1H-indole-2-carboxylic acid

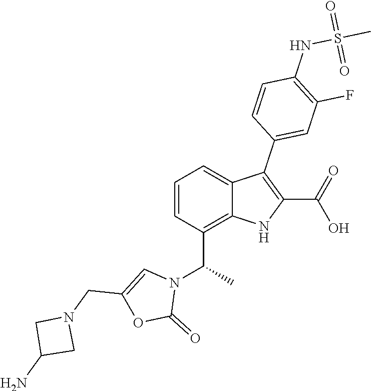 [SMILES: C[C@@H](c1cccc2c(-c3ccc(NS(C)(=O)=O)c(F)c3)c(C(=O)O)[nH]c12)n1cc(CN2CC(N)C2)oc1=O]

To the bromoindole S33 (563 mg, 1.00 mmol), N-[2-fluoro-4-(4,4,5,5-tetramethyl-1,3,2-dioxaborolan-2-yl)phenyl]methane sulfonamide (409 mg, 1.30 mmol), PdDPPFCl₂ (41 mg, 0.050 mmol) in a microwavable vial was added 1,4-dioxane (8 mL) and 2 M aq Na₂CO₃ (2 mL); the reaction mixture was degassed and then heated at 80° C. for 3 h. The reaction mixture was cooled and quenched by the addition of ammonium pyrrolidinedithiocarbamate (25 mg, 0.15 mmol, 1 mL H₂O) and then diluted with EtOAc and the aqueous layer was extracted with EtOAc; the combined organics washed with brine, dried (Na₂SO₄) and concentrated in vacuo. Purification by FCC (EtOAc/aq NH₃ 100:1 to EtOAc/EtOH/aq NH₃ 100:5:1) afforded the cross-coupled product in 89% yield (596 mg).

To the N-Boc amine (589 mg, 0.877 mmol) in CH₂Cl₂ (7 mL) at 0° C. was added TFA (2.00 mL, 26.1 mmol) and the reaction was warmed to RT and stirred for 1.5 h and the volatiles were removed in vacuo. The residue was partitioned with shaking between CH₂Cl₂ and 1 M Na₂CO₃, and the aqueous layer was extracted with CH₂Cl₂ (50 mL×3); the combined organics were dried (Na₂SO₄) and concentrated. Purification by FCC (CH₂Cl₂:MeOH/aq NH₃ 150/15/1 to 80:10:1), evaporation of the product fractions and the filtration through a 0.2 μm RC disc afforded the amine in 85% yield (426 mg).

To the ethyl ester (420 mg, 0.735 mmol) in THF (3.50 mL) was added a solution of LiOH·H₂O (154 mg, 3.67 mmol in 3.50 mL H₂O) at 0° C. and the reaction mixture was gradually warmed to 19° C. over a period of 24 h. The reaction was quenched at 19° C. by the dropwise addition of KHSO₄ (500 mg, 3.67 mmol in 1.5 mL H₂O) and then concentrated to a volume of 6 mL. The formed precipitate was filtered on a P3 frit and washed with H₂O (4×2 mL). Purification by preparative HPLC [Atlantis column, 5 μm, 30×100 mm, H₂O+0.1% AcOH/MeCN 10→50% gradient] afforded the indole carboxylate IC-57 in 54% yield (214 mg). ¹H NMR (400 MHz, DMSO-d6) 11.44-10.28 (bs, 1H), 7.52-7.43 (m, 2H), 7.41-7.31 (m, 2H), 7.18-7.10 (m, 2H), 7.04 (t, J=7.6 Hz, 1H), 5.81 (q, J=7.0 Hz, 1H), 3.62 (p, J=6.4 Hz, 1H, overlaps with H₂O), 3.44 (q, J=7.2 Hz, 2H, overlaps with H₂O), 3.32 (s, 2H, overlaps with H₂O), 3.13-2.96 (m, 5H, overlaps with H₂O), 1.71 (d, J=7.0 Hz, 3H), —NH₂, —CO₂H and —NH—SO₂Me signals not observed; HRMS (TOF, ESI⁺) m/z: [M+H]⁺ Calcd for C₂₅H₂₇FN₅O₆S 544.1661; Found 544.1678.

### IC-58: (S)-3-(3-fluoro-4-((methylsulfonyl)methyl)phenyl)-7-(1-(5-(guanidinomethyl)-2-oxooxazol-3(2H)-yl)ethyl)-1H-indole-2-carboxylic acid

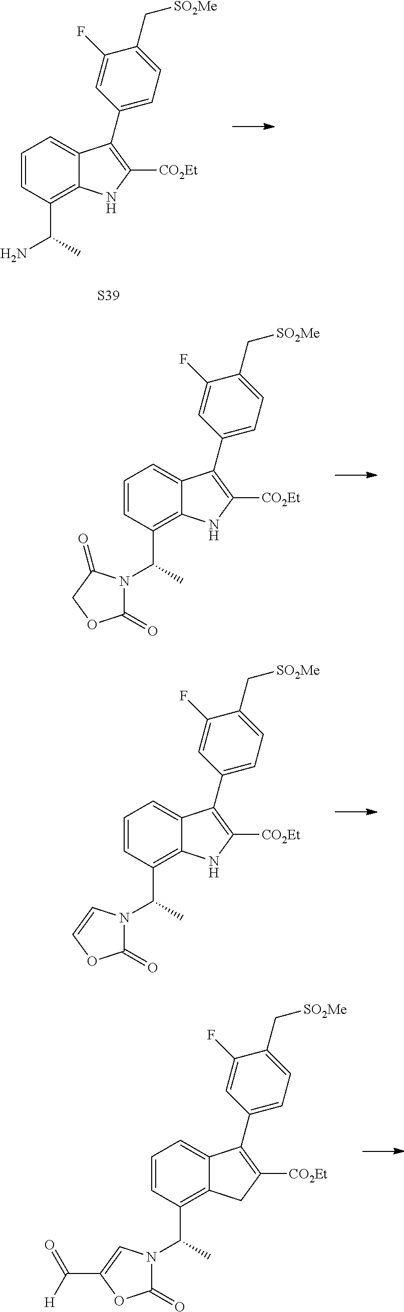 [SMILES: CCOC(=O)c1[nH]c2c([C@H](C)N)cccc2c1-c1ccc(CS(C)(=O)=O)c(F)c1 ; "S39" ; CCOC(=O)c1[nH]c2c([C@H](C)N3C(=O)COC3=O)cccc2c1-c1ccc(CS(C)(=O)=O)c(F)c1 ; CCOC(=O)c1[nH]c2c([C@H](C)n3ccoc3=O)cccc2c1-c1ccc(CS(C)(=O)=O)c(F)c1 ; CCOC(=O)C1=C(c2ccc(CS(C)(=O)=O)c(F)c2)c2cccc([C@H](C)n3cc(C=O)oc3=O)c2C1]

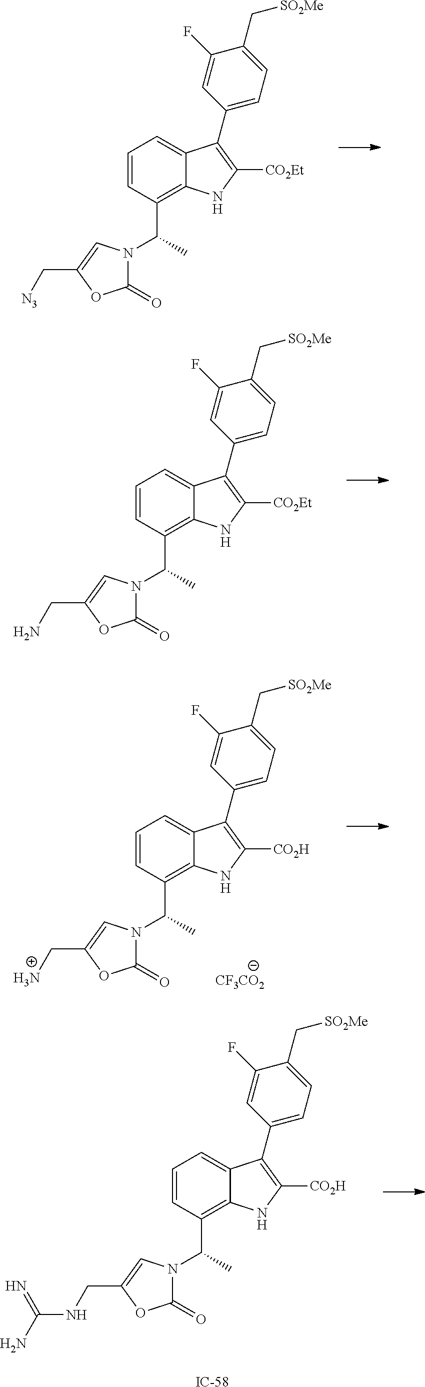

To S39 (418 mg, 1.00 mmol), 2-acetoxyacetic acid (130 mg, 1.1 mmol), DMAP (6.1 mg, 0.050 mmol) in CH₂Cl₂ (3 mL) at 0° C. was added EDC·HCl (230 mg, 1.2 mmol) and the reaction mixture was warmed to RT and stirred for 14 h. The reaction was diluted with CH₂Cl₂, washed sequentially with 1 M HCl, H₂O, sat. NaHCO₃, brine, dried (Na₂SO₄) and concentrated in vacuo (496 mg). The crude acetoxyamide was dissolved in EtOH/THF (3:1, 4 mL) and treated with K₂CO₃ (278 mg, 2.00 mmol) and the reaction was stirred for 1 h at RT; whereupon it was diluted with EtOAc, washed with H₂O, sat. NH₄Cl, brine (×2), dried (Na₂SO₄) and the volatiles were concentrated in vacuo. To the resulting crude mixture in MeCN (10 mL) was added CDI (243 mg, 1.50 mmol) and the reaction was stirred at RT for 8 h and then concentrated under reduced pressure. The residue was redissolved in EtOAc, washed sequentially with 4 M HCl, H₂O (×2), sat. NaHCO₃, brine, dried (Na₂SO₄) and concentrated in vacuo (512 mg). Purification by FCC (CH₂Cl₂/EtOAc 10:1 to 8:1) afforded the product in 95% yield over 3 steps as a colourless solid (476 mg).

To the above product (476 mg, 0.947 mmol) in THF (3.5 mL) at −78° C. was added LiBHEt₃ (1.7 M in THF, 0.724 mL, 1.23 mmol) dropwise and stirring was maintained at this temperature for an hour, followed by a second addition of LiBHEt₃ (0.11 mL) and stirring was maintained for another hour. DIPEA (0.247 mL, 1.42 mmol, 1.5 eq) and DMAP (12 mg, 0.0947 mmol) were then added dropwise as a solution in THF (0.5 mL) at −78° C. and then trifluoroacetic anhydride (0.17 mL, 1.23 mmol, 1.30 eq) was added dropwise. The cooling bath was removed and the reaction mixture was warmed to 0° C. and stirred for 1 h and then quenched with sat. NH₄Cl (2 mL) and then stirred vigorously for 30 min under air. The reaction mixture was diluted with EtOAc; the organic layer was washed with 1 M HCl, H₂O, sat NaHCO₃, brine, dried (Na₂SO₄) and the volatiles concentrated in vacuo. Purification by FCC (DCM:EtOAc 8:1) afforded the oxazolone in 77% yield (354 mg) as a colourless solid.

Oxalyl chloride (0.245 mL, 2.89 mmol, 4.0 eq) was added dropwise to DMF (0.224 mL, 2.89 mmol, 4.0 eq) at 0° C. under Ar atmosphere and the reaction mixture was stirred at RT for 30 min and then cooled to 0° C. To this solution was added oxazolone (352 mg, 0.724 mmol, 1.0 eq) in CH₂Cl₂ (3.6 mL) and the reaction mixture was warmed to RT and stirred for 7 h. The obtained yellow solution was quenched with 5 M NaOAc (2.9 mL, 20 equiv) at 0° C. and stirred for 10 min at RT and diluted with EtOAc. The organic layer was washed sequentially with 1 M HCl (×2), H₂O (×4), sat. NaHCO₃, brine, then dried (Na₂SO₄) and concentrated in vacuo (381 mg). Purification by FCC (CH₂Cl₂/EtOAc 5:1) afforded the aldehyde as a pale yellow solid in 89% yield (332 mg).

To the aldehyde (329 mg, 0.639 mmol) in CH₂Cl₂ (3 mL) and EtOH (3 mL) at 0° C. was added NaBH₄ (24 mg, 0.639 mmol) and the reaction was stirred at rt for 20 min. The reaction was quenched at 0° C. with sat NH₄Cl, diluted with EtOAc, washed with 1 M HCl, H₂O, sat. NaHCO₃, brine, dried (Na₂SO₄) and concentrated in vacuo. To the alcohol in DMF (1.2 mL) was added NaN₃ (62 mg, 0.959 mmol), PPh₃ (252 mg, 0.959 mmol) at RT and stirring was maintained for 30 min. The reaction was cooled to 10° C. and then CBr₄ (318 mg, 0.959 mmol) was added and the reaction was stirred at rt for 1 h. The reaction was diluted with EtOAc, washed with H₂O (×5), brine (×2), dried (Na₂SO₄) and then concentrated in vacuo. Purification by FCC (CH₂Cl₂:EtOAc 20:1 to 10:1) afforded the intermediate azide as a colourless solid in 63% yield (219 mg).

To the azide (216 mg, 0.399 mmol) in THF (3 mL) and H₂O (1 mL) at 0° C. was added PPh₃ (136 mg, 0.519 mmol) and the reaction was stirred at rt for 16 h. The reaction was diluted with EtOAc, washed with brine, then dried (Na₂SO₄) and concentrated in vacuo. Purification by FCC (EtOAc then CH₂Cl₂:MeOH 50:1 to 10:1) afforded the amine as a colourless solid in 93% yield (192 mg).

To the ethyl ester (189 mg, 0.367 mmol) in THF (1.8 mL) was added a solution of LiOH·H₂O (76.9, 1.83 mmol) in H₂O (1.8 mL) and the reaction mixture was warmed to RT over 20 h. The reaction was quenched by the dropwise addition of TFA (0.168 mL, 2.20 mmol) and evaporated with DMSO (0.4 mL) under reduced pressure at 40° C. bath temperature. Purification by FCC (RP C18 flash chromatography H₂O+0.1% TFA/MeCN 0→60%) then PrepHPLC chromatography (Atlantis, 5 μm, 30×100 mm, H₂O+0.02% TFA/MeCN 10→95% gradient) afforded the ammonium salt as a white foam in 77% yield (170 mg).

To the ammonium salt (169 mg, 0.278 mmol) in DMF (1 mL) was added [amino(pyrazol-1-yl)methylene]ammonium chloride (45 mg, 0.31 mmol) then DIPEA (0.121 mL, 0.69 mmol) at RT and the reaction was stirred for 15 h. The reaction was concentrated under reduced pressure then purified by FCC (RP C18 flash chromatography H₂O+0.1% TFA/MeCN 5→50%) then by PrepHPLC chromatography (Atlantis, 5 μm, 30×100 mm, H₂O+0.05% TFA/MeCN 10→50% gradient) to afford the indole carboxylate IC-58 in 29% yield (43 mg).

¹H NMR (400 MHz, DMSO-d6): 11.18-10.20 (bs, 1H), 8.62 (t, J=4.8 Hz, 1H), 8.36-7.67 (bs, 3H), 7.53-7.36 (m, 4H), 7.25 (d, J=7.2 Hz, 1H), 7.13 (s, 1H), 7.04 (t, J=7.7 Hz, 1H), 5.76 (q, J=7.0 Hz, 1H), 4.55 (s, 2H), 4.24-4.01 (m, 2H), 3.04 (s, 3H), 1.77 (d, J=7.4 Hz, 3H), —CO₂H signal not observed due to broadening; LRMS [M+H]⁺ 530.

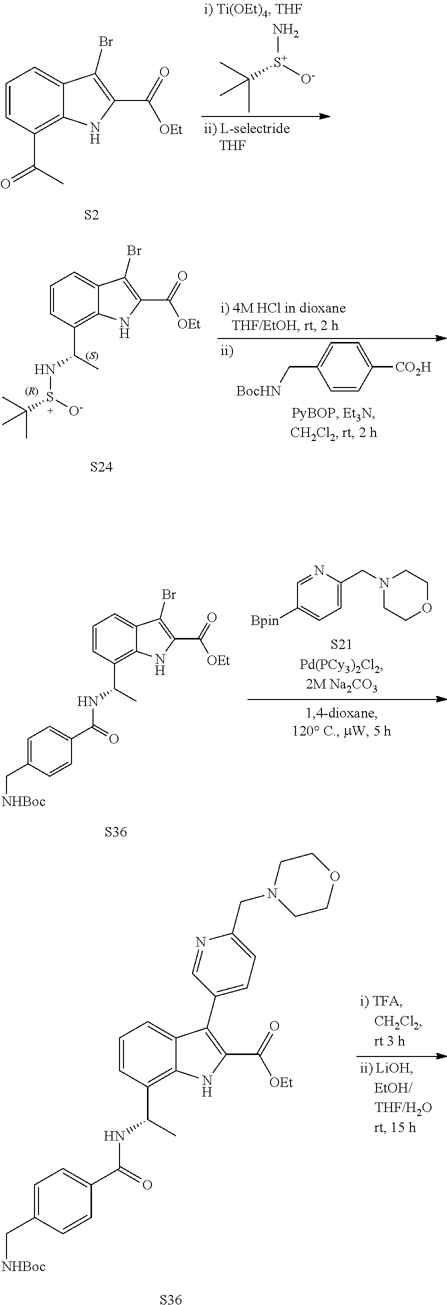

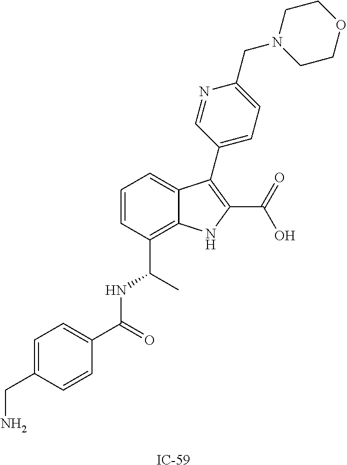 [SMILES: C[C@H](NC(=O)c1ccc(CN)cc1)c1cccc2c(-c3ccc(CN4CCOCC4)nc3)c(C(=O)O)[nH]c12 ; "IC-59"]

### Ethyl 3-bromo-7-((1 S)-1-((4-(((tert-butoxycarbonyl)amino)methyl)benzoyl)amino)ethyl)-1H-indole-2-carboxylate (S35)

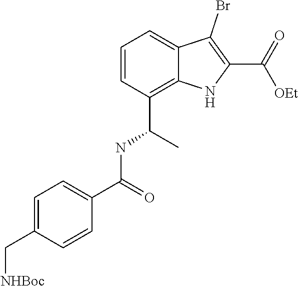 [SMILES: CCOC(=O)c1[nH]c2c([C@H](C)NC(=O)c3ccc(CNC(=O)OC(C)(C)C)cc3)cccc2c1Br]

To a solution of N-protected amine S24 (500 mg, 1.2 mmol, 1.0 eq) in anhydrous THE (4.3 mL) in EtOH (1.8 mL) was added 4 M aq HCl in dioxane (0.96 mL, 3.85 mmol, 3.2 eq) at room temperature. The reaction mixture was stirred for 1 h by which point complete conversion of starting material was observed. The resultant mixture was concentrated in vacuo and the solid obtained was suspended in Et₂O then filtered. The precipitate obtained was washed with Et₂O and air-dried to obtain the desired product as an off-white solid (380 mg, >99%). In a separate flask, to a solution of 4-[(tert-butoxycarbonylamino)methyl]benzoic acid (289 mg, 1.15 mmol, 1.2 eq) in CH₂Cl₂ (12 mL) was added the indole amine (299 mg, 0.96 mmol, 1.0 eq), benzotriazole-1-yl-oxy-tris-pyrrolidino-phosphonium hexafluoro phosphate (599 mg, 1.15 mmol, 1.2 eq) and Et₃N (0.4 mL, 2.88 mmol, 3.0 eq). The resultant mixture was stirred at rt for 1 h. Upon completion of the reaction, the solvents were concentrated in vacuo and the crude product was purified using a Biotage SNAP-ultra column, with gradient elution from 100% CH₂Cl₂ to 20% EtOAc in CH₂Cl₂ to afford the desired product S35 in >99% yield as a white solid (555 mg).

¹H NMR (500 MHz, DMSO) δ ppm 11.88 (1H, s, NH), 9.04 (1H, d, J=8.0 Hz, NH), 7.81 (2H, d, J=8.0 Hz, Ar—H), 7.44 (1H, d, J=8.0 Hz, NH), 7.38 (2H, d, J=7.5 Hz, Ar—H), 7.30 (2H, d, J=8.0 Hz, Ar—H), 7.19 (1H, t, J=7.5 Hz, Ar—H), 5.72 (1H, app p, J=7.0 Hz, CH₃CH), 4.58-4.31 (2H, m, OCH₂CH₃), 4.14 (2H, d, J=6.0 Hz, CH₂), 1.57 (3H, d, J=7.0 Hz, CH₃CH), 1.40-1.32 (12H, m, OCH₂CH₃ & C(CH₃)₃); ¹³C NMR (126 MHz, DMSO) δ ppm 166.6, 160.5, 156.3 (C═O), 144.1, 133.8, 132.8, 130.3, 127.7, 127.6, 127.1, 124.5, 122.8, 121.8, 119.3, 97.2 (Ar-CH+Ar-C), 78.5 (C(CH₃)₃), 61.4 (OCH₂CH₃), 44.8 (CH₃CH), 43.4 (CH₂), 28.5 (C(CH₃)₃), 20.7 (CH₃CH), 14.6 (OCH₂CH₃); HRMS (TOF, ESI⁺) m/z: [M+H]⁺ Calcd for C₂₆H₃₀O₅N₃⁷⁹BrNa 566.12610; Found 566.12605.

### Ethyl 7-((1S)-1-((4-(((tert-butoxycarbonyl)amino)methyl)benzoyl)amino)ethyl)-3-(6-(morpholin-4-ylmethyl)pyridin-3-yl)-1H-indole-2-carboxylate (S36)

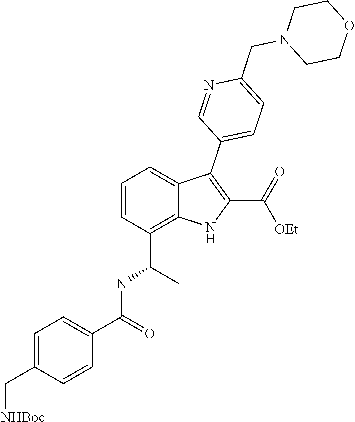 [SMILES: CCOC(=O)c1[nH]c2c([C@H](C)NC(=O)c3ccc(CNC(=O)OC(C)(C)C)cc3)cccc2c1-c1ccc(CN2CCOCC2)nc1]

To the boronate S21 (369 mg, 1.21 mmol, 1.2 eq) in 1,4-dioxane (9 mL) in a microwavable vial, were added bromo indole S35 (550 mg, 1.01 mmol, 1.0 eq), Pd(PCy₃)₂Cl₂ (37.3 mg, 0.051 mmol, 0.05 eq), 2 M aq Na₂CO₃ (2.02 mL, 4.04 mmol, 4.0 eq). The reaction mixture was purged with argon, then subjected to microwave irradiation at 120° C. for 6 h. The reaction mixture was filtered through Celite®, eluting with EtOAc (40 mL) and water (20 mL). The organics were separated from the filtrate and the aqueous layer further extracted with EtOAc (2×20 mL). The combined organic extracts were dried over Na₂SO₄, filtered and concentrated in vacuo. The product was purified using a BIOTAGE SNAP-Ultra 10 g column, with gradient elution from 100% EtOAc to 2% MeOH in EtOAc to give the desired product S36 in >99% yield as a yellow solid (655 mg).

¹H NMR (500 MHz, CDCl₃) δ ppm 11.73 (1H, s, NH), 9.01 (1H, d, J=8.0 Hz, NH), 8.57 (1H, dd, J=2.5, 1.0 Hz, Ar—H), 7.89-7.83 (3H, m, Ar—H), 7.52 (1H, dd, J=8.0, 1.0 Hz, Ar—H), 7.45-7.28 (5H, m, Ar-H+NH), 7.11 (1H, dd, J=8.0, 7.0 Hz, Ar—H), 5.80 (1H, app t, J=7.0 Hz, CH₃CH), 4.24 (2H, app qd, J=7.0, 2.0 Hz, OCH₂CH₃), 4.16 (2H, d, J=6.0 Hz, CH₂), 3.66 (2H, s, CH₂), 3.65-3.60 (4H, m, morpholino-CH₂), 2.49-2.44 (4H, m, morpholino-CH₂), 1.64 (3H, d, J=7.0 Hz, CH₃CH), 1.39 (9H, s, C(CH₃)₃), 1.17 (3H, t, J=7.0 Hz, OCH₂CH₃); ¹³C NMR (126 MHz, CDC₃) δ ppm 165.9, 161.1, 156.3 (C═O), 149.7, 143.7, 138.0, 133.8, 132.6, 129.7, 127.9, 127.4, 127.2, 126.6, 123.6, 122.8, 122.2, 121.8, 121.0, 119.2, 118.9 (Ar-C+Ar-CH), 77.9 (C(CH₃)₃), 66.2 (morpholino-CH₂), 64.0 (CH₂), 60.5 (OCH₂CH₃), 53.3.3, 53.3 (morpholino-CH₂), 44.7 (CH₃CH), 43.1 (CH₂), 28.2 (C(CH₃)₃), 20.5 (CH₃CH), 13.8 (OCH₂CH₃); HRMS (TOF, ESI⁻) m/z: [M+H]⁺ Calcd for C₃₆H₄₄O₆N₅ 642.32861; Found 642.32813.

### IC-59: 7-((1S)-1-((4-(aminomethyl)benzoyl)amino)ethyl)-3-(6-(morpholin-4-ylmethyl)pyridin-3-yl)-1H-indole-2-carboxylic acid

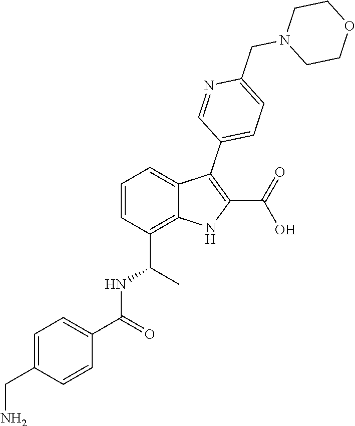 [SMILES: C[C@H](NC(=O)c1ccc(CN)cc1)c1cccc2c(-c3ccc(CN4CCOCC4)nc3)c(C(=O)O)[nH]c12]

To a solution of N-Boc amine S36 (303 mg, 0.47 mmol, 1.0 eq) in CH₂Cl₂ (2.8 mL), cooled to 0° C., was added TFA (0.36 mL, 4.7 mmol, 10.0 eq) dropwise. The reaction mixture was allowed to warm to room temperature and stirred overnight. The resultant mixture was concentrated in vacuo co-evaporating with toluene. The crude residue was dissolved in THF/EtOH/H₂O (12 mL, 2:1:1) and treated with LiOH·H₂O (198 mg, 4.7 mmol, 10.0 eq). The resultant solution was stirred at room temperature overnight. Upon completion, the resultant mixture was acidified to pH 2 with 2 M aq HCl, then concentrated in vacuo. The product was purified by preparative scale HPLC, with gradient elution from 2% MeCN in water to 40% MeCN in water over 17 min, then up to 98% MeCN in water over 3 min. Both solvents were acidified to a final concentration of 0.01% (v/v) formic acid. The fractions containing product were concentrated in vacuo, then re-suspended in 18 mL of water-MeCN. The solution was treated with 2 mL of 100 mM HCl, so the final HCl concentration was 10 mM. The sample was then lyophilized to obtain the indole carboxylate IC-59 in >99% yield as a HCl salt and as a yellow powder (298 mg).

¹H NMR (500 MHz, CD₃OD) δ ppm 8.84 (1H, d, J=2.0 Hz, Ar—H), 8.08 (1H, dd, J=8.0, 2.0 Hz, Ar—H), 7.95 (2H, d, J=8.5 Hz, Ar—H), 7.62 (1H, d, J=8.0 Hz, Ar—H), 7.56 (2H, d, J=8.5 Hz, Ar—H), 7.48-7.42 (2H, m, Ar—H), 7.17 (1H, t, J=7.5 Hz, Ar—H), 5.87 (1H, q, J=7.0 Hz, CH₃CH), 4.58 (2H, s, CH₂), 4.18 (2H, s, CH₂), 4.09-3.84 (4H, m, morpholino-CH₂), 3.46 (4H, t, J=5.0 Hz, morpholino-CH₂), 1.79 (3H, d, J=7.0 Hz, CH₃CH); ¹³C NMR (126 MHz, CD₃OD) δ ppm 167.2, 162.0, (C═O), 150.2, 138.6, 127.9, 127.2, 122.8, 120.9, 120.3, 118.3 (Ar—CH), 146.6, 135.9, 134.1, 133.6, 130.4, 126.8, 123.5, 117.8 (Ar—C), 62.8 (morpholino-CH₂), 59.5 (CH₂), 51.5 (morpholino-CH₂), 43.9 (CH₃CH), 41.7 (CH₂), 17.6 (CH₃CH); HRMS (TOF, ESI⁺) m/z: [M+H]⁺ Calcd for C₂₉H₃₂O₄N₅ 514.24488; Found 514.24467.

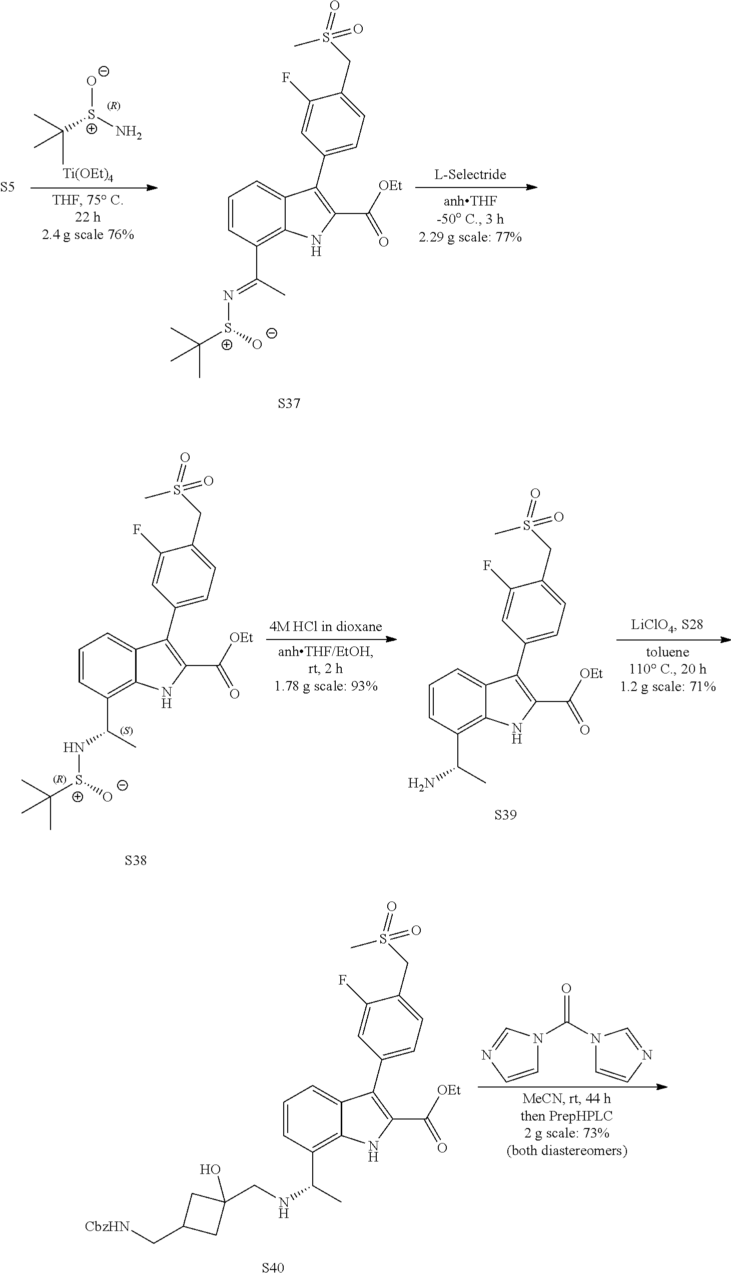

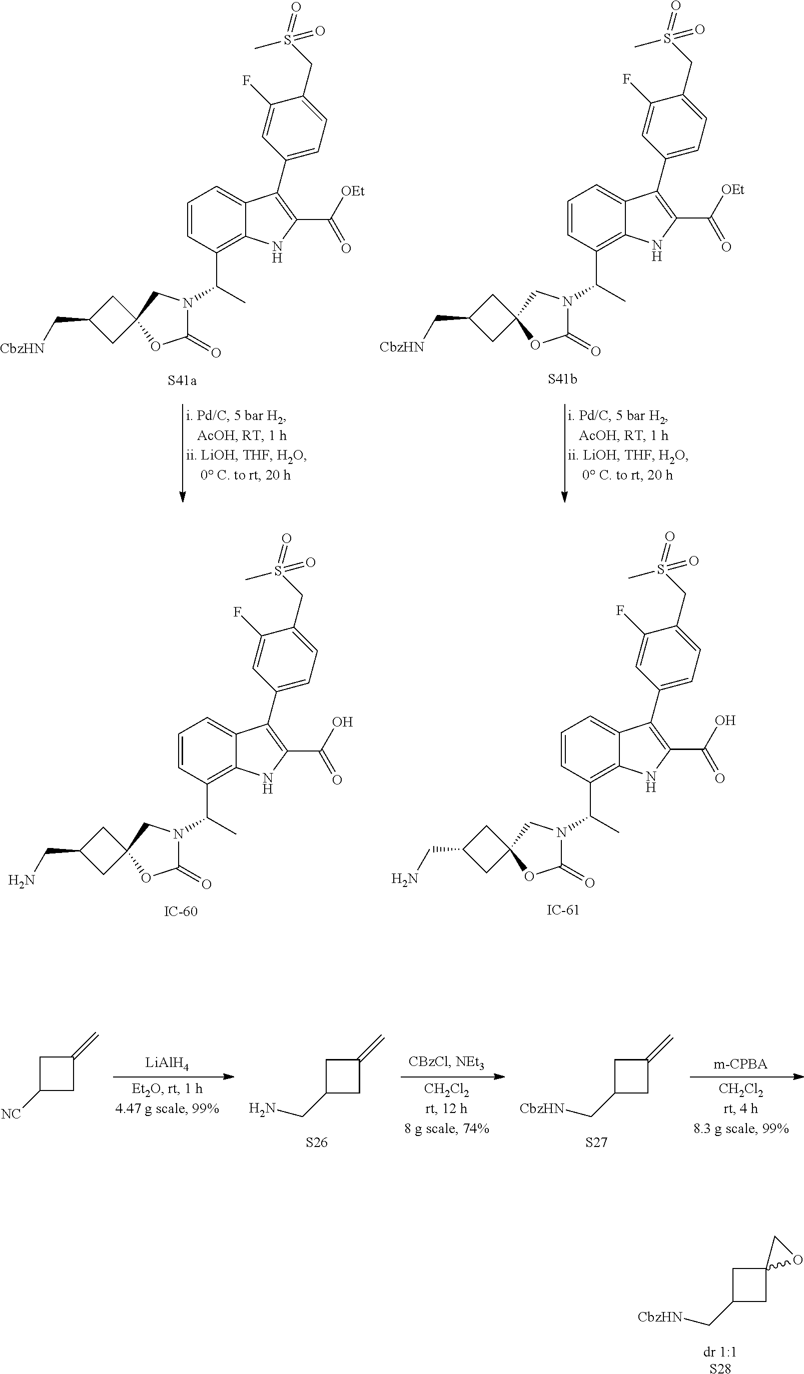

### (R)-tert-Butyl(((1E)-1-(2-(ethoxycarbonyl)-3-(3-fluoro-4-((methylsulfonyl)methyl)phenyl)-1H-indol-7-yl)ethylidene)amino)sulfaniumolate (S37)

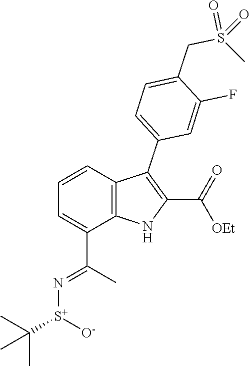 [SMILES: CCOC(=O)c1[nH]c2c(/C(C)=N/[S@+]([O-])C(C)(C)C)cccc2c1-c1ccc(CS(C)(=O)=O)c(F)c1]

To a suspension of ketone S5 (2.40 g, 5.75 mmol, 1.0 eq) in anhydrous THF (7.2 mL) under Ar atmosphere, was added Ti(OEt)₄ (2.41 mL, 11.5 mmol, 2.0 eq) followed by addition of (R)-tBu-sulfinimide (1.05 g, 8.60 mmol, 1.50 eq) at room temp and the resulting yellow suspension was refluxed at 80° C. for 26 h. The reaction mixture was cooled to RT, diluted with CH₂Cl₂ and poured into brine (30 mL) and the resulting slurry was stirred vigorously for 15 min then filtered through a pad of Celite® eluting with EtOAc. The organics were combined, washed with water, brine, dried over Na₂SO₄ and concentrated in vacuo. Purification by FCC (hexane:EtOAc 1:1 to EtOAc) afforded the desired compound S37 in 76% yield as a green solid (2.30 g).

¹H NMR (300 MHz, CDCl₃) δ ppm 11.29 (1H, s, N—H), 7.88 (1H, d, J=7.7 Hz, Ar—H), 7.83 (1H, d, J=8.0 Hz, Ar—H), 7.59 (1H, t, J=7.9 Hz, Ar—H), 7.47-7.34 (2H, m, Ar—H), 7.33-7.13 (1H, m, Ar—H), 4.40 (2H, s, CH₂SO₂CH₃), 4.34 (2H, qd, J=7.2, 1.2 Hz, OCH₂CH₃), 2.95 (3H, s, CH₃), 2.90 (3H, s, CH₃), 1.45 (9H, s, (CH₃)₃), 1.31 (3H, t, J=7.1 Hz, OCH₂CH₃); ¹³C NMR (101 MHz, CDCl₃) δ ppm 178.7, 160.9, 160.5 (d, J_{FC}=247.6 Hz), 136.9 (d, J_{FC}=8.9 Hz), 133.8, 132.3 (d, J_{FC}=3.3 Hz), 128.8, 127.7, 127.5 (d, J_{FC}=3.2 Hz), 126.7, 124.2, 122.0 (d, J_{FC}=2.0 Hz), 121.5, 121.0, 118.3 (d, J_{FC}=22.5 Hz), 114.9 (d, J_{FC}=14.6 Hz), 61.3, 56.8, 54.3 (d, J_{FC}=2.9 Hz), 39.6 (d, J_{FC}=2.5 Hz), 22.7, 20.0, 14.4; LCMS [M+H]⁺ 521.46.

### tert-Butyl(((1S)-1-(2-(ethoxycarbonyl)-3-(3-fluoro-4-((methylsulfonyl)methyl)phenyl)-1H-indol-7-yl)ethyl)amino)-(R)-sulfaniumolate (S38)

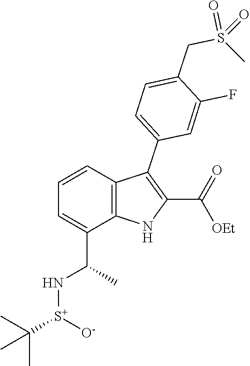 [SMILES: CCOC(=O)c1[nH]c2c([C@H](C)N[S@+]([O-])C(C)(C)C)cccc2c1-c1ccc(CS(C)(=O)=O)c(F)c1]

To the imine S37 (2.29 g, 4.35 mmol, 1.0 ea) in anhydrous THF (22 mL) cooled to −76° C. was added L-Selectride® (1 M in THF, 8.7 mL, 8.7 mmol, 2.0 eq) dropwise and the resulting brownish green suspension was stirred at this temperature for 5 h and then the reaction mixture was allowed to slowly warm to 0° C. and the reaction was quenched with the addition of 30 mL sat. aq. NH₄Cl. The mixture was extracted with EtOAc (250 mL×2); the organic layers were combined, washed with water (100 mL×2), brine, dried over Na₂SO₄ and filtered through a plug of silica, washed with EtOAc and concentrated in vacuo. Purification by FCC (hexane: EtOAc 1:1, then EtOAc) afforded the desired compound S38 in 77% yield (1.79 g).

[α]_{D}²⁰=−32.3 (c 0.96, MeOH); ¹H NMR (300 MHz, CDCl₃) δ ppm 9.87 (1H, s, N—H), 7.57 (2H, dt, J=7.9, 3.8 Hz, Ar—H), 7.47-7.35 (2H, m, Ar—H), 7.32 (1H, d, J=7.1, Ar—H), 7.15 (1H, dd, J=8.1, 7.2 Hz, Ar—H), 4.93 (1H, qd, J=6.7, 2.8 Hz, CHCH₃), 4.40 (2H, s, CH₂SO₂CH₃), 4.40-4.19 (2H, m, OCH₂CH₃), 3.60 (1H, d, J=2.8 Hz, N—H), 2.89 (3H, d, J=0.9 Hz, CH₂SO₂CH₃), 1.75 (3H, d, J=6.8 Hz, CH₃), 1.27 (12H, m, (CH₃)₃, OCH₂CH₃); ¹³C NMR (101 MHz, CDCl₃) δ ppm 161.5, 160.5 (d, J_{FC}=247.3 Hz), 137.5 (d, J_{FC}=9.0 Hz), 133.4, 132.2 (d, J_{FC}=3.2 Hz), 128.5, 127.5 (d, J_{FC}=3.3 Hz), 126.1, 124.7, 123.3, 121.9 (d, J_{FC}=2.0 Hz), 121.5, 121.2, 118.2 (d, J_{FC}=22.5 Hz), 114.6 (d, J_{FC}=14.7 Hz), 61.3, 55.9, 54.4 (d, J_{FC}=2.9 Hz), 52.9, 39.6 (d, J_{FC}=2.6 Hz), 22.9, 22.8, 14.2; LCMS [M+H]⁺ 523.51; HRMS (TOF, ESI⁺) m/z: [M+Na]⁺ Calcd for C₂₅H₃₁FN₂O₅S₂Na 545.1551; Found 545.1555.

### Ethyl 7-((1 S)-1-aminoethyl)-3-(3-fluoro-4-((methylsulfonyl)methyl)phenyl)-1H-indole-2-carboxylate (S39)

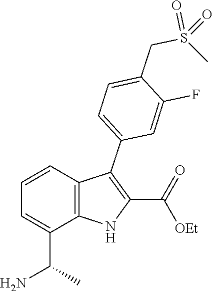 [SMILES: CCOC(=O)c1[nH]c2c([C@H](C)N)cccc2c1-c1ccc(CS(C)(=O)=O)c(F)c1]

To a solution of S38 (1.77 g, 3.33 mmol, 1.0 eq) in THF (24 mL) was added EtOH (10 mL) followed by 4 M HCl in dioxane (3.33 mL, 13.3 mmol, 4.0 eq) at room temperature and the reaction was stirred for 2 h. The resulting suspension was concentrated in vacuo, and the crude product triturated with Et₂O and the resulting solid was dissolved in CH₂Cl₂ (250 mL) and carefully extracted with sat. aq. NaHCO₃ (2×75 mL), brine (100 mL), dried (Na₂SO₄), filtered and the volatiles removed in vacuo. The resulting solid was washed with heptane to afford the desired product S39 in 93% yield as a beige solid (1.32 g).

[α]_{D}²⁰=+17.3 (c 1.0, DMSO); ¹H NMR (300 MHz, CDCl₃) δ ppm 10.80 (1H, s, N—H), 7.61-7.33 (4H, m, Ar—H), 7.19-7.04 (2H, m, Ar—H), 4.59 (1H, q, J=6.6 Hz, CHCH₃), 4.39 (2H, s, CH₂SO₂CH₃), 4.32 (2H, q, J=7.1 Hz, OCH₂CH₃), 2.88 (3H, d, J=0.9 Hz, CH₂SO₂CH₃), 1.55 (3H, d, J=6.6 Hz, CH₃), 1.28 (3H, t, J=7.1 Hz, OCH₂CH₃); ¹³C NMR (101 MHz, DMSO-d₆) δ ppm 161.1, 160.4 (d, J_{FC}=247.1 Hz), 136.2 (d, J_{FC}=8.8 Hz), 133.6, 132.5 (d, J_{FC}=3.6 Hz), 127.3, 126.6 (d, J_{FC}=2.6 Hz), 124.4, 124.2, 122.3, 121.2, 121.1 (d, J_{FC}=2.0 Hz), 120.7, 117.6 (d, J_{FC}=22.2 Hz), 114.8 (d, J_{FC}=15.4 Hz), 67.0, 60.7, 53.1, 44.9, 20.3, 13.8; LCMS [M+H]⁺ 419.30; HRMS (TOF, ESI⁺) m/z: [M+Na]⁺ Calcd for C₂₁H₂₃FN₂O₄Na 441.1255; Found 441.1252.

### Ethyl 7-((1S)-1-(((3-((((benzyloxy)carbonyl)amino)methyl)-1-hydroxycyclobutyl)methyl)amino)ethyl)-3-(3-fluoro-4-((methylsulfonyl)methyl)phenyl)-1H-indole-2-carboxylate (S40)

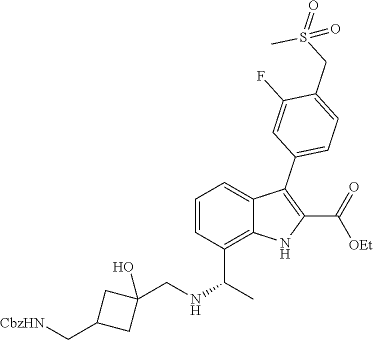 [SMILES: CCOC(=O)c1[nH]c2c([C@H](C)NCC3(O)CC(CNC(=O)OCc4ccccc4)C3)cccc2c1-c1ccc(CS(C)(=O)=O)c(F)c1]

A suspension of amine S39 (1.20 g, 2.87 mmol, 1.08 eq), the epoxide S28 (655 mg, 2.65 mmol, 1.0 eq) and LiClO₄ (284 mg, 2.52 mmol, 0.95 eq) in anhydrous toluene (12 mL) was heated at 110° C. for 20 h. The resulting yellow solution was cooled to rt and diluted with EtOAc and water; the layers were separated and the aqueous layer extracted with EtOAc (2 x) and the combined organics were washed with brine, dried over Na₂SO₄ and concentrated in vacuo. Purification by FCC (CH₂Cl₂: MeOH 50:1 to 20:1) afforded the desired compound S40 as a mixture of diastereomers in 71% yield (1.38 g) (which due to partial instability was quickly taken through to the oxazolidone ring forming reaction).

¹H NMR (400 MHz, CDCl₃) δ ppm 10.75 (1H, s, N—H), 7.59-7.47 (2H, m, Ar—H), 7.46-7.36 (2H, m, Ar—H), 7.36-7.26 (5H, m, Ar—H), 7.17 (1H, ddd, J=7.4, 2.4, 1.1 Hz, Ar—H), 7.10 (1H, dd, J=8.1, 7.1 Hz, Ar—H), 5.05 (2H, d, J=12.5 Hz, CH₂Ph), 4.93-4.82 (1H, m, CHCH₃), 4.39 (2H, s, CH₂SO₂CH₃), 4.34-4.13 (3H, m, OCH₂CH₃, N—H), 3.29-2.99 (2H, m), 2.88 (3H, dd, J=2.5, 0.8 Hz, CH₂SO₂CH₃), 2.78 (1H, t, J=12.8 Hz), 2.63-2.36 (2H, m), 2.25-2.07 (2H, m), 1.93 (1H, q, J=9.0 Hz), 1.76 (2H, td, J=12.8, 7.6 Hz), 1.54 (3H, dd, J=6.6, 2.7 Hz, CHCH₃), 1.20 (3H, t, J=7.1 Hz); LCMS [M+H]⁺ 666.62.

### Ethyl 7-((1S)-1-(2-((((benzyloxy)carbonyl)amino)methyl)-6-oxo-5-oxa-7-azaspiro[3.4]oct-7-yl)ethyl)-3-(3-fluoro-4-((methylsulfonyl)methyl)phenyl)-1H-indole-2-carboxylate (S41)

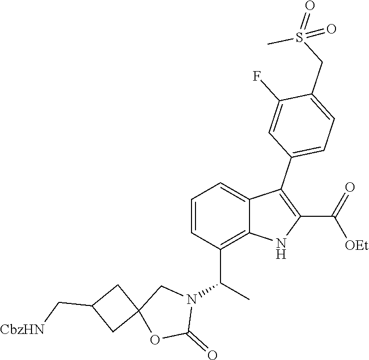 [SMILES: CCOC(=O)c1[nH]c2c([C@H](C)N3CC4(CC(CNC(=O)OCc5ccccc5)C4)OC3=O)cccc2c1-c1ccc(CS(C)(=O)=O)c(F)c1]

To a solution of the aminoalcohol S40 (2.00 g, 2.73 mmol, 1.0 eq) in MeCN (30 mL) was added 1,1′-carbonyldiimidazole (CDI, 887 mg, 5.47 mmol, 2.00 eq) and the reaction mixture was stirred at RT for 21 h. UPLC analysis of the reaction mixture showed a conversion of 65% and a further portion of CDI was added (443 mg, 1.0) and stirring was continued for a further 23 h; UPLC analysis at this stage showed 95% conversion. The reaction mixture was concentrated in vacuo, suspended in EtOAc and the precipitate was filtered off; the filtrate was washed with aq 1 M HCl (150 mL), the aqueous layer was extracted with EtOAc (×2) and the combined organics were washed with water (100 mL), sat. NaHCO₃ (100 mL), brine (100 mL), dried (Na₂SO₄) and concentrated in vacuo. Purification by FCC (CH₂Cl₂: MeOH 20:1) afforded the desired compound S41 as a mixture of diastereomers in 73% yield (1.38 g).

Further purification by preparative HPLC (Daicel Chiralpak ID, 250×30 mm, 5 μm particle size, 0-20 min heptane: CH₂Cl₂: iPrOH 55:20:25, then 20-40 min heptane: CH₂Cl₂: iPrOH 25:50:25, 30 mL/min) gave first S41a (679 mg, tᵣ=15.0 min) then S41b (595 mg, tᵣ=35.0 min).

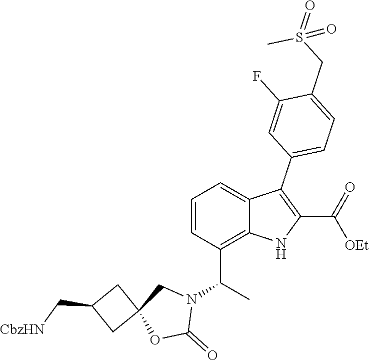 [SMILES: CCOC(=O)c1[nH]c2c([C@H](C)N3C[C@]4(C[C@@H](CNC(=O)OCc5ccccc5)C4)OC3=O)cccc2c1-c1ccc(CS(C)(=O)=O)c(F)c1]

Data S41a: [α]_{D}²⁰=+107.3 (c 0.5, CH₂Cl₂); ¹H NMR (300 MHz, CDCl₃) δ ppm 10.60 (1H, s), 7.63-7.51 (2H, m), 7.46-7.37 (2H, m), 7.37-7.26 (6H, m), 7.15 (1H, dd, J=8.1, 7.2 Hz), 5.61 (1H, q, J=7.0 Hz), 5.06 (2H, s), 4.86 (1H, s), 4.42-4.25 (4H, m), 3.59 (1H, d, J=8.9 Hz), 3.23 (2H, t, J=5.8 Hz), 3.07 (1H, d, J=8.8 Hz), 2.88 (3H, d, J=0.9 Hz), 2.32 (1H, dd, J=11.4 Hz, 7.1), 2.25-1.94 (4H, m), 1.74 (3H, d, J=7.1 Hz), 1.35 (3H, t, J=7.1 Hz); ¹³C NMR (101 MHz, CDC₃) δ ppm 161.7, 161.0, 159.2, 158.0, 156.8, 137.4, 137.3, 136.4, 134.5, 132.2, 132.2, 128.7, 128.4, 128.2, 127.9, 127.3, 124.0, 123.2, 122.4, 121.9, 121.4, 120.8, 118.2, 118.0, 114.6, 114.4, 79.0, 67.0, 61.2, 54.4, 53.6, 51.6, 47.1, 45.4, 39.5, 39.0, 37.5, 27.4, 15.9, 14.2; LRMS [M+H]⁺ 692.6.

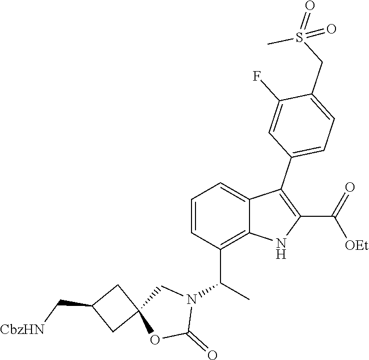 [SMILES: CCOC(=O)c1[nH]c2c([C@H](C)N3C[C@]4(C[C@H](CNC(=O)OCc5ccccc5)C4)OC3=O)cccc2c1-c1ccc(CS(C)(=O)=O)c(F)c1]

Data S41b: [α]_{D}²⁰=+62.0 (c 0.5, CH₂Cl₂); ¹H NMR (300 MHz, CDCl₃) δ ppm 10.62 (1H, s), 7.64-7.52 (2H, m), 7.48-7.37 (2H, m), 7.29 (1H, d, J=7.2 Hz), 7.19-7.11 (1H, m), 5.62 (1H, q, J=7.0 Hz), 4.34 (4H, dd, J=15.5, 8.4 Hz), 3.61 (1H, d, J=8.8 Hz), 3.10 (1H, d, J=8.8 Hz), 2.89 (3H, s), 2.37 (1H, d, J=5.8 Hz), 2.24-2.08 (3H, m), 2.01 (2H, s), 1.75 (3H, d, J=7.1 Hz), 1.35 (3H, t, J=7.1 Hz); ¹³C NMR (101 MHz, CDCl₃) δ ppm 161.7, 161.0, 159.3, 158.0, 156.6, 137.4, 137.3, 136.4, 134.5, 132.2, 128.7, 128.3, 128.3, 127.9, 127.4, 127.3, 124.0, 123.2, 122.3, 121.9, 121.4, 120.8, 118.2, 118.0, 114.6, 114.4, 76.2, 67.0, 61.2, 54.4, 53.6, 51.3, 47.1, 45.7, 39.5, 39.4, 38.2, 25.6, 15.9, 14.2; LRMS [M+H]⁺ 692.6.

### Ethyl 7-((1 S)-1-((2S,4r)-(2-(aminomethyl)-6-oxo-5-oxa-7-azaspiro[3.4]oct-7-yl)ethyl)-3-(3-fluoro-4-((methylsulfonyl)methyl)phenyl)-1H-indole-2-carboxylate (S42)

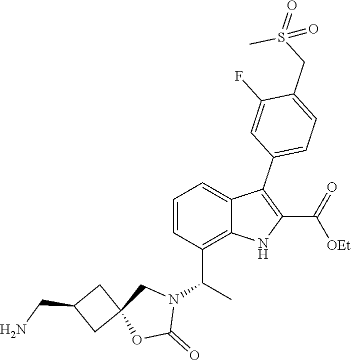 [SMILES: CCOC(=O)c1[nH]c2c([C@H](C)N3C[C@]4(C[C@@H](CN)C4)OC3=O)cccc2c1-c1ccc(CS(C)(=O)=O)c(F)c1]

N-CBz protected amine S41a (679 mg, 0.982 mmol) was dissolved in CH₂Cl₂ (45 mL) and was stirred with activated charcoal (300 mg) for 10 min, then filtered through a pad of silica gel and Celite®, the filter cake washed with CH₂Cl₂ then EtOAc; then the volatiles were concentrated in vacuo. The starting material was then dissolved in AcOH (45 mL) and 10% Pd/C (313 mg, 0.294 mmol, 0.30 eq) was added. The reaction mixture was then placed under 5 bar hydrogen pressure for 1 h; UPLC analysis showed full conversion so the reaction mixture was filtrated through a pad of Celite®, the filter cake washed with CH₂Cl₂, EtOAc, then EtOH, and the filtrate was concentrated in vacuo (with addition of toluene then CHCl₃ to remove residual AcOH) to afford the crude compound S42 in >99% yield as a yellow foam (660 mg) which was used directly in the subsequent ester hydrolysis step.

### IC-60: 7-((1S)-1-(2S,4r)-(2-(aminomethyl)-6-oxo-5-oxa-7-azaspiro[3.4]oct-7-yl)ethyl)-3-(3-fluoro-4-((methylsulfonyl)methyl)phenyl)-1H-indole-2-carboxylic acid (IC-60)

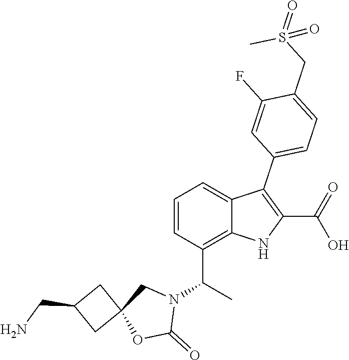 [SMILES: C[C@@H](c1cccc2c(-c3ccc(CS(C)(=O)=O)c(F)c3)c(C(=O)O)[nH]c12)N1C[C@]2(C[C@@H](CN)C2)OC1=O]

The ethyl ester S42 (606 mg, 0.961 mmol) was dissolved in THF (18 mL) and a solution of LiOH·H₂O (242 mg, 5.78 mmol, 6.0 eq) in H₂O (18 mL) was added at 0° C. and the reaction was slowly warmed to RT and stirred overnight. UPLC analysis after 14 h showed 90% conversion and after 20 h 98% conversion was reached, whereupon the reaction mixture was quenched by the addition of AcOH (0.39 mL, 6.73 mmol, 7 eq) and concentrated in vacuo. Purification by RP chromatography (Biotage KP-C18 HS 30 g column, MeCN: 0.1% AcOH in H₂O gradient 5:95% to 95:5%) and then by preparative RP-HPLC afforded the indole carboxylate IC-60 in 57% yield as a white solid (292 mg, 98% purity by HPLC at 210 nm).

[α]_{D}²⁰=+63.0 (c 1.0, DMSO); ¹H NMR (400 MHz, DMSO-d₆) δ ppm 10.30 (s, 1H), 8.88-7.70 (br s, 2H), 7.55-7.36 (m, 4H), 7.15 (d, J=7.2 Hz, 1H), 7.01 (t, J=7.7 Hz, 1H), 5.43 (q, J=7.0 Hz, 1H), 4.55 (s, 2H), 3.66 (d, J=9.6 Hz, 1H), 3.04 (s, 4H), 2.88-2.74 (m, 2H), 2.61-2.54 (m, 1H, overlapped with DMSO signal), 2.47-2.42 (m, 1H, overlapped with DMSO signal), 2.38-2.29 (m, 1H), 2.22-2.12 (m, 1H), 2.00-1.91 (m, 1H), 1.60 (d, J=7.0 Hz, 3H); ¹³C NMR (101 MHz, DMSO-d₆) δ ppm 164.5, 161.4, 159.0, 156.4, 138.5, 138.4, 133.3, 132.0, 131.8, 127.8, 126.3, 123.7, 119.7, 119.6, 119.3, 117.7, 117.5, 114.5, 112.8, 112.7, 78.2, 53.1, 51.5, 47.1, 43.0, 37.7, 37.2, 25.1, 16.5; LCMS [M+H]⁺ 530.44.

### Ethyl 7-((1S)-1-((2R,4s)-(2-(aminomethyl)-6-oxo-5-oxa-7-azaspiro[3.4]oct-7-yl)ethyl)-3-(3-fluoro-4-((methylsulfonyl)methyl)phenyl)-1H-indole-2-carboxylate (S43)

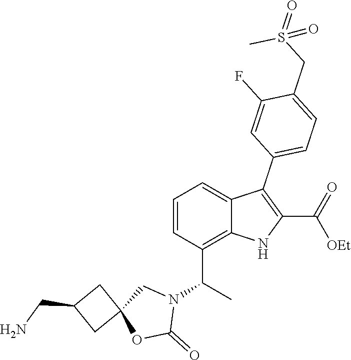 [SMILES: CCOC(=O)c1[nH]c2c([C@H](C)N3C[C@]4(C[C@H](CN)C4)OC3=O)cccc2c1-c1ccc(CS(C)(=O)=O)c(F)c1]

N-CBz protected amine S41b (595 mg, 0.860 mmol) was dissolved in CH₂Cl₂ (40 mL) and was stirred with activated charcoal (300 mg) for 10 min, then filtered through a pad of silica gel and Celite®, the filter cake washed with CH₂Cl₂ then EtOAc; then the volatiles were concentrated in vacuo. The starting material was then dissolved in AcOH (40 mL) and 10% Pd/C (275 mg, 0.258 mmol, 0.30 eq) was added. The reaction mixture was then placed under 5 bar hydrogen pressure for 1 h; UPLC analysis showed full conversion so the reaction mixture was filtrated through a pad of Celite®, the filter cake washed with CH₂Cl₂, EtOAc, then EtOH, and the filtrate was concentrated in vacuo (with addition of toluene then CHCl₃ to remove residual AcOH) to afford the crude compound S43 in 94% yield as an off-white foam (453 mg) which was used directly in the subsequent ester hydrolysis step.

### IC-61: 7-((1S)-1-(2R,4s)-(2-(aminomethyl)-6-oxo-5-oxa-7-azaspiro[3.4]oct-7-yl)ethyl)-3-(3-fluoro-4-((methylsulfonyl)methyl)phenyl)-1H-indole-2-carboxylic acid

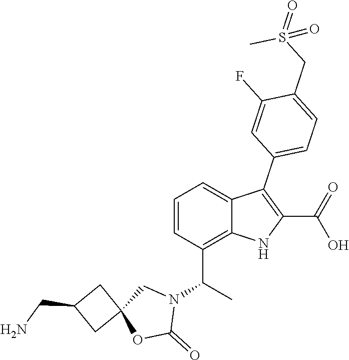 [SMILES: C[C@@H](c1cccc2c(-c3ccc(CS(C)(=O)=O)c(F)c3)c(C(=O)O)[nH]c12)N1C[C@]2(C[C@H](CN)C2)OC1=O]

The ethyl ester S43 (453 mg, 0.812 mmol) was dissolved in THF (2.5 mL) and a solution of LiOH·H₂O (136 mg, 0.325 mmol, 4.0 eq) in H₂O (2.5 mL) was added at 0° C. and the reaction was slowly warmed to RT and stirred for 22 h. UPLC analysis after 22 h showed full conversion, the reaction mixture was quenched by the addition of a solution of KHSO₄ (342 mg, 2.44 mmol, 3 eq) in H₂O (0.8 mL) and THF was partly concentrated and the resultant slurry was stirred overnight. The resultant suspension was filtrated, washed with H₂O (×3) and dried under reduced pressure over P₂O₅. The crude reaction mixture was dissolved in DMSO and TFA (1 eq) was added. Purification by RP chromatography afforded the desired compound indole carboxylate IC-61 in 50% yield as a yellow solid (215 mg, 98% purity by HPLC).

[α]_{D}²³=+47.1 (c 0.92, DMSO); ¹H NMR (400 MHz, DMSO-de) δ ppm 10.39 (1H, s), 8.28 (3H, s), 7.51-7.37 (4H, m), 7.20 (1H, d, J=7.3 Hz), 7.04 (1H, t, J=7.7 Hz), 5.48 (1H, q, J=7.0 Hz), 4.55 (2H, s), 3.71 (1H, d, J=9.4 Hz), 3.09 (1H, d, J=9.4 Hz), 3.04 (3H, s), 2.89 (2H, d, J=5.1 Hz), 2.39 (1H, dd, J=11.5, 6.6 Hz), 2.20-2.05 (4H, m), 1.63 (3H, d, J=7.0 Hz); ¹³C NMR (101 MHz, DMSO-d₆) δ ppm 164.3, 161.4, 159.0, 156.4, 138.5, 138.4, 133.2, 132.0, 131.7, 127.9, 126.3, 123.4, 119.8, 119.6, 119.4, 117.7, 117.5, 114.6, 112.8, 112.7, 75.5, 53.1, 51.0, 47.0, 43.4, 38.6, 38.1, 23.4, 16.3; LRMS [M+H]⁺ 530.346.

### IC-62: 7-[1-[5-(3-Aminopropyl)-1,2,4-oxadiazol-3-yl]ethyl]-3-[3-fluoro-4-(methylsulfonyl methyl)phenyl]-1H-indole-2-carboxylic acid

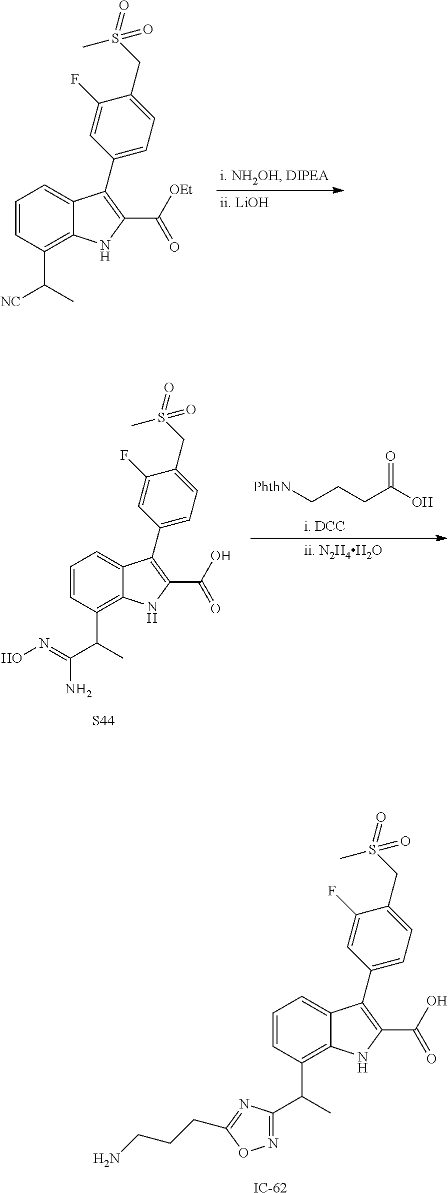

To a solution of the nitrile S14 (160 mg, 0.373 mmol) in 1:1 EtOH: CH₂Cl₂ (1.50 mL) in a microwavable vial was added 50% % in H₂O NH₂OH (0.0172 mmol, 0.280 mmol) and DIPEA (0.13 mL, 0.747 mmol) and the resultant mixture was heated in at 40° C. overnight. The reaction mixture was cooled, diluted with CH₂Cl₂ and washed with H₂O. The aqueous layer was extracted with CH₂Cl₂ and the combined organics were dried (Na₂SO₄) and concentrated in vacuo to afford a white foam which was used without further purification in >99% yield (172 mg).

To the indole ester (171 mg, 0.370 mmol) in 2:1:1 THF:EtOH:H₂O (5.2 mL) was added LiOH·H₂O (77.6 mg, 1.85 mmol) and the reaction mixture was stirred overnight at RT. The reaction was quenched by the addition of 2 M HCl to pH 2 and extracted with EtOAc (×3); the combined organic extracts were dried (Na₂SO₄), filtered and concentrated in vacuo to afford the amidoxime S44 in >99% yield as a colourless solid (160 mg).

To 4-(1,3-dioxoisoindolin-2-yl)butanoic acid (32.3 mg, 0.138 mmol) in dry acetone (0.15 mL) was added DCC (30.9 mg, 0.150 mmol), then stirred at rt for 40 min and then the solvents were removed in vacuo at rt. To a separate μW vial was added amidoxime S44 (50 mg, 0.115 mmol), then the crude activated ester followed by CH₂Cl₂ (0.5) H₂O (1.5 mL). The reaction mixture was then subjected to μW irradiation at 115° C. for 15 min. The resulting solution was diluted with CH₂Cl₂ and washed with water. The organics were dried over Na₂SO₄, filtered and concentrated in vacuo. Purification by FCC (KP-Sil 10 g, CH₂Cl₂ to CH₂Cl₂:MeOH 0-10%) and then repurification by FCC (KP-Sil 10 g, CH₂Cl₂ to CH₂Cl₂:MeOH 0-10%) afforded the oxadiazole in 22% yield (16 mg).

To the phthalimide protected amine (16 mg, 0.025 mmol) in 3:1 THF:EtOH (0.40 mL) was added hydrazine monohydrate (2.5 μL. 0.051 mmol) and the reaction was stirred at rt for 2 h and then concentrated in vacuo. Purification by PrepHPLC chromatography (ACE column, 5 μm, 30×100 mm, H₂O+0.1% FA/MeCN 2→45% gradient) afforded the indole carboxylate IC-62 in 47% yield (6.0 mg).

¹H NMR (500 MHz, MeOD): δ 8.37 (br s, 1H), 7.55-7.37 (m, 4H), 7.21 (d, J=7.1 Hz, 1H), 7.06 (dd, J=8.1, 7.1 Hz, 1H), 4.70 (q, J=7.3 Hz, 1H), 4.60 (s, 3H), 4.52 (br s, 2H), 3.21-3.00 (m, 4H), 2.97 (s, 3H), 2.38-2.21 (m, 2H), 1.82 (d, J=7.3 Hz, 3H); HRMS (TOF, ESI⁺) m/z: [M+H]⁺ Calcd for C₂₄H₂₆FN₄O₅S 501.16025; Found 501.16003.

### IC-63: 3-[3-Fluoro-4-(methylsulfonylmethyl)phenyl]-7-[1-[5-(5-oxopyrrolidin-3-yl)-1,2,4-oxadiazol-3-yl]ethyl]-1H-indole-2-carboxylic acid

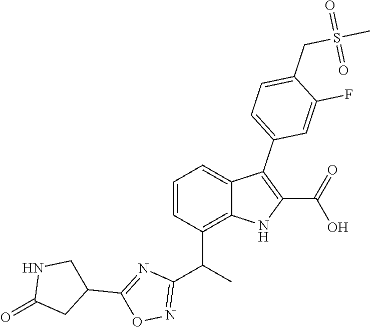 [SMILES: CC(c1noc(C2CNC(=O)C2)n1)c1cccc2c(-c3ccc(CS(C)(=O)=O)c(F)c3)c(C(=O)O)[nH]c12]

To the amidoxime S44 (70 mg, 0.16 mmol) in DMF (1.0 mL) in a microwavable vial was added ZnCl₂ (22 mg, 0.16 mmol), p-toluenesulfonic acid (p-TSA, 31 mg, 0.16 mmol) and 5-oxopyrrolidine-3-carbonitrile (178 mg, 1.61 mmol). The vial was sealed and heated for 2 h at 120° C. and then the reaction was cooled then concentrated in vacuo. Purification by preparative HPLC chromatography (ACE column, 5 μm, 30×100 mm, H₂O+0.01% FA/MeCN 2→45% gradient) afforded the indole carboxylate IC-63 in 26% yield (22 mg).

¹H NMR (600 MHz, DMSO-d6) δ 11.88 (s, 1H), 7.82 (s, 1H), 7.53 (t, J=7.8 Hz, 1H), 7.46-7.34 (m, 3H), 7.18-7.02 (m, 2H), 5.26 (q, J=7.0 Hz, 1H), 4.60 (s, 2H), 4.15-3.97 (m, 1H), 3.68 (ddd, J=10.3, 8.3, 5.7 Hz, 1H), 3.47 (ddd, J=11.6, 9.8, 5.9 Hz, 1H), 3.06 (s, 3H), 2.65 (ddd, J=16.5, 9.5, 4.8 Hz, 1H), 2.49-2.41 (m, 1H), 1.66 (d, J=7.0 Hz, 3H); HRMS (TOF, ESI⁺) m/z: [M+H]⁺ Calcd for C₂₅H₂₄FN₄O₆S 527.13951; Found 527.13940.

### IC-64: 7-[1-[5-(3-Aminopropyl)-2-oxo-1H-imidazol-3-yl]ethyl]-3-[3-fluoro-4-(methylsulfonylmethyl)phenyl]-1H-indole-2-carboxylic acid

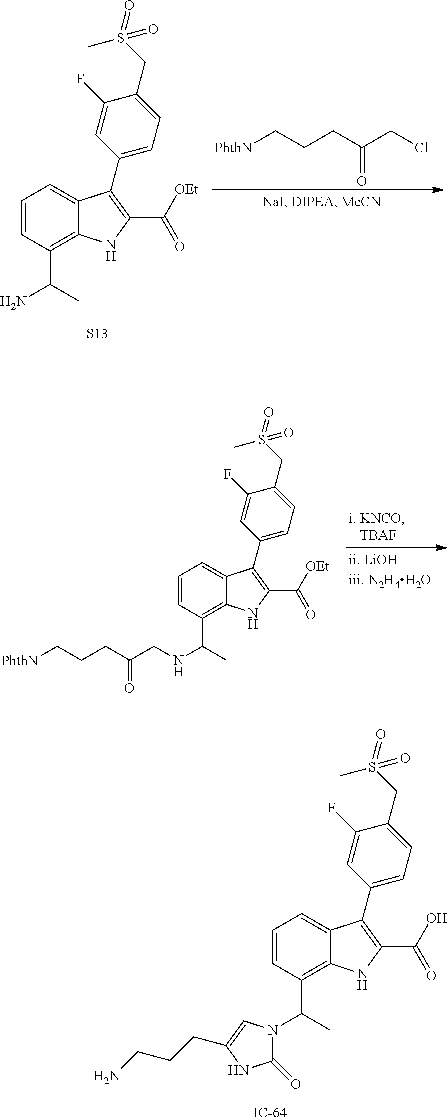

To the amine S13 (220 mg, 0.526 mmol) in MeCN (2.70 mL) was added 2-(5-chloro-4-oxo-pentyl)isoindoline-1,3-dione (168 mg, 0631 mmol), DIPEA (0.110 mL) and NaI (15.8 mg, 0.105 mmol); and the reaction was heated in a sealed vial at 50° C. overnight. The reaction mixture was cooled to rt then concentrated in vacuo. Purification by FCC (SNAP Ultra 10 g, CH₂Cl₂ to EtOAc 0-100%) afforded the α-aminoketone in 65% yield (222 mg).

To the α-aminoketone (220 mg, 0.340 mmol) in 2.5:2.2:1 THF:EtOH:H₂O (2.4 mL)

was added potassium cyanate (55.1 mg, 0.679 mmol) and TBAF (321 mg, 1.02 mmol) and the vial was heated at 60° C. overnight. The reaction was diluted with EtOAc and the aqueous layer extracted with EtOAc; the combined organics were dried (Na₂SO₄) and concentrated in vacuo. Purification by FCC (KP-Sil 10 g, CH₂Cl₂ to CH₂Cl₂: EtOAc 0-100% then EtOAc:MeOH 9:1) afforded the oxaimidazole in 33% yield (76 mg).

To the indole ester (76 mg, 0.113 mmol) in 2:1:1 THF:EtOH:H₂O (3 mL) was added LiOH·H₂O (23.7 mg, 0.565 mmol) and the reaction mixture was stirred overnight at RT. The reaction was quenched by the addition of 2 M HCl to pH 2 and extracted with EtOAc (×3); the combined organic extracts were dried (Na₂SO₄), filtered and concentrated in vacuo to afford the carboxylic acid in >99% yield (73 mg).

To the phthalimide protected amine (70.9 mg, 0.110 mmol) in 3:1 THF:EtOH (1.4 mL) was added hydrazine monohydrate (10.7 μL, 0.220 mmol) and the reaction was stirred at rt for 2 h and then concentrated in vacuo. Purification by RP-Biotage (C18 column, H₂O to MeOH MeCN 2→50% gradient) afforded the indole carboxylate IC-64 in 6% yield (3.4 mg).

¹H NMR (500 MHz, MeOD) δ 7.61-7.52 (m, 2H), 7.47 (d, J=7.2, 1H), 7.42-7.35 (m, 3H), 7.21-7.15 (m, 1H), 6.36 (s, 1H), 5.86 (q, J=7.0, 1H), 4.58 (s, 2H), 3.07-2.96 (m, 5H), 2.87 (dd, J=9.3, 6.5, 2H), 2.46 (t, J=7.4, 2H), 1.93-1.82 (m, 6H); HRMS (TOF, ESI⁺) m/z: [M+H]⁺ Calcd for C₂₅H₂₈FN₄O₅S 515.17590; Found 515.17584.

### IC-65: (S)-3-(3-fluoro-4-((methylsulfonyl)methyl)phenyl)-7-(1-(6-oxo-5-oxa-2,7-diazaspiro[3.4]octan-7-yl)ethyl)-1H-indole-2-carboxylic acid and

### IC-66: (S)-7-(1-(2-carbamimidoyl-6-oxo-5-oxa-2,7-diazaspiro[3.4]octan-7-yl)ethyl)-3-(3-fluoro-4-((methylsulfonyl)methyl)phenyl)-1H-indole-2-carboxylic acid

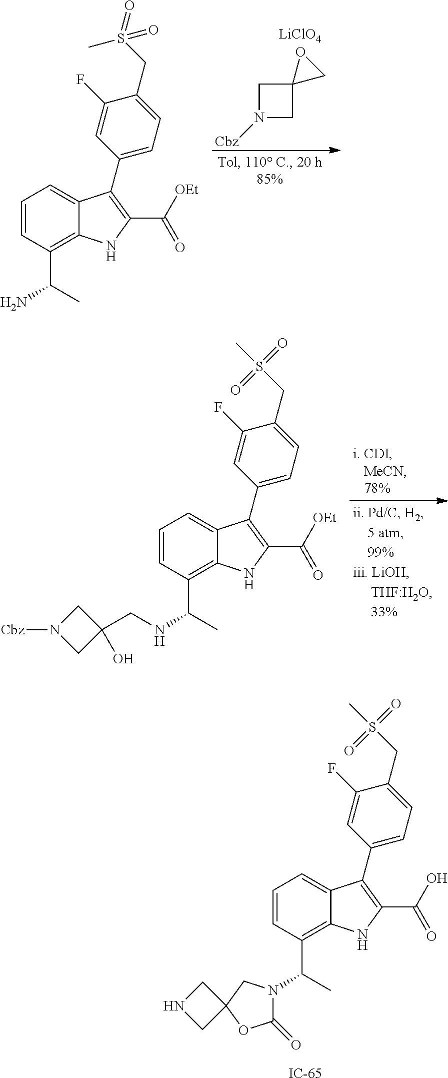

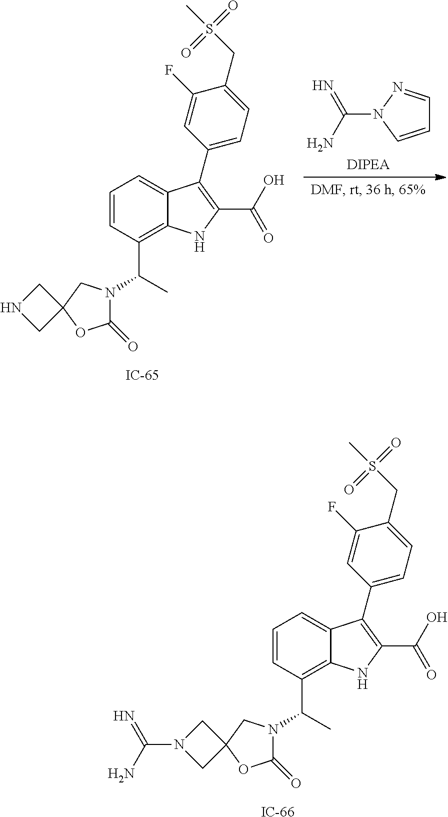

A beige suspension of the amine S39 (921 mg, 2.20 mmol, 1.10 eq), benzyl 1-oxa-5-azaspiro[2.3]hexane-5-carboxylate (438 mg, 2.00 mmol) and LiCIO₄ (226 mg, 2.00 mmol) in anh toluene (7 mL) was heated at 110° C. for 20 h. The resulting brown suspension was cooled to RT, diluted with EtOAc (40 mL), water was added (50 mL) and the layers were separated; the aqueous layer extracted with EtOAc (2×25 mL); the combined organics were washed with brine, dried over Na₂SO₄ and concentrated in vacuo to obtain brown oil. Purification by FCC (DCM:MeOH 50:1 to 20:1) afforded the amino alcohol as a yellow foam in 85% yield (1.08 g).

To a yellow solution of aminoalcohol (1.07 g, 1.64 mmol) in MeCN (15 mL) was added CDI (400 mg, 2.46 mmol, 1.50 eq) and the reaction was stirred at RT for 20 h and then another 0.5 equiv of CDI (133 mg) was added ar RT and stirred for a further 2 h. The resulting yellow solution was concentrated in vacuo, dissolved in EtOAc, washed with 1 M aqueous HCl, water, sat. aq. NaHCO₃, brine, dried over Na₂SO₄ and concentrated in vacuo. Purification by FCC (DCM:MeOH 20:1) afforded the oxazolidone as a yellow foam in 78% yield (865 mg).

To the N-Cbz azetidine (800 mg, 1.21 mmol) in acetic acid (62 mL) was added 10% Pd/C (769 mg, 0.36 mmol) and the reaction mixture was placed under a hydrogen atmosphere (5 bar) at RT for 1 h. The reaction mixture was filtered through a pad of Celite®, and the filter cake was washed with EtOH (5×10 mL) and the filtrate additionally filtered through a 0.2 μm syringe filter. The volatiles were removed in vacuo to afford the azetidine as a brown oil in quantitative yield (638 mg).

To the ethyl ester (361 mg, 0.648 mmol) in THF (5.7 mL) was added a solution of LiOH·H₂O (163 mg, 3.89 mmol in 5.7 mL H₂O) at 0° C. and the reaction mixture was gradually warmed to 19° C. over 14 h. The reaction was quenched at 19° C. by the dropwise addition of AcOH (260 μL, 4.53 mmol) and the volatiles removed in vacuo. Purification by preparative HPLC [Atlantis, 30×100 mm, 5 um, 0.1% AcOH water:MeCN=95:5 to 5:95] afforded the indole carboxylate IC-65 in 33% yield (113 mg).

¹H NMR (400 MHz, DMSO-d6): 10.92-10.74 (bs, 1H), 7.53-7.43 (m, 3H), 7.41 (dd, J=7.9, 1.6 Hz, 1H), 7.28 (d, J=7.2 Hz, 1H), 7.11 (dd, J=8.1, 7.2 Hz, 1H), 5.54 (q, J=7.0 Hz, 1H), 4.58 (s, 2H), 3.92 (dd, J=12.6, 10.0 Hz, 2H), 3.82 (dd, J=15.2, 10.0 Hz, 2H), 3.67 (d, J=10.0 Hz, 1H), 3.49 (d, J=10.0 Hz, 1H), 3.05 (s, 3H), 1.64 (d, J=7.0 Hz, 3H), —CO₂H and —NH signals not observed due to broadening; LRMS [M+H]⁺ 502.

To a suspension of azetidine IC-65 (90 mg, 0.172 mmol) and [amino(pyrazol-1-yl)methylene]ammonium chloride (27.8 mg, 0.190 mmol) in DMF (2.24 mL) was added DIPEA (75 μL, 0.431 mL) at RT and the reaction was stirred at RT for 15 h. The reaction mixture was directly purified by RP prepHPLC [Atlantis, Sum, 30×100 mm, H₂O+0.1% AcOH/MeCN 5→95% gradient] to afford the indole carboxylate IC-66 as a white solid in 65% yield (72 mg).

¹H NMR (400 MHz, DMSO-d6) 10.43-10.22 (br s, 1H), 8.39-7.80 (br s, 3H), 7.56-7.48 (m, 2H), 7.46-7.39 (m, 2H), 7.18 (d, J=7.2 Hz, 1H), 7.05 (t, J=7.6 Hz, 1H), 5.48 (q, J=7.0 Hz, 1H), 4.56 (s, 2H), 4.31 (d, J=10.4 Hz, 1H), 4.24 (d, J=10.4 Hz, 1H), 4.16 (dd, J=12.8, 10.4 Hz, 2H), 3.93 (d, J=10.2 Hz, 1H), 3.46 (d, J=10.2 Hz, 1H), 3.05 (s, 3H), 1.64 (d, J=7.0 Hz, 3H), —CO₂H signal not observed due to broadening; LRMS [M+H]⁺ 544.

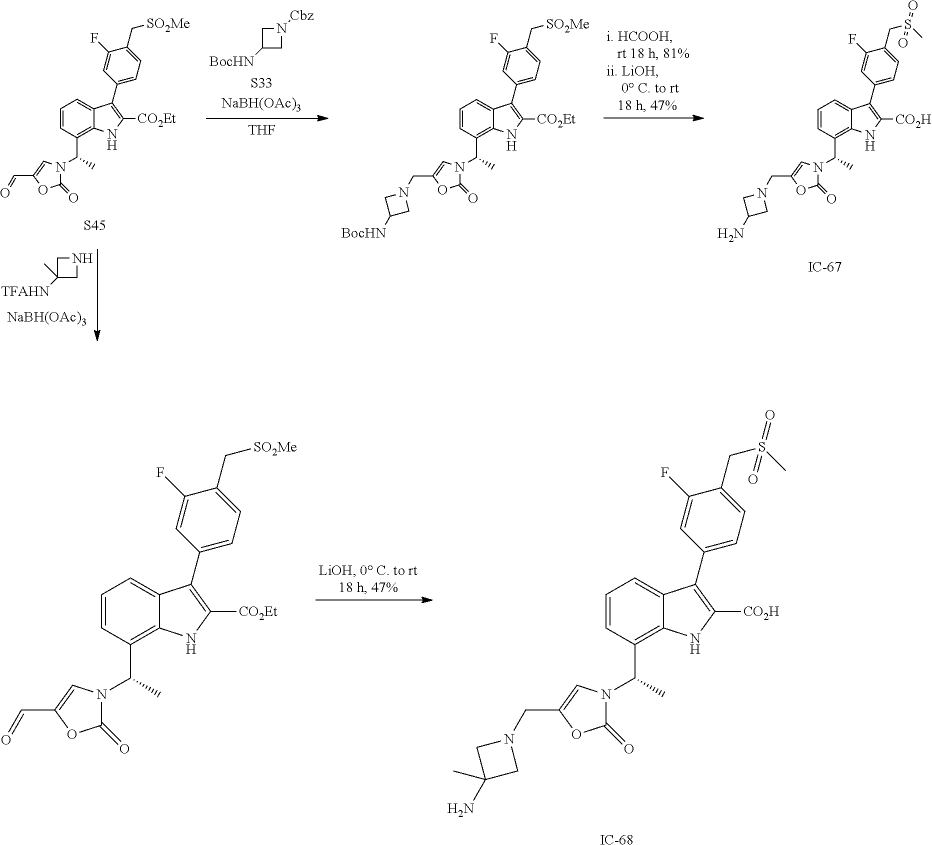

### IC-67: (S)-7-(1-(5-((3-aminoazetidin-1-yl)methyl)-2-oxooxazol-3(2H)-yl)ethyl)-3-(3-fluoro-4-((methylsulfonyl)methyl)phenyl)-1H-indole-2-carboxylic acid

To a solution of aldehyde S45 (161 mg, 0.313 mmol) in THE (5 mL) at 0° C. was added azetidine S33 (81 mg, 0.469 mmol) and the resulting solution was stirred at this temperature for 10 min and then NaBH(OAc)₃ (100 mg, 0.469 mmol) was added in one portion. The reaction mixture was allowed to warm up to RT and stirred for 18 h and then the volatiles were removed in vacuo. The crude reaction was diluted with EtOAc and sat. NaHCO₃ was added and the aqueous layer was extracted with EtOAc (3×30 mL); the combined organics were dried (Na₂SO₄) and concentrated in vacuo. Purification by FCC (KP-Sil 10 g silica, EtOAc/EtOH 95:5) afforded the azetidine in 83% yield (175 mg).

To the N-Boc amine (175 mg, 0.26 mmol) was added formic acid (3 mL) and the reaction was stirred at RT for 18 h. The reaction mixture was diluted with EtOAc (20 mL) and sat. NaHCO₃ (20 mL); the aqueous layer was extracted with EtOAc (3×20 mL) and the combined organics were dried (Na₂SO₄) and then concentrated. Purification by FCC (KP-Sil 10 g silica, DCM/MeOH 0-50%) afforded the amino azetidine in 85% yield (126 mg).

To the ethyl ester (126 mg, 0.221 mmol) in THF (2.5 mL) was added a solution of LiOH·H₂O (32 mg, 1.32 mmol in 2.5 mL H₂O) at 0° C. and the reaction mixture was gradually warmed to 19° C. over 14 h. The reaction was quenched at 19° C. by the dropwise addition of AcOH (139 μL, 2.43 mmol) and the volatiles removed in vacuo. Purification by preparative HPLC [Atlantis, 30×100 mm, Sum, 0.1% AcOH water:MeCN=95:5 to 20:80] afforded the indole carboxylate IC-67 in 47% yield (59 mg).

¹H NMR (400 MHz, DMSO-d6) 10.94-10.82 (1H, br s) 7.51-7.44 (3H, m) 7.42 (1H, dd, J=7.8, 1.5 Hz) 7.17-7.14 (2H, m) 7.09-7.04 (1H, m) 5.81 (1H, q, J=7.0 Hz) 4.57 (2H, s) 3.61-3.53 (1H, m, overlapped with water) 3.48-3.39 (4H, m overlapped with water) 3.05 (3H, s), 3.01-2.92 (2H, m), 1.72 (3H, d, J=7.0 Hz); LRMS [M+H]⁺ 543.

### IC-68: (S)-7-(1-(5-((3-amino-3-methylazetidin-1-yl)methyl)-2-oxooxazol-3(2H)-yl)ethyl)-3-(3-fluoro-4-((methylsulfonyl)methyl)phenyl)-1H-indole-2-carboxylic acid_

To a solution of S45 (86 mg, 0.164 mmol) in THF (3 mL) at 0° C. was added 3-methyl-3-trifluoroacetamido-azetidine hydrochloride (51 mg, 0.234 mmol) and the resulting solution was stirred at this temperature for 10 min and then NaBH(OAc)₃ (69 mg, 0.328 mmol) was added in one portion. The reaction mixture was allowed to warm up to RT and stirred for 8 h and then the reaction was quenched by the addition of sat NH₄Cl (10 mL) and EtOAc (10 mL). The aqueous layer was extracted with EtAOc (2×20 mL) and the combined organics were dried (Na₂SO₄), filtered and concentrated. Purification by FCC (CH₂Cl₂/EtOH 95:5 to 90:10) afforded the azetidine in 73% yield (82 mg).

To the ethyl ester (82 mg, 0.119 mmol) in THF (2.0 mL) was added a solution of LiOH·H₂O (30 mg, 0.716 mmol in 2.0 mL H₂O) at 0° C. and the reaction mixture was gradually warmed to 19° C. over 18 h. The reaction was quenched at 19° C. by the dropwise addition of AcOH (41 μL, 0.716 mmol) and the volatiles removed in vacuo. Purification by preparative HPLC [Atlantis, 30×100 mm, Sum, 0.1% AcOH water:MeCN=95:5 to 30:70], concentration then trituration (heptane) of product fractions afforded the indole carboxylate IC-68 in 77% yield (52 mg).

¹H NMR (400 MHz, DMSO-d6) 10.95-10.72 (bs, 1H), 7.52-7.43 (m, 3H), 7.41 (dd, J=7.9, 1.6 Hz, 1H), 7.16 (d, J=7.3 Hz, 1H), 7.11 (s, 1H), 7.05 (t, J=7.9 Hz, 1H), 5.81 (q, J=7.0 Hz, 1H), 4.57 (s, 2H), 3.20-3.09 (m, 6H), 3.05 (s, 3H), 1.72 (d, J=7.0 Hz, 3H), 1.38 (s, 3H), —CO₂H and —NH₂ signals not observed due to broadening; LRMS [M+H]⁺ 557.

### IC-69: (S)-7-(1-(5-(2-(1-Aminocyclopropyl)ethyl)-2-oxooxazol-3(2H)-yl)ethyl)-3-(3-fluoro-4-((methylsulfonyl)methyl)phenyl)-1H-indole-2-carboxylic acid

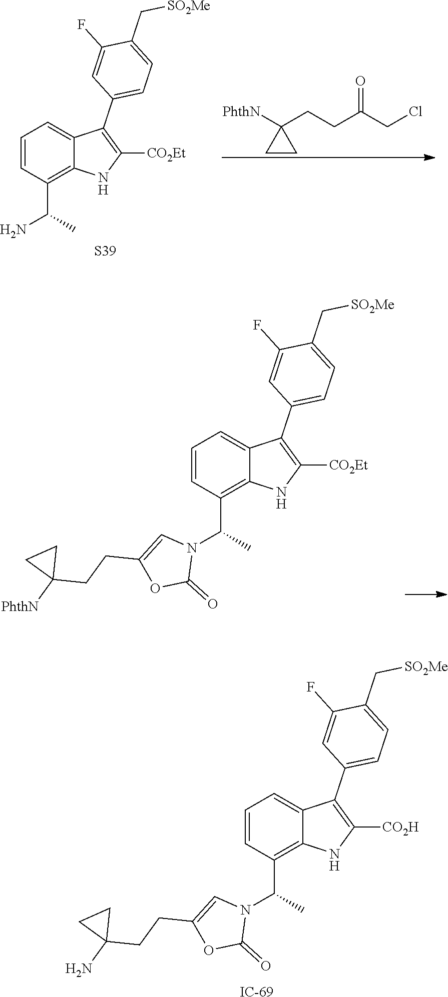

To the amine S39 (299 mg, 0.715 mmol), 2-[1-(4-chloro-3-oxo-butyl)cyclopropyl]isoindoline-1,3-dione (204 mg, 0.650 mmol) and NaI (19.5 mg, 0.130 mmol) in MeCN (1.6 mL) was added DIPEA (0.136 mL, 0.780 mmol) and the yellow solution was stirred for 23 h at 50° C. CDI (316 mg, 1.95 mmol) was then added and the mixture was heated at 80° C. for 24 h. The obtained yellow solution was diluted with EtOAc, washed with 1 M HCl (×2), H₂O, sat. NaHCO₃, brine, dried (Na₂SO₄) and filtered through a short silica plug. Purification by FCC (CH₂Cl₂/EtOAc 10:1) afforded the oxazolone in 38% yield (174 mg).

To a solution of the oxazolone (260 mg, 0.372 mmol) in THF (3.7 mL) was added N₂H₄·H₂O (0.18 mL, 3.72 mmol) under argon atmosphere and the reaction was stirred at 70° C. for 18 h. The reaction mixture was cooled and partitioned between DCM and H₂O; the organic layer was washed additionally with H₂O (×2), brine, then dried (Na₂SO₄) and concentrated in vacuo. The obtained intermediate aminoester was dissolved in THF (1.8 mL) and a solution of LiOH·H₂O (78 mg, 1.86 mmol) in H₂O (1.8 mL) was added at 0° C. amd the reaction was stirred at RT for 19 h. The reaction was quenched by the addition of AcOH (0.128 mL, 2.23 mmol) and evaporated with DMSO (0.7 mL). Purification by RP FCC (C18, H₂O+0.5% AcOH/MeCN, 0→60% gradient) then by preparative HPLC [XBridge, 30×100 mm, 5 μm, 0.1% AcOH water:MeCN=95:5 to 5:95] afforded the indole carboxylate IC-69 as a yellowish powder in 33% yield (67 mg).

¹H NMR (400 MHz, DMSO-d6) 11.78-10.30 (bs, 1H), 7.51-7.42 (m, 3H), 7.39 (dd, J=7.9, 1.6 Hz, 1H), 7.17 (d, J=7.2 Hz, 1H), 7.05 (t, J=7.7 Hz, 1H), 7.01 (s, 1H), 5.82 (q, J=7.0 Hz, 1H), 4.57 (s, 2H), 3.05 (s, 3H), 2.60-2.53 (m, 2H), 1.79-1.59 (m, 5H), 0.73-0.56 (m, 2H), 0.56-0.39 (m, 2H), —CO₂H and —NH₂ signals not observed due to broadening; LRMS [M+H]⁺ 542.

### IC-70: (S)-7-(1-(5-(3-aminopropyl)-2-oxooxazol-3(2H)-yl)ethyl)-3-(3-fluoro-4-((methylsulfonyl)methyl)phenyl)-1H-indole-2-carboxylic acid

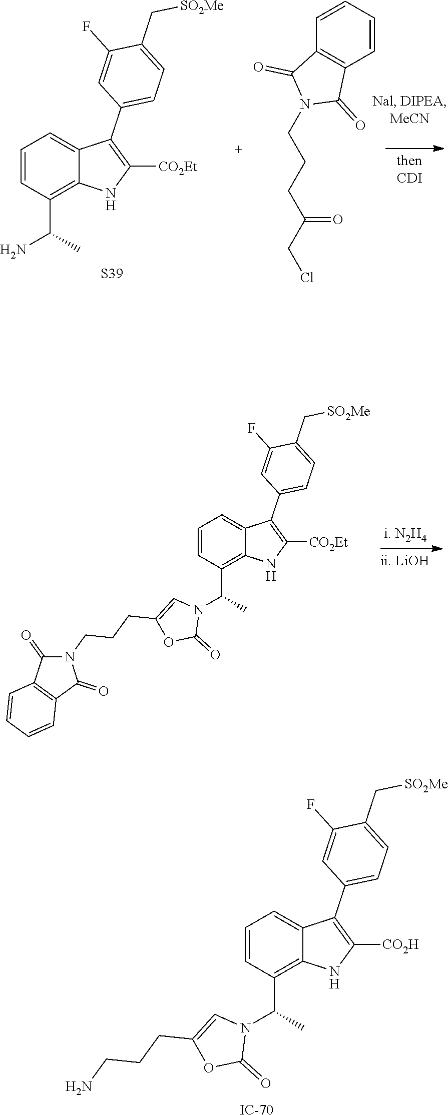

To the amine S39 (298 mg, 0.712 mmol) in MeCN (2.00 mL) was added 2-(5-chloro-4-oxo-pentyl)isoindoline-1,3-dione (172 mg, 0.647 mmol), and NaI (19.4 mg, 0.129 mmol) followed by DIPEA (0.135 mL, 0.777 mmol); and the reaction was heated in a sealed vial at 50° C. for 21 h. To the reaction mixture was added CDI (315 mg, 1.94 mmol) and then the reaction mixture was heated to 80° C. for 24 h. The reaction was cooled to RT and diluted with CH₂Cl₂, washed with 1 M HCl (×2), H₂O, sat. NaHCO₃, brine then dried (Na₂SO₄) and concentrated in vacuo. Purification by FCC (CH₂Cl₂ to EtOAc 10:1) afforded the oxazolone in 38% yield (167 mg).

To a solution of the oxazolone (167 mg, 0.248 mmol) in THF (2.5 mL) was added N₂H₄·H₂O (0.096 mL, 1.98 mmol) under argon atmosphere and the reaction was stirred at 60° C. for 16 h. The reaction mixture was cooled and partitioned between DCM and H₂O; the organic layer was washed additionally with H₂O (×2), brine, then dried (Na₂SO₄) and concentrated in vacuo. The obtained intermediate aminoester was dissolved in THF (1.2 mL) and a solution of LiOH·H₂O (52 mg, 1.86 mmol) in H₂O (1.2 mL) was added at 0° C. and the reaction was stirred at RT for 18 h. The reaction was quenched by the addition of AcOH (0.085 mL, 1.49 mmol) and evaporated with DMSO (0.5 mL). Purification by RP FCC (C18, H₂O+0.5% AcOH/MeCN, 0-50% gradient); product fractions were combined, concentrated and suspended in H₂O (0.50 mL) and the suspension was sonicated then centrifuged to afford the indole carboxylate IC-70 as a yellowish powder in 69% yield (88 mg).

¹H NMR (300 MHz, DMSO-d6):11.42-9.81 (bs, 1H), 9.07-7.58 (bs, 2H), 7.53-7.37 (m, 4H), 7.16 (d, J=7.3 Hz, 1H), 7.06-6.96 (m, 2H), 5.73 (q, J=7.0 Hz, 1H), 4.55 (s, 2H), 3.04 (s, 3H), 2.79 (t, J=7.4 Hz, 2H), 2.46 (t, J=7.4 Hz, 2H), 1.83-1.66 (m, 5H), —CO₂H signal not observed; LRMS [M+H]⁺ 516.

### IC-71: (S)-7-(1-(8-amino-2-oxo-1-oxa-3-azaspiro[4.5]decan-3-yl)ethyl)-3-(3-fluoro-4-((methylsulfonyl)methyl)phenyl)-1H-indole-2-carboxylic acid

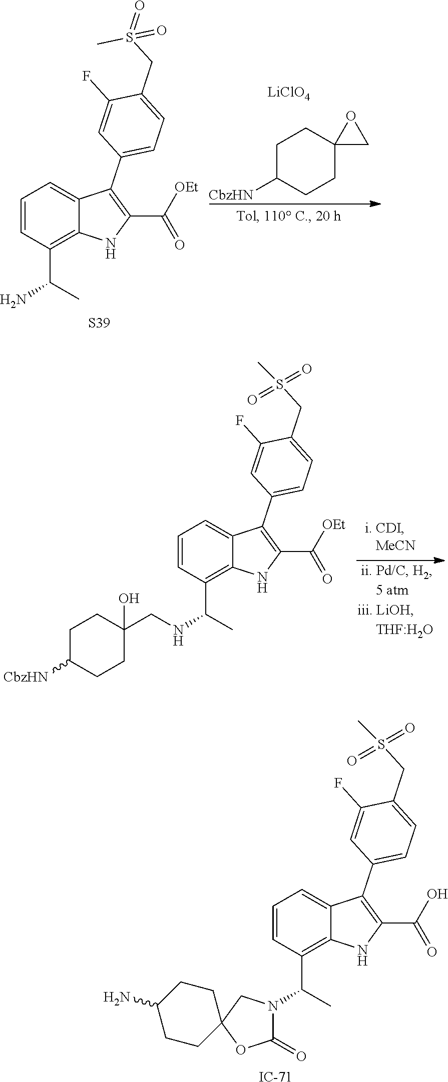

The amine S39 (150 mg, 0.358 mmol, 1.10 eq), benzyl N-(1-oxaspiro[2.5]octan-6-yl)carbamate (85 mg, 0.325 mmol) and LiClO₄ (35 mg, 0.325 mmol) in anh toluene (1.00 mL) was heated at 110° C. for 18 h in a sealed vial. The reaction mixture was cooled to RT, diluted with EtOAc and water and the layers were separated; the aqueous layer extracted with EtOAc (2×25 mL); the combined organics were washed with brine, dried over Na₂SO₄ and concentrated in vacuo to obtain the amino alcohol which was used in the next step without further purification in quantitative yield (221 mg).

To the crude amino alcohol (221 mg, 0.325 mmol) in MeCN (4 mL) was added CDI (158 mg, 0.992 mmol) and the reaction was stirred at 85° C. for 18 h. The crude reaction mixture was concentrated in vacuo, dissolved in DCM and washed with H₂O (20 mL) and then the organic layer was dried (Na₂SO₄) and then concentrated in vacuo. Purification by preparative HPLC [XBridge eluent 0.1% HCOOH in water 90-5%; MeCN 5-95%] afforded the oxazolidone in 31% yield (70 mg).

To the N-Cbz amine (70 mg, 0.099 mmol) in EtOH (10 mL) was added 10% Pd/C (20 mg, 0.0093 mmol) and the reaction mixture was placed under a hydrogen atmosphere at RT for 3 h. The reaction mixture was filtered through a pad of Celite®, and the filter cake was washed with EtOH. The volatiles were removed in vacuo to afford the amine which used without further purification as a brown oil in 86% yield (49 mg).

To the ethyl ester (66 mg, 0.0115 mmol) in THF (2.0 mL) was added a solution of LiOH·H₂O (29.1 mg, 0.0700 mmol in 2.0 mL H₂O) at 0° C. and the reaction mixture was gradually warmed to 19° C. over 16 h. The reaction was quenched at 19° C. by the dropwise addition of AcOH and the volatiles removed in vacuo. Purification by RP FCC (KP-C18-HS 10 g, 0.02% AcOH water:MeCN=95:5 to 30:70] afforded the indole carboxylate IC-71 in 61% yield (38 mg).

¹H NMR (400 MHz, DMSO-d6) 10.00 (1H, s) 8.8-8.1 (3H, br s) 7.56-7.51 (2H, m) 7.47-7.39 (2H, m) 7.19-7.15 (1H, m) 7.08-7.02 (1H, m) 5.44 (1H, q, J=7.0 Hz) 4.54 (2H, s) 3.30-3.23 (2H, m) 3.03 (3H, s) 2.67-2.61 (1H, m) 1.97-1.91 (1H, m) 1.86-1.78 (1H, m) 1.76-1.68 (1H, m) 1.62 (3H, d, J=7.0 Hz) 1.65-1.38 (5H, m); LRMS [M+H]⁺ 544.

### IC-72: 7-(1-((5-(3-aminopropyl)oxazol-2-yI)oxy)ethyl)-3-(3-fluoro-4-((methylsulfonyl)methyl)phenyl)-1H-indole-2-carboxylic acid

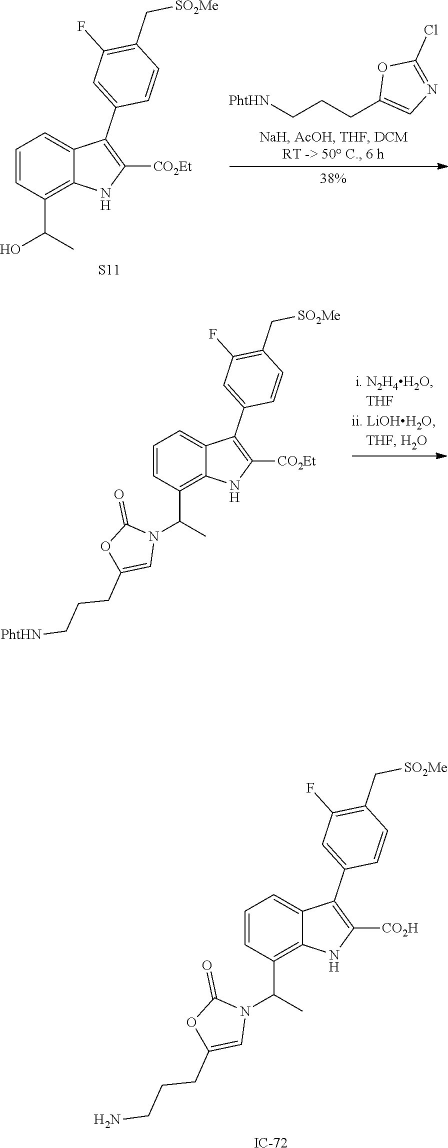

A solution of the alcohol S11 (996 mg, 2.37 mmol, 20 mL THF) was added dropwise at 0° C. to a suspension of NaH (759 mg, 10.9 mmol, 60% in mineral oil) in THF (2.5 mL) under argon atmosphere. The reaction mixture was warmed to RT and stirred for 20 min and then a solution of 2-[3-(2-chlorooxazol-5-yl)propyl]isoindoline-1,3-dione (1.04 g, 3.56 mmol in 7.5 mL THF) was added dropwise and the resulting orange suspension was stirred at 50° C. for 6 h. The reaction mixture was cooled to 0° C. and quenched with AcOH (1.90 mL, 33.2 mmol) then diluted with EtOAc and brine. The organic layer was washed additionally with brine and then dried (Na₂SO₄) and concentrated. Purification by RP FCC (0.1% formic acid water:MeCN=90:10 to 5:95) afforded the product as an orange solid in 38% yield (974 mg).

To the phthalimide protected amine (974 mg, 0.889 mmol) in THF (45 mL) was added hydrazine monohydrate (0.863 mL, 17.8 mmol) and the reaction was stirred at 60° C. for 6 h and then partitioned between CH₂Cl₂ and H₂O. The organic layer was washed additionally with H₂O (×2), brine, dried (Na₂SO₄) and concentrated in vacuo. Purification by RP FCC (0.5% FA H₂O/MeCN 10->95% gradient) afforded the amine S46 in 37% yield (190 mg).

To the amino ester S46 (190 mg, 0.329 mmol) in THF (7.5 mL) was added a solution of LiOH·H₂O (83 mg, 1.97 mmol in 7.5 mL H₂O) at 0° C. and the reaction mixture was gradually warmed to 19° C. over 14 h. The reaction was quenched at 19° C. by the dropwise addition of AcOH (0.132 mL, 2.30 mmol) and the volatiles removed in vacuo. Purification by preparative HPLC [Atlantis, 30×100 mm, 5 μm, 0.1% AcOH water:MeCN=95:5 to 5:95] and then trituration in heptane (2 mL) of the concentrated product fraction afforded the indole carboxylate IC-72 as a beige solid in 87% yield (152 mg).

¹H NMR (300 MHz, DMSO-d6):10.70-10.36 (bs, 1H), 8.63-7.64 (bs, 2H), 7.52 (d, J=8.8 Hz, 1H), 7.49 (d, J=4.9 Hz, 1H), 7.43 (d, J=4.1 Hz, 2H), 7.17 (d, J=7.2 Hz, 1H), 7.03 (t, J=7.7 Hz, 1H), 7.00 (s, 1H), 5.71 (q, J=6.9 Hz, 1H), 4.55 (s, 2H), 3.04 (s, 3H), 2.78 (t, J=7.5 Hz, 2H), 2.73 (p, J=1.8 Hz, 1H), 2.27 (p, J=1.8 Hz, 1H), 1.81-1.66 (m, 5H), one exchangeable proton not observed; LRMS [M+H]⁺ 516.

### IC-73: 3-(3-Fluoro-4-((methylsulfonyl)methyl)phenyl)-7-(1-(5-(3-guanidinopropyl)-2-oxooxazol-3(2H)-yl)ethyl)-1H-indole-2-carboxylic acid hydrochloride

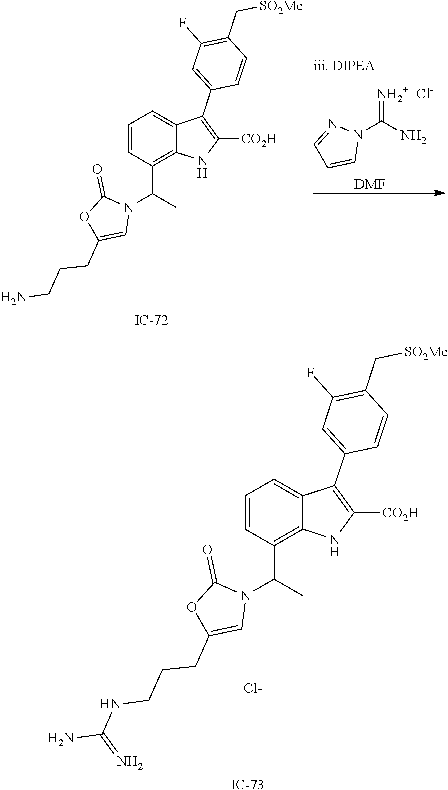

To the amine IC-72 (17.5 mg, 0.039 mmol) in DMF (0.50 mL) was added [amino(pyrazol-1-yl)methylene]ammonium chloride (5.5 mg, 0.037 mmol) then DIPEA (8.8 μL, 0.051 mmol) at RT and the reaction was stirred for 36 h. The reaction mixture was directly purified by Preparative HPLC (Atlantis, 5 μm, 30×100 mm, H₂O+0.01% TFA/MeCN 5-80% gradient) and fractions containing product were evaporated, then coevaporated with 1 M HCl (×3), the residue was suspended in MeCN, heptane, sonicated and scratched with a spatula and dried to afford the indole carboxylate IC-73 in 71% yield as a pale pink solid (15 mg).

¹H NMR (400 MHz, DMSO-d6) 11.64 (s, 1H), 7.57-7.50 (m, 2H), 7.46 (d, J=8.0 Hz, 1H), 7.42-7.34 (m, 2H), 7.22 (d, J=7.2 Hz, 1H), 7.16 (s, 1H), 7.13 (dd, J=8.0, 7.2 Hz, 1H), 5.91 (q, J=6.9 Hz, 1H), 4.61 (s, 2H), 3.19-3.12 (m, 2H), 3.07 (s, 3H), 2.46-2.41 (m, 2H), 1.76-1.69 (m, 5H), five exchangeable proton not observed; LRMS [M+H]⁺ 558.

### IC-74: 7-(1-(5-(2-(1-aminocyclopropyl)ethyl)-2-oxooxazol-3(2H)-yl)ethyl)-3-(3-fluoro-4-((methylsulfonyl)methyl)phenyl)-1H-indole-2-carboxylic acid

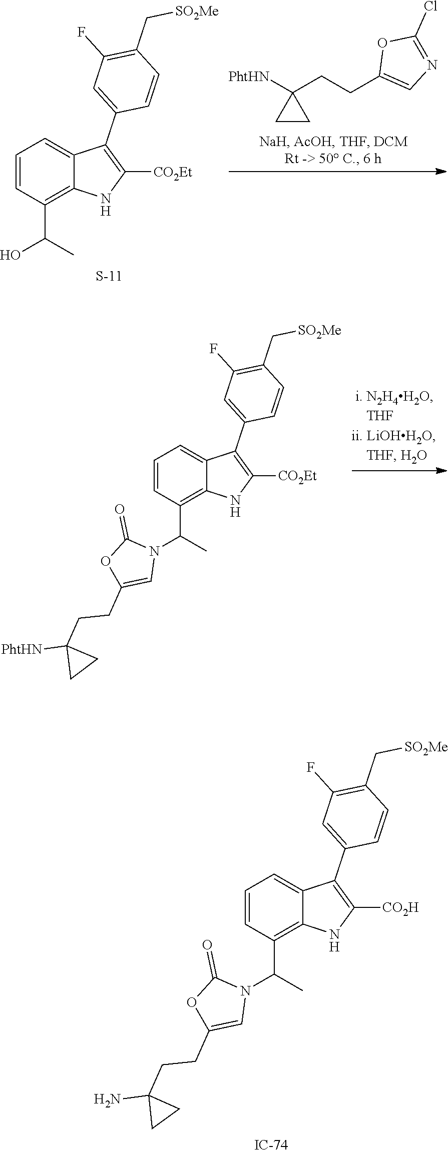

A solution of the alcohol S11 (268 mg, 0.639 mmol, 3 mL THF) was added dropwise at 0° C. to a suspension of NaH (204 mg, 5.11 mmol, 60% in mineral oil, washed off oil with THF 4×2 mL) in THF (0.5 mL) under argon atmosphere. The reaction mixture was warmed to RT and stirred for 10 min and then at 50° C. followed by the addition of a solution of 2-[1-[2-(2-chlorooxazol-5-yl)ethyl]cyclopropyl]isoindoline-1,3-dione (263 mg, 0.831 mmol in 3 mL THF) dropwise. The resulting orange suspension was stirred at 50° C. for 3 h and then for 3 h at 60° C. The reaction mixture was cooled to 0° C. and quenched with AcOH (0.585 mL, 10.2 mmol) then diluted with EtOAc and brine. The organic layer was washed additionally with H₂O, sat NaHCO₃, brine and then dried (Na₂SO₄) and concentrated. Purification by FCC (DCM:EtOAc 10:1 to 7:1) then by RP FCC (0.1% formic acid water:MeCN=95:5 to 5:95) afforded the product as a colourless film 49% yield (221 mg).

To the phthalimide protected amine (221 mg, 0.316 mmol) in THF (3 mL) was added hydrazine monohydrate (0.123 mL, 2.53 mmol) and the reaction was stirred at 60° C. for 23 h and then partitioned between CH₂Cl₂ and H₂O. The organic layer was washed additionally with H₂O (×2), brine, dried (Na₂SO₄) and concentrated in vacuo to afford the amino ester as a yellowish oil (205 mg). To the crude amino ester in THF (1.5 mL) was added a solution of LiOH·H₂O (66 mg, 1.58 mmol in 1.5 mL H₂O) at 0° C. and the reaction mixture was gradually warmed to RT over 24 h. The reaction was quenched at 19° C. by the dropwise addition of AcOH (0.108 mL, 1.89 mmol) and the volatiles removed in vacuo. Purification by RP-FCC (C18, 0.5% AcOH water:MeCN=0 to 40:60] and then trituration in MeCN of the concentrated product fractions afforded the indole carboxylate IC-74 as a greenish solid in 51% yield (88 mg).

¹H NMR (400 MHz, DMSO-d6): 11.26-10.79 (bs, 1H), 7.51-7.42 (m, 3H), 7.39 (dd, J=7.9, 1.5 Hz, 1H), 7.17 (d, J=7.2 Hz, 1H), 7.05 (t, J=7.7 Hz, 1H), 7.01 (s, 1H), 5.82 (q, J=6.9 Hz, 1H), 4.57 (s, 2H), 3.05 (s, 3H), 2.61-2.53 (m, 2H), 1.79-1.60 (m, 5H), 0.76-0.59 (m, 2H), 0.58-0.40 (m, 2H), —CO₂H and —NH₂ signals not observed due to broadening; LRMS [M+H]⁺ 542.

### IC-75: 7-(1-(5-(3-(Dimethylamino)propyl)-2-oxooxazol-3(2H)-yl)ethyl)-3-(3-fluoro-4-((methylsulfonyl)methyl)phenyl)-1H-indole-2-carboxylic acid

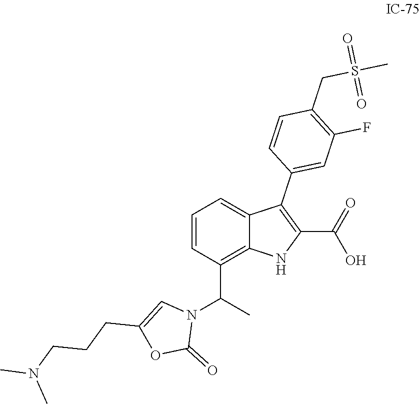

To a solution of primary amine S46 (15 mg, 0.0248 mmol), AcOH (3.7 μL, 0.0646 mmol) and NaBH₃CN (2.8 mg, 0.0447 mmol) in MeOH (0.5 mL) at 0° C. was added 37% w/w solution of formaldehyde (6.7 μL, 0.0829 mmol) in MeOH (0.5 mL). The solution was warmed to 25° C. and stirred for 2.5 h before adding 1 mL of a 10% aqueous K₂CO₃ solution. The MeOH was removed under vacuum, 2 mL of H₂O were added to the residue, and it was extracted with 3×1 mL of EtOAc. Organics combined and dried over Na₂SO₄, filtered and evaporated to obtain the tertiary amine as a yellow oil (15 mg). To the crude amino ester in THF (3 mL) was added a solution of LiOH·H₂O (6.3 mg, 0.15 mmol in 3 mL H₂O) at 0° C. and the reaction mixture was gradually warmed to RT over 48 h. The reaction was quenched at 19° C. by the dropwise addition of AcOH (0.010 mL, 0.174 mmol) and the volatiles removed in vacuo. Purification by RP-FCC (C18, 0.5% AcOH water:MeCN=90:10 to 10:90] afforded the indole carboxylate IC-75 as a beige solid in 29% yield (4.1 mg).

¹H NMR (300 MHz, MeCN-d3/D₂O (v/v=4:1)): 8.30 (1H, s), 7.54 (1H, dd, J=8.1, 1.0 Hz), 7.47-7.26 (4H, m), 7.10 (1H, dd, J=8.1, 7.3 Hz), 6.38 (1H, s), 5.58 (1H, q, J=7.1 Hz), 4.45 (2H, s), 2.96 (3H, s), 2.94-2.86 (2H, m), 2.68 (6H, s), 2.29 (2H, t, J=7.3 Hz), 1.84-1.69 (5H, m); LRMS [M+H]⁺ 544.

### IC-76: 3-(3-(1-(2-carboxy-3-(3-fluoro-4-((methylsulfonyl)methyl)phenyl)-1H-indol-7-yl)ethyl)-2-oxo-2,3-dihydrooxazol-5-yl)-N,N,N-trimethylpropan-1-aminium

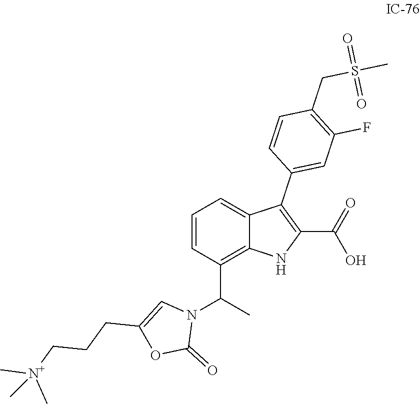

To a suspension of the amine IC-75 (54 mg, 0.0983 mmol) in MeCN/H₂O (1:1, 10 mL) was added Mel (0.10 mL) and the reaction was heated at 50° C. for 20 h. The reaction mixture was concentrated in vacuo and diethyl ether (10 mL) and 2 mL of dry MeCN were added and the reaction was sonicated for 10 min. The ammonium salt was isolated by filtration as a pale, yellow solid. Purification by preparative HPLC [C18 Xbridge, 30×100 mm, 5 μm, 0.1% HCOOH water:MeCN=90:10 to 50:50] and then trituration and sonication in heptane of the concentrated product fractions afforded the indole carboxylate IC-76 as a colourless solid in 30% yield (18 mg).

¹H NMR (300 MHz, D₂O) 8.37 (1H, s), 7.27 (1H, dd, J=7.4, 1.0 Hz), 7.12 (1H, d, J=7.2 Hz), 7.00-6.90 (3H, m), 6.71 (1H, dd, J=7.3, 1.0 Hz), 6.24 (1H, s), 5.53 (1H, q, J=6.9 Hz), 4.56 (2H, s), 3.19-3.03 (5H, m), 2.91 (9H, s), 2.18 (2H, t, J=7.1 Hz), 1.84-1.69 (2H, m), 1.63 (3H, d, J=7.1 Hz); LRMS [M+H]⁺ 558.

### IC-77: 7-(1-((5-(2-aminoethyl)oxazol-2-yl)oxy)ethyl)-3-(3-fluoro-4-((methylsulfonyl)methyl)phenyl)-1H-indole-2-carboxylic acid

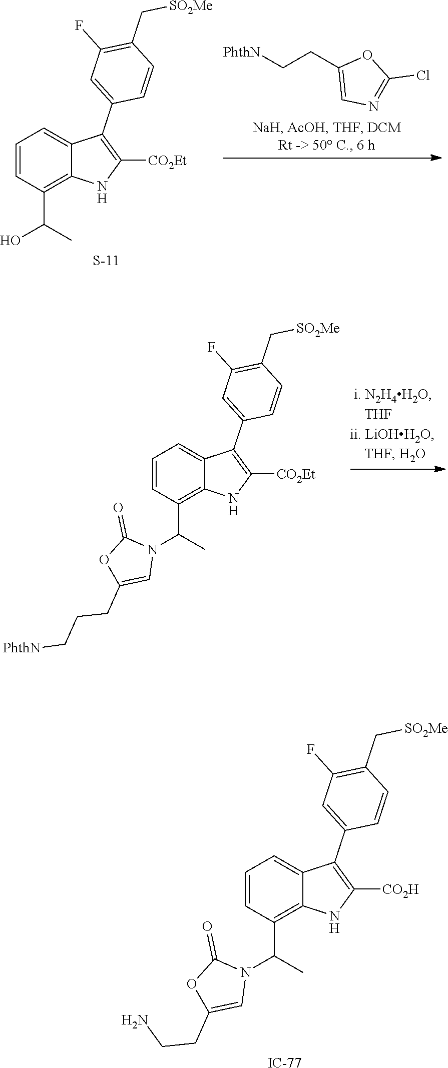

A solution of the alcohol S11 (252 mg, 0.60 mmol, 4 mL THF) was added dropwise at 0° C. to a suspension of NaH (160 mg, 4.0 mmol, 60% in mineral oil) in THF (0.5 mL) under argon atmosphere. The reaction mixture was warmed to RT and stirred for 20 min and then a solution 2-[2-(2-chlorooxazol-5-yl)ethyl]isoindoline-1,3-dione (138 mg, 0.50 mmol in 1.5 mL THF) was added dropwise and the resulting orange suspension was stirred at 50° C. for 8 h. The reaction mixture was cooled to 0° C. and quenched with AcOH (0.40 mL, 7.0 mmol) then diluted with EtOAc and 0.5 M HCl. The organic layer was washed additionally with 0.5 M HCl, then brine and then dried (Na₂SO₄) and concentrated. The obtained material containing the product with semihydrolyzed phthalimide and the starting alcohol was dissolved in DCM (5 mL) and treated with DMAP (6.1 mg, 0.050 mmol) and EDC·HCl (115 mg, 0.60 mmol) at RT and the reaction mixture was stirred at RT for 15 h. The mixture was diluted with DCM, washed with 0.5 M HCl (×2), H₂O, sat. NaHCO₃, brine, dried (MgSO₄) and filtered through a short silica plug eluting with DCM/EtOAc (1:1). Purification by FCC (DCM:EtOAc 6:1) then RP-FCC (C18 5-90% MeCN in H₂O) afforded the product in 13% yield as a colourless film (42 mg).

To the phthalimide protected amine (42 mg, 0.064 mmol) in THF (2 mL) was added hydrazine monohydrate (62 μL, 1.27 mmol) and the reaction was stirred at 60° C. for 5 h and then partitioned between CH₂Cl₂ and H₂O. The organic layer was washed additionally with H₂O (×2), brine, dried (Na₂SO₄) and concentrated in vacuo to afford the amino ester. To the amino ester in THF (0.4 mL) was added a solution of LiOH·H₂O (16 mg, 0.38 mmol in 0.4 mL H₂O) at 0° C. and the reaction mixture was gradually warmed to 19° C. over 21 h. The reaction was quenched at 19° C. by the dropwise addition of AcOH (0.026 mL, 0.46 mmol) and the volatiles removed in vacuo. Purification by RP-FCC [C18, 0.5% AcOH water:MeCN 0 to 60%] and then trituration in MeCN of the concentrated product fraction afforded the indole carboxylate IC-77 as a yellowish solid in 60% yield (19 mg).

¹H NMR (300 MHz, DMSO-d6) 11.12-10.30 (bs, 1H), 9.00-7.58 (bs, 2H), 7.56-7.36 (m, 4H), 7.21 (d, J=7.3 Hz, 1H), 7.11 (s, 1H), 7.04 (t, J=7.7 Hz, 1H), 5.79 (q, J=6.9 Hz, 1H), 4.56 (s, 2H), 3.04 (s, 3H), 2.97 (t, J=6.3 Hz, 2H), 2.70 (t, J=6.7 Hz, 2H), 1.72 (d, J=7.0 Hz, 3H), one exchangeable proton not observed; LRMS [M+H]⁺ 502.

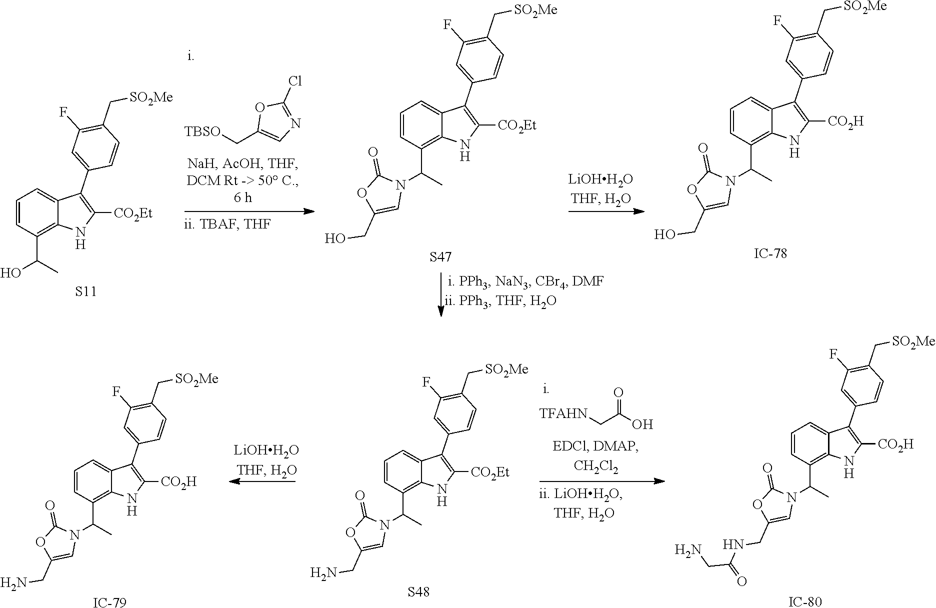

### IC-78: 3-(3-Fluoro-4-((methylsulfonyl)methyl)phenyl)-7-(1-(5-(hydroxymethyl)-2-oxooxazol-3(2H)-yl)ethyl)-1H-indole-2-carboxylic acid

A solution of the alcohol S11 (131 mg, 0.312 mmol, 1.6 mL THF) was added dropwise at 0° C. to a suspension of NaH (100 mg, 2.50 mmol, 60% in mineral oil, washed off oil with THF 3×1 mL) in THF (0.5 mL) under argon atmosphere. The reaction mixture was warmed to RT and stirred for 20 min and then a solution of tert-butyl-[(2-chlorooxazol-5-yl)methoxy]-dimethyl-silane (108 mg, 0.437 mmol in 1.6 mL THF) dropwise. The resulting orange suspension was stirred at 50° C. for 3 h. The reaction mixture was cooled to 0° C. and quenched with AcOH (0.268 mL, 4.68 mmol) then diluted with EtOAc and brine. The organic layer was washed additionally with H₂O, sat NaHCO₃, brine and then dried (Na₂SO₄) and concentrated. Purification by FCC (DCM:EtOAc 15:1 to 10:1) afforded the product as a white solid in 80% yield (158 mg).

To a solution (obtained by heating) of the TBS protected alcohol (515 mg, 0.816 mmol) in THF (5 mL) at 0° C. was added 1 M TBAF in THF (0.90 mL, 0.90 mmol) and the reaction was warmed to RT and stirred for 30 min. The reaction mixture was quenched by the addition of sat. NH₄Cl and extracted with EtOAc; the organic layer was washed with H₂O (×3), brine, dried (Na₂SO₄). Purification by trituration in refluxing EtOAc then filtration, and washing with EtOAc (×3) afforded the alcohol S47 as a white solid in 93% yield (392 mg).

To the ethyl ester S47 (77 mg, 0.149 mmol) in THF (0.7 mL) was added a solution of LiOH·H₂O (37.5 mg, 0.894 mmol in 0.7 mL H₂O) at 0° C. and the reaction mixture was stirred at rt for 42 h. The reaction was quenched at 19° C. by the dropwise addition of AcOH (0.051 mL, 0.89 mmol) and the volatiles removed in vacuo. Purification by RP-FCC [C18, 0.5% AcOH water:MeCN 0 to 70%] afforded the indole carboxylate IC-78 as a greenish solid in 59% yield (43 mg).

¹H NMR (400 MHz, DMSO-d6): 11.82-11.28 (bs, 1H), 7.52 (t, J=7.9 Hz, 1H), 7.46 (dd, J=8.1, 1.0 Hz, 1H), 7.42 (dd, J=11.0, 1.6 Hz, 1H), 7.38 (dd, J=7.8, 1.7 Hz, 1H), 7.29 (s, 1H), 7.23 (d, J=7.2 Hz, 1H), 7.12 (dd, J=8.1, 7.3 Hz, 1H), 5.93 (q, J=7.0 Hz, 1H), 5.34-5.18 (bs, 1H), 4.60 (s, 2H), 4.18 (s, 2H), 3.06 (s, 3H), 1.73 (d, J=7.0 Hz, 3H), CO₂H signal not observed; LRMS [M+NH₄]⁺ 506.

### IC-79: 7-(1-(5-(Aminomethyl)-2-oxooxazol-3(2H)-yl)ethyl)-3-(3-fluoro-4-((methylsulfonyl)methyl)phenyl)-1H-indole-2-carboxylic acid

The alcohol S47 (207 mg, 0.401 mmol), PPh₃ (158 mg, 0.601 mmol), NaN₃ (39 mg, 0.601 mmol) were stirred in DMF (0.80 mL) at rt for 30 min and then CBr₄ (199 mg, 0.601 mmol) was added in one portion and stirring was maintained for 1 h at RT. The reaction was diluted with EtOAc, washed with H₂O (×5), brine (×2), dried (Na₂SO₄) and then concentrated in vacuo. Purification by FCC (DCM:EtOAc 20:1 to 10:1) afforded the azide as a colourless solid in 71% yield (153 mg).

To the azide (295 mg, 0.545 mmol) in THF (4.5 mL) and H₂O (1.5 mL) at 0° C. was added PPh₃ (186 mg, 0.708 mmol) and the reaction was stirred at rt for 17 h. The reaction was diluted with EtOAc, washed with brine (×2), dried (Na₂SO₄) and concentrated. Purification by FCC (EtOAc then DCM:MeOH 50:1 to 10:1) afforded the amine S48 as a white foam in 93% yield (261 mg).

To the amino ester S48 (77 mg, 0.149 mmol) in THF (0.7 mL) was added a solution of LiOH·H₂O (37.6 mg, 0.896 mmol in 0.7 mL H₂O) at 0° C. and the reaction mixture was stirred at rt for 18 h. The reaction was quenched at 19° C. by the dropwise addition of AcOH (0.060 mL, 1.05 mmol) and the volatiles removed in vacuo. Purification by RP-FCC [C18, 0.5% AcOH water:MeCN 0 to 70%] afforded the indole carboxylate IC-79 as a white solid in 78% yield (57 mg).

¹H NMR (300 MHz, DMSO-d6) 11.61-10.68 (bs, 1H), 7.52-7.43 (m, 3H), 7.40 (dd, J=7.8, 1.6 Hz, 1H), 7.26 (s, 1H), 7.22 (d, J=7.2 Hz, 1H), 7.07 (t, J=7.7 Hz, 1H), 5.88 (q, J=6.9 Hz, 1H), 4.58 (s, 2H), 3.70 (s, 2H), 3.05 (s, 3H), 1.73 (d, J=7.0 Hz, 3H), —CO₂H and —NH₂ signals not observed; LRMS [M+NH₄]⁺ 488.

### IC-80: 7-(1-(5-((2-Aminoacetamido)methyl)-2-oxooxazol-3(2H)-yl)ethyl)-3-(3-fluoro-4-((methylsulfonyl)methyl)phenyl)-1H-indole-2-carboxylic acid

To the amine S48 (101 mg, 0.196 mmol), 2-[(2,2,2-trifluoroacetyl)amino]acetic acid (40.2 mg, 0.235 mmol), DMAP (1.20 mg, 0.0098 mmol), EDC (56.3 mg, 0.294 mmol) was added DCM (2 mL) at 0° C. and the reaction was warmed to rt and stirred for 2 h. The reaction was diluted with EtOAc, washed with 0.5 M HCl, H₂O, sat NaHCO₃, brine, then dried (Na₂SO₄) and concentrated in vacuo. Purification by FCC (DCM:EtOAc 1:2) afforded the amide in 93% yield as a pale yellow foam (122 mg).

To the ester (122 mg, 0.182 mmol) in THF (0.9 mL) was added a solution of LiOH·H₂O (45.9 mg, 1.09 mmol in 0.9 mL H₂O) at 0° C. and the reaction mixture was stirred at rt for 18 h. The reaction was quenched at 19° C. by the dropwise addition of AcOH (0.073 mL, 1.28 mmol) and the volatiles removed in vacuo. Purification by RP-FCC [C18, 0.5% AcOH water:MeCN 0 to 60%] then by preparative HPLC [Atlantis, 30×100 mm, 5 μm, 0.1% AcOH water:MeCN=90:10 to 50:50] afforded the indole carboxylate IC-80 as a yellowish solid in 41% yield (41 mg).

¹H NMR (400 MHz, DMSO-d6): 11.02-10.34 (bs, 1H), 8.67 (t, J=5.5 Hz, 1H), 7.51-7.44 (m, 3H), 7.42 (dd, J=7.9, 1.6 Hz, 1H), 7.27 (s, 1H), 7.21 (d, J=7.2 Hz, 1H), 7.06 (t, J=7.7 Hz, 1H), 5.78 (q, J=7.0 Hz, 1H), 4.57 (s, 2H), 4.19-4.05 (m, 2H), 3.42 (s, 2H), 3.05 (s, 3H), 1.74 (d, J=7.0 Hz, 3H), —CO₂H and —NH₂ signals not observed due to broadening; LRMS [M+H]⁺ 545.

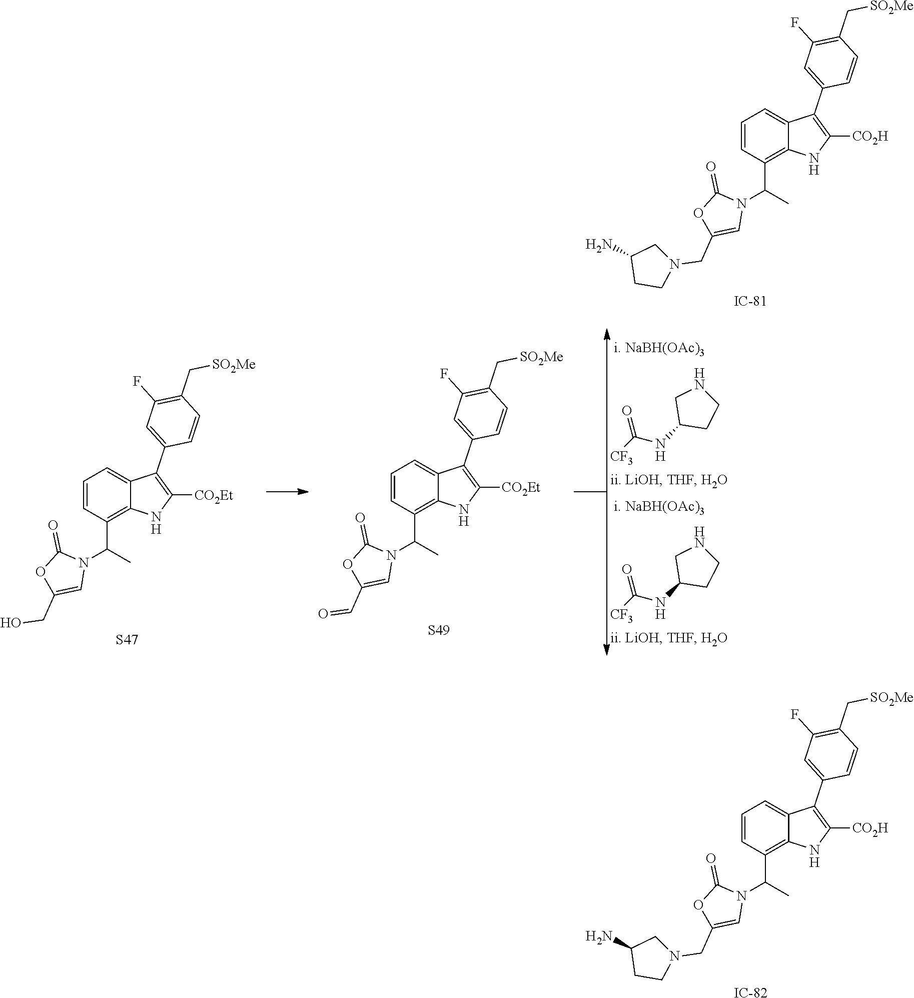

To the alcohol S47 (749 mg, 1.45 mmol) and IBX (487 mg, 1.74 mmol) in a vial was added DMSO (15 mL) and the reaction was stirred at RT for 18 h. The reaction mixture was diluted with H₂O and extracted with EtOAc; the combined organics were washed with NaHCO₃, dried (Na₂SO₄) and then concentrated in vacuo. Purification by FCC (PE:EtOAc 9:1 to 1:4) afforded the aldehyde S49 in 87% yield (648 mg).

### IC-81: 7-[1-[5-[[(3S)-3-Aminopyrrolidin-1-yl]methyl]-2-oxo-oxazol-3-yl]ethyl]-3-[3-fluoro-4-(methylsulfonylmethyl)phenyl]-1H-indole-2-carboxylic acid

To the aldehyde S49 (200 mg, 0.389 mmol), 2,2,2-trifluoro-N-[(3S)-pyrrolidin-3-yl]acetamide hydrochloride (93.5 mg, 0.428 mmol) in THF (20 mL) was added NaB(OAc)₃H (165 mg, 0.777 mmol) and the reaction was stirred at rt for 18 h. The reaction mixture was evaporated under reduced pressure and then dissolved in THF (20 mL) and a solution of LiOH·H₂O (93.2 mg, 3.89 mmol in 3 mL H₂O) was added and the reaction mixture was stirred at rt for 18 h. The reaction was quenched by the dropwise addition of AcOH and the volatiles removed in vacuo. Purification by preparative HPLC [XBridge, 30×100 mm, 5 μm, 0.1% AcOH water:MeCN=95:5 to 5:95] afforded the indole carboxylate IC-81 in 21% yield (46 mg).

¹H NMR (400 MHz, DMSO-d6) δ 10.31-9.96 (1H, m) 9.17-8.06 (2H, m) 7.56-7.49 (2H, m) 7.48-7.41 (2H, m) 7.23-7.18 (1H, m) 7.16-7.03 (2H, m) 5.71-5.60 (1H, m) 4.56 (2H, s) 3.63-3.40 (4H, m) 3.04 (3H, s) 2.83-2.69 (2H, m) 2.42-2.33 (1H, m) 2.14-2.03 (1H, m) 1.76 (3H, d, J=7.0 Hz) 1.71-1.57 (1H, m); LRMS [M+H]⁺ 557.

### IC-82: 7-[1-[5-[[(3R)-3-Aminopyrrolidin-1-yl]methyl]-2-oxo-oxazol-3-yl]ethyl]-3-[3-fluoro-4-(methylsulfonylmethyl)phenyl]-1H-indole-2-carboxylic acid

To the aldehyde S49 (230 mg, 0.447 mmol), 2,2,2-trifluoro-N-[(3R)-pyrrolidin-3-yl]acetamide hydrochloride (107 mg, 0.492 mmol) in THF (20 mL) was added NaB(OAc)₃H (189 mg, 0.894 mmol) and the reaction was stirred at rt for 18 h. The reaction mixture was evaporated under reduced pressure and then dissolved in THF (20 mL) and a solution of LiOH·H₂O (107 mg, 4.47 mmol in 3 mL H₂O) was added and the reaction mixture was stirred at rt for 18 h. The reaction was quenched by the dropwise addition of AcOH and the volatiles removed in vacuo. Purification by preparative HPLC [XBridge, 30×100 mm, 5 μm, 0.1% AcOH water:MeCN=95:5 to 5:95] afforded the indole carboxylate IC-82 in 14% yield (34 mg).

¹H NMR (400 MHz, DMSO-d6; 10.31-9.96 (1H, m) 9.17-8.06 (2H, m) 7.56-7.49 (2H, m) 7.48-7.41 (2H, m) 7.23-7.18 (1H, m) 7.16-7.03 (2H, m) 5.71-5.60 (1H, m) 4.56 (2H, s) 3.63-3.40 (4H, m) 3.04 (3H, s) 2.83-2.69 (2H, m) 2.42-2.33 (1H, m) 2.14-2.03 (1H, m) 1.76 (3H, d, J=7.0 Hz) 1.71-1.57 (1H, m); LRMS [M+H]⁺ 557.

### 7-(1-(5-(3-Aminopropyl)-2-oxooxazolidin-3-yl)ethyl)-3-(3-fluoro-4-((methylsulfonyl)methyl)phenyl)-1H-indole-2-carboxylic acid: separated diastereomers

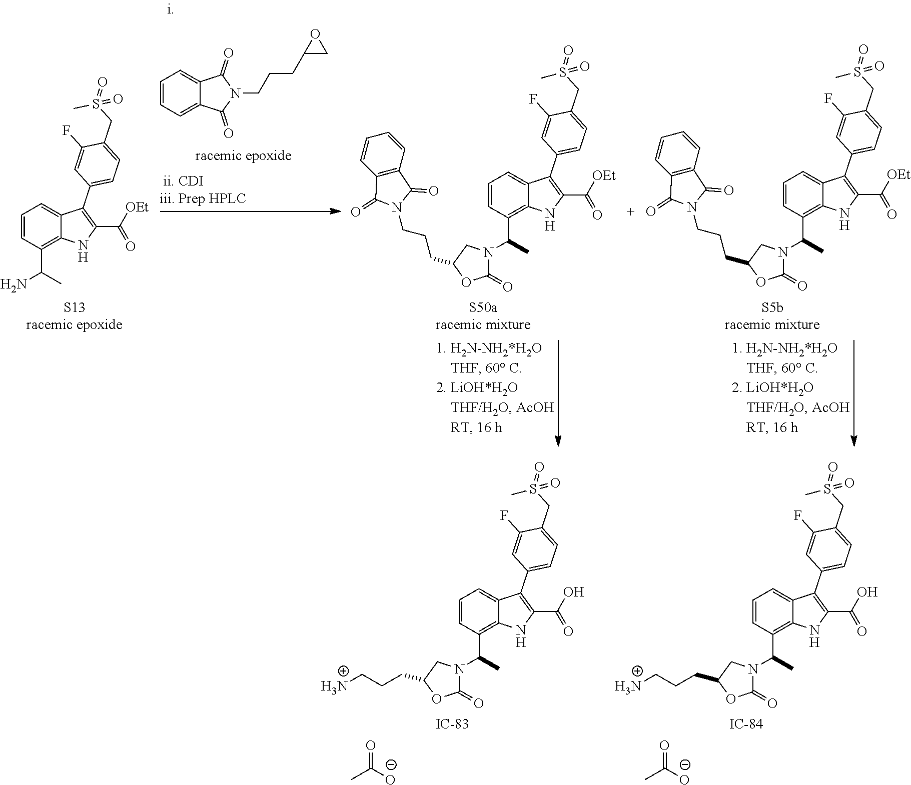

To a white suspension of amine S13 (400 mg, 0.908 mmol) inⁱPrOH (1 mL) was added 2-[3-(oxiran-2-yl)propyl]isoindoline-1,3-dione (140 mg, 0.605 mmol) inⁱPrOH (1 mL) and the resulting suspension heated in a sealed tube at 100° C. for 18 h. The reaction mixture was cooled to RT and the resulting suspension was diluted with EtOAc, water was added; layers separated and the aqueous layer was extracted with EtOAc (×2), the organic layers combined, washed with brine, dried over Na₂SO₄ and concentrated in vacuo. Purification by FCC (DCM:MeOH 50:1 to 20:1) and then by RP-FCC (H₂O:MeCN 90:10 to 5:95, 0.1% TFA) afforded the aminoalcohol as a mixture of diastereomers in 39% yield (393 mg).

To the amino alcohol (276 mg, 0.425 mmol) in MeCN (4 mL) was added CDI (207 mg, 1.27 mmol) at RT and the resulting solution was stirred at 80° C. for 16 h and then concentrated in vacuo. The residue was dissolved in DCM, washed with H₂O (×2), brine, dried (Na₂SO₄) and concentrated in vacuo. Purification by FCC (DCM:MeOH 50:1) afforded the oxazolidinone S50 as a mixture of diastereomers in 57% yield (165 mg). Further purification by prep-HPLC (H₂O:MeCN, 0.1% HCOOH, 90:10 to 5:95) enabled the separation of diastereomers S50a 48 mg and S50b 58 mg.

### IC-83: 7-(1-(5-(3-Aminopropyl)-2-oxooxazolidin-3-yl)ethyl)-3-(3-fluoro-4-((methylsulfonyl)methyl)phenyl)-1H-indole-2-carboxylic acid

To the phthalimide protected amine S50a (73 mg, 0.108 mmol) in THF (3.4 mL) was added hydrazine monohydrate (0.105 mL, 2.16 mmol) and the reaction was stirred at 60° C. for 5 h and then partitioned between CH₂Cl₂ and H₂O. The organic layer was washed additionally with H₂O (×2), brine, dried (Na₂SO₄) and concentrated in vacuo to afford the amino ester. To the amino ester in THF (0.7 mL) was added a solution of LiOH·H₂O (27 mg, 0.648 mmol in 0.7 mL H₂O) at 0° C. and the reaction mixture was gradually warmed to 19° C. over 16 h. The reaction was quenched at 19° C. by the dropwise addition of AcOH (0.043 mL, 0.756 mmol) and the volatiles removed in vacuo. Purification by RP-FCC [C18, 0.5% AcOH water:MeCN 0 to 60%] afforded the indole carboxylate IC-83 in 57% yield (33 mg).

¹H NMR (400 MHz, DMSO-d6) 10.08-9.92 (br s, 1H), 7.60-7.54 (m, 2H), 7.51-7.41 (m, 2H), 7.19 (d, J=7.4 Hz, 1H), 7.11-7.04 (m, 1H), 5.43 (q, J=6.8 Hz, 1H), 4.62-4.51 (m, 3H), 3.60 (t, J=8.8 Hz, 1H), 3.35-3.19 (m, 1H, overlapped with water signal), 3.04 (s, 3H), 2.82-2.63 (m, 2H), 1.90 (s, 3H), 1.70-1.44 (m, 7H); LRMS [M+H]⁺ 518.

### IC-84: 7-(1-(5-(3-Aminopropyl)-2-oxooxazolidin-3-yl)ethyl)-3-(3-fluoro-4-((methylsulfonyl)methyl)phenyl)-1H-indole-2-carboxylic acid

To the phthalimide protected amine S50b (79 mg, 0.117 mmol) in THF (3.4 mL) was added hydrazine monohydrate (0.113 mL, 2.34 mmol) and the reaction was stirred at 60° C. for 5 h and then partitioned between CH₂Cl₂ and H₂O. The organic layer was washed additionally with H₂O (×2), brine, dried (Na₂SO₄) and concentrated in vacuo to afford the amino ester. To the amino ester in THF (0.7 mL) was added a solution of LiOH·H₂O (29 mg, 0.701 mmol in 0.7 mL H₂O) at 0° C. and the reaction mixture was gradually warmed to 19° C. over 16 h. The reaction was quenched at 19° C. by the dropwise addition of AcOH (0.047 mL, 0.818 mmol) and the volatiles removed in vacuo. Purification by RP-FCC [C18, 0.5% AcOH water:MeCN 0 to 60%] afforded the indole carboxylate IC-84 in 57% yield (33 mg).

¹H NMR (400 MHz, DMSO-d6) 10.35-10.05 (br s, 1H), 7.57-7.49 (m, 2H), 7.48-7.41 (m, 2H), 7.14 (d, J=7.2 Hz, 1H), 7.05-6.98 (m, 1H), 5.42 (q, J=7.0 Hz, 1H), 4.56 (s, 2H), 4.42-4.33 (m, 1H), 3.25-3.09 (m, 2H overlapped with water signal), 3.05 (s, 3H), 2.83-2.74 (m, 2H), 1.90 (s, 3H), 1.74-1.52 (m, 7H); LRMS [M+H]⁺ 518.

### IC-85: 7-(((4-(Ammoniomethyl)benzyl)oxy)methyl)-3-(3-fluoro-4-((methylsulfonyl)methyl)phenyl)-1H-indole-2-carboxylate_(Comparative Example)

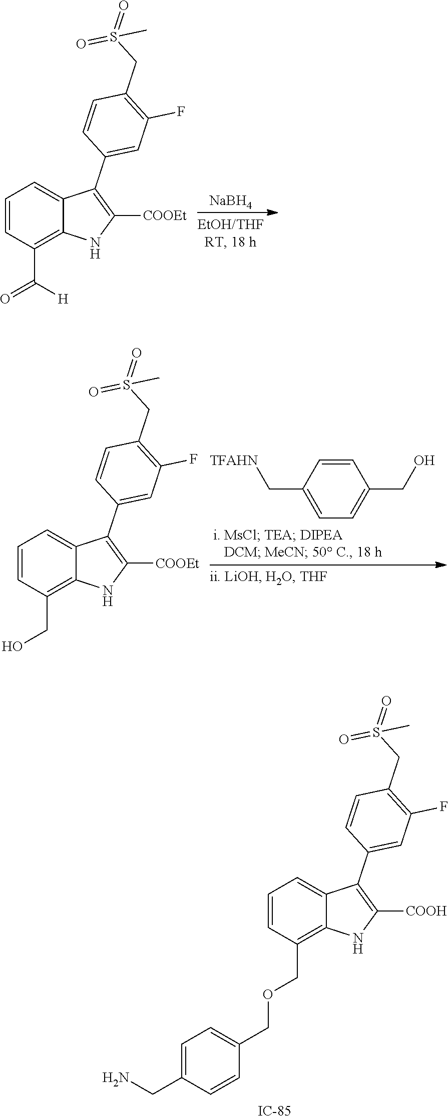

To ethyl 3-[3-fluoro-4-(methylsulfonylmethyl)phenyl]-7-[[4-[[(2,2,2-trifluoro acetyl)amino]methyl]phenyl]methoxymethyl]-1H-indole-2-carboxylate (2 mg, 0.0419 mmol) in THF (0.60 mL) was added LiOH·H₂O (11 mg, 0.251 mmol in 0.6 mL H₂O) at 0° C. and the reaction mixture was gradually warmed to 19° C. over 42 h. The reaction was quenched at 19° C. by the dropwise addition of AcOH and the volatiles removed in vacuo. Purification by the addition of MeCN and H₂O, then by centrifuging the suspension and decanting of the aqueous layer afforded the indole carboxylate IC-85 in quantitative yield (20 mg).

¹H NMR (300 MHz; DMSO-d6) 10.01 (1H, br s), 7.55-7.41 (8H, m), 7.16 (1H, d, J=6.9 Hz), 7.02 (1H, dd, J=7.2, 7.2 Hz), 4.87 (2H, s), 4.59 (2H, s), 4.56 (2H, s), 4.00 (2H, s), 3.04 (3H, s); LRMS [M+H]⁺ 497.

### IC-86: 7-(1-((4-(Aminomethyl)benzyl)oxy)ethyl)-3-(3-cyano-4-(methylsulfonamido)phenyl)-1H-indole-2-carboxylic acid

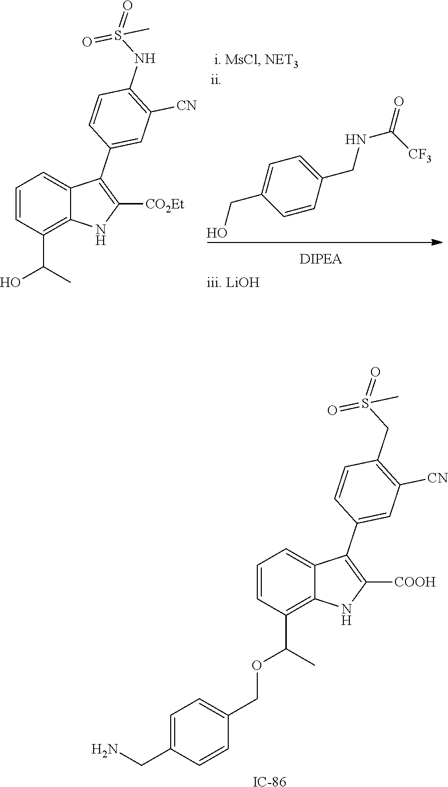

To ethyl 3-[3-cyano-4-(methanesulfonamido)phenyl]-7-(1-hydroxyethyl)-1H-indole-2-carboxylate (400 mg, 0.936 mmol) in DCM at 0° C. (5 mL) was added NEt₃ (0.26 mL, 1.87 mmol) and MsCl (0.145 mL, 1.87 mmol) and stirring was maintained at 0° C. for 30 min and then quenched with H₂O and extracted with DCM; the organics were dried (Na₂SO₄), filtered and concentrated. To the crude mesylate in DCM (4.7 mL) was added DIPEA (0.327 mL, 1.88 mmol) and 2,2,2-trifluoro-N-[[4-(hydroxymethyl)phenyl]methyl]acetamide (438 mg, 1.88 mmol) and the reaction was heated in a sealed vial at 60° C. overnight. The crude reaction mixture was diluted with DCM and H₂O and the aqueous layer extracted with DCM; the combined organics were dried (Na₂SO₄) and concentrated in vacuo. Purification by FCC (DCM: EtOAc gradient) afforded the ether in 33% yield (202 mg).

To the ester (200 mg, 0.311 mmol) in 2:1:1 THF, EtOH, H₂O (4 mL) was added LiOH·H₂O (78.4 mg, 1.87 mmol) and the reaction was stirred overnight at RT. The reaction mixture was neutralised by the addition of 2 M HCl and then concentrated in vacuo. Purification by preparative HPLC [ACE, 30×100 mm, 5 μm, 0.1% HCOOH water:MeCN=98:2 to 2:98] afforded the indole carboxylate IC-86 in 9% yield (15 mg).

¹H NMR (500 MHz, MeOD) δ 7.57 (s, 1H), 7.14 (d, J=2.0, 1H), 7.08 (dd, J=8.4, 2.1, 1H), 6.81 (d, J=8.4, 1H), 6.73-6.69 (m, 1H), 6.60-6.52 (m, 5H), 6.39 (d, J=7.0, 1H), 6.34-6.28 (m, 1H), 3.70 (d, J=11.7, 1H), 3.62 (d, J=11.7, 1H), 3.27 (‘s’, 2H), 2.33 (s, 3H), 0.83 (d, J=6.5, 3H); HRMS (TOF, ESI⁺) m/z: [M+H]⁺ Calcd for C₂₇H₂₇N₄O₅S 519.16967; Found 519.16933.

### 7-(1-((4-(Aminomethyl)benzyl)oxy)ethyl)-3-(3-chloro-4-((methylsulfonyl)methyl)phenyl)-1H-indole-2-carboxylic acid

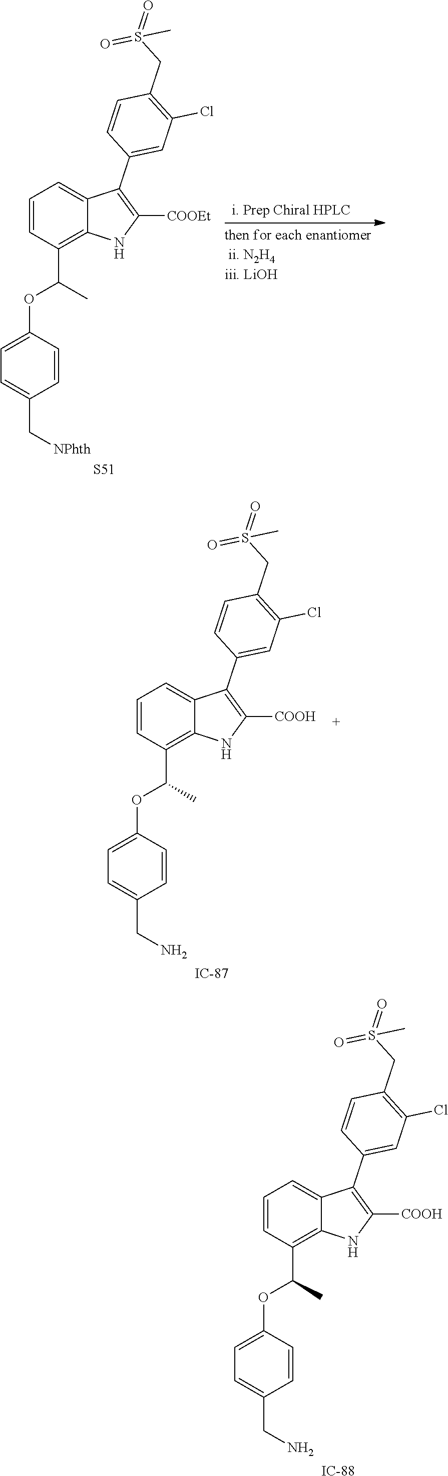

The racemic ether S51 was separated by preparative chiral HPLC [Chiralpak IG column, 30×250 mm, 5 μm, Heptane:DCM 30:70 to 20:80].
Fraction 1: S51a 5 mg
Fraction 2: S51 b 6.20 mg

### IC-87: (S)-7-(1-((4-(Aminomethyl)benzyl)oxy)ethyl)-3-(3-chloro-4-((methylsulfonyl)methyl)phenyl)-1H-indole-2-carboxylic acid

To the phthalimide protected amine S51a (5 mg, 0.0072 mmol) in THF (1 mL) was added methylhydrazine (2 μL, 0.34 mmol) and the reaction was stirred at rt for 4 days with daily addition of methylhydrazine (2 μL, 0.34 mmol) until full conversion. The reaction was then concentrated in vacuo. To the crude amine in THE (2 mL) was added a solution of LiOH·H₂O (5.4 mg, 0.128 mmol in 0.128 mL H₂O) at RT and the reaction mixture was stirred for 14 h. The reaction was quenched by the dropwise addition of AcOH and the volatiles removed in vacuo. Purification by preparative HPLC [XBridge, 10×150 mm, 5 μm, 0.02% AcOH water:MeCN=90:10 to 20:80] afforded the indole carboxylate IC-87 in 54% yield (2.46 mg).

¹H NMR (300 MHz, Methanol-d4): 7.74 (1H, s), 7.60 (2H, d, J=0.8 Hz), 7.52 (1H, dd, J=8.0, 1.2 Hz), 7.39-7.26 (4H, m), 7.16 (1H, dd, J=7.1, 0.8 Hz), 7.08 (1H, dd, J=8.0, 7.2 Hz), 4.95 (1H, q, J=6.5 Hz), 4.66 (2H, s), 4.47 (2H, dd, J=35.1, 11.7 Hz), 4.06 (2H, s), 2.97 (3H, s), 1.65 (3H, d, J=6.5 Hz); LRMS [M+H]⁺ 527 and 529.

### IC-88: (R)-7-(1-((4-(Aminomethyl)benzyl)oxy)ethyl)-3-(3-chloro-4-((methylsulfonyl)methyl)phenyl)-1H-indole-2-carboxylic acid

To the phthalimide protected amine S51b (6 mg, 0.0088 mmol) in THF (1 mL) was added methylhydrazine (2 μL, 0.343 mmol) and the reaction was stirred at rt for 6 days with daily addition of methylhydrazine (2 μL, 0.343 mmol) until full conversion. The reaction was then concentrated in vacuo. To the crude amine in THF (2 mL) was added a solution of LiOH·H₂O (5.4 mg, 0.128 mmol in 0.128 mL H₂O) at RT and the reaction mixture was stirred for 16 h. The reaction was quenched by the dropwise addition of 1 M HCl and the volatiles removed in vacuo. Purification by preparative HPLC [XBridge, 30×150 mm, 5 μm, 0.01% TFA water:MeCN=90:10 to 10:90] and then by preparative HPLC [Atlantis T3, 30×150 mm, 5 μm, 0.01% AcOH water:MeCN=95:5 to 10:90] afforded the indole carboxylate IC-88 in 13% yield (0.64 mg).

¹H NMR (400 MHz, Methanol-d4): 7.74 (1H, s), 7.60 (2H, s), 7.52 (1H, dd, J=8.0, 1.0 Hz), 7.38-7.28 (4H, m), 7.16 (1H, d, J=7.0 Hz), 7.09 (1H, dd, J=8.0, 7.2 Hz), 4.96 (1H, q, J=6.6 Hz), 4.66 (2H, s), 4.48 (2H, dd, J=47.7, 11.6 Hz), 4.06 (2H, s), 2.97 (3H, s), 1.97 (3H, s), 1.65 (3H, d, J=6.5 Hz); LRMS [M+H]⁺ 527 and 529.

### IC-89: 3-(3-Fluoro-4-((methylsulfonyl)methyl)phenyl)-7-(1-(piperidin-4-ylmethoxy)ethyl)-1H-indole-2-carboxylic acid

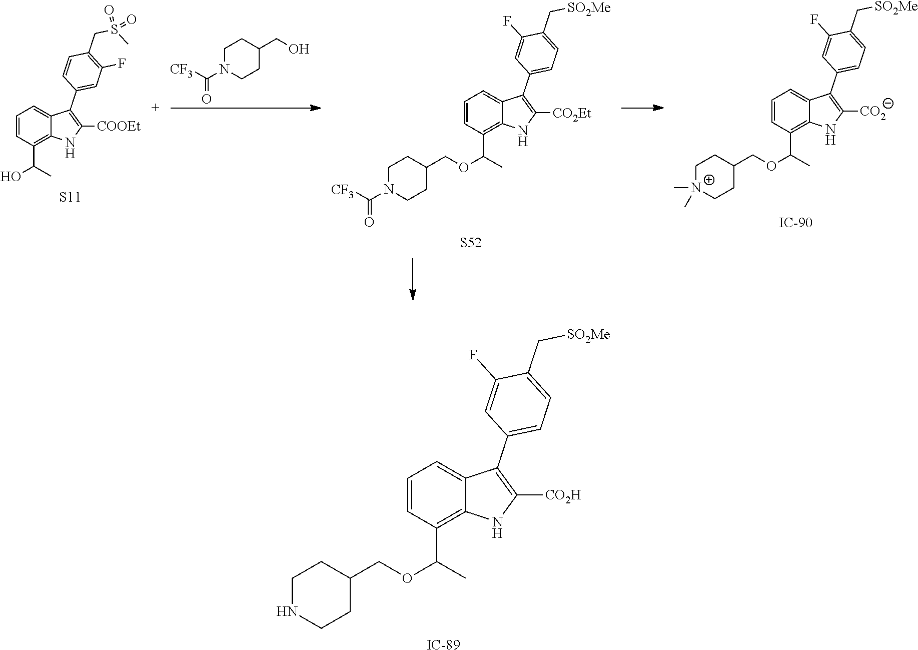

To a solution of the alcohol S11 (210 mg, 0.501 mmol) and DIPEA (113 μL, 0.651 mmol) in DCM (2 mL) at 0° C. was added dropwise MsCl (50 μL, 0.651 mmol) and stirring was maintained for 30 mins. To the reaction mixture was added a solution of 2,2,2-trifluoro-1-[4-(hydroxymethyl)-1-piperidyl]ethenone (529 mg, 2.50 mmol) in MeCN (0.50 mL) then DIPEA (87 μL, 0.501 mmol) and the reaction was heated at 50° C. for 6 h. The reaction mixture was cooled to RT and diluted with DCM, then washed with H₂O (×2), brine, dried (Na₂SO₄) and concentrated. Purification by FCC (DCM:EtOAc 10:1) afforded the ether S52 as a white foam in 61% yield (187 mg).

To the amino ester S52 (69 mg, 0.113 mmol) in THF (0.6 mL) was added a solution of LiOH·H₂O (28 mg, 0.676 mmol in 0.6 mL H₂O) at 0° C. and the reaction mixture was gradually warmed to 19° C. over 18 h. The reaction was quenched at 19° C. by the dropwise addition of AcOH (0.039 mL, 0.676 mmol) and the volatiles removed in vacuo. Purification by RP-FCC [C18, 0.5% AcOH water:MeCN 0 to 80%] afforded the indole carboxylate IC-89 as a yellowish solid in 84% yield (46 mg).

¹H NMR (400 MHz, Methanol-d4) 7.50 (dd, J=7.9, 1.2 Hz, 1H), 7.48-7.44 (m, 1H), 7.44-7.40 (m, 2H), 7.10 (ddd, J=7.2, 1.2, 0.5 Hz, 1H), 7.04 (dd, J=7.9, 7.1 Hz, 1H), 4.81 (q, J=6.6 Hz, 1H, partially overlaps with H2O signal), 4.50 (s, 2H), 3.43-3.33 (m, 3H, partially overlaps with residual CD₃OD signal), 3.22 (dd, J=9.1, 6.1 Hz, 1H), 3.01-2.89 (m, 5H), 2.07-1.85 (m, 3H, overlaps with residual MeCN and AcOH signals), 1.60 (d, J=6.6 Hz, 3H), 1.58-1.42 (m, 2H), three exchangeable protons not observed; LRMS [M+H]⁺ 489.

### IC-90: 7-(1-((1,1-Dimethylpiperidin-1-ium-4-yl)methoxy)ethyl)-3-(3-fluoro-4-((methylsulfonyl)methyl)phenyl)-1H-indole-2-carboxylate

To the ether S52 (183 mg, 0.299 mmol) in EtOH (3 mL) at RT was added K₂CO₃ (82.6 mg, 0.597 mmol) and the reaction was stirred at RT for 22 h and then at 40° C. for 3 h. The reaction was evaporated, suspended in DCM and filtered through Celite® then concentrated.

The amino ester was dissolved in DCM (3 mL) and NaBH(OAc)₃ (317 mg, 1.49 mmol) was added followed by 37% aq formaldehyde (67 μL, 0.896 mmol) at 0° C. and stirring maintained at this temperature for 1 h. The reaction was quenched by the addition of sat. NaHCO₃, extracted with DCM, washed with brine, dried (Na₂SO₄) and concentrated. Purification by FCC (DCM:MeOH 50:1 to DCM:MeOH aq NH₃ 150:15:1) to afford the tertiary amine as a colourless oil in 86% yield (136 mg).

To the tertiary amine (104 mg, 0.185 mmol) and DIPEA (35 μL, 0.203 mmol) in MeCN (2 mL) was added Mel (23 μL, 0.369 mmol) at 0° C. and the reaction was stirred at 0° C. for 1 h and then 2 h at RT and then concentrated in vacuo. Purification by RP-FCC [C18, 0.5% AcOH water:MeCN 0 to 70%] then by preparative HPLC [Atlantis, 30×100 mm, 5 μm, 0.05% AcOH water:MeCN=90:10 to 60:40] afforded the indole carboxylate IC-90 as a white powder in 57% yield (54 mg).

¹H NMR (300 MHz, D₂O): 7.31-7.19 (m, 2H), 7.15-7.03 (m, 2H), 6.85 (t, J=7.6 Hz, 1H), 6.64 (d, J=7.1 Hz, 1H), 4.50-4.31 (m, 3H), 3.30-3.15 (m, 2H), 3.13-2.89 (m, 9H), 2.82 (dd, J=9.0, 7.0 Hz, 1H), 2.73 (s, 3H), 1.70-1.44 (m, 3H), 1.39-1.08 (m, 5H), one exchangeable proton not observed; LRMS [M+H]⁺ 517.

### IC-91: 7-(1-((1H-1,2,3-triazol-5-yl)methoxy)ethyl)-3-(3-fluoro-4-((methylsulfonyl)methyl)phenyl)-1H-indole-2-carboxylic acid

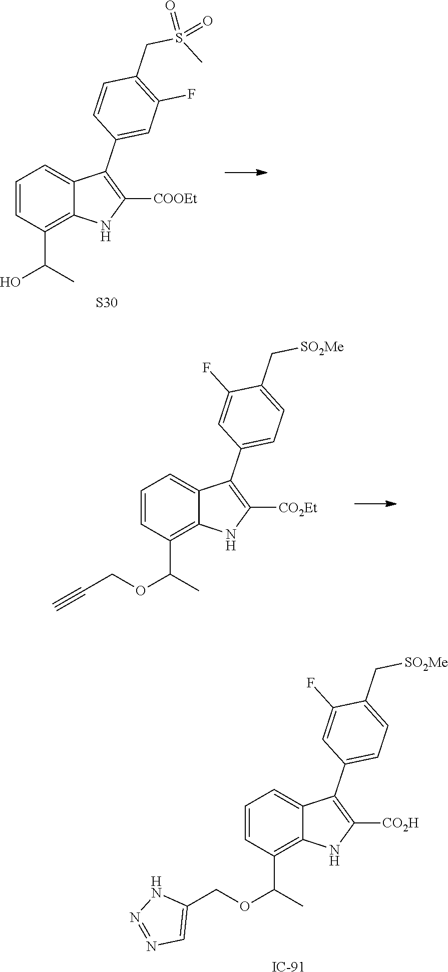 [SMILES: CCOC(=O)c1[nH]c2c(C(C)O)cccc2c1-c1ccc(CS(C)(=O)=O)c(F)c1 ; "S30" ; C#CCOC(C)c1cccc2c(-c3ccc(CS(C)(=O)=O)c(F)c3)c(C(=O)OCC)[nH]c12 ; CC(OCc1cnn[nH]1)c1cccc2c(-c3ccc(CS(C)(=O)=O)c(F)c3)c(C(=O)O)[nH]c12 ; "IC-91"]

To a solution of the alcohol S11 (1686 mg, 0.401 mmol) and DIPEA (83 μL, 0.481 mmol) in DCM (2 mL) at 0° C. was added dropwise MsCl (37 μL, 0.481 mmol) and stirring was maintained for 30 mins. To the reaction mixture was added sequentially propargyl alcohol (0.233 mL, 4.01 mmol) in MeCN (0.50 mL) then DIPEA (70 μL, 0.401 mmol) and the reaction was heated at 50° C. for 2 h. The reaction mixture was cooled to RT and diluted with DCM, then washed with 1 M HCl, H₂O, brine, dried (Na₂SO₄) and concentrated. Purification by FCC (DCM:EtOAc 30:1) afforded the ether as a colourless film in 78% yield (157 mg).

To the alkyne (157 mg, 0.312 mmol) and CuI (5.95 mg, 0.031 mmol) in degassed DMF EtOH (9:1, 0.6 mL) under argon atmosphere was added TMSN₃ (62 μL, 0.468 mmol) and the reaction was heated at 100° C. for 1.5 h. The reaction mixture was cooled, diluted with EtOAc and filtered through a pad of silica and concentrated. Purification by FCC [DCM: MeCN 3:1] afforded the triazole as a colourless film in 64% yield (122 mg, 82% purity).

To the amino ester (122 mg, 0.200 mmol, 82% purity) in THF (1.0 mL) was added a solution of LiOH·H₂O (50 mg, 1.20 mmol in 1.0 mL H₂O) at 0° C. and the reaction mixture was gradually warmed to 15° C. over 18 h. The reaction was quenched at 19° C. by the dropwise addition of AcOH (0.069 mL, 1.20 mmol) and the volatiles removed in vacuo. Purification by RP-FCC [C18, 0.5% AcOH water:MeCN 0 to 80%] afforded the indole carboxylate IC-91 as a white solid in 72% yield (68 mg).

¹H NMR (300 MHz, DMSO-d6): 15.55-14.45 (bs, 1H), 13.46-12.75 (bs, 1H), 8.20-7.62 (bs, 1H) 11.48 (s, 1H), 7.54 (t, J=8.0 Hz, 1H), 7.48-7.35 (m, 4H), 7.17 (dd, J=8.1, 7.2 Hz, 1H), 5.38 (q, J=6.2 Hz, 1H), 4.61 (s, 2H), 4.50 (s, 2H), 3.07 (s, 3H), 1.45 (d, J=6.3 Hz, 3H); LRMS [M+H]⁺ 473.

### IC-92: 7-(1-(4-(Ammoniomethyl)phenoxy)ethyl)-3-(3-fluoro-4-((methylsulfonyl)methyl)phenyl)-1H-indole-2-carboxylate

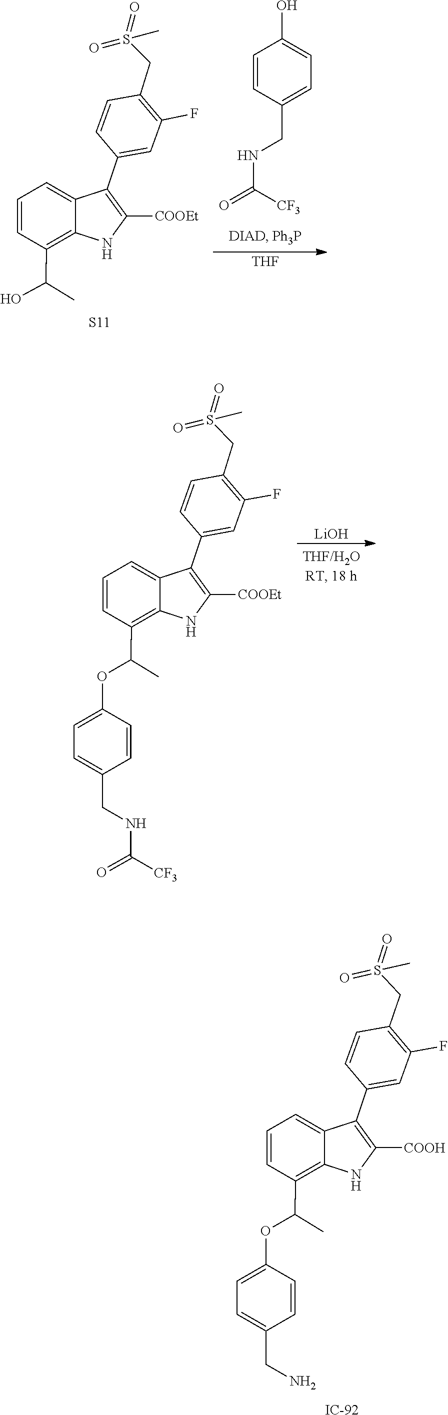

To a solution of the alcohol S11 (500 mg, 1.19 mmol), PPh₃ (625 mg, 2.38 mmol) and 2,2,2-trifluoro-N-[(4-hydroxyphenyl)methyl]acetamide (287 mg, 1.31 mmol) in THF (1 mL) at 0° C. was added DIAD (0.516 mL, 2.62 mmol) dropwise. The reaction was stirred at RT for 2 h and was quenched by the addition of H₂O and EtOAc; the aqueous layer was extracted with EtOAc (3×5 mL); the combined organics were washed with brine, dried (Na₂SO₄) and concentrated. Purification by RP-FCC (H₂O:MeCN 90:10 to 5:95) afforded the ether as a yellow film. To the ether in THF (2 mL) was added a solution of LiOH·H₂O (285 mg, 11.9 mmol in 2.0 mL H₂O) at 0° C. and the reaction mixture was gradually warmed to RT over 18 h. The reaction was quenched at 19° C. by the dropwise addition of AcOH (0.68 mL, 12 mmol) and the volatiles removed in vacuo. Purification by trituration in MeOH afforded the indole carboxylate IC-92 as a white solid in 7% yield over 2 steps (42 mg).

¹H NMR (400 MHz, Acetic acid-d4): 7.60 (1H, dd, J=7.8, 7.8 Hz), 7.53 (1H, dd, J=8.0 Hz), 7.48-7.40 (3H, m), 7.34-7.26 (2H, m), 7.17 (1H, dd, J=7.6 Hz), 7.07-7.00 (2H, m), 6.00 (1H, q, J=6.2 Hz), 4.54 (2H, s), 4.09 (2H, s), 3.00 (3H, s), 1.81 (3H, d, J=6.2 Hz); LRMS [M+H]⁺ 497.

### IC-93: 7-(1-((5-(3-Aminopropyl)oxazol-2-yl)amino)ethyl)-3-(3-fluoro-4-((methylsulfonyl)methyl)phenyl)-1H-indole-2-carboxylic acid

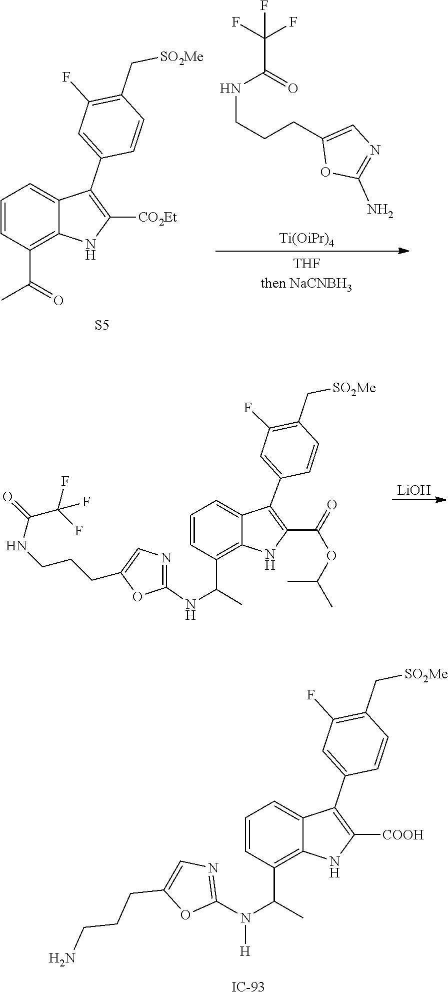

To ketone S5 (200 mg, 0.479 mmol) and N-[3-(2-aminooxazol-5-yl)propyl]-2,2,2-trifluoro-acetamide (455 mg, 0.958 mmol) was added THF (1 mL) and Ti(OiPr)₄ (0.567 mL, 1.92 mmol) and the reaction mixture was heated at 100° C. for 18 h. The reaction mixture was cooled and diluted with brine and EtOAc (1:1); the precipitates were filtered; the aqueous layer was extracted with EtOAc; the combined organics were dried (Na₂SO₄) and evaporated. The residue was dissolved in MeOH (10 mL) and AcOH (0.5 mL) was added followed by NaBH₃CN (301 mg, 4.79 mmol) and stirring was maintained for 2 h at RT. The reaction was concentrated and then purified by preparative HPLC [XBridge, 30×100 mm, 5 μm, 0.02% AcOH, water:MeCN=95:5 to 5:95] to afford the amine in 11% yield (44 mg).

To the ether (44 mg, 0.0506 mmol) in THF (2 mL) and EtOH (1 mL) was added a solution of LiOH·H₂O (12.1 mg, 0.506 mmol in 2.0 mL H₂O) at 0° C. and the reaction mixture was stirred at RT for 18 h. The reaction was acidified by the dropwise addition of AcOH to pH 4-5 and the volatiles removed in vacuo. Purification by preparative HPLC [XBridge, 30×100 mm, 5 μm, 0.02% AcOH, water:MeCN=95:5 to 5:95] afforded the indole carboxylate IC-93 in 77% yield (20 mg).

¹H NMR (300 MHz, DMSO-d6+1 drop of acetic acid-d4): 7.52-7.44 (2H, m) 7.43-7.37 (3H, m) 7.28-7.23 (1H, m) 7.07-7.01 (1H, m) 6.34 (1H, s) 5.39-5.27 (1H, m) 4.57 (2H, s) 3.04 (3H, s) 2.80-2.70 (2H, m) 1.90-1.85 (2H, m) 1.79-1.68 (2H, m) 1.55 (3H, d, J=6.8 Hz); LRMS [M+H]⁺ 515.

### IC-94: 7-(1-((5-(2-aminoethyl)oxazol-2-yl)amino)ethyl)-3-(3-fluoro-4-((methylsulfonyl)methyl)phenyl)-1H-indole-2-carboxylic acid

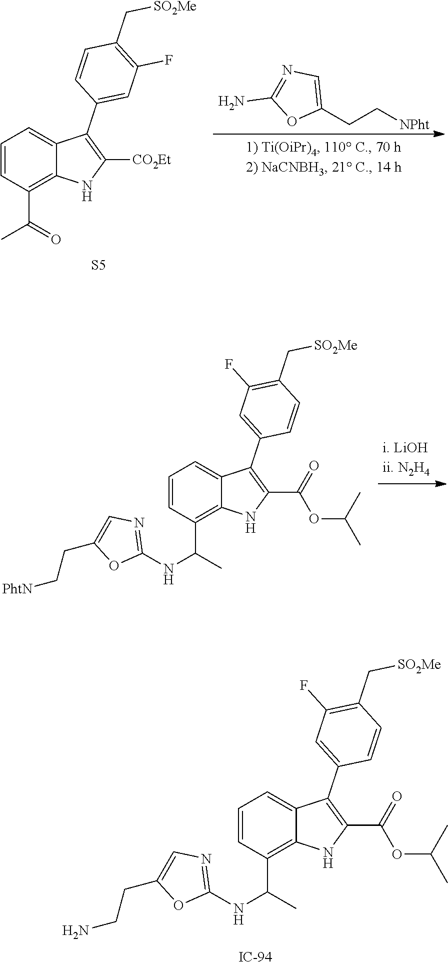

To ketone S5 (120 mg, 0.282 mmol) and 2-(2-(2-aminooxazol-5-yl)ethyl)isoindoline-1,3-dione (163 mg, 0.620 mmol) was added THF (1 mL) and Ti(OⁱPr)₄ (0.334 mL, 1.13 mmol) and the reaction mixture was heated at 110° C. for 70 h. The reaction mixture was cooled to RT and NaBH₃CN (89 mg, 1.41 mmol) and AcOH (0.3 mL) were added. After 3 h, AcOH (0.3 mL) and MeOH (0.2 mL) were added and stirring maintained for 14 h. The reaction was quenched with brine (30 mL), extracted with EtOAc; the combined organics washed with NaHCO₃ aq, brine, dried (Na₂SO₄) and concentrated. Purification by FCC (DCM:EtOH 99:1 to 90:10 then by preparative HPLC [XBridge, 30×100 mm, 5 μm, 0.02% AcOH, water:MeCN=90:10 to 5:95] afforded the amine as a beige solid in 45% yield (87 mg).

To the ether (87 mg, 0.127 mmol) in THF (3 mL) was added a solution of LiOH·H₂O (80 mg, 1.90 mmol in 2.0 mL H₂O) at 0° C. and the reaction mixture was stirred at RT for 48 h. The reaction was acidified by the dropwise addition of AcOH to pH 4-5 and the volatiles removed in vacuo to afford the acid in 72% yield (60 mg). To the protected amine (60 mg, 0.0906 mmol) in THF (2 mL) was added hydrazine monohydrate (18 μL, 0.363 mmol) and the reaction was stirred at rt for 14 h then at 80° C. for 14 h. The reaction was quenched with AcOH (2 eq) and concentrated. Purification by preparative HPLC [XBridge, 30×100 mm, 5 μm, 0.02% AcOH, water:MeCN=90:10 to 10:90] afforded the indole carboxylate IC-94 in 37% yield (17 mg).

¹H NMR (300 MHz, DMSO-d6+1 drop of acetic acid-d4): 7.50 (1H, t, J=8.1 Hz), 7.44-7.35 (3H, m), 7.28 (1H, d, J=7.1 Hz), 7.05 (1H, dd, J=7.3, 0.7 Hz), 6.46 (1H, s), 5.36 (1H, q, J=6.9 Hz), 4.58 (2H, s), 3.05 (3H, s), 2.95-2.90 (2H, m), 2.80-2.75 (2H, m), 1.56 (3H, d, J=6.9 Hz); LRMS [M+H]⁺ 501.

### IC-95: 7-(1-((4-(Aminomethyl)piperidine-1-carbonyl)oxy)ethyl)-3-(3-fluoro-4-((methylsulfonyl)methyl)phenyl)-1H-indole-2-carboxylic acid

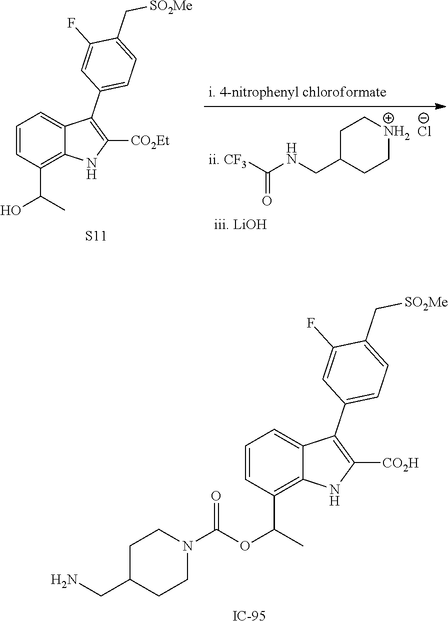

To a solution of the alcohol S11 (159 mg, 0.379 mmol) and pyridine (0.037 mL, 0.455 mmol) in DCM (4 mL) at −15° C. was added portionwise 4-nitrophenyl chloroformate (84 mg, 0.417 mmol) and the reaction was warmed to RT and stirred 30 min. The reaction was diluted with DCM, washed with 0.5 M HCl, H₂O, brine, dried (Na₂SO₄) then concentrated. Purification by precipitation (Et₂O, DCM) and filtration afforded the carbonate in 92% yield (203 mg). To the carbonate (241 mg, 0.412 mmol) and 2,2,2-trifluoro-N-(piperidin-1-ium-4-ylmethyl)acetamide chloride (122 mg, 0.494 mmol) in DCM (4 mL) was added dropwise NEt₃ (75 μL, 0.536 mmol) at 0° C. and the reaction was stirred at RT for 16 h. The reaction was diluted with DCM, washed with 5% aqueous citric acid, H₂O, sat. NaHCO₃ (×2), brine, dried (Na₂SO₄) and concentrated. Purification by FCC (DCM:MeCN 10:1 to 5:1) afforded the carbonate as a white solid in 92% yield (249 mg). To the ester (213 mg, 0.325 mmol) in THF (1.6 mL) was added a solution of LiOH·H₂O (82 mg, 1.95 mmol in 1.6 mL H₂O) at 0° C. and the reaction mixture was gradually warmed to 14° C. over 20 h. The reaction was quenched at 19° C. by the dropwise addition of AcOH (0.13 mL, 7 eq) and the volatiles removed in vacuo.

Purification by RP-FCC (H₂O MeCN, 0.1% TFA 0-60%) then by preparative HPLC [XBridge, 30×100 mm, 5 μm, 0.03% AcOH, water:MeCN=90:10 to 40:60] afforded the indole carboxylate IC-95 in 56% yield (97 mg).

¹H NMR (300 MHz, DMSO-d6):11.11-9.66 (bs, 1H), 9.21-7.70 (bs, 2H), 7.60-7.40 (m, 4H), 7.15 (d, J=7.1 Hz, 1H), 7.02 (t, J=7.6 Hz, 1H), 6.26 (q, J=6.4 Hz, 1H), 4.56 (s, 2H), 4.25-3.83 (bs, 2H), 3.04 (s, 3H), 2.95-2.59 (m, 4H), 1.84-1.50 (m, 6H), 1.34-1.00 (m, 2H), one exchangeable proton (probably —CO2H) not observed due to broadening; LRMS [M+H]⁺ 532.

### IC-96: 7-(1-(((4-(Aminomethyl)phenyl)carbamoyl)oxy)ethyl)-3-(3-fluoro-4-((methylsulfonyl)methyl)phenyl)-1H-indole-2-carboxylic acid

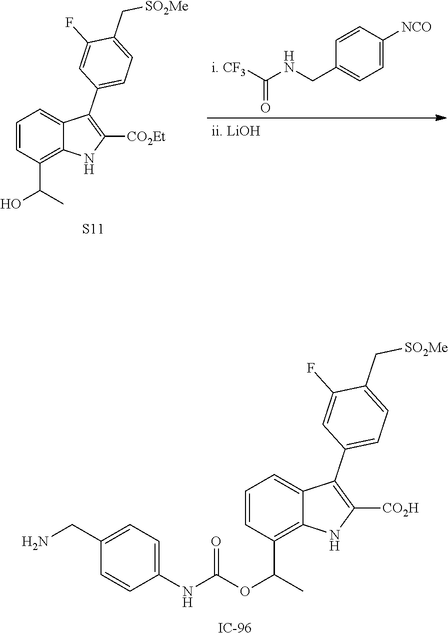

To the alcohol S11 (136 mg, 0.324 mmol), 2,2,2-trifluoro-N-[(4-isocyanatophenyl)methyl]acetamide (119 mg, 0.486 mmol) and DMAP (4 mg, 0.032 mmol) was added DCM (3.2 mL) at 0° C. and the reaction was warmed to RT and stirred 15 h. The reaction was quenched with H₂O, stirred vigorously and extracted with DCM, washed with brine, dried (Na₂SO₄) and concentrated. Purification by FCC (DCM:MeCN 10:1 to 7:1) afforded the carbonate as a white solid in 97% yield (209 mg).

To the ester (174 mg, 0.262 mmol) in THF (1.3 mL) was added a solution of LiOH·H₂O (66 mg, 1.57 mmol in 1.3 mL H₂O) at 0° C. and the reaction mixture was gradually warmed to 14° C. over 19 h. The reaction was quenched at 19° C. by the dropwise addition of TFA (0.14 mL, 7 eq) and the volatiles removed in vacuo. Purification by RP-FCC (H₂O MeCN, 0.1% TFA 0-60%) and then by RP-FCC (H₂O MeCN, 0.5% AcOH 0-80%) and trituration (MeCN) afforded the indole carboxylate IC-96 in 40% yield (56 mg).

¹H NMR (300 MHz, DMSO-d6): 11.58-10.23 (bs, 1H), 10.15-9.51 (bs, 1H), 7.57-7.30 (m, 8H), 7.23 (d, J=7.2 Hz, 1H), 7.04 (t, J=7.6 Hz, 1H), 6.54-6.44 (m, 1H), 4.55 (s, 2H), 3.91 (s, 2H), 3.04 (s, 3H), 1.61 (d, J=6.5 Hz, 3H), exchangeable —NH₂ and —CO₂H signals not observed due to broadening; ¹⁹F NMR (376 MHz, DMSO-d6) −117.81; LRMS [M+H]⁺ 540.

### IC-97: 7-(1-(4-(Aminomethyl)-N-methylbenzamido)ethyl)-3-(3-fluoro-4-((methylsulfonyl)methyl)phenyl)-1H-indole-2-carboxylic acid

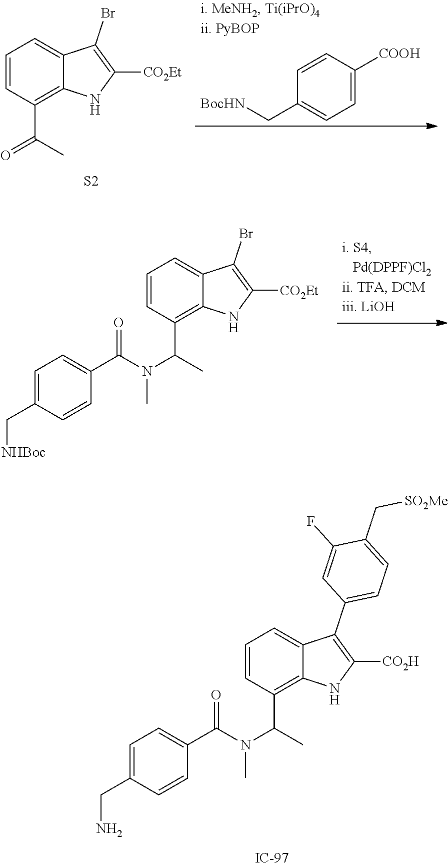

To 8 M MeNH₂ in EtOH (0.725 mL, 5.80 mmol) was added titanium(IV) isopropoxide 0.745 mL, 2.51 mmol) and ketone S2 (600 mg, 1.93 mmol) and the resultant mixture stirred at rt overnight. NaBH₄ (73 mg, 1.93 mmol) was then added and the mixture stirred at rt for 3 h.

The reaction mixture was quenched with water and the resulting precipitate washed with EtOAc; the organic layer was separated and the aqueous layer further extracted with EtOAc (×2); The combined organic extracts were dried over Na₂SO₄, filtered and concentrated in vacuo. Purification by FCC (SNAP-NH 28 g column, cyclohexane:DCM 0-100%) afforded the methyl amine in 87% yield (545 mg).

To 4-[(tert-butoxycarbonylamino)methyl]benzoic acid (185 mg, 0.738 mmol) in DCM (1.8 mL) was added NEt₃ (0.256 mL, 1.85 mmol), the amine (200 mg, 0.615 mmol) and PyBOP (384 mg, 0.738 mmol). The reaction mixture was left to stir at rt for 2 h then diluted with DCM and washed with 1 M HCl; the combined organic fractions were dried over Na₂SO₄, filtered and concentrated in vacuo. Purification by FCC (KP-Sil 25 g column, DCM:EtOAc 0-10%) afforded the tertiary amide in 97% yield (333 mg).

To the bromoindole (270 mg, 0.483 mmol) was added pinacol ester S4 (182 mg, 0.580 mmol), PdDPPFCl₂ (17 mg, 0.0208 mmol) and 1,4-dioxane (8 mL) and 2 M Na₂CO₃ (2 mL); the reaction was degassed and heated at 110° C. for 4 h. The reaction was cooled, filtered through Celite® rinsing with EtOAc and H₂O. the filtrate was partitioned between EtOAc and 1 M HCl, aqueous layer extracted and the combined organics were washed (brine), dried (Na₂SO₄) and concentrated in vacuo. Purification by FCC (KP-Sil 25 g column, DCM:EtOAc 0-20%) afforded the product in 83% yield (267 mg).

To the N-Boc amine (250 mg, 0.376 mmol) in DCM (2.2 mL) at 0° C. was added TFA (0.287 mL, 3.76 mmol) and the reaction was stirred at RT overnight, then concentrated in vacuo. To the residue in THF:EtOH:H₂O 2:1:1 (5 mL) was added LiOH·H₂O (159 mg, 3.80 mmol) and the reaction was stirred overnight at RT then acidified to pH 1 with 2 M HCl and concentrated in vacuo. Purification by RP-FCC (H₂O MeOH, 0-100%, 0.002 M HCl) afforded the indole carboxylate IC-97 in 50% yield (102 mg).

¹H NMR (500 MHz, MeOD) δ 7.71-7.36 (m, 9H), 7.26-7.11 (m, 1H), 6.54 (q, J=7.1, 1H), 4.57 (s, 2H), 4.16 (s, 2H), 3.01 (s, 3H), 2.69 (s, 3H), 1.84 (d, J=7.1, 3H); HRMS (TOF, ESI⁺) m/z: [M+H]⁺ Calcd for C₂₈H₂₉FN₃O₅S 538.18065; Found 538.18044.

### IC-98: 7-[1-(5,6-Dihydroxy-1,3-dioxo-2,3-dihydro-1H-isoindol-2-yl)ethyl]-3-[3-fluoro-4-(methanesulfonylmethyl)phenyl]-1H-indole-2-carboxylic acid

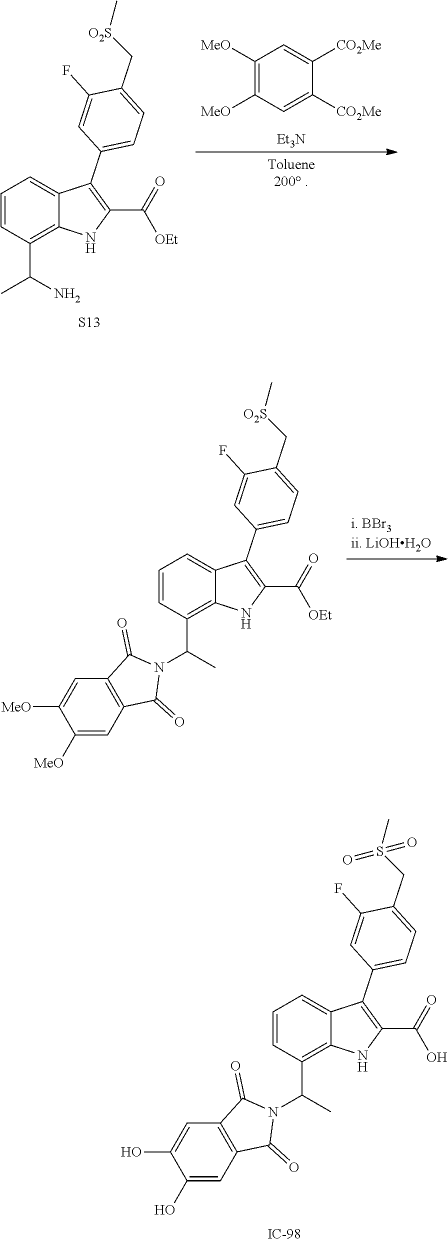

To amine S13 (25 mg, 0.0597 mmol) and dimethyl 4,5-dimethoxybenzene-1,2-dicarboxylate (15.9 mg, 0.0627 mmol) in 1:1 toluene:MeCN (1 mL) was added NEt₃ (50 mol %) and the reaction was heated at 200° C. for 5 h under microwave heating. The reaction was cooled to RT and concentrated; the residue was dissolved in EtOAc and washed with 1 M aq HCl, brine; dried (Na₂SO₄) and concentrated. Purification by FCC (cyclohexane:EtOAc 0-25%) afforded the product in 47% yield (17 mg).

To a stirred solution of the dimethoxyphthalimide derivative (70 mg, 1 eq) in anhydrous CH₂Cl₂ (2 mL) was added boron tribromide (1 M in CH₂Cl₂, 25 eq) under Ar to give a cloudy yellow solution. After completion of the reaction (1 hour; as assessed by TLC), excess BBr₃ was quenched by pouring the reaction mixture into cold aq. NaHCO₃ solution and left to stir vigorously for 1 hour. The aqueous layer was extracted with EtOAc (×3) and the combined organic extracts were dried over Na₂SO₄, filtered and concentrated under vacuo. The crude product was redissolved in THF:EtOH:H₂O (2:1:1) and LiOH·H₂O (48 mg, 10 eq) was added.

The resultant mixture was stirred at room temperature for 24 hours and then acidified to pH 2 with 2 M aq HCl and extracted with EtOAc (×2). The combined organic extracts were dried over Na₂SO₄, filtered, then concentrated in vacuo. Purification by RP-FCC afforded the indole carboxylate IC-98 in 10% yield over 2 steps (10 mg).

¹H NMR (400 MHz, Methanol-d4) δ 8.02 (d, J=7.5 Hz, 1H), 7.80 (d, J=8.0 Hz, 1H), 7.50 (t, J=8.0 Hz, 1H), 7.37-7.27 (m, 3H), 7.22 (t, J=8.0 Hz, 1H), 4.47 (bs, 3H), 2.90 (bs, 3H), 2.64 (s, 3H), 1.57 (d, J=7.0 Hz, 3H).

### IC-99: 7-[1-[[4-(Aminomethyl)cyclohexanecarbonyl]amino]ethyl]-3-[3-fluoro-4-(methylsulfonylmethyl)phenyl]-1H-indole-2-carboxylic acid

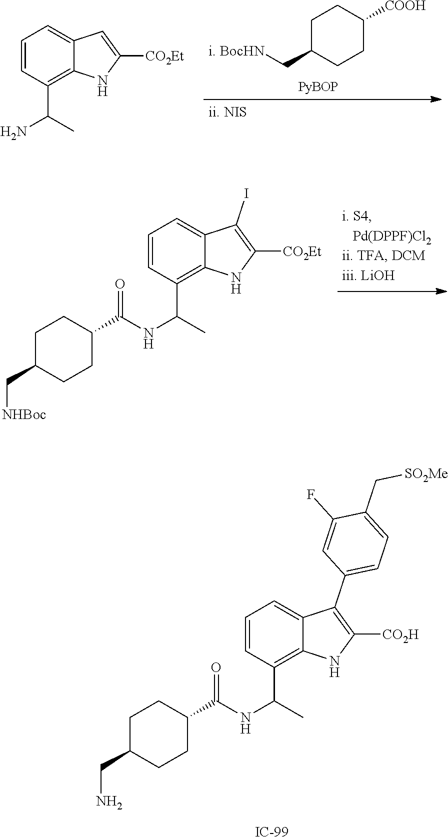

To 4-[(tert-butoxycarbonylamino)methyl]cyclohexanecarboxylic acid (278 mg, 1.08 mmol) in DCM (8.7 mL) was added NEt₃ (0.375 mL, 2.70 mmol), ethyl 7-(1-aminoethyl)-1H-indole-2-carboxylate (209 mg, 0.900 mmol) and PyBOP (562 mg, 1.08 mmol). The reaction mixture was left to stir at rt for 2 h then diluted with DCM and washed with 1 M HCl; the combined organic fractions were dried over Na₂SO₄, filtered and concentrated in vacuo. Purification by FCC (KP-Sil 25 g column, DCM:EtOAc 0-50%) afforded the amide in 99% yield (418 mg).

To the indole (318 mg, 0.674 mmol) in 5:1 DCM:DMF (12 mL) was added NIS (167 mg, 0.742 mmol) and the reaction was stirred at rt for 3 h. Upon completion, the reaction mixture was partitioned between DCM and sat. aq. NaHCO₃, the organics were separated and the aqueous phase further extracted with DCM (×2); the combined DCM extracts were dried over Na₂SO₄, filtered and concentrated in vacuo. Purification by FCC (KP-Sil 25 g column, DCM:EtOAc 0-50%) afforded the iodoindole in 72% yield (290 mg).

To the iodoindole (250 mg, 0.418 mmol) was added pinacol ester S4 (158 mg, 0.502 mmol), PdDPPFCl₂ (15 mg, 0.018 mmol) and 1,4-dioxane (10 mL) and 2 M Na₂CO₃ (2 mL); the reaction was degassed and heated at 110° C. for 1.5 h. The reaction was cooled, filtered through Celite® rinsing with EtOAc and H₂O. the filtrate was partitioned between EtOAc and 1 M HCl, aqueous layer extracted and the combined organics were washed (brine), dried (Na₂SO₄) and concentrated in vacuo. Purification by FCC (KP-Sil 25 g column, DCM:EtOAc 0-100%) afforded the product in 73% yield (201 mg).

To the N-Boc amine (180 mg, 0.274 mmol) in DCM (10 mL) at 0° C. was added TFA (0.21 mL, 2.74 mmol) and the reaction was stirred at RT overnight, then concentrated in vacuo. To the residue in THF:EtOH:H₂O 2:1:1 (4 mL) was added LiOH·H₂O (57 mg, 1.35 mmol) and the reaction was stirred overnight at RT then concentrated in vacuo. Purification by RP-FCC (H₂O MeOH, 0-100%, 0.01% FA) afforded the indole carboxylate IC-99 as a colourless solid in 71% yield over two steps (101 mg).

¹H NMR (600 MHz, MeOD) 5=8.53 (s, 1H), 7.51 (dd, J=8.2, 6.9, 2H), 7.48-7.40 (m, 2H), 7.21 (d, J=7.2, 1H), 7.06 (t, J=7.7, 1H), 5.51 (q, J=7.0, 1H), 4.89-4.72 (m, 1H), 4.52 (s, 2H), 2.97 (s, 3H), 2.81-2.62 (m, 2H), 2.20 (tt, J=12.1, 3.3, 1H), 2.02-1.69 (m, 4H), 1.71-1.44 (m, 5H), 1.07-0.92 (m, 2H); HRMS (TOF, ESI⁺) m/z: [M+H]⁺ Calcd for C₂₇H₃₃FN₃O₅S 530.21195; Found 530.21189.

### IC-100: 7-[1-[[2-(4-Aminocyclohexyl)acetyl]amino]ethyl]-3-[3-fluoro-4-(methylsulfonylmethyl)phenyl]-1H-indole-2-carboxylic acid_

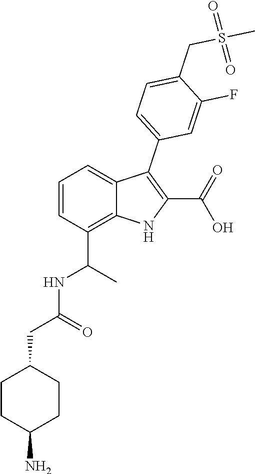 [SMILES: CC(NC(=O)C[C@H]1CC[C@H](N)CC1)c1cccc2c(-c3ccc(CS(C)(=O)=O)c(F)c3)c(C(=O)O)[nH]c12]

Indole carboxylate IC-100 was synthesised analogously to IC-99 using 2-[4-(tert-butoxycarbonylamino)cyclohexyl]acetic acid.

¹H NMR (500 MHz, MeOD) δ=7.51 (q, J=7.9, 2H), 7.45-7.37 (m, 2H), 7.27 (d, J=7.2, 1H), 7.12-7.05 (m, 1H), 5.56 (q, J=7.0, 1H), 4.52 (s, 2H), 2.98 (s, 3H), 2.90 (tt, J=12.0, 4.0, 1H), 2.19-2.06 (m, 2H), 1.95-1.53 (m, 9H), 1.30 (dqd, J=16.3, 12.4, 3.7, 3H), 1.11-0.95 (m, 2H); HRMS (TOF, ESI⁺) m/z: [M+H]⁺ Calcd for C₂₇H₃₃FN₃O₅S 530.21195; Found 530.21211.

### IC-101: 7-(1-(((4-(aminomethyl)phenoxy)carbonyl)amino)ethyl)-3-(3-fluoro-4-((methylsulfonyl)methyl)phenyl)-1H-indole-2-carboxylic acid

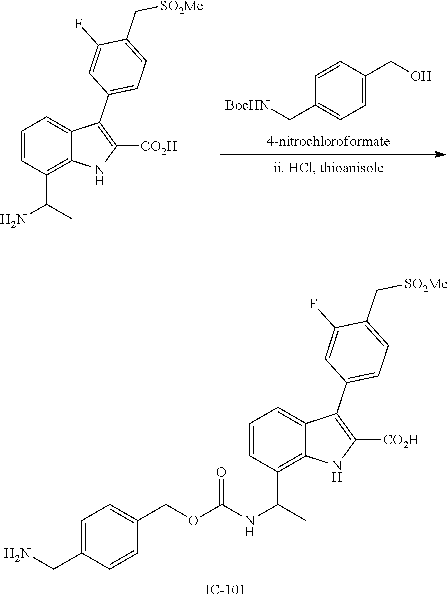 [SMILES: CC(N)c1cccc2c(-c3ccc(CS(C)(=O)=O)c(F)c3)c(C(=O)O)[nH]c12 ; CC(C)(C)OC(=O)NCc1ccc(CO)cc1 ; CC(NC(=O)OCc1ccc(CN)cc1)c1cccc2c(-c3ccc(CS(C)(=O)=O)c(F)c3)c(C(=O)O)[nH]c12]

To tert-butyl (4-hydroxybenzyl)carbamate (223 mg, 1.00 mmol), pyridine (0.121 mL, 1.50 mmol) in DCM (5 mL) at 0° C. was added portionwise 4-nitrophenyl chloroformate (242 mg, 1.20 mmol) and the reaction was warmed to RT and stirred 2 h. The reaction was diluted with EtOAc, washed with 0.5 M HCl, H₂O, brine, dried (MgSO₄) then concentrated. Purification by recrystalisation (hexane:EtOAc) afforded the carbamate in 76% yield (295 mg).

To the carbamate (106 mg, 0.274 mmol), 7-(1-aminoethyl)-3-(3-fluoro-4-((methylsulfonyl)methyl)phenyl)-1H-indole-2-carboxylic acid (107 mg, 0.274 mmol) was added DMF (2.7 mL) and the reaction was stirred at RT for 16 h. The reaction mixture was concentrated in vacuo and then purified by RP-FCC (C18, H₂O MeCN, 0.1% TFA 0-80%) to afford the carbamate in 70% yield (122 mg).

To N-Boc protected amine (119 mg, 0.186 mmol) and thioanisole (0.109 mL, 0.930 mmol) in EtOAc (3 mL) was added 4 M HCl in dioxane (0.70 mL, 2.79 mmol) and the reaction was stirred at rt for 20 h. The reaction was diluted with EtOAc and the volatiles removed in vacuo. Purification by RP-FCC (H₂O MeCN, 0.1% TFA 0-60%) and then counterion exchange with anhydrous HCl afforded the indole carboxylate IC-101 in 79% yield (85 mg).

¹H NMR (300 MHz, DMSO-d6): 13.63-12.55 (bs, 1H), 11.67 (s, 1H), 8.62 (d, J=8.1 Hz, 1H), 8.24 (s, 3H), 7.54 (t, J=8.0 Hz, 1H), 7.49-7.33 (m, 6H), 7.22-7.06 (m, 3H), 5.52-5.34 (m, 1H), 4.61 (s, 2H), 4.00 (q, J=5.8 Hz, 2H), 3.07 (s, 3H), 1.52 (d, J=6.9 Hz, 3H); LRMS [M+H]⁺ 540.

### IC-102: 7-(1-(3-(4-(aminomethyl)phenyl)ureido)ethyl)-3-(3-fluoro-4-((methylsulfonyl)methyl)phenyl)-1H-indole-2-carboxylic acid

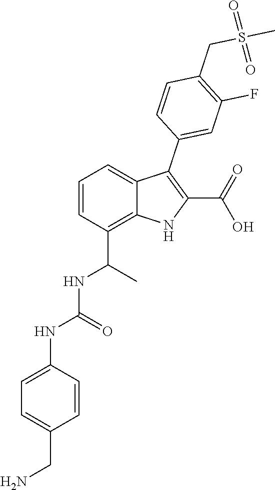 [SMILES: CC(NC(=O)Nc1ccc(CN)cc1)c1cccc2c(-c3ccc(CS(C)(=O)=O)c(F)c3)c(C(=O)O)[nH]c12]

To the amine S13 (105 mg, 0.250 mmol) and tert-butyl (4-(((4-nitrophenoxy)carbonyl)amino)benzyl)carbamate (97 mg, 0.25 mmol) was added DCM (2.5 mL) and the reaction was stirred at RT for 22 h and then concentrated. Purification by FCC (PE:EtOAc, 2:3) afforded the urea in 85% yield as a white solid (142 mg).

To the urea (145 mg, 0.217 mmol) in EtOH (2 mL) was added an aq solution of NaOH (1 mL, 43.5 mg) and the reaction was stirred at 60° C. for 1 h. EtOH was removed under reduced pressure, the reaction was diluted with MeCN (1 mL) and acidified with 1 M HCl (1 mL) and then concentrated in vacuo. To the residue as a suspension in MeCN (2 mL) was added 1 M HCl (2 mL) and the mixture was heated at 50° C. for 3 h. The mixture was concentrated then purified by RP-FCC (H₂O MeCN, 0.1% TFA 0-60%) and then counterion exchange with aq HCl afforded the indole carboxylate IC-102 in 73% yield (91 mg).

¹H NMR (300 MHz, DMSO-d6): 14.03-12.10 (bs, 1H), 11.76 (s, 1H), 8.95 (s, 1H), 8.16 (s, 3H), 7.53 (t, J=8.0 Hz, 1H), 7.47-7.26 (m, 8H), 7.20-7.04 (m, 2H), 5.68-5.42 (m, 1H), 4.61 (s, 2H), 3.91 (q, J=5.4 Hz, 2H), 3.07 (s, 3H), 1.52 (d, J=6.8 Hz, 3H); LRMS [M+H]⁺ 539.

### General Procedures

### Method B: C3-Indole Halogenation

N-Halosuccinimide (1.05 equiv) was added to the stirred solution of corresponding indole (1 mmol, 1.0 equiv) in MeCN (4 mL/mmol) at 0° C. A cooling bath was removed, and the reaction mixture was warmed to rt, stirred for 1 h and concentrated under reduced pressure. The residue was partitioned between EtOAc (20 mL) and 10% Na₂S₂O₃ (20 mL). Organic layer was sequentially washed with 10% Na₂S₂O_{3aq} (10 mL), followed by H₂O (3×10 mL) and brine (10 mL), dried over Na₂SO₄, filtered, and concentrated in vacuo to give 3-haloindole. The product was used in the next step without any further purification.

### Method C:Suzuki-Miyaura Cross-Coupling Reaction

A microwavable vial charged with corresponding 3-haloindole (1 mmol, 1 equiv), 2-(3-fluoro-4-((methylsulfonyl)methyl)phenyl)-4,4,5,5-tetramethyl-1,3,2-dioxaborolane (1.2 equiv), Pd(dppf)Cl₂·DCM (5 mol %), degassed dioxane (3 mL/mmol) and 2M aqueous Na₂CO₃ (2 equiv) was purged with argon, sealed and stirred at 90° C. for 1 h. The reaction mixture was cooled to room temperature and filtered over Celite®, eluting with EtOAc (10 mL). A filtrate was concentrated in vacuo; the residue was taken up in EtOAc (20 mL), washed with H₂O (10 mL) and brine (10 mL), dried over Na₂SO₄ and concentrated under reduced pressure. A dry residue was further purified by column chromatography on SiO₂.

### Method D: Acid-Catalysed Hydrolysis of Tert-Butyl Ester/N-Boc-Deprotection

Trifluoroacetic acid (2.5 mL/mmol) was added to the solution of t-Bu-ester or N-Boc derivative (1 mmol, 1 equiv) in DCM (2.5 mL/mmol). The reaction mixture was stirred at room temperature for 1 h and concentrated in vacuo. The product was used directly in the next step.

### Method E: Amide Synthesis

A solution of acid (1 mmol, 1 equiv), relevant amine (1.1 equiv), HATU (65.5 mg, 0.172 mmol, 1.1 equiv) and DIPEA (82 μL, 0.470 mmol, 3.0 equiv) in MeCN (4 mL/mmol) was stirred 1 h at rt and evaporated under reduced pressure. The dry residue was taken up in EtOAc (20 mL), washed with water (4×8 mL) and brine (8 mL). The organic phase was dried over Na₂SO₄ and concentrated in vacuo to give crude amide, which was subjected to the next step without further purification.

### Method F: Ethyl Ester Hydrolysis

Lithium hydroxide monohydrate (3 equiv) was added to solution of ethyl ester (1 mmol, 1 equiv) in MeOH—H₂O (10 mL/mmol, 2:1), and the obtained mixture was stirred for 1 h at 60° C. The reaction was then cooled to rt, acidified with 1M HCl till pH 2 and further purified by preparative HPLC.

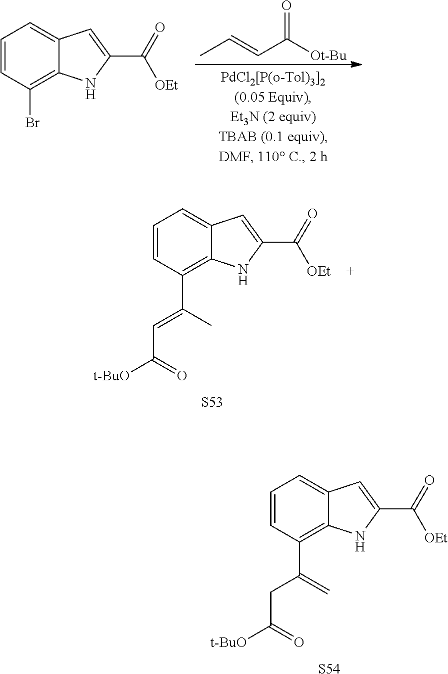

To a solution of ethyl 7-bromo-1H-indole-2-carboxylate (2 g, 7.46 mmol), tert-butyl crotonate (954 mg, 6.71 mmol) and TBAB (240 mg, 0.746 mmol) in 20 mL DMF was added TEA (2 mL, 14.9 mmol) under nitrogen atmosphere. The reaction mixture was degassed for 5 min and added dichlorobis(tri-o-tolylphosphine)palladium(II) (293 mg, 0.373 mmol) and the reaction was stirred under nitrogen condition at 110° C. for 2 h. The contents were cooled and poured into ice cold water and extracted with ethyl acetate (3×50 mL), organic layer washed with water (3×50 mL) dried (Na₂SO₄) and concentrated under reduced pressure. The crude product was purified by FCC on SiO₂ (0->25% EtOAc-Hept gradient) provided: intermediates S53 in 1.32 g in 54% and S54 as 680 mg in 28% yields.

Compound S53: ¹H NMR (400 MHz, CDCl₃) δ 8.99 (s, 1H), 7.64 (d, J=8.0 Hz, 1H), 7.28-7.23 (m, 2H), 7.16 (t, J=7.7 Hz, 1H), 6.14 (d, J=1.5 Hz, 1H), 4.42 (q, J=7.1 Hz, 2H), 2.62 (d, J=1.4 Hz, 3H), 1.55 (s, 9H), 1.42 (t, J=7.1 Hz, 3H). ¹³C NMR (101 MHz, CDCl₃) δ 166.0, 161.8, 152.0, 133.6, 128.1, 127.9, 127.8, 123.6, 122.7, 120.9, 120.8, 108.9, 80.4, 61.1, 28.3, 19.5, 14.3.

Compound S54: ¹H NMR (400 MHz, CDCl₃) δ ¹H NMR (400 MHz, CDCl₃) δ 9.96 (s, 1H), 7.76-7.66 (m, 2H), 7.32-7.15 (m, 2H), 5.58 (d, J=8.3 Hz, 2H), 4.47 (q, J=7.1 Hz, 2H), 3.52 (s, 2H), 1.44 (m, 12H); ¹³C NMR (101 MHz, CDCl₃) δ 171.1, 161.6, 139.8, 134.3, 127.7, 127.6, 126.3, 123.2, 121.8, 120.3, 117.8, 108.5, 81.3, 60.7, 42.9, 27.6, 14.1.

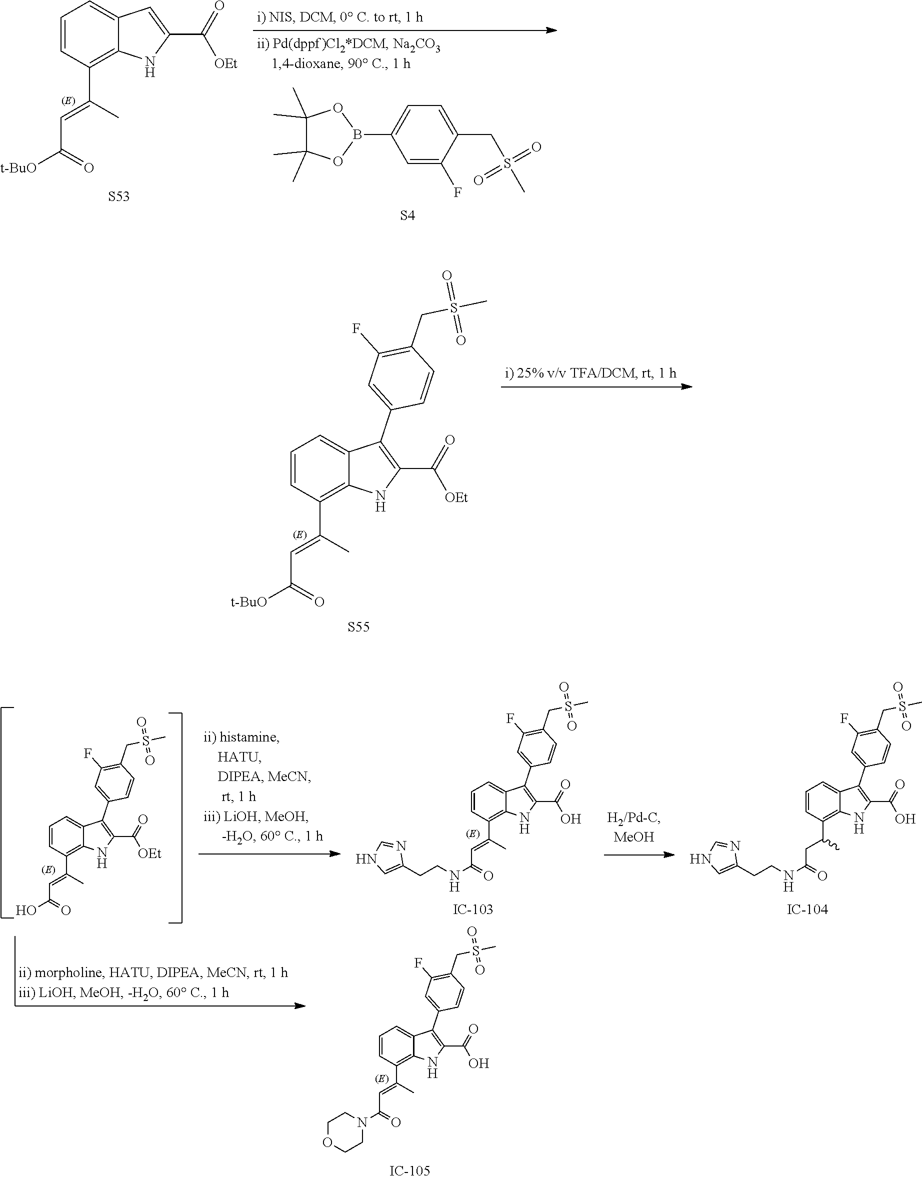

### Ethyl 7-[(2E)-4-(tert-butoxy)-4-oxobut-2-en-2-yl]-3-[3-fluoro-4-(methanesulfonylmethyl)phenyl]-1H-indole-2-carboxylate

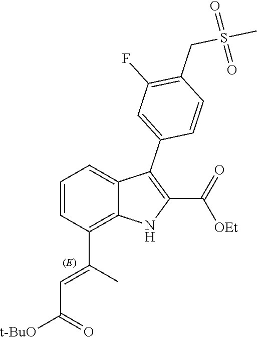 [SMILES: CCOC(=O)c1[nH]c2c(/C(C)=C/C(=O)OC(C)(C)C)cccc2c1-c1ccc(CS(C)(=O)=O)c(F)c1]

The ester was synthesized via 2-step synthetic sequence: C3-iodonation of S53 with N-iodosuccinimide (Method B) followed by Suzuki-Miyaura cross-coupling (Method C). Purification by FCC on SiO₂ (0→35% EtOAc—Hept gradient) provided S55 (88 mg, 86%) as a white solid. ¹H NMR (400 MHz, CDCl₃) δ 9.17 (s, 1H), 7.57 (t, J=8.1 Hz, 2H), 7.44-7.28 (m, 3H), 7.19 (dd, J=8.1, 7.3 Hz, 1H), 6.17 (q, J=1.4 Hz, 1H), 4.40 (s, 2H), 4.32 (q, J=7.1 Hz, 2H), 2.89 (s, 3H), 2.65 (d, J=1.4 Hz, 3H), 1.55 (s, 9H), 1.26 (s, 3H). ¹³C NMR (101 MHz, CDCl₃) δ 166.0, 160.4 (d, J_{CF}=247.7 Hz), 161.3, 151.1, 137.0 (d, J_{CF}=8.8 Hz), 132.4, 132.1 (d, J_{CF}=3.8 Hz), 128.0 (d, J_{CF}=1.5 Hz), 127.3, (d, J_{CF}=3.1 Hz), 124.4, 123.5, 121.9 (d, J_{CF}=1.9 Hz), 121.5, 121.3, 121.2, 118.0 (d, J_{CF}=22.8 Hz), 114.6, (d, J_{CF}=15.4 Hz), 8.06, 61.3, 54.2 (d, J_{CF}=3.0 Hz), 39.4 (d, J_{CF}=4.7 Hz), 31.8, 29.0, 28.3, 24.8, 22.6, 19.8, 141 (d, J_{CF}=10.4 Hz). LCMS [M+H]⁺ 460.4.

### IC-103: 3-[3-Fluoro-4-(methanesulfonylmethyl)phenyl]-7-[(1E)-1-{[2-(1H-imidazol-4-yl)ethyl]carbamoyl}prop-1-en-2-yl]-1H-indole-2-carboxylic acid (Comparative Example)

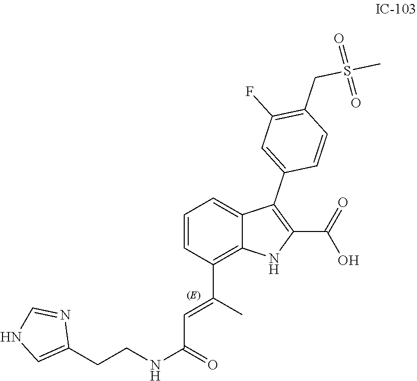 [SMILES: C/C(=C\C(=O)NCCc1c[nH]cn1)c1cccc2c(-c3ccc(CS(C)(=O)=O)c(F)c3)c(C(=O)O)[nH]c12]

The indole carboxylate was obtained from S55 by successive acid-catalysed hydrolysis of t-butyl ester (Method D), amidation with histamine (Method E) and ethyl ester cleavage (Method F). The corresponding trans-alkene was isolated after purification by preparative HPLC to get TFA salt as a white solid which was converted to hydrochloride salt using 5% HCl solution to obtain IC-103 (30 mg, 88%) as a white solid.

¹H NMR (600 MHz, DMSO-d6) δ 11.33 (s, 1H), 8.97 (d, J=1.7 Hz, 1H), 8.15 (q, J=5.5 Hz, 1H), 7.54 (t, J=7.9 Hz, 1H), 7.49-7.45 (m, 2H), 7.40-7.36 (m, 2H), 7.21 (dd, J=7.2, 1.1 Hz, 1H), 7.16-7.10 (m, 1H), 6.08 (d, J=1.9 Hz, 1H), 4.61 (s, 2H), 3.47 (t, J=6.5 Hz, 2H), 3.07 (s, 3H), 2.87 (t, J=7.0 Hz, 2H), 2.53 (d, J=1.3 Hz, 3H). ¹³C NMR (151 MHz, DMSO-d6) b 166.2, 162.4, 160.8, (d, J_{CF}=247.0 Hz), 146.7, 136.6 (d, J_{CF}=8.8 Hz), 133.8, 132.6, 132.5 (d, J_{CF}=3.8 Hz), 131.1, 129.4, 127.6, 126.6 (d, J_{CF}=3.1 Hz), 125.1, 123.4, 122.7, 120.8, 120.5, 120.0, 117.6 (d, J_{CF}=22.5 Hz), 116.2, 114.4 ((d, J_{CF}=15.4 Hz), 53.0, 40.1, 37.3, 24.6, 18.9. LRMS (ESI) m/z: [M+H]⁺ calcd for C₂₆H₂₆FN₄O₅S 525.4, found 525.5.

### IC-104: 3-[3-Fluoro-4-(methanesulfonylmethyl)phenyl]-7-(1-{[2-(1H-imidazol-4-yl)ethyl]carbamoy}propan-2-yl)-1H-indole-2-carboxylic acid

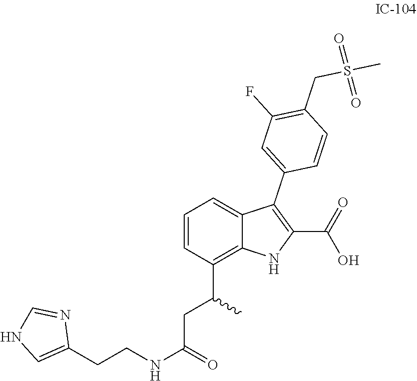 [SMILES: CC(CC(=O)NCCc1c[nH]cn1)c1cccc2c(-c3ccc(CS(C)(=O)=O)c(F)c3)c(C(=O)O)[nH]c12]

The indole carboxylate was obtained from IC-103 (18 mg, 0.108 mmol) in 1 mL of MeCOH, was added Pd-C(11.4 mg, 0.742 mmol) and the reaction was stirred at rt under H₂ gas atmosphere for 12 h. The reaction was concentrated in vacuo to get the crude mass which was further purified by Preparative HPLC to obtain IC-104 as the TFA salt which was converted to the hydrochloride salt using 5% HCl solution as a white solid (16 mg, 88%).

¹H NMR (600 MHz, DMSO-d6) δ 11.57 (s, 1H), 8.93 (d, J=1.3 Hz, 1H), 8.09 (t, J=5.8 Hz, 1H), 7.52 (t, J=8.0 Hz, 1H), 7.40-7.29 (m, 4H), 7.16 (d, J=7.2 Hz, 1H), 7.06 (q, J=7.4 Hz, 1H), 4.60 (s, 2H), 3.96 (q, J=7.1 Hz, 1H), 3.33 (d, J=6.4 Hz, 2H), 3.07 (s, 3H), 2.72 (t, J=7.0 Hz, 2H), 2.56 (dd, J=14.2, 7.0 Hz, 1H), 2.40 (dd, J=14.2, 7.9 Hz, 1H), 1.24 (d, J=6.8 Hz, 3H). ¹³C NMR (151 MHz, DMSO-d6) δ 171.4, 162.6, 161.2 (d, J_{CF}=247.0 Hz), 159.5, 136.9 (d, J_{CF}=8.6 Hz), 134.3, 133.7, 132.4 (d, J_{CF}=3.3 Hz), 131.3, 131.0, 126.9, 126.5 (d, J_{CF}=3.3 Hz), 124.3, 121.0 (d, J_{CF}=5.3 Hz), 120.5 (d, J_{CF}=1.8 Hz), 117.9, 117.4 (d, J_{CF}=22.0 Hz), 116.1, 114.2 (d, J_{CF}=15.7 Hz), 50.0, 42.7, 40.1, 37.2, 29.9, 24.5, 21.2. LRMS (ESI) m/z: [M+H]⁺ calcd for C₂₅H₂₈FN₄O₅S 527.2, found 527.4.

### IC-105: 3-[3-Fluoro-4-(methanesulfonylmethyl)phenyl]-7-[(2E)-4-(morpholin-4-yl)-4-oxobut-2-en-2-yl]-1H-indole-2-carboxylic acid (Comparative Example)

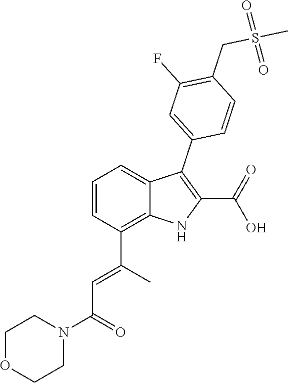 [SMILES: C/C(=C\C(=O)N1CCOCC1)c1cccc2c(-c3ccc(CS(C)(=O)=O)c(F)c3)c(C(=O)O)[nH]c12]

The indole carboxylate was obtained from S55 by successive acid-catalysed hydrolysis of t-butyl ester (Method D), amidation with morpholine (Method E) and ethyl ester cleavage (Method F). The corresponding product was isolated after purification by Preparative HPLC to get TFA salt as a white solid which was converted to hydrochloride salt using 5% HCl solution to obtain IC-105 in 71% over two steps as a white solid (31 mg).

¹H NMR (600 MHz, DMSO-d6) δ 11.32 (s, 1H), 7.54 (t, J=7.8 Hz, 1H), 7.47 (d, J=8.1 Hz, 1H), 7.38 (t, J=9.5 Hz, 2H), 7.25 (d, J=7.1 Hz, 1H), 7.14 (t, J=7.6 Hz, 1H), 6.37 (s, 1H), 4.61 (s, 2H), 3.58 (ddd, J=28.5, 11.4, 5.2 Hz, 8H), 3.07 (s, 3H), 2.35 (s, 3H). ¹³C NMR (151 MHz, DMSO-d6) δ 165.7, 162.4, 160.5 (d, J_{CF}=247.6 Hz), 144.6, 136.5 (d, J_{CF}=8.8 Hz), 132.7, 132.4 (d, J_{CF}=3.7 Hz), 128.8, 127.6, 126.5 (d, J_{CF}=3.3 Hz), 125.2, 123.8, 121.3, 120.9, 120.5 (d, J_{CF}=1.8 Hz), 120.0, 117.44 (d, J_{CF}=22.3 Hz), 114.4 (d, J_{CF}=15.1 Hz), 66.27 (d, J_{CF}=28.2 Hz), 53.0, 46.1, 41.3, 19.3. LRMS (ESI) m/z: [M+H]⁺ calcd for C₂₆H₂₈FN₂O₅S 499.5, found 499.7.

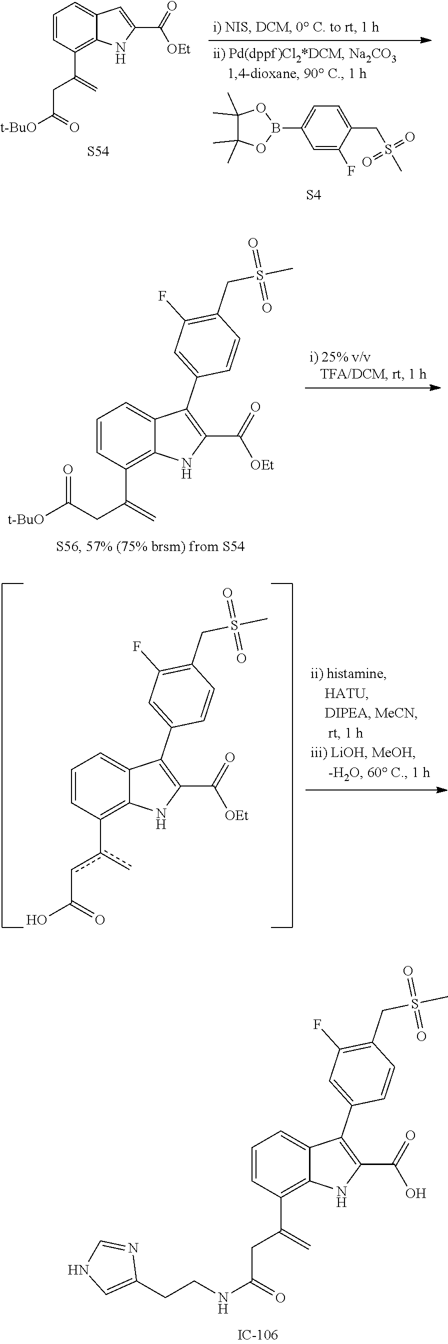

### Ethyl 7-(4-(tert-butoxy)-4-oxobut-1-en-2-yl)-3-(3-fluoro-4-((methylsul-fonyl)methyl)phenyl)-1H-indole-2-carboxylate

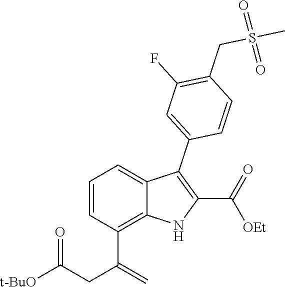 [SMILES: C=C(CC(=O)OC(C)(C)C)c1cccc2c(-c3ccc(CS(C)(=O)=O)c(F)c3)c(C(=O)OCC)[nH]c12]

Synthesised via 2-step synthetic sequence: C3-bromination of ethyl 7-(4-(tert-butoxy)-4-oxobut-1-en-2-yl)-1H-indole-2-carboxylate S54 with N-bromosuccinimide (Method B) followed by Suzuki-Miyaura cross-coupling (Method C). Purification by FCC on SiO₂ (5→35% EtOAc—Hept gradient) provided 187 mg (57%, 75% brsm) of S56 as a yellow foam.

¹H NMR (600 MHz, CDCl₃) δ 10.34 (s, 1H), 7.58-7.53 (m, 2H), 7.44-7.42 (m, 1H), 7.40-7.37 (m, 1H), 7.25 (d, J=7.1 Hz, 1H), 7.14 (t, J=7.6 Hz, 1H), 5.57 (s, 1H), 5.55 (s, 1H), 4.39 (s, 2H), 4.32 (q, J=7.1 Hz, 2H), 3.49 (s, 2H), 2.89 (s, 3H), 1.41 (s, 9H), 1.29 (t, J=7.2 Hz, 3H); ¹³C NMR (151 MHz, CDCl₃) δ 171.9, 161.5, 160.5 (d, J_{CF}=247.1 Hz), 140.0, 137.6 (d, J_{CF}=8.9 Hz), 133.4, 132.15, 132.1 (d, J_{CF}=3.4 Hz), 127.9, 127.5 (d, J_{CF}=2.9 Hz), 127.0, 124.4, 121.9 (d, J_{CF}=2.0 Hz), 121.3, 120.8, 118.7, 118.2 (d, J_{CF}=22.4 Hz), 114.5 (d, J_{CF}=14.8 Hz), 81.9, 61.2, 54.4 (d, J_{CF}=2.9 Hz), 42.9, 39.5 (d, J_{CF}=2.3 Hz), 28.0, 14.2; LRMS (ESI) m/z: [M-t-Bu+2H]⁺ calcd for C₂₃H₂₃FNO₆S 460.1, found 460.2.

### IC-106: 7-(4-((2-(1H-Imidazol-4(5)-yl)ethyl)amino)-4-oxobut-1-en-2-yl)-3-(3-fluoro-4-((methylsulfonyl)-methyl)phenyl)-1H-indole-2-carboxylic acid (Comparative Example)

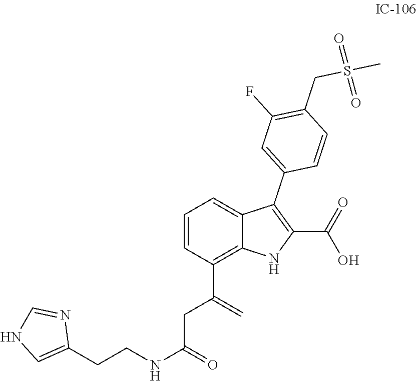 [SMILES: C=C(CC(=O)NCCc1c[nH]cn1)c1cccc2c(-c3ccc(CS(C)(=O)=O)c(F)c3)c(C(=O)O)[nH]c12]

Synthesised from S56 by successive acid-catalysed hydrolysis of t-butyl ester (Method D; NOTE: partial isomerization of gem-disubstituted alkene to trans-takes place at this step), amidation with histamine (Method E) and ethyl ester cleavage (Method F). Two major products were isolated after purification by preparative HPLC:

1. 8.6 mg (10% over 3 steps) of trans-alkene IC-103 as the TFA salt as a white solid. The analytical data are consistent with those reported previously.

2. 14.8 mg (17% over 3 steps) of gem-disubstituted alkene IC-106 as the TFA salt as a white solid; ¹H NMR (600 MHz, DMSO-d₆) δ 14.27 (br s, 2H), 12.30 (s, 1H), 8.97 (d, J=1.4 Hz, 1H), 8.54 (t, J=5.8 Hz, 1H), 7.54 (t, J=7.8 Hz, 1H), 7.47 (d, J=8.0 Hz, 1H), 7.42-7.37 (m, 2H), 7.35 (d, J=1.3 Hz, 1H), 7.19 (dd, J=7.1, 1.1 Hz, 1H), 7.09 (dd, J=8.1, 7.1 Hz, 1H), 5.44 (d, J=0.9 Hz, 1H), 5.38 (s, 1H), 4.61 (s, 2H), 3.45 (q, J=6.6 Hz, 2H), 3.35 (s, 2H), 3.07 (s, 3H), 2.82 (t, J=6.8 Hz, 2H); ¹³C NMR (151 MHz, DMSO-d₆) δ 171.9, 162.4, 160.4 (d, J_{CF}=247.0 Hz), 141.2, 136.6 (d, J_{CF}=8.8 Hz), 133.8, 132.8, 132.5 (d, J_{CF}=3.8 Hz), 130.8, 127.5, 127.4, 126.6 (d, J_{CF}=3.0 Hz), 124.7, 124.0, 120.8, 120.4 (d, J_{CF}=1.8 Hz), 120.1, 118.2, 117.5 (d, J_{CF}=22.2 Hz), 116.2, 114.35 (d, J_{CF}=15.4 Hz), 53.0, 41.7, 40.2, 37.5, 24.3; ¹⁹F NMR (376 MHz, DMSO-d₆) δ −116.9; HPLC analysis t_{R} 9.2 min, purity >99.0%; LRMS (ESI) m/z: [M+H]⁺ calcd for C₂₆H₂₆FN₄O₅S 525.2, found 525.3; [M-H]⁻ calcd for C₂₆H₂₄FN₄O₅S 523.1, found 523.1.

### IC-107:(2S,4r)-2-(Aminomethyl)-7-((S)-1-(3-(3-fluoro-4-((methylsulfonyl)methyl)phenyl)-2-(1H-tetrazole-5-yl)-1H-indol-7-yl)ethyl)-5-oxa-7-azaspiro[3.4]octan-6-one

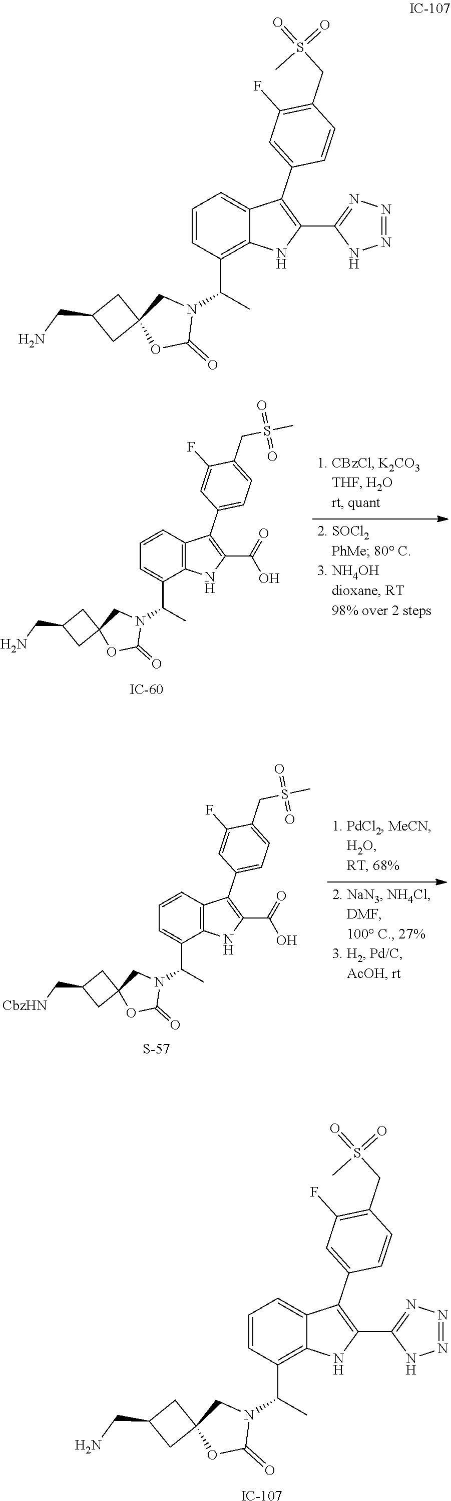

IC-107 was prepared from IC-60 by N-CBz protection of the primary amine, conversion of the carboxylic acid to the nitrile via the primary amide S-57; followed by tetrazole formation then subsequent deprotection of the amine. The product was obtained following purification by chromatography as an amorphous solid (6.30 mg).

¹H NMR (400 MHz, DMSO-d₆) δ 10.78 (br s, 1H), 7.74-7.55 (m, 5H), 7.54-7.50 (m, 1H), 7.50-7.43 (m, 1H), 7.19-7.14 (m, 1H), 7.12-7.05 (m, 1H), 5.53 (q, J=7.0 Hz, 1H), 4.57 (s, 2H), 3.70 (d, J=9.7 Hz, 1H), 3.27 (d, J=9.7 Hz, 1H), 3.05 (s, 3H), 2.86 (dt, J=7.1, 4.3 Hz, 2H), 2.54-2.51 (m, 2H, overlapped with DMSO signal), 2.44-2.35 (m, 1H), 2.23-2.11 (m, 1H), 2.09-2.00 (m, 1H), 1.65 (d, J=7.0 Hz, 3H); HPLC analysis t_{R} 7.60 min, purity 99.0%; LRMS (ESI) m/z: [M+H]⁺ calcd for C₂₆H₂₈FN₇O₄S 554.2, found 554.2.

### Biological Data

Inhibitors were tested using a panel of the clinically relevant B1 MBLs (NDM-1, VIM-1, -2 and IMP-1). Activities were assayed by monitoring the release of a fluorophore following the enzymatic breakdown of the cephalosporin FC-5 (https://pubs.acs.org/doi/abs/10.1021/jm400769b). FC-5 assays were conducted in clear bottomed 384 black well microplates (Greiner); the initial rate of reaction was assessed by monitoring the fluorescence intensity (λex=380 nm and λem=460 nm) using a Pherstar FS (BMG LabTech) plate reader. Initial rates of reaction were determined and dose-response analyses conducted in GraphPad Prism. Dose-response curves were fitted using the log(inhibitor) vs normalized response—variable slope model of GraphPad Prism 12. Each concentration point for routine screening was conducted with 4 intra-plate replicates.

TABLE 1
Inhibition values are represented as ranges of the obtaiend pIC₅₀ values. A =
pIC₅₀ values <8, B = pIC₅₀ values 8-9, C = pIC₅₀ values 9-10 and pIC₅₀ values D = >10.

| Compound | VIM-2 | NDM-1 | IMP-1 | VIM-1 |
|---|---|---|---|---|
| Prior Art | C | B | A | A |
| Compound 76 | | | | |
| IC-43 | B | C | A | A |
| IC-42 | C | C | A | B |
| IC-41 | B | C | A | B |
| IC-40 | A | A | B | A |
| IC-39 | B | C | A | C |
| IC-38 | C | C | A | B |
| IC-37 | B | B | A | A |
| IC-36 | A | A | A | A |
| IC-35 | C | A | A | B |
| IC-34 | B | A | A | B |
| IC-33 | C | B | A | B |
| IC-32 | C | C | A | B |
| IC-31 | B | B | A | A |
| IC-30 | A | A | A | A |
| IC-29 | B | A | A | A |
| IC-28 | C | B | A | B |
| IC-27 | D | C | A | B |
| IC-26 | C | B | A | A |
| IC-25 | C | A | A | A |
| IC-24 | C | D | A | B |
| IC-23 | C | B | A | B |
| IC-22 | D | C | A | B |
| IC-51 | B | B | A | A |
| IC-21 | B | B | A | A |
| IC-20 | C | C | A | B |
| IC-52 | B | D | A | A |
| IC-53 | B | D | A | B |
| IC-19 | C | D | A | C |
| IC-54 | B | C | A | A |
| IC-18 | C | C | A | B |
| IC-17 | D | C | A | B |
| IC-16 | C | D | A | B |
| IC-55 | C | D | A | A |
| IC-56 | C | D | A | A |
| IC-14 | C | D | A | C |
| IC-57 | C | D | A | B |
| IC-62 | C | D | B | C |
| IC-66 | C | C | A | C |
| IC-4 | B | B | B | B |
| IC-65 | C | C | A | B |
| IC-67 | C | D | A | B |
| IC-68 | C | D | A | B |
| IC-69 | C | D | A | C |
| IC-63 | D | B | A | A |
| IC-5 | D | C | A | B |
| IC-6 | A | B | A | A |
| IC-59 | D | D | A | A |
| IC-61 | D | D | B | C |
| IC-7 | B | C | B | B |
| IC-60 | C | D | A | C |
| IC-73 | B | C | A | B |
| IC-104 | A | B | A | A |
| IC-105 | A | A | A | A |
| IC-103 | A | A | A | A |
| IC-106 | A | A | A | A |
| IC-3 | B | B | A | A |
| IC-70 | C | D | A | C |
| IC-71 | B | B | A | A |
| IC-64 | B | B | A | A |
| IC-74 | C | D | A | C |
| IC-85 | A | C | A | A |
| IC-76 | A | B | A | A |
| IC-81 | B | C | A | B |
| IC-82 | C | C | A | B |
| IC-9 | A | A | A | A |
| IC-75 | B | C | A | B |
| IC-84 | C | C | A | C |
| IC-83 | B | C | A | B |
| IC-80 | B | C | A | B |
| IC-79 | B | C | A | B |
| IC-78 | A | B | A | A |
| IC-86 | B | D | A | A |
| IC-98 | A | A | A | A |
| IC-90 | B | B | A | B |
| IC-13 | A | D | A | B |
| IC-12 | B | A | A | A |
| IC-8 | B | A | A | A |
| IC-89 | B | C | A | B |
| IC-91 | A | A | A | A |
| IC-77 | B | C | A | B |
| IC-72 | C | D | A | C |
| IC-93 | B | B | A | A |
| IC-94 | B | B | A | A |
| IC-92 | B | B | B | B |
| IC-88 | A | C | B | A |
| IC-87 | C | D | B | B |
| IC-95 | B | C | A | A |
| IC-96 | B | B | A | A |
| IC-97 | B | B | A | A |
| IC-99 | D | B | A | A |
| IC-101 | B | B | A | A |
| IC-102 | B | B | A | A |
| IC-100 | C | A | A | A |
| IC-11 | B | D | A | A |
| IC-10 | D | C | A | A |
| IC-1 | A | A | A | A |
| IC-2 | A | A | A | A |
| IC-107 | A | B | A | B |

Note:
Prior Art Compound 76 is a compound disclosed within WO2017093727, and has the structure below:

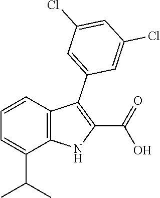 [SMILES: CC(C)c1cccc2c(-c3cc(Cl)cc(Cl)c3)c(C(=O)O)[nH]c12]

Antibiotic susceptibility testing was performed using the agar or broth dilution method, in line with the Clinical and Laboratory Standards Institute (CLSI) and European Committee on Antimicrobial Susceptibility Testing (EUCAST) requirements.

TABLE 2
MIC values A = MIC values ≤ 1, B MIC values 2 ÷ 8
and C = MIC values > 8. KP-Klebsiella pneumoniae, AB-
Acinetobacter baumannii and EC-Escherichia coli

| | KP | KP | KP | AB | EC | EC |
|---|---|---|---|---|---|---|
| Cmpd | IMP-4 | VIM-1 | NDM-1 | NDM-1 | NDM-7 | NDM-1 |
| DMSO | C | C | C | C | C | C |
| IC-39 | A | A | A | B | A | A |
| IC-38 | B | A | A | B | A | A |
| IC-37 | B | A | B | B | A | A |
| IC-36 | B | A | A | B | A | A |
| IC-35 | B | A | B | C | A | A |
| IC-34 | B | A | B | C | A | B |
| IC-33 | B | A | A | B | A | A |
| IC-32 | B | A | B | B | A | B |
| IC-29 | C | A | B | C | A | B |
| IC-28 | B | A | B | C | A | B |
| IC-27 | C | A | B | B | A | B |
| IC-26 | C | A | B | C | A | B |
| IC-25 | C | A | B | C | B | C |
| IC-24 | B | A | B | B | A | A |
| IC-15 | C | A | B | C | A | B |
| IC-23 | C | A | A | B | A | A |
| IC-22 | B | A | A | B | A | A |
| IC-51 | C | B | B | C | B | C |
| IC-21 | C | B | C | C | B | C |
| IC-20 | B | A | A | B | A | A |
| IC-52 | B | B | B | C | B | B |
| IC-53 | B | A | C | B | A | A |
| IC-19 | B | A | B | B | A | B |
| IC-54 | B | A | B | B | A | A |
| IC-18 | B | A | A | B | A | A |
| IC-17 | B | A | A | B | A | A |
| IC-16 | B | A | A | B | A | A |
| IC-55 | B | B | B | B | B | B |
| IC-56 | B | A | A | B | A | A |
| IC-14 | B | A | A | B | A | A |
| IC-57 | A | A | A | A | A | A |
| IC-62 | A | A | A | A | A | A |
| IC-66 | A | A | B | B | A | A |
| IC-65 | B | A | A | B | A | A |
| IC-67 | B | A | A | B | A | A |
| IC-68 | B | A | A | B | A | A |
| IC-69 | B | A | A | B | A | A |
| IC-59 | B | A | A | A | A | A |
| IC-61 | A | A | A | B | A | A |
| IC-60 | B | A | A | A | A | A |
| IC-105 | C | B | C | C | C | C |
| IC-103 | A | C | C | C | C | C |
| IC-106 | C | B | C | C | C | C |
| IC-70 | A | A | A | A | A | A |
| IC-71 | B | A | B | C | A | B |
| IC-74 | B | A | A | B | A | A |
| IC-80 | A | A | B | B | A | A |
| IC-79 | B | A | A | A | A | A |

Metabolic stability assays were performed in cryopreserved human and mouse hepatocytes. The hepatocytes were thawed and added to “Thawing media”, and centrifuged to pellet the hepatocytes. The cell pellet was taken up in Williams medium E (Invitrogen A1217601), the number of cells was calculated and adjusted to 1.0×10⁶ cells/mi. The incubation was carried out on a heater-shaker at 3700, in a 24-well Picoplate (round bottom, PerkinElmer), using an incubation volume of 770 μl. Thus, to obtain a final compound concentration of 1 μM, 0.7 μl of the 1 mM compound stock solution was pipetted to the wells of the plate. Reaction was initiated by adding 700 μl of cell suspension. Samples were incubated at 37° C. for ˜60 see and a zero sample was taken, i.e. 100 μl sample was removed to a 96-well plate and the reaction was quenched by adding 100 μl of 100% MeCN containing 50 nM Warfarin as an internal control. Consecutive samples were taken at time-points 5 min, 15 min, 30 min, 60 min, and 90 min. After finishing the experiment, samples were centrifuged at 3000 rpm for 15 min and before analysis by LC-MS/MS (Waters Corp, Milford, MA, USA).

TABLE 3
Metabolic stability in hepatocytes, values are represented as ranges of the
obtaiend in vitro t_{1/2} (min) A = <150 min, B = 150-400 and C = >400.

| Compound | Human | Mouse |
|---|---|---|
| Prior Art Compound 76 | A | A |
| IC-18 | A | C |
| IC-17 | C | B |
| IC-16 | C | C |
| IC-55 | B | B |
| IC-56 | C | A |
| IC-14 | C | B |
| IC-57 | B | A |
| IC-62 | C | B |
| IC-66 | B | C |
| IC-67 | C | C |
| IC-68 | C | C |
| IC-69 | C | A |
| IC-61 | C | C |
| IC-60 | C | C |
| IC-70 | C | B |
| IC-71 | C | B |
| IC-74 | C | A |
| IC-80 | C | A |
| IC-79 | C | A |
| IC-86 | C | A |
| IC-89 | C | C |
| IC-77 | C | C |
| IC-72 | C | C |
| IC-92 | C | B |
| IC-88 | C | A |
| IC-87 | C | A |
| IC-95 | C | C |
| IC-97 | C | A |
| IC-99 | C | C |
| IC-11 | C | C |

The fraction of unbound compound (fu) in plasma from human or other animal species was determined by equilibrium dialysis at 37° C. for 4 hours using the Rapid Equilibrium Dialysis (RED) device (ThermoFisher Scientific). The test compound at a concentration of 10 μM was added to plasma and dialyzed against isotonic phosphate buffer (67 mM, pH 7.4) over 4 hours at 37° C. After dialysis, compound concentrations in buffer and plasma were quantified by LC-MS/MS. In parallel the stability of the compound in plasma was determined by incubating drug-spiked plasma (10 μM) at 37° C. for 4 hours, meanwhile the control plasma sample was kept in the freezer. The concentration of the compound in both samples was quantified by LC-MS/MS (Waters Corp, Milford, MA, USA).

TABLE 4
Plasma protein binding, fu values are represented as ranges of the
obtaiend values, fu (%) A = <0.5, B = 0.5-5 and C = 5-25, D = >25.

| Compound | Human | Mouse |
|---|---|---|
| Prior Art Compound 76 | A | A |
| IC-39 | B | — |
| IC-38 | B | — |
| IC-37 | C | — |
| IC-36 | C | — |
| IC-35 | D | — |
| IC-34 | D | — |
| IC-33 | C | — |
| IC-32 | D | — |
| IC-29 | B | — |
| IC-28 | C | — |
| IC-27 | C | — |
| IC-26 | C | — |
| IC-25 | D | — |
| IC-24 | C | C |
| IC-15 | C | B |
| IC-23 | C | B |
| IC-22 | C | B |
| IC-51 | C | B |
| IC-21 | C | B |
| IC-19 | D | C |
| IC-54 | D | D |
| IC-18 | D | B |
| IC-17 | C | B |
| IC-16 | C | C |
| IC-55 | C | B |
| IC-56 | C | B |
| IC-14 | C | C |
| IC-57 | C | B |
| IC-62 | C | B |
| IC-66 | D | B |
| IC-67 | C | B |
| IC-69 | C | B |
| IC-61 | C | B |
| IC-60 | D | C |
| IC-70 | C | B |
| IC-71 | D | D |
| IC-74 | C | C |
| IC-80 | D | C |
| IC-79 | D | C |
| IC-86 | B | C |
| IC-90 | D | C |
| IC-13 | C | B |
| IC-8 | C | — |
| IC-89 | C | C |
| IC-77 | C | C |
| IC-72 | C | C |
| IC-92 | C | B |
| IC-88 | B | A |
| IC-87 | B | C |
| IC-95 | C | B |
| IC-97 | C | C |
| IC-99 | C | C |
| IC-11 | D | B |
| IC-2 | A | A |

### Thigh Model of Infection.

Female NMRI mice (5-6 weeks old, 26-30 gram, n=5) were rendered neutropenic via intraperitoneal injection of two doses of cyclophosphamide on day 4 and day 1 before infection. The animals were infected via intramuscular injection of an inoculum containing 10⁶ CFU/mL of bacteria in the left thigh. The animals were treated with a single dose of meropenem subcutaneous +/− inhibitor 1 h after inoculation, and animals were either euthanised at the start of treatment or at the study endpoint at 3 h after treatment initiation. Clinical scores were monitored at 1 and 4 hours post inoculation and were in all cases mild or moderate. Thighs were aseptically removed from the animals, homogenised, diluted, and plated for incubation. Bacterial counting was performed after 18-22 h of incubation at 35° C. in ambient air.

### Septicaemia Model of Infection.

Immunocompetent female NMRI mice mice (5-6 weeks old, 26-30 gram, n=5) were inoculated intraperitoneally with 0.5 ml inoculum containing 5×10⁵ CFU suspended in 5% porcine mucin.

The animals were treated with a single dose of meropenem subcutaneous+/−metallo-b-lactamase inhibitor intravenous 1 h after inoculation. Animals were either euthanised at the start of treatment or at the study endpoint at 3 h after treatment initiation. Clinical scores were monitored at 1 and 4 hours post inoculation and were in all cases mild or moderate. Mice were anaesthetised anesthetized with a buprenorphine/tiletamine/zolazepam cocktail and bled, then euthanized and the peritoneum flushed with 2 ml sterile 0.9% NaCl and collected. The blood and/or peritoneal flush were serially diluted and plated for incubation. Bacterial counting was performed after 18-22 h of incubation at 35° C. in ambient air.

TABLE 5
In vivo activity of the metallo-beta-lactamase inhibitors. Dose required to
reduce minim 1 log fold CFU in a thigh and or a sepsis animal model are
defined in ranges A = >100 mg/kg, B = 30-10 mg/kg, C = 5-10 mg/kg.

| Compound | Category |
|---|---|
| Prior Art Compound 76 | A |
| IC-23 | C |
| IC-54 | C |
| IC-18 | C |
| IC-17 | B |
| IC-14 | C |
| IC-56 | C |
| IC-57 | B |
| IC-61 | C |
| IC-60 | C |
| IC-59 | B |
| IC-11 | B |

### REFERENCES

1. Livermore D M, Canton R, Gniadkowski M, Nordmann P, Rossolini G M, Arlet G, et al. CTX-M: changing the face of ESBLs in Europe. J Antimicrob Chemother. 2007; 59(2):165-74.
2. Kumarasamy K K, Toleman M A, Walsh T R, Bagaria J, Butt F, Balakrishnan R, et al. Emergence of a new antibiotic resistance mechanism in India, Pakistan, and the UK: a molecular, biological, and epidemiological study. The Lancet Infectious diseases. 2010; 10(9):597-602.
3. Liu Y Y, Wang Y, Walsh T R, Yi L X, Zhang R, Spencer J, et al. Emergence of plasmid-mediated colistin resistance mechanism MCR-1 in animals and human beings in China: a microbiological and molecular biological study. The Lancet Infectious diseases. 2016; 16(2):161-8.
4. He T, Wang R, Liu D, Walsh T R, Zhang R, Lv Y, et al. Emergence of plasmid-mediated high-level tigecycline resistance genes in animals and humans. Nat Microbiol. 2019; 4(9):1450-6.
5. Yahav D, Giske C G, Gramatniece A, Abodakpi H, Tam V H, Leibovici L. New β-Lactam-β-Lactamase Inhibitor Combinations. Clinical microbiology reviews. 2020; 34(1):e00115-20.
6. Bush K. Past and Present Perspectives on β-Lactamases. Antimicrobial agents and chemotherapy. 2018; 62(10):e01076-18.
7. Rotondo C M, Wright G D. Inhibitors of metallo-β-lactamases. Current Opinion in Microbiology. 2017; 39:96-105.
8. Bonomo R A, Burd E M, Conly J, Limbago B M, Poirel L, Segre J A, et al. Carbapenemase-Producing Organisms: A Global Scourge. Clin Infect Dis. 2018; 66(8):1290-7.
9. Wang D Y, Abboud M I, Markoulides M S, Brem J, Schofield C J. The road to avibactam: the first clinically useful non-beta-lactam working somewhat like a beta-lactam. Future medicinal chemistry. 2016; 8(10):1063-84.
10. Ehmann D E, Jahid H, Ross P L, Gu R-F, Hu J, Kern G, et al. Avibactam is a covalent, reversible, non-β-lactam β-lactamase inhibitor. Proceedings of the National Academy of Sciences. 2012; 109(29):11663-8.
11. Lomovskaya O, Sun D, Rubio-Aparicio D, Nelson K, Tsivkovski R, Griffith D C, et al. Vaborbactam: Spectrum of Beta-Lactamase Inhibition and Impact of Resistance Mechanisms on Activity in Enterobacteriaceae. Antimicrobial agents and chemotherapy. 2017; 61(11).
12. Langley G W, Cain R, Tyrrell J M, Hinchliffe P, Calvopina K, Tooke C L, et al. Profiling interactions of vaborbactam with metallo-β-lactamases. Bioorg Med Chem Lett. 2019; 29(15):1981-4.
13. Papp-Wallace K M, Mack A R, Taracila M A, Bonomo R A. Resistance to Novel β-Lactam-β-Lactamase Inhibitor Combinations: The “Price of Progress”. Infect Dis Clin North Am. 2020.
14. Lohans C T, Brem J, Schofield C J. New Delhi Metallo-β-Lactamase 1 Catalyzes Avibactam and Aztreonam Hydrolysis. Antimicrobial agents and chemotherapy. 2017; 61(12):e01224-17.
15. Meini M R, Llarrull L I, Vila A J. Overcoming differences: The catalytic mechanism of metallo-beta-lactamases. FEBS letters. 2015; 589(22):3419-32.
16. Drawz S M, Bonomo R A. Three decades of beta-lactamase inhibitors. Clinical microbiology reviews. 2010; 23(1):160-201.
17. Davies D T, Leiris S, Sprynski N, Castandet J, Lozano C, Bousquet J, et al. ANT2681: SAR Studies Leading to the Identification of a Metallo-β-lactamase Inhibitor with Potential for Clinical Use in Combination with Meropenem for the Treatment of Infections Caused by NDM-Producing Enterobacteriaceae. ACS Infectious Diseases. 2020; 6(9):2419-30.
18. King A M, Reid-Yu S A, Wang W, King D T, De Pascale G, Strynadka N C, et al. Aspergillomarasmine A overcomes metallo-β-lactamase antibiotic resistance. Nature. 2014; 510(7506):503-6.
19. Liu B, Trout R E L, Chu G H, McGarry D, Jackson R W, Hamrick J C, et al. Discovery of Taniborbactam (VNRX-5133): A Broad-Spectrum Serine- and Metallo-β-lactamase Inhibitor for Carbapenem-Resistant Bacterial Infections. J Med Chem. 2020; 63(6):2789-801.
20. Bush K, Bradford P A. Epidemiology of β-Lactamase-Producing Pathogens. Clinical microbiology reviews. 2020; 33(2).
21. Sauvage E, Terrak M. Glycosyltransferases and Transpeptidases/Penicillin-Binding Proteins: Valuable Targets for New Antibacterials. Antibiotics (Basel). 2016; 5(1).
22. Bush K, Bradford P A. Interplay between β-lactamases and new β-lactamase inhibitors. Nature Reviews Microbiology. 2019; 17(5):295-306.
23. Dik D A, Fisher J F, Mobashery S. Cell-Wall Recycling of the Gram-Negative Bacteria and the Nexus to Antibiotic Resistance. Chem Rev. 2018; 118(12):5952-84.
24. Tipper D J, Strominger J L. Mechanism of action of penicillins: a proposal based on their structural similarity to acyl-D-alanyl-D-alanine. Proceedings of the National Academy of Sciences of the United States of America. 1965; 54(4):1133-41.
25. Karawajczyk A, Orrling K M, de Vlieger J S B, Rijnders T, Tzalis D. The European Lead Factory: A Blueprint for Public-Private Partnerships in Early Drug Discovery. Frontiers in Medicine. 2017; 3(75).

1. A compound of formula II, or a pharmaceutically acceptable salt or solvate thereof, as shown below: 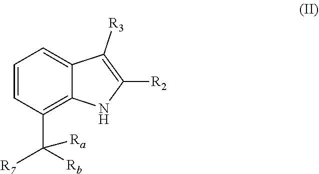
wherein
R₂ is selected from:
ix. —C(O)OH;
x. —C(O)OR_{2A}, wherein R_{2A} is selected from (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
xi. —C(O)NR_{2B}R_{2C}; wherein R_{2B} and R_{2C} are each independently selected from hydrogen, (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
xii. —C(O)NR_{2D}NR_{2B}R_{2E}; wherein R_{2D} is selected from hydrogen or (1-6C)alkyl and R_{2B} and R_{2C} are as defined above;
xiii. tetrazolyl;
xiv. triazolyl;
xv. —B(OR_{2F})(OR_{2G}), wherein R_{2F} and R_{2G} are each independently selected from hydrogen, (1-6C)alkyl or R_{2F} and R_{2G} are linked such that, together with the B and O atoms, they form a 5 or 6-membered heterocyclic ring, which is optionally substituted by (1-2C)alkyl;
xvi. trifluoromethylketone;
and wherein R^{A} is selected from halo, cyano, nitro or a group of the formula: 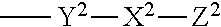
wherein
Y² is absent or a linker group of the formula —[CR^{A1}R^{A2}]ₘ— in which m is an integer selected from 1, 2, 3 or 4, and R^{A1} and R^{A2} are each independently selected from hydrogen or (1-2C)alkyl;
X² is absent or —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —S—, —SO—, —SO₂—, —S(O)₂N(R^{A3})—, or —N(R^{A3})SO₂— wherein R^{A3} is selected from hydrogen or methyl; and
Z² is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z² is optionally further substituted by one or more substituent groups independently selected from oxo, halo, cyano, nitro, hydroxy, carboxy, NR^{A4}R^{A5} (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, heterocyclyl, heterocyclyloxy, heterocyclyl-(1-2C)alkyl, heteroaryl, heteroaryloxy, heteroaryl-(1-2C)alkyl, C(O)NR^{A4}R^{A5} NR^{A4}C(O)R^{A5}, NR^{A4}S(O)₂R^{A5} and S(O)₂NR^{A4}R^{A5}; wherein R^{A4} and R^{A5} are each independently selected from hydrogen, (1-4C)alkyl or (3-6C)cycloalkyl or (3-6C)cycloalkyl(1-2C)alkyl; or R^{A4} and R^{A5} can be linked such that, together with the nitrogen atom to which they are attached, they form a 4-6 membered heterocyclic ring;
and wherein any alkyl, aryl, heterocyclyl or heteroaryl group present in a substituent group on Z² is optionally further substituted by halo, cyano, nitro, hydroxy, caboxy, NR^{A6}R^{A7}, (1-20)alkoxy, or (1-2C)alkyl; wherein R^{A5} and R^{A7} are selected from hydrogen or (1-2C)alkyl;
R₃ is selected from halo, aryl, (4-6C)cycloalkyl, 5- to 12-membered heteroaryl, 5- to 12-membered heterocyclyl, wherein said aryl, (4-6C)cycloalkyl, 5- to 12-membered heteroaryl, 5- to 12-membered heterocyclyl, ring system is optionally substituted by one or more R^{3A};
wherein each R^{3A} is independently halo, oxo, cyano, nitro, hydroxy or a group: 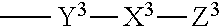
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3}), —S—, —SO—, —SO₂—, —S(O)₂N(R^{B3})—, —C(═NR^{B4})N(R^{B3})—, —C(═O)N(R^{B3})—, —C(═NR^{B4}—, —S(O)(═NR^{B4})N(R^{B3})—, —S(O)(═NR^{B4})— or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
and wherein any alkyl, aryl, heterocyclyl or heteroaryl group present in a substituent group on Z³ is optionally further substituted by halo, cyano, nitro, hydroxy, caboxy, NR^{B7}R^{B8}, (1-20)alkoxy, or (1-2C)alkyl; wherein R^{B7} and R^{B8} are selected from hydrogen or (1-2C)alkyl;
or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
R₇ is a group: 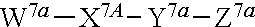
wherein:
W^{7a} is absent or a linker group of the formula —[CR^{7A}R^{7B}]q in which q is an integer selected from 1, 2, 3 or 4, and each occurrence of R^{7A} and R^{7B} is each independently selected from hydrogen or (1-2C)alkyl, and
X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —S—, —SO—, —SO₂—, —S(O)₂N(R^{7C})—, —N(R^{7D})SO₂—, —N(R^{7D})SO₂N(R^{7C})—, —C(NR^{7E})N(R^{7C})—, or —N(R^{7D})C(NR^{7E})N(R^{7C})—, wherein R^{7C}, R^{7D} and R^{7E} are each independently selected from hydrogen or (1-2C)alkyl, wherein any (1-2C)alkyl) is optionally substituted by oxo, halo, cyano, nitro, hydroxy, carboxy, amino, (1-2C)alkoxy; and
Y^{7a} is absent or a linker group of the formula —[CR^{7F}R^{7G}]ᵣ— in which r is an integer selected from 1, 2, 3 or 4, and each occurrence of R^{7F} and R^{7G} is each independently selected from hydrogen or (1-2C)alkyl, and
Z^{7a} is either:
(i) (1-6C)alkyl optionally substituted by oxo, halo, cyano, nitro, hydroxy, carboxy, NR^{7H}R^{7I}, (1-4C)alkoxy; wherein R^{7H} and R^{7I} are each independently selected from hydrogen, (1-4C)alkyl or (3-6C)cycloalkyl or (3-6C)cycloalkyl(1-2C)alkyl
with the proviso that if Z^{7a} is (1-6C)alkyl, then X^{7a} is not absent;
or
ii) (3-8C)cycloalkyl, aryl, 5- to 12-membered heterocyclyl or 5- to 12-membered heteroaryl; wherein each of (3-8C)cycloalkyl, aryl, 5- to 12-membered heterocyclyl, 5- to 12-membered heteroaryl is optionally substituted by:
a) one or more of a substituent group independently selected from oxo, halo, cyano, nitro, hydroxy, carboxy, NR^{7H}R^{7I}, (1-4C)alkoxy, (1-4C)alkyl, (1-4C)alkylamino, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{7H}R^{7I}, NR⁷HC(O)R^{7I}, NR^{7H}S(O)₂R^{7I} and S(O)₂NR^{7H}R^{7I}; wherein R^{7H} and R^{7I} are each independently selected from hydrogen, (1-4C)alkyl or (3-6C)cycloalkyl or (3-6C)cycloalkyl(1-2C)alkyl; or R^{7H} and R^{7I} can be linked such that, together with the nitrogen atom to which they are attached, they form a 4-7 membered heterocyclic ring;
wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, C(O)NR^{7J}R^{7K}, NR^{7J}C(O)R^{7K}, NR^{7J}S(O)₂R^{7K} and S(O)₂NR^{7J}R^{7K}; wherein R^{7J} and R^{7K} are each independently selected from hydrogen or (1-2C)alkyl; and/or
b) one or more of a group R^{Z}; wherein R^{Z} is a group of the formula: 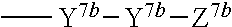
wherein:
X^{7b} is absent or a linker group of the formula —[CR^{7L}R^{7M}]ₓ—in which x is an integer selected from 1, 2, 3 or 4, and each occurrence of R^{7L} and R^{7M} is each independently selected from hydrogen or (1-2C)alkyl, and
Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N})—, —C(NR^{7P})N(R^{7N})—, or —N(R^{7P})C(═NR^{7Q})N(R^{7N})—, wherein R^{7N}, R^{7P} and R^{7Q} are each independently selected from hydrogen or methyl; and
Z^{7b} is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z^{7b} is optionally further substituted by one or more substituent groups independently selected from oxo, halo, cyano, nitro, hydroxy, carboxy, NR^{7R}R^{7S}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{7R}R^{7S}, NR^{7R}C(O)R^{7S}, NR^{7R}S(O)₂R^{7S} and S(O)₂NR^{7R}R^{7S}; wherein R^{7R} and R^{7S} are each independently selected from hydrogen, (1-4C)alkyl or (3-6C)cycloalkyl or (3-6C)cycloalkyl(1-2C)alkyl; or R^{7R} and R^{7S} can be linked such that, together with the nitrogen atom to which they are attached, they form a 4-7 membered heterocyclic ring;
and wherein any alkyl, aryl, heterocyclyl or heteroaryl group present in a substituent group on Z^{7b} is optionally further substituted by halo, cyano, nitro, hydroxy, caboxy, NR^{7T}R^{7U}, (1-2C)alkoxy, or (1-2C)alkyl; wherein R^{7T} and R^{7U} are selected from hydrogen or (1-2C)alkyl;
or R^{7T} and Z^{7U} can be linked such that, together with the nitrogen atom to which they are attached, they form a 4-7 membered heterocyclic ring, which is optionally substituted by oxo, halo, cyano, nitro, hydroxy, carboxy, NR^{7V}R^{7W}, (1-4C)alkoxy, (1-4C)alkyl, (1-4C)aminoalkyl, (3-8C)cycloalkyl, (3-8C)cycloalkyl-(1-3C)alkyl, (1-4C)alkanoyl, (1-4C)alkylsulphonyl, or C(O)NR^{7V}R^{7W}, NR^{7V}C(O)R^{7W}, NR^{7V}S(O)₂R^{7W} and S(O)₂NR^{7V}R^{7W}; wherein R^{7V} and R^{7W} are each independently selected from hydrogen or (1-2C)alkyl; and
Rₐ is hydrogen or (1-4C)alkyl; and
R_{b} is selected from hydrogen or (1-4C)alkyl;
with the proviso that only one of Rₐ and R_{b} can be hydrogen.

2. A compound of formula I, or a pharmaceutically acceptable salt or solvate thereof, as shown below: 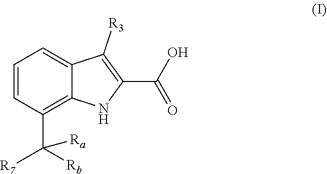
wherein
R₃ is selected from halo, aryl, (4-6C)cycloalkyl, 5- to 12-membered heteroaryl, 5- to 12-membered heterocyclyl, wherein said aryl, (4-6C)cycloalkyl, 5- to 12-membered heteroaryl, 5- to 12-membered heterocyclyl, ring system is optionally substituted by one or more R^{3A} wherein each R^{3A} is independently halo, oxo, cyano, nitro, hydroxy or a group: 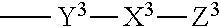
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
and wherein any alkyl, aryl, heterocyclyl or heteroaryl group present in a substituent group on Z³ is optionally further substituted by halo, cyano, nitro, hydroxy, caboxy, NR^{B7}R^{B8}, (1-2C)alkoxy, or (1-2C)alkyl; wherein R^{B7} and R^{B8} are selected from hydrogen or (1-2C)alkyl;
or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
R₇ is a group:
$${W}^{7 a} - {X}^{7 a} - {Y}^{7 a} - {Z}^{7 a}$$
wherein:
W^{7a} is absent or a linker group of the formula —[CR^{7A}R^{7B}]q in which q is an integer selected from 1, 2, 3 or 4, and each occurrence of R^{7A} and R^{7B} is each independently selected from hydrogen or (1-2C)alkyl, and
X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —S—, —SO—, —SO₂—, —S(O)₂N(R^{7C})—, —N(R^{7D})SO₂—, —N(R^{7D})SO₂N(R^{7C})—, —C(NR^{7E})N(R^{7C})—, or —N(R^{7D})C(NR^{7E})N(R^{7C})—, wherein R^{7C}, R^{7D} and R^{7E} are each independently selected from hydrogen or (1-2C)alkyl, wherein any (1-2C)alkyl) is optionally substituted by oxo, halo, cyano, nitro, hydroxy, carboxy, amino, (1-2C)alkoxy; and
Y⁷a is absent or a linker group of the formula —[CR^{7F}R^{7G}]ᵣ— in which r is an integer selected from 1, 2, 3 or 4, and each occurrence of R^{7F} and R^{7G} is each independently selected from hydrogen or (1-2C)alkyl, and
Z^{7a} is either:
(i) (1-6C)alkyl optionally substituted by oxo, halo, cyano, nitro, hydroxy, carboxy, NR^{7H}R^{7I}, (1-4C)alkoxy; wherein R^{7H} and R^{7I} are each independently selected from hydrogen, (1-4C)alkyl or (3-6C)cycloalkyl or (3-6C)cycloalkyl(1-2C)alkyl
with the proviso that if Z^{7a} is (1-6C)alkyl, then X^{7a} is not absent;
or
ii) (3-8C)cycloalkyl, aryl, 5- to 12-membered heterocyclyl or 5- to 12-membered heteroaryl; wherein each of (3-8C)cycloalkyl, aryl, 5- to 12-membered heterocyclyl, 5- to 12-membered heteroaryl is optionally substituted by:
a) one or more of a substituent group independently selected from oxo, halo, cyano, nitro, hydroxy, carboxy, NR^{7H}R^{7I}, (1-4C)alkoxy, (1-4C)alkyl, (1-4C)alkylamino, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{7H}R^{7I}, NR^{7H}C(O)R^{7I}, NR^{7H}S(O)₂R^{7I} and S(O)₂NR^{7H}R^{7I}; wherein R^{7H} and R^{7I} are each independently selected from hydrogen, (1-4C)alkyl or (3-6C)cycloalkyl or (3-6C)cycloalkyl(1-2C)alkyl; or R^{7H} and R^{7I} can be linked such that, together with the nitrogen atom to which they are attached, they form a 4-7 membered heterocyclic ring;
wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, C(O)NR^{7J}R^{7K}, NR^{7J}C(O)R^{7K}, NR^{7J}S(O)₂R^{7K} and S(O)₂NR^{7J}R^{7K}; wherein R^{7J} and R^{7K} are each independently selected from hydrogen or (1-2C)alkyl; and/or
b) one or more of a group R^{Z}; wherein R^{Z} is a group of the formula:
$$- {X}^{7 b} - {Y}^{7 b} - {Z}^{7 b}$$
wherein:
X^{7b} is absent or a linker group of the formula —[CR^{7L}R^{7M}]ₓ— in which x is an integer selected from 1, 2, 3 or 4, and each occurrence of R^{7L} and R^{7M} is each independently selected from hydrogen or (1-2C)alkyl, and
Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N})—, —C(NR^{7P})N(R^{7N})—, or —N(R^{7P})C(═NR^{7Q})N(R^{7N})—, wherein R^{7N}, R^{7P} and R^{7Q} are each independently selected from hydrogen or methyl; and
Z^{7b} is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z^{7b} is optionally further substituted by one or more substituent groups independently selected from oxo, halo, cyano, nitro, hydroxy, carboxy, NR^{7R}R^{7S}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{7R}R^{7S}, NR⁷RC(O)R^{7S}, NR^{7R}S(O)₂R^{7S} and S(O)₂NR^{7R}R^{7S}; wherein R^{7R} and R^{7S} are each independently selected from hydrogen, (1-4C)alkyl or (3-6C)cycloalkyl or (3-6C)cycloalkyl(1-2C)alkyl; or R^{7R} and R^{7S} can be linked such that, together with the nitrogen atom to which they are attached, they form a 4-7 membered heterocyclic ring;
and wherein any alkyl, aryl, heterocyclyl or heteroaryl group present in a substituent group on Z^{7b} is optionally further substituted by halo, cyano, nitro, hydroxy, caboxy, NR^{7T}R^{7U}, (1-2C)alkoxy, or (1-2C)alkyl; wherein R^{7T} and R^{7U} are selected from hydrogen or (1-2C)alkyl;
or R^{7T} and Z^{7U} can be linked such that, together with the nitrogen atom to which they are attached, they form a 4-7 membered heterocyclic ring, which is optionally substituted by oxo, halo, cyano, nitro, hydroxy, carboxy, NR^{7V}R^{7W}, (1-4C)alkoxy, (1-4C)alkyl, (1-4C)aminoalkyl, (3-8C)cycloalkyl, (3-8C)cycloalkyl-(1-3C)alkyl, (1-4C)alkanoyl, (1-4C)alkylsulphonyl, or C(O)NR^{7V}R^{7W}, NR^{7V}C(O)R^{7W}, NR^{7V}S(O)₂R^{7W} and S(O)₂NR^{7V}R^{7W}; wherein R^{7V} and R^{7W} are each independently selected from hydrogen or (1-2C)alkyl; and
Rₐ is hydrogen or (1-4C)alkyl; and
R_{b} is selected from hydrogen or (1-4C)alkyl;
with the proviso that only one of Rₐ and R_{b} can be hydrogen.

3. A compound according to claim 1 or claim 2, wherein R₃ is selected from halo, phenyl, 5- or 6-membered heteroaryl or 5- or 6-membered heterocyclyl, fused heteroaryl or fused heterocyclyl; wherein said phenyl, 5- or 6-membered heteroaryl, 5- or 6-membered heterocyclyl, fused heteroaryl or fused heterocyclyl is optionally substituted by one or more R^{3A};
wherein R^{3A} is halo, cyano, nitro, oxo, hydroxy or a group:
$$- {Y}^{3} - {X}^{3} - {Z}^{3}$$
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, —C(═NR^{B4})N(R^{B3})—, —C(═O)N(R^{B3})—, —C(═NR^{B4}—, —S(O)(═NR^{B4})N(R^{B3})—, —S(O)(═NR^{B4})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-80)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
and wherein any alkyl, aryl, heterocyclyl or heteroaryl group present in a substituent group on Z³ is optionally further substituted by halo, cyano, nitro, hydroxy, caboxy, NR^{B7}R^{B8}, (1-20)alkoxy, or (1-20)alkyl; wherein R^{B7} and R^{B8} are selected from hydrogen or (1-20)alkyl;
or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
with the proviso that R^{3A} is not hydrogen.

4. A compound according to claim 1, 2 or 3, wherein R₃ is selected from halo, phenyl, 6-membered heteroaryl or 6-membered heterocyclyl, 8-10 membered fused heteroaryl or 8-10 membered fused heterocyclyl wherein said hydrogen, halo, phenyl, 6-membered heteroaryl or 6-membered heterocyclyl, 8-10 membered fused heteroaryl or 8-10 membered fused heterocyclyl is optionally substituted by one or more R^{3A} wherein each R^{3A} is independently selected from halo, cyano, nitro, oxo, hydroxy or a group:
$$- {Y}^{3} - {X}^{3} - {Z}^{3}$$
wherein
Y³ is absent or a linker group of the formula —[CR^{B1}R^{B2}]ₙ— in which n is an integer selected from 1 or 2, and R^{B1} and R^{B2} are each independently selected from hydrogen or (1-2C)alkyl;
X³ is selected from —SO—, —SO₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})SO₂— wherein R^{B3} and R^{B4} are each independently selected from hydrogen or methyl; and
Z³ is hydrogen, (1-6C)alkyl or heterocyclyl;
and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryloxy, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring.

5. A compound according to any one of claims 1 to 4, wherein R₃ is selected from: 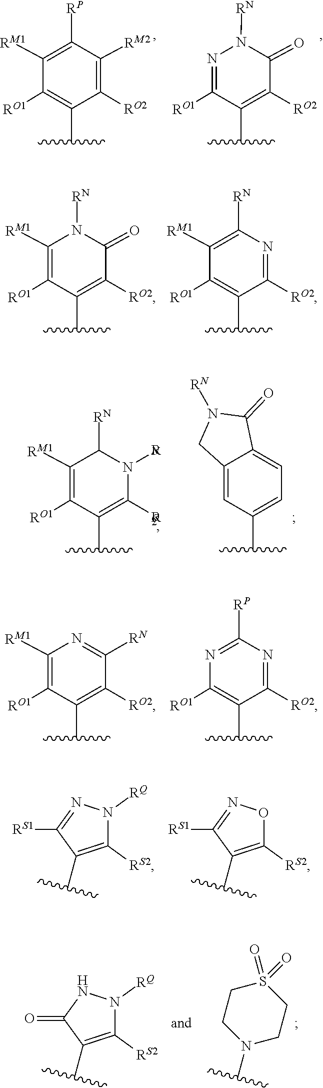
wherein:
R^{N} and R^{Q} are either hydrogen or methyl;
R^{O1} and R^{O2} are each independently selected from hydrogen or fluoro;
R^{M1} and R^{M2} are each independently selected from hydrogen, halo, cyano, hydroxy, (1-2C)alkyl, (1-2C)alkoxy; (1-2C)haloalkyl, (1-2C)haloalkoxy; and
R^{P} is a group: 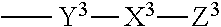
wherein
Y³ is absent or a linker group of the formula —[CR^{B1}R^{B2}]ₙ— in which n is an integer selected from 1 or 2, and R^{B1} and R^{B2} are each independently selected from hydrogen or methyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})—, or —N(R^{B4})SO₂— wherein R^{B3} and R^{B4} are each independently selected from hydrogen or methyl; and
Z³ is hydrogen, (1-6C)alkyl, (3-6C)cycloalkyl, (3-6C)cycloalkenyl, heteroaryl or heterocyclyl;
R^{S1} and R^{S2} are each independently selected from methyl, hydroxy or fluoro.

6. A compound according to any one of claims 1 to 5, wherein R₃ is a group: 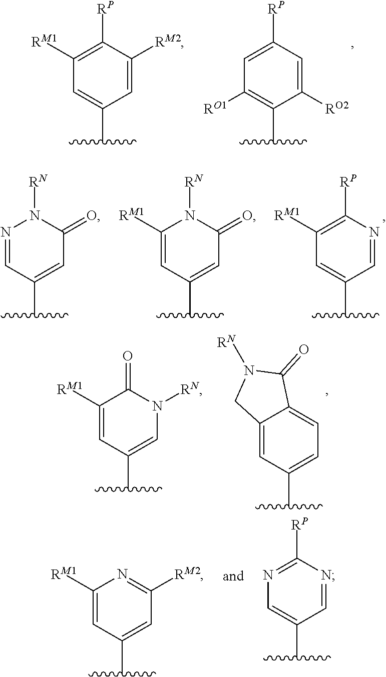
wherein:
R^{N} is either hydrogen or methyl;
R^{O1} and R^{O2} are each independently selected from hydrogen or fluoro;
R^{M1} and R^{M2} are each independently selected from hydrogen, halo, cyano, hydroxy, (1-2C)alkyl, (1-2C)alkoxy; (1-2C)haloalkyl, (1-2C)haloalkoxy; and
R^{P} is a group:
—Y³—X³—Z³
wherein
Y³ is absent or a linker group of the formula —[CR^{B1}R^{B2}]ₙ— in which n is an integer selected from 1 or 2, and R^{B1} and R^{B2} are each independently selected from hydrogen or methyl;
X³ is selected from or —N(R^{B3})—, —N(R^{B4})—C(O)—, —N(R^{B4})—C(O)O—, —C(O)—N(R^{B3})—, —N(R^{B4})C(O)N(R^{B3}), —S—, —S(O), —S(O)₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})S(O)₂— wherein R^{B3} and R^{B4} are each independently selected from hydrogen or methyl; and
Z³ is hydrogen, (1-2C)alkyl, (3-6C)cycloalkyl, or heterocyclyl.

7. A compound according to any one of claims 1 to 6, wherein R₃ is a group: 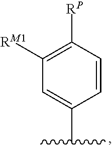
wherein
R^{P} is a group: 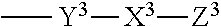
wherein
Y³ is absent or a linker group of the formula —[CR^{B1}R^{B2}]ₙ— in which n is an integer selected from 1 or 2, and R^{B1} and R^{B2} are each independently selected from hydrogen or methyl;
X³ is selected from or —N(R^{B3})—, —N(R^{B4})—C(O)—, —N(R^{B4})—C(O)O—, —C(O)—N(R^{B3})—, —N(R^{B4})C(O)N(R^{B3})—, —S—, —S(O)—, —S(O)₂—, —S(O)₂N(R^{B3})—, —C(═NR^{B4})N(R^{B3})—, —C(═O)N(R^{B3})—, —C(═NR^{B4})—, —S(O)(═NR^{B4})N(R^{B3})—, —S(O)(═NR^{B4})— or —N(R^{B4})S(O)₂— wherein R^{B3} and R^{B4} are each independently selected from hydrogen or methyl; and
Z³ is (1-2C)alkyl or heterocyclyl;
R^{BM} is selected from halo, cyano, methoxy or hydroxy.

8. A compound according to any one of claims 1 to 6, wherein R₃ is selected from:

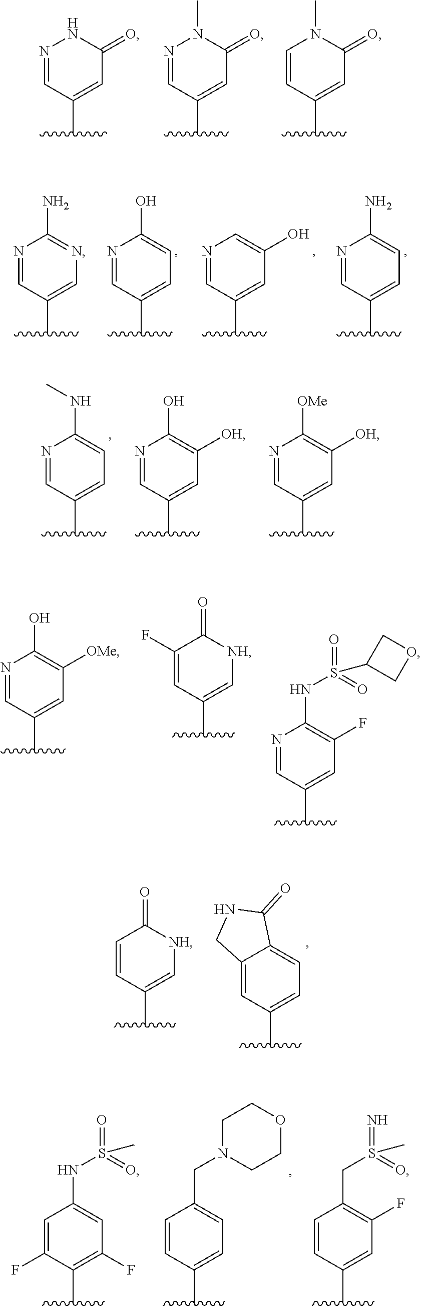

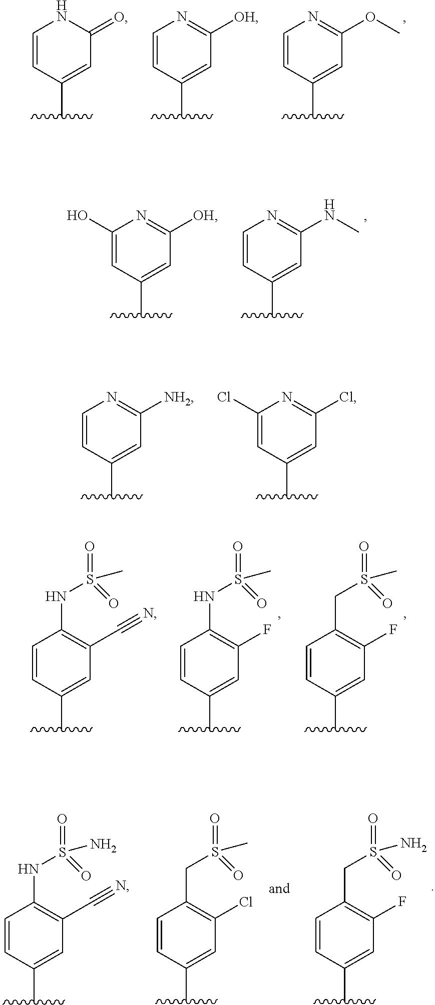

9. A compound according to any one of claims 1 to 8, wherein R₃ is selected from:

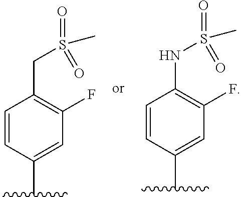 [SMILES: *c1ccc(CS(C)(=O)=O)c(F)c1 ; *c1ccc(NS(C)(=O)=O)c(F)c1]

10. A compound according to any one of the preceding claims, wherein R₇ is a group: 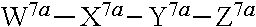
wherein:
W^{7a} is absent or a linker group of the formula —[CR^{7A}R^{7B}]q in which q is an integer selected from 1 or 2, and each occurrence of R^{7A} and R^{7B} is each independently selected from hydrogen or methyl, and
X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —N(R^{7D})SO₂—, —S—C(NR^{7E})N(R^{7C})—, or —N(R^{7D})C(═NR^{7E})N(R^{7C})—, wherein R^{7C}, R^{7D} and R^{7E} are each independently selected from hydrogen or methyl; and
Y^{7a} is absent or a linker group of the formula —[CR^{7F}R^{7G}]ᵣ in which r is an integer selected from 1 or 2, and each occurrence of R^{7F} and R^{7G} is each independently selected from hydrogen or methyl, and
Z^{7a} is (3-8C)cycloalkyl, phenyl, 5- or 6-membered heterocyclyl, a fused 8-12 membered heterocyclic or heteroaryl ring system, 5- or 6-membered heteroaryl, a spirocyclic 8-10 membered heterocyclic ring system, a bridged (3-8C)cycloalkyl, or a bridged heterocyclic ring system, each of which is optionally substituted by:
a) one or more of a substituent group independently selected from oxo, halo, cyano, nitro, hydroxy, carboxy, NR^{7H}R^{7I}, (1-40)alkoxy, (1-4C)alkyl, (1-4C)alkylamino, heterocyclyl-(1-2C)alkyl, heteroaryl, or heteroaryl-(1-2C)alkyl;
wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, C(O) NR^{7J}R^{7K}, NR^{7J}C(O)R^{7K}, NR^{7J}S(O)₂R^{7K} and S(O)₂NR^{7J}R^{7K}; wherein R^{7J} and R^{7K} are each independently selected from hydrogen or (1-2C)alkyl; and/or
b) one or more of a group R^{Z}; wherein R^{Z} is a group of the formula: 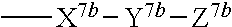
wherein:
X^{7b} is absent or a linker group of the formula —[CR^{7L}R^{7M}]ₓ in which x is an integer selected from 1, 2 or 3, and each occurrence of R^{7L} and R^{7M} is each independently selected from hydrogen or (1-2C)alkyl, and
Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N})—, —C(NR^{7P})N(R^{7N})—, or —N(R^{7P})C(═NR^{7Q})N(R^{7N})—, wherein R^{7N}, R^{7P} and R^{7Q} are each independently selected from hydrogen or methyl; and
Z^{7b} is hydrogen, (1-6C)alkyl, aryl, (3-6C)cycloalkyl, heteroaryl or heterocyclyl;
and wherein Z^{7b} is optionally further substituted by one or more substituent groups independently selected from oxo, halo, cyano, nitro, hydroxy, carboxy, NR^{7R}R^{7S}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl; wherein R^{7R} and R^{7S} are each independently selected from hydrogen or (1-4C)alkyl;
and wherein any alkyl, aryl, heterocyclyl or heteroaryl group present in a substituent group on Z^{7b} is optionally further substituted by halo, cyano, nitro, hydroxy, caboxy, NR^{7T}R^{7U}, (1-2C)alkoxy, or (1-2C)alkyl; wherein R^{7T} and R^{7U} are selected from hydrogen or (1-2C)alkyl.

11. A compound according to any one of the preceding claims, wherein R₇ is a group: 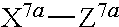
wherein
X^{7a} is absent or —O—, —C(O)—, —C(O)O—, —OC(O)—, —OC(O)N(R^{7C})—, —CH(OR^{7C})—, —N(R^{7C})—, —N(R^{7D})—C(O)—, —N(R^{7D})—C(O)O—, —C(O)—N(R^{7C})—, —N(R^{7D})C(O)N(R^{7C})—, —N(R^{7D})SO₂—, —S—C(NR^{7E})N(R^{7C})—, or —N(R^{7D})C(NR^{7E})N(R^{7C})—, wherein R^{7C}, R^{7D} and R^{7E} are each independently selected from hydrogen or methyl; and
Z^{7a} is (3-8C)cycloalkyl, phenyl, 5- or 6-membered heterocyclyl, a fused heterocyclic ring system, 5- or 6-membered heteroaryl or a spirocyclic heterocyclic ring system, each of which is optionally substituted by:
a) one or more of a substituent group independently selected from oxo, halo, cyano, nitro, hydroxy, carboxy, NR^{7H}R^{7I}, (1-4C)alkoxy, (1-4C)alkyl, (1-4C)alkylamino, heterocyclyl-(1-2C)alkyl, heteroaryl, or heteroaryl-(1-2C)alkyl;
wherein any alkyl, cycloalkyl, heterocyclyl, aryl or heteroaryl is optionally further substituted with one or more substituent groups independently selected from oxo, halo, cyano, nitro, hydroxy, carboxy, NR^{7J}R^{7K}, C(O) NR^{7J}R^{7K}, NR^{7J}C(O)R^{7K}, NR^{7J}S(O)₂R^{7K} and S(O)₂NR^{7J}R^{7K}; wherein R^{7J} and R^{7K} are each independently selected from hydrogen or (1-2C)alkyl; and/or
b) a group R^{Z}, having the formula: 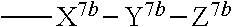
wherein:
X^{7b} is absent or a linker group of the formula —[CR^{7L}R^{7M}]ₓ— in which x is an integer selected from 1, 2 or 3, and each occurrence of R^{7L} and R^{7M} is each independently selected from hydrogen or (1-2C)alkyl, and
Y^{7b} is absent or —O—, —C(O)—, —C(O)O—, —OC(O)—, —CH(OR^{7N})—, —N(R^{7N})—, —N(R^{7P})—C(O)—, —N(R^{7P})—C(O)O—, —C(O)—N(R^{7N})—, —N(R^{7P})C(O)N(R^{7N})—, —S—, —SO—, —SO₂—, —S(O)₂N(R^{7N})—, —N(R^{7P})SO₂—, —N(R^{7P})SO₂N(R^{7N})—, —C(NR^{7P})N(R^{7N})—, or —N(R^{7P})C(═NR^{7L})N(R^{7N})—, wherein R^{7N}, R^{7P} and R^{7Q} are each independently selected from hydrogen or methyl; and
Z^{7b} is hydrogen, (1-6C)alkyl, (2-6C)alkenyl, (2-6C)alkynyl, aryl, (3-6C)cycloalkyl, (3-6C)cycloalkenyl, 5- or 6 membered heteroaryl or 4- to 6-membered heterocyclyl; and
and wherein Z^{7b} is optionally further substituted by one or more substituent groups independently selected from oxo, halo, cyano, nitro, hydroxy, carboxy, NR^{7R}R^{7S}, (1-40)alkoxy, (1-40)alkyl; wherein R^{7R} and R^{7S} are each independently selected from hydrogen or (1-2C)alkyl.

12. A compound according to any one of the preceding claims, wherein R₇ is a group:
—Z^{7a}
wherein
Z^{7a} is 5-membered heterocyclyl, 5- or 6-membered heteroaryl or a spirocyclic heterocyclic ring system;
each of which is optionally substituted by one or more of a substituent group independently selected from oxo or NR^{7H}R^{7I}, wherein R^{7H} and R^{7I} are independently selected from hydrogen or methyl;
and/or
Z^{7a} is optionally substituted by one or more R^{Z}, wherein R^{Z} has the formula: 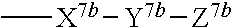
wherein:
X^{7b} is absent or a linker group of the formula —[CR^{7L}R^{7M}]ₓ— in which x is an integer selected from 1, 2 or 3, and each occurrence of R^{7L} and R^{7M} is each independently selected from hydrogen or methyl, and
Y^{7b} is absent or —N(R^{7N})—, —N(R^{7P})—C(O)—, —N(R^{7P})—C(O)O—, —C(O)—N(R^{7N})—, —N(R^{7P})C(O)N(R^{7N})—, —N(R^{7P})SO₂N(R^{7N})—, —C(NR^{7P})N(R^{7N})—, or —N(R^{7P})C(═NR^{7Q})N(R^{7N})—, wherein R^{7P}, R^{7P} and R^{7Q} are each independently selected from hydrogen or methyl; and
Z^{7b} is hydrogen, (1-4C)alkyl, (3-6C)cycloalkyl, or 4 to 6 membered heterocyclyl; and
and wherein Z^{7b} is optionally further substituted by one or more substituent groups independently selected from oxo, halo, cyano, NR^{7R}R^{7S}, (1-2C)alkoxy, (1-2C)alkyl; wherein R^{7R} and R^{7S} are each independently selected from hydrogen or methyl.

13. A compound according to any one of claims 1 to 12, wherein R₇ is selected from a group of the formula:
wherein R^{Z} is as defined in any one of the preceding claims.

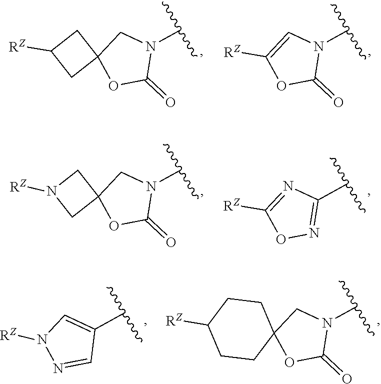

14. A compound according to any one of claims 1 to 13, wherein R₇ is selected from a group of the formula:

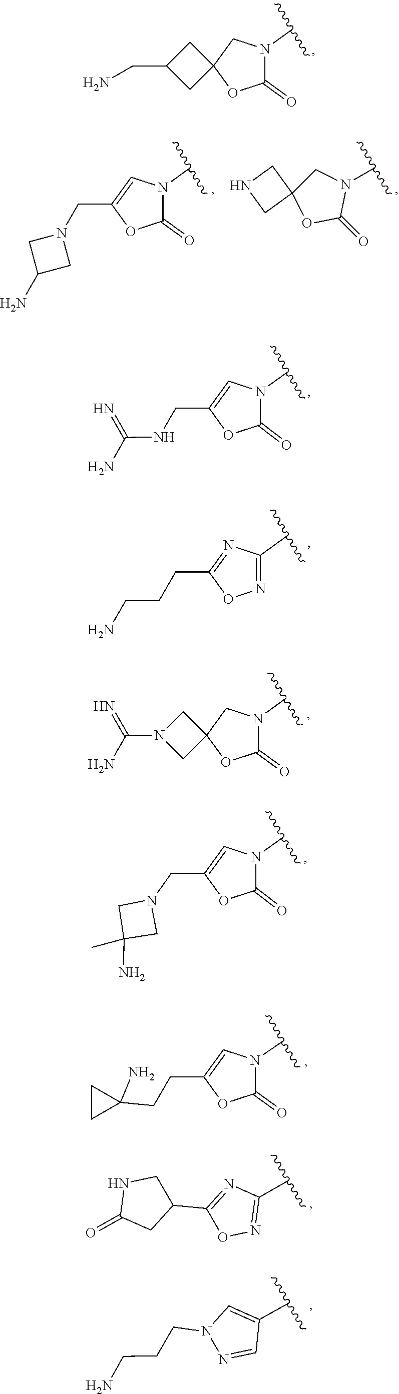

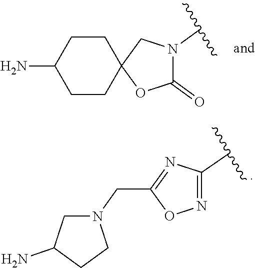 [SMILES: *N1CC2(CCC(N)CC2)OC1=O ; *c1noc(CN2CCC(N)C2)n1]

15. A compound according to any one of claims 1 to 14, wherein R₇ is selected from a group of the formula:

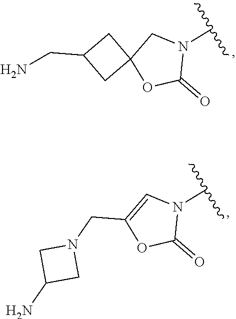 [SMILES: *N1CC2(CC(CN)C2)OC1=O ; *n1cc(CN2CC(N)C2)oc1=O]

16. A compound according to any one of the preceding claims, wherein the compound is a compound according to Formula Ia: 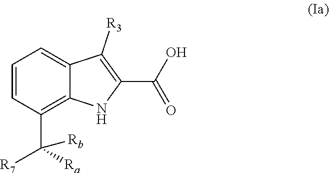
wherein R₃, R₇, Rₐ and R_{b} are as defined in any one of the preceding claims.

17. A compound according to any one of the preceding claims, wherein the compound is a compound according to Formula Id: 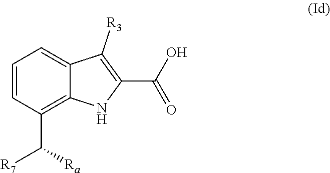
wherein R₃, R₇ and Rₐ are as defined in any one of the preceding claims.

18. A compound according to any one of the preceding claims, wherein the compound is a compound according to Formula If: 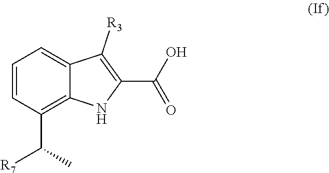
wherein R₃ and R₇ are as defined in any one of the preceding claims.

19. A compound according to any one of the preceding claims, wherein the compound is a compound according to Formula Ii or Ij: 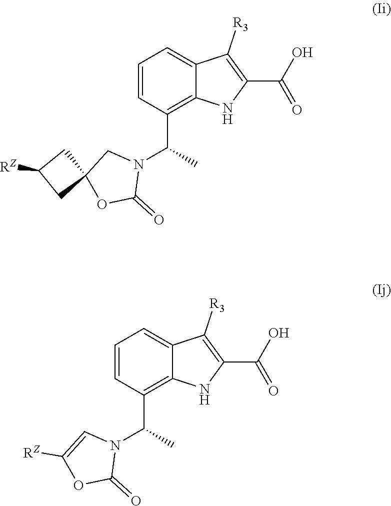
wherein R₃ and R^{Z} are as defined in any one of the preceding claims.

20. A compound according to any one of claims 1 and 3-19, wherein R₂ is selected from —C(O)OH and tetrazolyl.

21. A compound or a pharmaceutically acceptable salt or solvate thereof, selected from:
3-(3,5-Dichlorophenyl)-7-(1-(phenylsulfonamido)ethyl)-1H-indole-2-carboxylic acid
3-(3,5-Dichlorophenyl)-7-(1-(methylsulfonamido)ethyl)-1H-indole-2-carboxylic acid
3-(3-Fluoro-4-((methylsulfonyl)methyl)phenyl)-7-(1-(1-methyl-1H-pyrazol-4-yl)ethyl)-1H-indole-2-carboxylic acid
3-(3-Fluoro-4-((methylsulfonyl)methyl)phenyl)-7-(1-(thiophen-2-yl)ethyl)-1H-indole-2-carboxylic acid
7-(1-(1-(3-Aminopropyl)-1H-pyrazol-4-yl)ethyl)-3-(3-fluoro-4-((methylsulfonyl) methyl)phenyl)-1H-indole-2-carboxylic acid
3-(3-Fluoro-4-((methylsulfonyl)methyl)phenyl)-7-(1-(2-oxo-2H-pyran-3-yl)ethyl)-1H-indole-2-carboxylic acid
7-(1-(2-Chlorothiazol-5-yl)ethyl)-3-(3-fluoro-4-((methylsulfonyl)methyl)phenyl)-1H-indole-2-carboxylic acid
3-(3-Fluoro-4-((methylsulfonyl)methyl)phenyl)-7-(1-(4-((sulfamoylamino)methyl)-1H-1,2,3-triazol-1-yl)ethyl)-1H-indole-2-carboxylic acid
7-(1-(2H-Tetrazol-5-yl)ethyl)-3-(3-fluoro-4-((methylsulfonyl)methyl)phenyl)-1H-indole-2-carboxylic acid
7-[1-[4-(3-Aminopropyl)triazol-1-yl]ethyl]-3-[3-chloro-4-(methylsulfonylmethyl)phenyl]-1H-indole-2-carboxylic acid
7-(1-((4-(Aminomethyl)benzoyl)amino)ethyl)-3-(6-(morpholin-4-ylmethyl)pyridin-3-yl)-1H-indole-2-carboxylic acid
3-(3-Fluoro-4-((methylsulfonyl)methyl)phenyl)-7-(1-(piperazin-1-yl)ethyl)-1H-indole-2-carboxylic acid
7-[1-[3-(3-Aminopropyl)-2,5-dioxo-imidazolidin-1-yl]ethyl]-3-[3-fluoro-4-(methylsulfonylmethyl)phenyl]-1H-indole-2-carboxylic acid
7-((S)-1-((2S,4r)-2-(Aminomethyl)-6-oxo-5-oxa-7-azaspiro[3.4]octan-7-yl)ethyl)-3-(3-fluoro-4-(methylsulfonamido)phenyl)-1H-indole-2-carboxylic acid
7-((S)-1-((2S,4r)-2-(aminomethyl)-6-oxo-5-oxa-7-azaspiro[3.4]octan-7-yl)ethyl)-3-(6-oxo-1,6-dihydropyridin-3-yl)-1H-indole-2-carboxylic acid
7-((1S)-1-(2-(aminomethyl)-6-oxo-5-oxa-7-azaspiro[3.4]octan-7-yl)ethyl)-3-(3-cyano-4-(methylsulfonamido)phenyl)-1H-indole-2-carboxylic acid
7-((1S)-1-(2-(aminomethyl)-6-oxo-5-oxa-7-azaspiro[3.4]octan-7-yl)ethyl)-3-(2-methoxypyridin-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(2-hydroxypyridin-4-yl)-1H-indole-2-carboxylic acid
7-((1S)-1-(2-(aminomethyl)-6-oxo-5-oxa-7-azaspiro[3.4]octan-7-yl)ethyl)-3-(3-fluoro-4-((S-methylsulfonimidoyl)methyl)phenyl)-1H-indole-2-carboxylic acid
7-((1S)-1-(2-(aminomethyl)-6-oxo-5-oxa-7-azaspiro[3.4]octan-7-yl)ethyl)-3-(2-methoxypyridin-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(2,6-difluoro-4-(methylsulfonamido)phenyl)-1H-indole-2-carboxylic acid
7-((S)-1-((2R,4s)-2-(aminomethyl)-6-oxo-5-oxa-7-azaspiro[3.4]octan-7-yl)ethyl)-3-(1-oxoisoindolin-5-yl)-1H-indole-2-carboxylic acid
7-((S)-1-((2S,4r)-2-(aminomethyl)-6-oxo-5-oxa-7-azaspiro[3.4]octan-7-yl)ethyl)-3-(5-hydroxypyridin-3-yl)-1H-indole-2-carboxylic acid
7-((S)-1-((2S,4r)-2-(aminomethyl)-6-oxo-5-oxa-7-azaspiro[3.4]octan-7-yl)ethyl)-3-(3-fluoro-4-(oxetane-3-sulfonamido)phenyl)-1H-indole-2-carboxylic acid
7-((S)-1-((2S,4r)-2-(aminomethyl)-6-oxo-5-oxa-7-azaspiro[3.4]octan-7-yl)ethyl)-3-(6-hydroxy-5-methoxypyridin-3-yl)-1H-indole-2-carboxylic acid
7-((1S)-1-(2-(aminomethyl)-6-oxo-5-oxa-7-azaspiro[3.4]octan-7-yl)ethyl)-3-(5-fluoro-6-oxo-1,6-dihydropyridin-3-yl)-1H-indole-2-carboxylic acid
(S)-7-(1-(2-(aminomethyl)-6-oxo-5-oxa-7-azaspiro[3.4]octan-7-yl)ethyl)-3-(5-hydroxy-6-methoxypyridin-3-yl)-1H-indole-2-carboxylic acid
7-((S)-1-((2S,4r)-2-(aminomethyl)-6-oxo-5-oxa-7-azaspiro[3.4]octan-7-yl)ethyl)-3-(1-methyl-6-oxo-1,6-dihydropyridazin-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(5,6-dihydroxypyridin-3-yl)-1H-indole-2-carboxylic acid
3-(2,6-dihydroxypyridin-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
3-(1-methyl-2-oxo-1,2-dihydropyridin-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
3-(6-oxo-1,6-dihydropyridazin-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
3-(2-Aminopyridin-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
3-[2-(methylamino)pyridin-4-yl]-7-[(1S)-1-[(2r,4r)-2-(aminomethyl)-6-oxo-5-oxa-7-azaspiro[3.4]octan-7-yl]ethyl]-1H-indole-2-carboxylic acid
3-[6-(methylamino)pyridin-3-yl]-7-[(1S)-1-[(2r,4r)-2-(aminomethyl)-6-oxo-5-oxa-7-azaspiro[3.4]octan-7-yl]ethyl]-1H-indole-2-carboxylic acid
3-(6-aminopyridin-3-yl)-7-[(1S)-1-[(2r,4r)-2-(aminomethyl)-6-oxo-5-oxa-7-azaspiro[3.4]octan-7-yl]ethyl]-1H-indole-2-carboxylic acid
3-(pyridin-4-yl)-7-[(1S)-1-[(2r,4r)-2-(aminomethyl)-6-oxo-5-oxa-7-azaspiro[3.4]octan 7-yl]ethyl]-1H-indole-2-carboxylic acid
3-(pyridin-3-yl)-7-[(1S)-1-[(2r,4r)-2-(aminomethyl)-6-oxo-5-oxa-7-azaspiro[3.4]octan-7-yl]ethyl]-1H-indole-2-carboxylic acid
7-((S)-1-((2S,4r)-2-(aminomethyl)-6-oxo-5-oxa-7-azaspiro[3.4]octan-7-yl)ethyl)-3-(1,1-dioxidothiomorpholino)-1H-indole-2-carboxylic acid
7-((S)-1-((2S,4r)-2-(aminomethyl)-6-oxo-5-oxa-7-azaspiro[3.4]octan-7-yl)ethyl)-3-(2-oxoindolin-5-yl)-1H-indole-2-carboxylic acid
7-((S)-1-((2S,4r)-2-(aminomethyl)-6-oxo-5-oxa-7-azaspiro[3.4]octan-7-yl)ethyl)-3-(2-aminopyrimidin-5-yl)-1H-indole-2-carboxylic acid
7-((S)-1-((2S,4r)-2-(aminomethyl)-6-oxo-5-oxa-7-azaspiro[3.4]octan-7-yl)ethyl)-3-(1H-pyrazol-4-yl)-1H-indole-2-carboxylic acid
3-(1-methyl-1H-pyrazol-4-yl)-7-[(1S)-1-[(2r,4r)-2-(aminomethyl)-6-oxo-5-oxa-7-azaspiro[3.4]octan-7-yl]ethyl]-1H-indole-2-carboxylic acid
7-((S)-1-((2S,4r)-2-(aminomethyl)-6-oxo-5-oxa-7-azaspiro[3.4]octan-7-yl)ethyl)-3-(1-(cyanomethyl)-1H-pyrazol-4-yl)-1H-indole-2-carboxylic acid
3-(1-(2-amino-2-iminoethyl)-1H-pyrazol-4-yl)-7-((S)-1-((2S,4r)-2-(aminomethyl)-6-oxo-5-oxa-7-azaspiro[3.4]octan-7-yl)ethyl)-1H-indole-2-carboxylic acid
3-(1-(2-amino-2-oxoethyl)-1H-pyrazol-4-yl)-7-((S)-1-((2S,4r)-2-(aminomethyl)-6-oxo-5-oxa-7-azaspiro[3.4]octan-7-yl)ethyl)-1H-indole-2-carboxylic acid
7-((S)-1-((2S,4r)-2-(aminomethyl)-6-oxo-5-oxa-7-azaspiro[3.4]octan-7-yl)ethyl)-3-(isoxazol-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(3,5-dimethyl-1H-pyrazol-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(3-oxo-2,3-dihydro-1H-pyrazol-4-yl)-1H-indole-2-carboxylic acid
7-[(1S)-1-{5-[(3-Aminoazetidin-1-yl)methyl]-2-oxo-2,3-dihydro-1,3-oxazol-3-yl}ethyl]-3-(2,6-difluoro-4-methanesulfonamidophenyl)-1H-indole-2-carboxylic acid
7-[(1S)-1-{5-[(3-aminoazetidin-1-yl)methyl]-2-oxo-2,3-dihydro-1, 3-oxazol-3-yl}ethyl]-3-{6-[(morpholin-4-yl)methyl]pyridin-3-yl}-1H-indole-2-carboxylic acid
7-[(1S)-1-{5-[(3-aminoazetidin-1-yl)methyl]-2-oxo-2,3-dihydro-1, 3-oxazol-3-yl}ethyl]-3-(3-fluoro-4-{[imino(methyl)oxo-λ⁶-sulfanyl]methyl}phenyl)-1H-indole-2-carboxylic acid
7-[(1S)-1-{5-[(3-Aminoazetidin-1-yl)methyl]-2-oxo-2,3-dihydro-1,3-oxazol-3-yl}ethyl]-3-(2-oxo-1,2-dihydropyridin-4-yl)-1H-indole-2-carboxylic acid
7-[(1S)-1-{5-[(3-aminoazetidin-1-yl)methyl]-2-oxo-2,3-dihydro-1,3-oxazol-3-yl}ethyl]-3-(3-cyano-4-methanesulfonamidophenyl)-1H-indole-2-carboxylic acid
7-[(1S)-1-{5-[(3-aminoazetidin-1-yl)methyl]-2-oxo-2,3-dihydro-1,3-oxazol-3-yl}ethyl]-3-(2-methoxypyridin-4-yl)-1H-indole-2-carboxylic acid
7-[(1S)-1-{5-[(3-Aminoazetidin-1-yl)methyl]-2-oxo-2,3-dihydro-1,3-oxazol-3-yl}ethyl]-3-(3-fluoro-4-methanesulfonamidophenyl)-1H-indole-2-carboxylic acid
(S)-3-(3-fluoro-4-((methylsulfonyl)methyl)phenyl)-7-(1-(5-(guanidinomethyl)-2-oxooxazol-3(2H)-yl)ethyl)-1H-indole-2-carboxylic acid
7-((1S)-1-((4-(aminomethyl)benzoyl)amino)ethyl)-3-(6-(morpholin-4-ylmethyl)pyridin-3-yl)-1H-indole-2-carboxylic acid
7-((1S)-1-(2S,4r)-(2-(aminomethyl)-6-oxo-5-oxa-7-azaspiro[3.4]oct-7-yl)ethyl)-3-(3-fluoro-4-((methylsulfonyl)methyl)phenyl)-1H-indole-2-carboxylic acid
7-((1S)-1-(2R,4s)-(2-(aminomethyl)-6-oxo-5-oxa-7-azaspiro[3.4]oct-7-yl)ethyl)-3-(3-fluoro-4-((methylsulfonyl)methyl)phenyl)-1H-indole-2-carboxylic acid
7-[1-[5-(3-Aminopropyl)-1,2,4-oxadiazol-3-yl]ethyl]-3-[3-fluoro-4-(methylsulfonyl methyl)phenyl]-1H-indole-2-carboxylic acid
3-[3-Fluoro-4-(methylsulfonylmethyl)phenyl]-7-[1-[5-(5-oxopyrrolidin-3-yl)-1,2,4-oxadiazol-3-yl]ethyl]-1H-indole-2-carboxylic acid
7-[1-[5-(3-Aminopropyl)-2-oxo-1H-imidazol-3-yl]ethyl]-3-[3-fluoro-4-(methylsulfonylmethyl) phenyl]-1H-indole-2-carboxylic acid
(S)-3-(3-fluoro-4-((methylsulfonyl)methyl)phenyl)-7-(1-(6-oxo-5-oxa-2,7-diazaspiro[3.4]octan-7-yl)ethyl)-1H-indole-2-carboxylic acid
(S)-7-(1-(2-carbamimidoyl-6-oxo-5-oxa-2,7-diazaspiro[3.4]octan-7-yl)ethyl)-3-(3-fluoro-4-((methylsulfonyl)methyl)phenyl)-1H-indole-2-carboxylic acid
(S)-7-(1-(5-((3-aminoazetidin-1-yl)methyl)-2-oxooxazol-3(2H)-yl)ethyl)-3-(3-fluoro-4-((methylsulfonyl)methyl)phenyl)-1H-indole-2-carboxylic acid
(S)-7-(1-(5-((3-amino-3-methylazetidin-1-yl)methyl)-2-oxooxazol-3(2H)-yl)ethyl)-3-(3-fluoro-4-((methylsulfonyl)methyl)phenyl)-1H-indole-2-carboxylic acid
(S)-7-(1-(5-(2-(1-Aminocyclopropyl)ethyl)-2-oxooxazol-3(2H)-yl)ethyl)-3-(3-fluoro-4-((methylsulfonyl)methyl)phenyl)-1H-indole-2-carboxylic acid
(S)-7-(1-(5-(3-aminopropyl)-2-oxooxazol-3(2H)-yl)ethyl)-3-(3-fluoro-4-((methylsulfonyl)methyl)phenyl)-1H-indole-2-carboxylic acid
(S)-7-(1-(8-amino-2-oxo-1-oxa-3-azaspiro[4.5]decan-3-yl)ethyl)-3-(3-fluoro-4-((methylsulfonyl)methyl)phenyl)-1H-indole-2-carboxylic acid
7-(1-((5-(3-aminopropyl)oxazol-2-yl)oxy)ethyl)-3-(3-fluoro-4-((methylsulfonyl)methyl)phenyl)-1H-indole-2-carboxylic acid
3-(3-Fluoro-4-((methylsulfonyl)methyl)phenyl)-7-(1-(5-(3-guanidinopropyl)-2-oxooxazol-3(2H)-yl)ethyl)-1H-indole-2-carboxylic acid
7-(1-(5-(2-(1-aminocyclopropyl)ethyl)-2-oxooxazol-3(2H)-yl)ethyl)-3-(3-fluoro-4-((methylsulfonyl)methyl)phenyl)-1H-indole-2-carboxylic acid
7-(1-(5-(3-(Dimethylamino)propyl)-2-oxooxazol-3(2H)-yl)ethyl)-3-(3-fluoro-4-((methylsulfonyl)methyl)phenyl)-1H-indole-2-carboxylic acid
3-(3-(1-(2-carboxy-3-(3-fluoro-4-((methylsulfonyl)methyl)phenyl)-1H-indol-7-yl)ethyl)-2-oxo-2,3-dihydrooxazol-5-yl)-N, N, N-trimethylpropan-1-aminium
7-(1-((5-(2-aminoethyl)oxazol-2-yl)oxy)ethyl)-3-(3-fluoro-4-((methylsulfonyl)methyl)phenyl)-1H-indole-2-carboxylic acid
3-(3-Fluoro-4-((methylsulfonyl)methyl)phenyl)-7-(1-(5-(hydroxymethyl)-2-oxooxazol-3(2H)-yl)ethyl)-1H-indole-2-carboxylic acid
7-(1-(5-(Aminomethyl)-2-oxooxazol-3(2H)-yl)ethyl)-3-(3-fluoro-4-((methylsulfonyl)methyl)phenyl)-1H-indole-2-carboxylic acid
7-(1-(5-((2-Aminoacetamido)methyl)-2-oxooxazol-3(2H)-yl)ethyl)-3-(3-fluoro-4-((methylsulfonyl)methyl)phenyl)-1H-indole-2-carboxylic acid
7-[1-[5-[[(3S)-3-Aminopyrrolidin-1-yl]methyl]-2-oxo-oxazol-3-yl]ethyl]-3-[3-fluoro-4-(methylsulfonylmethyl)phenyl]-1H-indole-2-carboxylic acid
7-[1-[5-[[(3R)-3-Aminopyrrolidin-1-yl]methyl]-2-oxo-oxazol-3-yl]ethyl]-3-[3-fluoro-4-(methylsulfonylmethyl)phenyl]-1H-indole-2-carboxylic acid
7-(1-(5-(3-Aminopropyl)-2-oxooxazolidin-3-yl)ethyl)-3-(3-fluoro-4-((methylsulfonyl)methyl)phenyl)-1H-indole-2-carboxylic acid
7-(1-(5-(3-Aminopropyl)-2-oxooxazolidin-3-yl)ethyl)-3-(3-fluoro-4-((methylsulfonyl)methyl)phenyl)-1H-indole-2-carboxylic acid
7-(1-((4-(Aminomethyl)benzyl)oxy)ethyl)-3-(3-cyano-4-(methylsulfonamido)phenyl)-1H-indole-2-carboxylic acid
(S)-7-(1-((4-(Aminomethyl)benzyl)oxy)ethyl)-3-(3-chloro-4-((methylsulfonyl)methyl)phenyl)-1H-indole-2-carboxylic acid
(R)-7-(1-((4-(Aminomethyl)benzyl)oxy)ethyl)-3-(3-chloro-4-((methylsulfonyl)methyl)phenyl)-1H-indole-2-carboxylic acid
3-(3-Fluoro-4-((methylsulfonyl)methyl)phenyl)-7-(1-(piperidin-4-ylmethoxy)ethyl)-1H-indole-2-carboxylic acid
7-(1-((1,1-Dimethylpiperidin-1-ium-4-yl)methoxy)ethyl)-3-(3-fluoro-4-((methylsulfonyl)methyl)phenyl)-1H-indole-2-carboxylate
7-(1-((1H-1,2,3-triazol-5-yl)methoxy)ethyl)-3-(3-fluoro-4-((methylsulfonyl)methyl)phenyl)-1H-indole-2-carboxylic acid
7-(1-(4-(Ammoniomethyl)phenoxy)ethyl)-3-(3-fluoro-4-((methylsulfonyl)methyl)phenyl)-1H-indole-2-carboxylate
7-(1-((5-(3-Aminopropyl)oxazol-2-yl)amino)ethyl)-3-(3-fluoro-4-((methylsulfonyl)methyl)phenyl)-1H-indole-2-carboxylic acid
7-(1-((5-(2-aminoethyl)oxazol-2-yl)amino)ethyl)-3-(3-fluoro-4-((methylsulfonyl)methyl)phenyl)-1H-indole-2-carboxylic acid
7-(1-((4-(Aminomethyl) piperidine-1-carbonyl)oxy)ethyl)-3-(3-fluoro-4-((methylsulfonyl)methyl)phenyl)-1H-indole-2-carboxylic acid
7-(1-(((4-(Aminomethyl)phenyl)carbamoyl)oxy)ethyl)-3-(3-fluoro-4-((methylsulfonyl)methyl)phenyl)-1H-indole-2-carboxylic acid
7-(1-(4-(Aminomethyl)-N-methylbenzamido)ethyl)-3-(3-fluoro-4-((methylsulfonyl)methyl)phenyl)-1H-indole-2-carboxylic acid
7-[1-(5,6-Dihydroxy-1,3-dioxo-2,3-dihydro-1H-isoindol-2-yl)ethyl]-3-[3-fluoro-4-(methanesulfonylmethyl)phenyl]-1H-indole-2-carboxylic acid
7-[1-[[4-(Aminomethyl)cyclohexanecarbonyl]amino]ethyl]-3-[3-fluoro-4-(methylsulfonylmethyl) phenyl]-1H-indole-2-carboxylic acid
7-[1-[[2-(4-Aminocyclohexyl)acetyl]amino]ethyl]-3-[3-fluoro-4-(methylsulfonylmethyl)phenyl]-1H-indole-2-carboxylic acid 7-(1-(((4-(aminomethyl) phenoxy)carbonyl)amino)ethyl)-3-(3-fluoro-4-((methylsulfonyl)methyl)phenyl)-1H-indole-2-carboxylic acid
(4-(3-(1-(2-Carboxy-3-(3-fluoro-4-((methylsulfonyl)methyl)phenyl)-1H-indol-7-yl)ethyl)ureido)phenyl)methanaminium chloride
3-[3-Fluoro-4-(methanesulfonylmethyl)phenyl]-7-(1-{[2-(1H-imidazol-4-yl)ethyl]carbamoyl}propan-2-yl)-1H-indole-2-carboxylic acid
7-(1-{[4-(aminomethyl)phenyl]formamido}ethyl)-3-{6-[(morpholin-4-yl)methyl]pyridin-3-yl}-1H-indole-2-carboxylic acid
7-{1-[4-(3-aminopropyl)-1H-1,2,3-triazol-1-yl]ethyl}-3-[3-chloro-4-(methanesulfonylmethyl)phenyl]-1H-indole-2-carboxylic acid
7-(1-benzenesulfonamidoethyl)-3-(3,5-dichlorophenyl)-1H-indole-2-carboxylic acid
3-(3,5-dichlorophenyl)-7-(1-methanesulfonamidoethyl)-1H-indole-2-carboxylic acid
7-((S)-1-((2S,4r)-2-(aminomethyl)-6-oxo-5-oxa-7-azaspiro[3.4]octan-7-yl)ethyl)-3-(1-methyl-1H-pyrazol-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(1-(cyanomethyl)-1H-pyrazol-4-yl)-1H-indole-2-carboxylic acid
3-(1-(2-amino-2-iminoethyl)-1H-pyrazol-4-yl)-7-((S)-1-((2S,4r)-2-(aminomethyl)-6-oxo-5-oxa-7-azaspiro[3.4]octan-7-yl)ethyl)-1H-indole-2-carboxylic acid
3-(1-(2-amino-2-oxoethyl)-1H-pyrazol-4-yl)-7-((S)-1-((2S,4r)-2-(aminomethyl)-6-oxo-5-oxa-7-azaspiro[3.4]octan-7-yl)ethyl)-1H-indole-2-carboxylic acid
7-((S)-1-((2S,4r)-2-(aminomethyl)-6-oxo-5-oxa-7-azaspiro[3.4]octan-7-yl)ethyl)-3-(isoxazol-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(3,5-dimethyl-1H-pyrazol-4-yl)-1H-indole-2-carboxylic acid
7-((S)-1-((2S,4r)-2-(aminomethyl)-6-oxo-5-oxa-7-azaspiro[3.4]octan-7-yl)ethyl)-3-(3-oxo-2,3-dihydro-1H-pyrazol-4-yl)-1H-indole-2-carboxylic acid; or
(2S,4r)-2-(Aminomethyl)-7-((S)-1-(3-(3-fluoro-4-((methylsulfonyl)methyl)phenyl)-2-(1H-tetrazole-5-yl)-1H-indol-7-yl)ethyl)-5-oxa-7-azaspiro[3.4]octan-6-one.

22. A pharmaceutical composition comprising a compound according to claims 1 to 21, or a pharmaceutically acceptable salt or solvate thereof, in admixture with a pharmaceutically acceptable diluent or carrier.

23. A compound according to any of claims 1 to 21, or a pharmaceutically acceptable salt or solvate thereof, or a pharmaceutical composition according to claim 22, for use as a medicament.

24. A compound according to any of claims 1 to 21, or a pharmaceutically acceptable salt or solvate thereof, or a pharmaceutical composition according to claim 22, for use in the treatment of a bacterial infection.

25. The compound or pharmaceutical composition for the use according to claim 24, in combination with a beta-lactam antibiotic.
